(19) United States
(12) Patent Application Publication
(10) Pub. No.: US 2024/0360422 A1
(43) Pub. Date: Oct. 31, 2024

(54) STABLE PRODUCTION SYSTEMS FOR ADENO-ASSOCIATED VIRUS PRODUCTION

(71) Applicants: MICHAEL T. LEONARD, Boston, MA (US); Jeremy j. Gam, Boston, MA (US); Christopher S. Stach, Boston, MA (US); Alec A.K. Nielsen, Boston, MA (US)

(72) Inventors: MICHAEL T. LEONARD, Boston, MA (US); Jeremy j. Gam, Boston, MA (US); Christopher S. Stach, Boston, MA (US); Alec A.K. Nielsen, Boston, MA (US)

(21) Appl. No.: 18/287,734

(22) PCT Filed: Apr. 21, 2022
(86) PCT No.: PCT/US2022/025755

§ 371 Date: Oct. 20, 2023

(51) Int. Cl. C12N 7/00; C07K 14/005; C07K 14/435; C12N 9/00; C12N 15/86

(52) U.S. Cl. CPC C12N 7/00; C07K 14/005; C07K 14/435; C12N 9/93; C12N 15/86; C12Y 601/01026; C12N 2750/14122; C12N 2750/14143; C12N 2750/14152

(57) ABSTRACT

Disclosed herein are cell genetically engineered cell for AAV production. The genetically engineered cell comprises molecular systems for temporal control of expression of genes required for AAV production. Also disclosed herein are methods of using genetically engineered cells for AAV production.

### FIELD

Described herein are Adeno-Associated Virus (AAV) production systems. Also described herein are engineered cells and kits comprising an AAV production system and methods of using the same for AAV production.

### RELATED APPLICATIONS

This application claims the benefit under 35 U.S.C. § 119 of U.S. provisional application Ser. No. 63/177,760, filed Apr. 21, 2021, the entire contents of which are incorporated by reference herein.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Apr. 21, 2022 is named A121070007WO00-SEQ-ARM and is 347,698 bytes in size.

### BACKGROUND

AAV are a promising gene delivery modality for cell and gene therapy. AAV can be modified to carry therapeutic genetic payloads to cells within a subject. The production of AAV normally entails transient transfection of plasmids containing genes required for viral vector production into cell culture. However, transient transfection has several shortfalls. Large quantities of DNA and transfection reagent must be procured for the transfection process, which is costly. Also, poor transfection efficiency can result in minimal numbers of ‘transfected’ cells and increased variation associated with transfection steps and viral production.

### SUMMARY

Described herein are AAV production systems that introduce inducible control of gene products required for AAV production including cytostatic or cytotoxic gene products. This inducible control can be mediated at the genomic level (i.e., inducible control of genomic modification) or at the translational level (i.e., inducible control of altered translation). Each of the described AAV production systems can be integrated into the genome using random integration, targeted integration, or transposon-mediated integration.

In some embodiments, the application discloses an engineered cell for AAV production, comprising one or more stably integrated nucleic acid molecules collectively comprising a nucleic acid sequence encoding for each of: a noncanonical tRNA synthetase; a noncanonical tRNA corresponding to the noncanonical tRNA synthetase; NC-Rep 78; and NC-Rep52; each of which is operably linked to a promoter; wherein the nucleic acid sequence encoding NC-Rep78 and the nucleic acid sequence encoding NC-Rep52 each comprises a codon that is both a premature stop codon and an amino acid codon corresponding to the noncanonical tRNA. In some embodiments, the engineered cell comprises one or more stably integrated nucleic acid molecules comprises a first stably integrated nucleic acid molecule comprising the nucleic acid sequence encoding for the noncanonical tRNA synthetase. In some embodiments, the engineered cell comprises a noncanonical tRNA synthetase that is Pyrrolysyl-tRNA synthetase (pylRS). In some embodiments, the engineered cell comprises a pylRS comprising the amino acid sequence of any one of SEQ ID NOs: 20 and 21. In some embodiments, the engineered cell comprises a PylRS comprising the amino acid sequence of SEQ ID NO: 21.

In some embodiments, the engineered cell comprising the first stably integrated nucleic acid molecule further comprises a selection marker that is operably linked to a promoter.

In some embodiments, the engineered cell comprising the one or more stably integrated nucleic acid molecules comprises a second stably integrated nucleic acid molecule comprising the nucleic acid sequence encoding for the noncanonical tRNA. In some embodiments, the engineered cell comprises a noncanonical tRNA that charges H-Lys(Boc)-OH. In some embodiments, the noncanonical tRNA comprised within the engineered cell is PylT U25C. In some embodiments, the engineered cell comprises a PylT U25C comprising the nucleic acid sequence of SEQ ID NO: 22. In some embodiments, the engineered cell comprising the second stably integrated nucleic acid molecule comprises four nucleic acid sequences, each comprising the nucleic acid sequences encoding for PylT U25C and each operably linked to a promoter. In some embodiments, the engineered cell comprising the second stably integrated nucleic acid molecule further comprises a selection marker that is operably linked to a promoter.

In some embodiments, the engineered cell comprising the one or more stably integrated nucleic acid molecules comprises a third stably integrated nucleic acid molecule comprising the nucleic acid sequences encoding for NC-Rep78 and NC-Rep52. In some embodiments, NC-Rep78 comprises a premature stop codon at position 17; NC-Rep52 comprises a premature stop codon at position 233; or a combination thereof. In some embodiments, the engineered cell comprises a pylRS noncanonical tRNA synthetase and a PylT U25C noncanonical tRNA. In some embodiments, the engineered cell comprises the nucleic acid sequence encoding NC-Rep78 and the nucleic acid sequence encoding NC-Rep52 encoded as a single transcript. In some embodiments, the single transcript comprises a nucleic acid sequence encoding for an amino acid sequence of any one of SEQ ID NOs: 26-27. In some embodiments, the engineered cell comprising the third stably integrated nucleic acid molecule further comprises: a nucleic acid sequence encoding for NC-Rep40; a nucleic acid sequence encoding for NC-Rep68; or both.

In some embodiments, the engineered cell is HEK293 cell, HeLa cell, BHK cell, or SB9 cell.

In some embodiments, the application discloses a kit comprising any one of the engineered cells as described above. In some embodiments, the kit further comprises a polynucleotide comprising, from 5′ to 3′: (i) a nucleic acid sequence of a 5′ inverted terminal repeat; (ii) a multiple cloning site; and (iii) a nucleic acid sequence of a 3′ inverted terminal repeat. In some embodiments, the polynucleotide comprised within the kit is a plasmid or a vector.

In some embodiments, the application discloses a method for AAV production, comprising contacting any one of the engineered cells as described above with a noncanonical amino acid. In some embodiments, the noncanonical amino acid is H-Lys(Boc)-OH.

In some aspects, the application discloses an engineered cell comprising one or more stably integrated nucleic acid molecules collectively comprising a nucleic acid sequence encoding for each of: Rep52, DA-Rep52, Rep40, or DA-Rep40; Rep78, DA-Rep78, Rep68, or DA-Rep68; E2A or DA-E2A; E4ORF6 or DA-E4ORF6; VARNA or DA-VARNA; VP1 or DA-VP1; VP2 or DA-VP2; VP3 or DA-VP3; AAP; and L4 100K or DA-L4 100K and a Base Editor, each nucleic acid molecule being operably linked to a promoter; wherein the cell comprises the nucleic acid sequence of at least one of DA-Rep52, DA-Rep40, DA-Rep78, DA-Rep68, DA-E2A, DA-E4ORF6, DA-VP1, DA-VP2, DA-V3, and DA-L4 100K; wherein the nucleic acid sequences of DA-Rep52, DA-Rep40, DA-Rep78, DA-Rep68, DA-E2A, DA-E4ORF6, DA-VP1, DA-VP2, DA-V3, and DA-L4 100K each comprises a modified codon.

In some embodiments, the modified codon encodes for a missense codon, and wherein deamination of a cytosine or an adenine in the modified codon converts the encoded amino acid into another amino acid.

In some embodiments, the modified codon encodes for a premature stop codon, and wherein deamination of an adenine in the modified codon converts the modified codon into a tryptophan codon, a glutamine codon or an arginine.

In some embodiments, the modified codon encodes for a premature stop codon, and wherein deamination of a cytosine in the modified codon converts the encoded amino acid into a proline.

In some embodiments, the engineered cell comprises one or more stably integrated nucleic acid molecules each comprising a nucleic acid sequence encoding one or more CTCF insulators.

In some embodiments, the engineered cell comprising one or more stably integrated nucleic acid molecules comprises a first stably integrated nucleic acid molecule comprising the nucleic acid sequence encoding DA-E2A, the nucleic acid sequence encoding DA-E4ORF6, and the nucleic acid sequence encoding VARNA. In some embodiments, the first stably integrated nucleic acid molecule further comprises a nucleic acid sequence encoding L4 100K or DA-L4 100K. In some embodiments, the first stably integrated nucleic acid molecule further comprises a selection marker that is operably linked to a promoter.

In some embodiments, the first stably integrated nucleic acid molecule comprises the nucleic acid sequence of DA-E2A comprising one or more mutations to adenine or cytosine resulting in one or more premature stop codons. In some embodiments, the nucleic acid sequence encoding for DA-E2A comprises the amino acid sequence of SEQ ID NOs: 39, or 40. In some embodiments, positions 181 and/or 324 of DA-E2A (SEQ ID NOs: 39 or 40) correspond with mutations to adenine resulting in premature stop codons.

In some embodiments, the first stably integrated nucleic acid molecule comprises the nucleic acid sequence of DA-E4ORF6 comprising one or more mutations to adenine resulting in one or more premature stop codons. In some embodiments, the nucleic acid sequence encoding for DA-E4ORF6 comprises the amino acid sequence of SEQ ID NOs: 41 or 42. In some embodiments, positions 77 and/or 192 of DA-E4ORF6 (SEQ ID NOs: 41, or 42) correspond with a modified codon comprising an adenine resulting in a premature stop codon.

In some embodiments, the one or more stably integrated nucleic acid molecules comprises a second stably integrated nucleic acid molecule comprising the nucleic acid sequence encoding DA-Rep52 or DA-Rep40, the nucleic acid sequence encoding DA-Rep78 or DA-Rep68, the nucleic acid sequence encoding VP1 or DA-VP1, the nucleic acid sequence encoding VP2 or DA-VP2, and the nucleic acid sequence encoding VP3 or DA-VP3. In some embodiments, the second integrated nucleic acid molecule further comprises a selection marker that is operably linked to a promoter.

In some embodiments, the second stably integrated nucleic acid molecule comprises the nucleic acid sequence encoding for DA-Rep52 or DA-Rep40. In some embodiments, the nucleic acid sequence encoding for DA-Rep52 comprises an amino acid sequence of SEQ ID NOs: 43 or 47. In some embodiments, the nucleic acid sequence encoding for DA-Rep40 comprises an amino acid sequence of SEQ ID NOs: 44 or 48. In some embodiments, the second stably integrated nucleic acid molecule comprises a nucleic acid sequence encoding for DA-Rep78 or DA-Rep68. In some embodiments, the nucleic acid sequence encoding for DA-Rep78 comprises an amino acid sequence of any one of SEQ ID NOs: 45, 49 and 51.

In some embodiments, the second stably integrated nucleic acid molecule comprises the nucleic acid sequence encoding for DA-Rep68 comprising an amino acid sequence of SEQ ID NOs: 46, 50 or 52. In some embodiments, the second stably integrated nucleic acid molecule comprises an amino acid sequence encoding for Rep52 or DA-Rep52; Rep40 or DA-Rep40; Rep68 or DA-Rep68; and Rep78 or DA-Rep78. In some embodiments, the nucleic acid sequence encoding for Rep52 or DA-Rep52; Rep40 or, DA-Rep40; Rep68 or, DA-Rep68; and Rep78 or DA-Rep78 comprises a nucleic acid sequence of any one of SEQ ID NOs: 53-55, 113-115. In some embodiments, the nucleic acid sequence encoding for DA-Rep52, DA-Rep40, DA-Rep68 and DA-Rep78 comprises one or more mutations to adenine or cytosine resulting in one or more premature stop codons. In some embodiments, one adenine mutation in the nucleotide sequence is at a position that corresponds to amino acid positions 67, 262, and/or 319 of DA-Rep78 (SEQ ID NOs: 45, 49 and 51).

In some embodiments, the second stably integrated nucleic molecule further comprises a nucleic acid sequence encoding for one or more sgRNAs. In some embodiments, the one or more sgRNAs each comprise a nucleic acid sequence that is complementary to the nucleic acid sequences comprising one or more mutations to adenine or cytosine. In some embodiments, the one or more sgRNAs each comprise a nucleic acid sequence of any one of SEQ ID NOs: 56-81. In some embodiments, the one or more sgRNAs are operably linked to a chemically inducible promoter. In some embodiments, the chemically inducible promoter operably linked to the one or more sgRNAs is selected from the group consisting of pTRE3G, pTREtight, or a promoter containing at least one of VanR, TtgR, PhlF, CymR, or the Gal4 UAS operator sequences. In some embodiments, the nucleic acid sequence encoding the chemically inducible promoter operably linked to the one or more sgRNAs is any one of SEQ ID NOs: 1 and 2 or comprises any one of SEQ ID NOs: 86-91.

In some embodiments, the second stably integrated nucleic acid molecule comprises nucleic acid sequences encoding for VP1 or DA-VP1, VP2 or DA-VP2, and VP3 or DA-VP3. In some embodiments, the nucleic acid sequence encoding for VP1 comprises the amino acid sequence of SEQ ID NO: 14. In some embodiments, the nucleic acid sequence encoding for DA-VP1 comprises the amino acid sequence of SEQ ID NO: 99 or 102. In some embodiments, the nucleic acid sequence encoding for VP2 comprises the amino acid sequence of SEQ ID NO: 15. In some embodiments, the nucleic acid sequence encoding for DA-VP2 comprises the amino acid sequence of SEQ ID NO: 100 or 103. In some embodiments, the nucleic acid sequence encoding for VP3 comprises the amino acid sequence of SEQ ID NO: 16. In some embodiments, the nucleic acid sequence encoding for DA-VP3 comprises the amino acid sequence of SEQ ID NO: 101 or 104.

In some embodiments, the second stably integrated nucleic acid molecule comprises a nucleic acid sequence encoding for AAP. In some embodiments, the nucleic acid sequence encoding for AAP comprises the amino acid sequence of SEQ ID NO: 17.

In some embodiments, the engineered cell comprising one or more stably integrated nucleic acid molecules comprises a third stably integrated nucleic acid molecule comprising a nucleic acid sequences encoding for a transcriptional activator that, when expressed in the presence of a small molecule inducer, binds to a chemically inducible promoter of the engineered cell, and the nucleic acid sequences encoding for a Base Editor. In some embodiments, the third stably integrated nucleic acid molecule further comprises a selection marker that is operably linked to a promoter.

In some embodiments, the third stably integrated nucleic acid molecule comprising a Base Editor comprises an Adenine Base Editor (ABE) or a Cytosine Base Editor (CBE). In some embodiments, the CBE is a Cas9 CBE or a Cas13 CBE. In some embodiments, the ABE is a Cas9 ABE or a Cas13 ABE. In some embodiments, Cas9 ABE is encoded for by an amino acid sequence comprising SEQ ID NO: 82 or 83. In some embodiments, the Cas13 ABE is encoded for by an amino acid sequence comprising SEQ ID NO: 84 or 85. In some embodiments, the nucleic acid sequences encoding for the ABE is operably linked to a third chemically inducible promoter. In some embodiments, ABE is operably linked to the third chemically inducible promoter selected from the group consisting of pTRE3G, pTREtight, or a promoter containing at least one of VanR, TtgR, PhlF, or CymR, or the Gal4 UAS operator sequences. In some embodiments, the nucleic acid sequence encoding the third chemically inducible promoter is any one of SEQ ID NOs: 1 and 2 or comprises any one of SEQ ID NOs: 86-91. In some embodiments, the engineered cell comprises a transcriptional activator selected from the group consisting of TetOn-3G, TetOn-V16, TetOff-Advanced, VanR-VP16, TtgR-VP16, PhlF-VP16, and the cumate cTA and rcTA. In some embodiments, the transcriptional activator is activated by a small molecule inducer selected from the group consisting of doxycycline, vanillate, phloretin, rapamycin, abscisic acid, gibberellic acid acetoxymethyl ester, and cumate. In some embodiments, the transcriptional activator is TetOn 3G and the small molecule inducer is doxycycline.

In some embodiments, the engineered cell is HEK293 cell or HeLa cell.

In some aspects, the application discloses a kit comprising the engineered cell as described herein. In some embodiments, the kit further comprising a polynucleotide comprising, from 5′ to 3′: (i) a nucleic acid sequence of a 5′ inverted terminal repeat; (ii) a multiple cloning site; and (iii) a nucleic acid sequence of a 3′ inverted terminal repeat. In some embodiments, the polynucleotide comprised within the kit is a plasmid or a vector.

In some aspects, this application discloses a method for AAV production, comprising contacting the engineered cell as described above with a small molecule inducer that binds to the chemically inducible promoter. In some embodiments, the small molecule inducer is selected from the group consisting of doxycycline, vanillate, phloretin, rapamycin, abscisic acid, gibberellic acid acetoxymethyl ester, and cumate.

### BRIEF DESCRIPTION OF THE DRAWINGS

FIG. 1 is a plasmid schematic for ncAA AAV plasmids. The archaebacteria Methanosarcina mazei orthogonal tRNA synthetase (“pylRS”) is expressed constitutively using hEF1a. Cognate tRNA (“PylT”) is expressed using the U6 RNA polymerase III promoter in a multi-copy context because efficiency of ncAA incorporation has been linked to ncAA tRNA abundance. RepAAV2 Rep78+52 only constructs contain point mutations ablating the Rep68/40 splice site in addition to D233X and E17X TAG stop codon mutations. AAV2 Rep52/40-IRES-Rep78/68 constructs contain point mutations eliminating/minimizing the activity of the p19 AAV2 promoter and contain D233X and E17X. AAV WT Rep constructs encode for Rep78/68/52/40 and contain D233X and E17X TAG stop codon mutations. Transient testing using these ncAA plasmids in the context of Cap and Helper gene expressing constructs can be used to characterize ncAA inducible AAV production.

FIG. 2 is a plasmid schematic for transient transfection plasmids. A premature stop codon is made by mutating a tryptophan (W), glutamine (Q) or arginine (R) codon in the coding sequence of Rep, Cap, E2A, L4 100K, and/or E4 ORF6. A constitutively expressed ABE and single guide RNA repair these stop codons during transfection to produce AAV. In the absence of the ABE or single guide RNA, no AAV is produced.

FIG. 3 is a plasmid schematic for stable integration of plasmids. Transposon IR/DRs, CTCF insulators, and an antibiotic resistance selection cassette flank the AAV payload, mutant Rep/Cap, and mutant helper genes. One or more premature stop codons can be introduced to Rep, E2A, and E4 ORF6. The ABE is expressed by an inducible TRE promoter, with the rtTA (TetOn) gene fused to an antibiotic resistance gene on the same plasmid.

FIG. 4 depicts individual premature stop mutants of Rep, Cap, E2A, E4 ORF6, and L4 100K, with or without ABE8.17-m to restore viral titer. All Rep and Cap mutants tested were able to diminish AAV titers in the absence of an ABE to levels comparable with the negative control (“No Editor”). Mutants Rep78 W319* and Rep78 Q262* were able to be recovered with ABE8.17-m to titers comparable with ‘wild type’ AAV (“ABE8.17-m [V106W]”). However, single mutations in either E2A, E4 ORF6, or L4 100K alone were not enough to fully diminish AAV titer in the absence of an ABE.

FIG. 5 shows combinations of various pHelper mutations combined with the mutation Rep W319* or a “wild-type” pRepCap plasmid, and co-transfected with or without ABE8.17-m. Replacement of the pHelper plasmid with an inert plasmid acted as a negative control. All triple mutations in the absence of an ABE (“RepW319*,ABE−”) show comparable reduction of AAV titers to the level of the negative control. When only looking at the double pHelper mutations in the absence of an ABE (“wtRep,ABE−”), AAV titers are reduced, but not completely abolished. Co-transfection of an ABE (“wtRep,ABE+” and “RepW319*,ABE+”) recovers titers to levels near ‘wild-type’ AAV (the first “wt pHelper” and “wtRep,ABE+” bars), within 2-fold for every mutant combination tested.

FIG. 6 shows combinations of various stable AAV plasmids co-transfected with or without doxycycline. A co-transfection without the ABE plasmid served as a negative control. When using an inducible guide RNA, the resulting AAV titers are comparable to the level of the negative control in the absence of doxycycline, and comparable to the level of the wildtype AAV titer in the presence of doxycycline, both within 4-fold. However, when using a constitutive guide RNA, the resulting AAV titers are comparable to the level of the wildtype control in both the presence and absence of doxycycline, within 2-fold, indicating a lack of inducibility in the plasmid combination tested in transient.

### DETAILED DESCRIPTION OF CERTAIN EMBODIMENTS

AAV are a promising gene delivery modality for cell and gene therapy. The production of AAV normally entails transient transfection of plasmids into cell culture. However, stable integration of genes necessary to produce therapeutic AAV into the genome offers several advantages compared to traditional production via transient transfection. Since cells amplify the viral genes during their own cell division, large quantities of DNA and transfection reagent no longer need to be procured for the transfection process, reducing costs. Also, since the DNA is already within the nucleus, viral titers may be higher and more consistent due to minimal numbers of “untransfected” cells and reduced variation associated with transfection steps. The simpler production process also saves scientist time.

However, several genes required for adeno-associated viral (AAV) vector production have been demonstrated by others to be cytostatic or cytotoxic, namely Rep, E2A and E4. The cytotoxic and cytostatic nature of these proteins has hampered the development of stable AAV producer cell lines in the widely used HEK293 cell line, since the native expression of adenovirus E1 genes in HEK293 cells upregulates expression of these toxic genes. Cells stably transfected with these genes fail to survive selection steps or have silenced expression, resulting in an inability to produce relevant quantities of AAV.

### I. Adeno-Associated Virus Production Systems

In some aspects, the disclosure relates to adeno-associated virus (AAV) production systems. In some embodiments, AAV production systems allow for inducible control of a gene product(s) required for AAV production, including a product(s) that is cytotoxic or cytostatic to a cell. This inducible control can be mediated at the genomic level (i.e., inducible control of genomic modification) or at the translational level (i.e., inducible control of altered translation).

An AAV production system, as described herein, comprises one or more polynucleic acids collectively comprising: (a) an AAV production component and (b) an activity control component. As used herein, the term “AAV production component” refers to one or more polynucleic acids that collectively encode the gene products required for generation of AAV in a recombinant host cell, wherein at least one gene required for AAV production is modified to comprise a mutation that decreases the activity of the gene required for AAV production. In some embodiments, the mutation results in a premature stop codon.

In some embodiments, the AAV production component comprises one or more polynucleotides that collectively encode the gene products required to generate an AAV vector in a recombinant host cell. Exemplary AAV gene products include Rep52, Rep40, Rep78, Rep68, E2A, E4Orf6, VARNA, CAP (VP1, VP2, VP3), and AAP. The Rep gene products (comprising Rep52, Rep40, Rep78 and Rep68) are involved in AAV genome replication. The E2A gene product is involved in aiding DNA synthesis processivity during AAV replication. The E4Orf6 gene product supports AAV replication. The VARNA gene product plays a role in regulating translation. The CAP gene products (comprising VP1, VP2, VP3) encode viral capsid proteins. The AAP gene product plays a role in capsid assembly. In some embodiments, an AAV component comprises one or more polynucleotides that collectively encode the gene products: Rep52 or Rep40; Rep78 or Rep68; E2A; E4Orf6; VARNA; VP1; VP2; VP3; and AAP. In some embodiments, a AAV component comprises one or more polynucleotides that collectively encode the gene products: Rep52, Rep40, Rep78, Rep68, E2A, E4Orf6, VARNA, VP1, VP2, VP3, and AAP.

In some embodiments, the AAV production component comprises a heterologous polynucleic acid comprising a nucleic acid sequence encoding for a gene product(s) required for AAV production (e.g., a product(s) that is cytotoxic or cytostatic to the cell, such as Rep, E2A and/or E4), wherein the gene product(s) is modified to comprise a mutation that decreases the activity of the gene required for AAV production. In some embodiments, the mutation results in a premature stop codon.

In some embodiments, the heterologous polynucleic acid comprising a nucleic acid sequence encoding for a gene product(s) required for AAV production comprises at least 1 mutation (e.g. at least 1, at least 2, at least 3, at least 4, at least 5, at least 6, at least 7, at least 8, at least 9, or at least 10 mutations). In some embodiments, the heterologous polynucleic acid comprising a nucleic acid sequence encoding for a gene product(s) required for AAV production comprises 1, 2, 3, 4, 5, 6, 7, 8, 9, or 10 mutation(s). In some embodiments, the heterologous polynucleic acid comprising a nucleic acid sequence encoding for a gene product(s) required for AAV production comprises 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 mutations. In some embodiments, any codon within the heterologous polynucleic acid comprising a nucleic acid sequence encoding for a gene product(s) required for AAV production can be mutated.

In some embodiments, the heterologous polynucleic acid comprising a nucleic acid sequence encoding for a gene product(s) required for AAV production comprises at least 1 premature stop codon (e.g. at least 1, at least 2, at least 3, at least 4, at least 5, at least 6, at least 7, at least 8, at least 9, or at least 10 premature stop codons). In some embodiments, the heterologous polynucleic acid comprising a nucleic acid sequence encoding for a gene product(s) required for AAV production comprises 1, 2, 3, 4, 5, 6, 7, 8, 9, or 10 premature stop codon(s). In some embodiments, the heterologous polynucleic acid comprising a nucleic acid sequence encoding for a gene product(s) required for AAV production comprises 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 premature stop codon(s). In some embodiments, any codon within the heterologous polynucleic acid comprising a nucleic acid sequence encoding for a gene product(s) required for AAV production can be modified to a premature stop codon.

As used herein, the term “premature stop codon” refers to a stop codon added to the coding sequence of a gene by mutating one or more nucleic acid residues in the coding sequence such that the sequence of a given codon becomes TAG, TAA, or TGA.

In some embodiments, the AAV production component is (i.e., the gene products of the AAV component are) encoded on a single polynucleic acid. In other embodiments, multiple polynucleic acids collectively comprise the AAV component (i.e., at least two of the gene products of the AAV component are encoded on different polynucleic acids). For example, an AAV component may comprise at least 2, at least 3, at least 4, or at least 5 polynucleic acids. In some embodiments, a AAV component comprises 2, 3, 4, or 5 polynucleic acids.

As used herein, the term “activity control component” refers to one or more polynucleic acids that collectively encode the gene products required for inducing production of genes required for AAV production that comprise one or more mutations that decrease the activity of the gene product. In some embodiments, the one or more mutations decrease the activity of the gene product required for AAV production by at least 10% (e.g. at least 10%, at least 20%, at least 30%, at least 40%, at least 50%, at least 60%, at least 70%, at least 80%, at least 90%, at least 95%, at least 98%, or at least 99%) compared to the wildtype gene product. In some embodiments, the one or more mutations decrease the activity of the gene product required for AAV production by 10%-20%, 10%-30%, 10%, 50%, 10%-70%, 10%-90%, 10%-99%, 30%-50%, 30%-70%, 30%-90%, 30%-99%, 50%-70%, 50%-90%, 50%-99%, 70%-90%, or 70%-99%. In some embodiments, the one or more mutations in the gene required for AAV production result in loss of function of the gene product. In some embodiments, the one or more mutations decrease AAV production in a cell by at least 1-fold (e.g. at least 1-fold, at least 2-fold, at least 5-fold, at least 10-fold, at least 50-fold, at least 100-fold, at least 500-fold, at least 1000-fold, at least 10000-fold. In some embodiments, the one or more mutations decrease AAV production in a cell 1-2, 1-5, 1-10, 1-50, 1-100, 1-1000, 5-10, 5-50, 5-100, 5-1000, 10-20, 10-50, 10-100, 10-1000, 10-10000, 100-1000, or 100-10000 fold.

In some embodiments, the gene required for AAV production is mutated to comprise a premature stop codon(s). In some embodiments, an activity control component comprises one or more polynucleic acids that collectively encode the gene products required for inducing expression of genes that comprise a premature stop codon(s). Exemplary activity control components described herein include a non-canonical tRNA synthetase/tRNA system and Base Editor system. In some embodiments, the activity control component comprises a Base Editor (e.g. an ABE or CBE) capable of correcting one or more mutations in a gene required for AAV production. In some embodiments, the activity control component comprises a Base Editor (e.g. an ABE or CBE) capable of correcting 1, 2, 3, 4, 5, 6, 7, 8, 9, 10, or 20 mutations in a gene required for AAV production. In some embodiments, the activity control component comprises a Base Editor (e.g. an ABE) capable of editing a premature stop codon(s) such that it encodes a canonical codon. In some embodiments, the activity control component comprises a Base Editor system capable of editing the premature stop codon(s) such that it encodes the original wildtype canonical codon. In some embodiments, the activity control component comprises a non-canonical tRNA synthetase/tRNA system comprising a non-canonical tRNA anticodon that is complementary to the premature stop codon. In some embodiments, the non-canonical tRNA synthetase/tRNA system charges a non-canonical amino acid such that when the non-canonical amino acid is present, the noncanonical amino acid is incorporated into the protein required for AAV production during translation. In some embodiments, the non-canonical tRNA synthetase/tRNA system is chemically inducible.

In some embodiments, the activity control component is encoded on a single polynucleic acid. In some embodiments, multiple polynucleic acids collectively comprise the activity control component. For example, an activity control component may comprise at least 2, at least 3, at least 4, or at least 5 polynucleic acids. In some embodiments, an activity control component comprises 2, 3, 4, 5, 6, 7, 8, 9, 10, 11, 12, 13, 14, 15, 16, 17, 18, 19 or 20 polynucleic acids.

As used herein, the term “promoter” refers to a nucleic acid sequence that is capable of being bound by a protein to initiate transcription of RNA from DNA. A promoter may be a constitutive promoter (i.e., an unregulated promoter that allows for continual transcription). Examples of constitutive promoters are known in the art and include, but are not limited to, cytomegalovirus (CMV) promoters, elongation factor 1 α (EF1α) promoters, simian vacuolating virus 40 (SV40) promoters, ubiquitin-C (UBC) promoters, U6 promoters, and phosphoglycerate kinase (PGK) promoters. See e.g., Ferreira et al., Tuning gene expression with synthetic upstream open reading frames. Proc. Natl. Acad. Sci. U.S.A. 2013 July; 110 (28): 11284-89; Pub. No.: US 2014/377861 A1—the entireties of which are incorporated herein by reference. Alternatively, a promoter may be an inducible promoter (i.e., only activates transcription under specific circumstances). An inducible promoter may be a chemically inducible promoter, a temperature inducible promoter, or a light inducible promoter. Examples of inducible promoters are known in the art and include, but are not limited to, tetracycline/doxycycline inducible promoters, cumate inducible promoters, ABA inducible promoters, CRY2-CIB1 inducible promoters, DAPG inducible promoters, and mifepristone inducible promoters. See e.g., Stanton et al., ACS Synth. Biol. 2014 Dec. 19; 3 (12): 880-91; Liang et al., Sci. Signal. 2011 Mar. 15; 4 (164): rs2; U.S. Pat. No. 7,745,592 B2; U.S. Pat. No. 7,935,788 B2—the entireties of which are incorporated herein by reference.

In some embodiments, a AAV production system described herein further comprises an engineered cell. The engineered cell may comprise any part (and any combination of parts) of the AAV production systems described herein.

For example, an engineered cell may comprise at least a portion of the AAV production component. For example, and as described above, a AAV production component may comprise multiple polynucleic acids. In such embodiments, an engineered cell comprises one or more of said multiple polynucleic acids—each of which may be located extra-chromosomally or stably integrated into the genome of the engineered cell. In some embodiments, an engineered cell comprises the entire AAV production component.

Alternatively, or in addition, an engineered cell may comprise the activity control component of the AAV production system.

In some embodiments, a AAV production system comprises: (a) an engineered cell comprising an AAV production component comprising one or more heterologous polynucleic acids that collectively encode the genes required for AAV production, wherein at least one gene comprises a mutation; (b) an activity control component capable of inducing production and/or correcting the mutation of the at least one gene comprising a mutation. In some embodiments, the mutation results in a premature stop codon.

### A. Landing Pad

An engineered cell described herein may further comprise a landing pad. As used herein, the term “landing pad” refers to a heterologous polynucleic acid sequence that facilitates the targeted insertion of a “payload” sequence into a specific locus (or multiple loci) of the cell's genome. Accordingly, the landing pad is integrated into the genome of the cell. A fixed integration site is desirable to reduce the variability between experiments that may be caused by positional epigenetic effects or proximal regulatory elements. The ability to control payload copy number is also desirable to modulate expression levels of the payload without changing any genetic components.

In some embodiments, the landing pad is located at a safe harbor site in the genome of the engineered cell. As used herein, the term “safe harbor site” refers to a location in the genome where genes or genetic elements can be introduced without disrupting the expression or regulation of adjacent genes and/or adjacent genomic elements do not disrupt expression or regulation of the introduced genes or genetic elements. Examples of safe harbor sites are known to those having skill in the art and include, but are not limited to, AAVS1, ROSA26, COSMIC, H11, CCR5, and LiPS-A3S. See e.g., Gaidukov et al., Nucleic Acids Res. 2018 May 4; 46 (8): 4072-4086; U.S. Pat. Nos. 8,980,579 B2; 10,017,786 B2; 9,932,607 B2; Pub. No.: US 2013/280222 A; Pub. No.: WO 2017/180669 A1—the entireties of which are incorporated herein. In some embodiments, the safe harbor site is a known site. In other embodiments, the safe harbor site is a previously undisclosed site. See “Methods of Identifying High-Expressing Genomic Loci and Uses Thereof” herein. In some embodiments, an engineered cell described herein comprises a landing pad that is integrated at a safe harbor locus selected from the group consisting of AAVS1, ROSA26, COSMIC, H11, CCR5, and LiPS-A3S.

In some embodiments, the engineered cell is derived from a HEK293 cell. In some embodiments, the engineered HEK293 cell comprises a landing pad that is integrated at a safe harbor locus selected from the group consisting of AAVS1, ROSA26, CCR5, and LiPS-A3S.

In some embodiments, the engineered cell is derived from a CHO cell. In some embodiments, the engineered CHO cell comprises a landing pad that is integrated at a safe harbor locus selected from the group consisting of ROSA26, COSMIC, and H11.

Each of the landing pads described herein comprises at least one recombination site. Recombination sites for various integrases have been identified previously. For example, a landing pad may comprise recombination sites corresponding to a Bxb1 integrase, lambda-integrase, Cre recombinase, Flp recombinase, gamma-delta resolvase, Tn3 resolvase, φC31 integrase, or R4 integrase. Exemplary recombination site sequences are known in the art (e.g., attP, attB, attR, attL, Lox, and Frt).

The landing pads described herein may comprise one or more expression cassettes.

In some embodiments, the payload sequence comprises a nucleic acid molecule encoding a first inverted terminal repeat (ITR), a second ITR and a gene operably linked to a promoter (as described herein). In some embodiments, the payload comprises a nucleic acid molecule encoding 5′-ITR-promoter-gene-ITR-3′, where the gene is a gene for AAV delivery. In some embodiments, the gene is a fluorescent protein. In some embodiments, the gene is a green fluorescent protein. In some embodiments, the payload sequence comprises a multiple cloning site.

### B. Transcriptional Activator

In some embodiments, the AAV production system further comprises a nucleic acid sequence encoding a transcriptional activator. In some embodiments, the transcriptional activator is selected from the group consisting of TetOn-3G, rtTA-V16, TetOff-Advanced, VanR-VP16, TtgR-VP16, PhlF-VP16, and the cumate cTA and rcTA. In some embodiments, the transcriptional activator is a rtTA/TetOn variant selected from the group consisting of rtTA-V1, rtTA-V2, rtTA-V3, rtTA-V4, rtTA-V5, rtTA-V7, rtTA-V8, rtTA-V9, rtTA-V10, rtTA-V11, rtTA-V12, rtTA-V13, rtTA-V14, rtTA-V15, rtTA-V16, rtTA-V17, and rtTA-V18 as described in Das et al. Curr. Gene Therapy 2016; 16 (3): 156-67, which is incorporated by reference in its entirety. In some embodiments, the nucleic acid sequence encoding the transcriptional activator fused to a selection marker. In some embodiments, the transcriptional activator is operably linked to a promoter. In some embodiments, the transcriptional activation is operably linked to a constitutively active promoter. In some embodiments, the transcriptional activator is operably linked to its corresponding chemically inducible promoter. In a non-limiting example, a TetOn-3G transcriptional activator may be operably linked to a TRE promoter. In some embodiments, the transcriptional activation is operably linked to a hEF1a promoter. In some embodiments, the transcriptional activator, when exposed to a small molecule inducer, induces the expression of corresponding chemically inducible promoters within the engineered cell. In some embodiments, the small molecule inducer is selected from the group consisting of doxycycline, vanillate, phloretin, rapamycin, abscisic acid, gibberellic acid acetoxymethyl ester, and cumate.

### II. AAV Production Systems for Introducing Amino Acid(s) in Place of a Premature Stop Codon(s) During mRNA Translation

A system for introducing an amino acid(s) in place of a premature stop codon(s) during mRNA translation may comprise a noncanonical tRNA synthetase, a noncanonical tRNA, or combination thereof. In some embodiments, the system for introducing an amino acid(s) in place of a premature stop codon(s) further comprises a noncanonical amino acid.

As used herein, the term “noncanonical tRNA synthetase” refers to an tRNA synthetase that is not naturally present in the cell from which the engineered cell is derived. A tRNA synthetase is an enzyme that catalyzes the covalent attachment of an amino acid to a cognate tRNA during translation.

As used herein, the term “noncanonical tRNA” refers to a tRNA that has an anticodon, which is not used by a naturally occurring tRNA of the cell from which the engineered cell is derived. In some embodiments, a noncanonical tRNA comprises an anti-codon that corresponds with a premature stop codon (TAG, TAA or TGA) of the engineered cell. In some embodiments, a noncanonical tRNA is charged by a corresponding noncanonical tRNA synthetase; in reference to a specific tRNA synthetase, a noncanonical tRNA may be referred to as a conjugate tRNA.

In some embodiments, the activity control component comprises a noncanonical tRNA synthetase and its conjugate noncanonical tRNA. In some embodiments, the noncanonical tRNA synthetase and its conjugate noncanonical tRNA are selected from the group consisting of E. coli GlnRS-tRNAGln, E. coli TyrRS & Bst tRNATyr, E. coli TyrRS-RNATyr, B. subtilis TrpRS-tRNATrp, E. coli TrpRS-tRNATrp, E. coli LeuRS-tRNALeu, M. bareri PylRS (b)-tRNAPyl, M. bareri PylRS & D. hafniense tRNAPyl, E. coli TyrRS & G. stearothermophilus tRNATyr, as described in Mukai, Takahito, et al. Annual review of microbiology 71 (2017): 557-577 which is incorporated herein in its entirety by reference. In some embodiments, the noncanonical tRNA synthetase and its conjugate noncanonical tRNA is M. mazei Pyrrolysyl-tRNA synthetase (PylRS)-tRNAPyl, which incorporate the noncanonical amino acid H-Lys(Boc)-OH, an l-lysine derivative, during mRNA translation.

A. Exemplary Noncanonical tRNA Synthetases

In some embodiments, a system for introducing an amino acid in place of a premature stop codon during mRNA translation comprises a heterologous polynucleotide comprising a nucleic acid sequence encoding for a noncanonical tRNA synthetase operably linked to a promoter (constitutive or inducible, as described herein). Exemplary noncanonical tRNA synthetases are known in the art and included, but are not limited to E. coli GlnRS, E. coli TyrRS, B. subtilis TrpRS, E. coli TrpRS, E. coli LeuRS, M. bareri PylRS, E. coli TyrRS, and M. mazei PylRS. In some embodiments, the activity control component comprises a heterologous polynucleic acid comprising a nucleic acid sequence encoding a tRNA synthetase selected from the group consisting of E. coli GlnRS, E. coli TyrRS, B. subtilis TrpRS, E. coli TrpRS, E. coli LeuRS, M. bareri PylRS, E. coli TyrRS, and M. mazei PylRS.

In some embodiments, a noncanonical tRNA synthetase of the activity control component described herein comprises an amino acid sequence having at least 80% identity (e.g., at least 80%, at least 85%, at least 90%, at least 95%, or at least 99% identity) with SEQ ID NO: 20 (“M. mazei PylRS”). In some embodiments, a non-canonical tRNA synthetase comprises the amino acid sequence of SEQ ID NO: 20. In some embodiments, a noncanonical tRNA consists of the amino acid sequence of SEQ ID NO: 20.

In some embodiments, a noncanonical tRNA synthetase comprises an amino acid sequence having at least 80% identity (e.g., at least 80%, at least 85%, at least 90%, at least 95%, or at least 99% identity) with SEQ ID NO: 21 (“MmPylRS (Y384F)”), wherein the amino acid at position 384 is F. In some embodiments, a noncanonical tRNA synthetase comprises the amino acid sequence of SEQ ID NO: 21. In some embodiments, a noncanonical tRNA synthetase consists of the amino acid sequence of SEQ ID NO: 21.

In some embodiments, a system for introducing a noncanonical amino acid in place of a premature stop codon during mRNA translation comprises one or more heterologous polynucleotides that collectively comprise nucleic acid sequences encoding for at least two noncanonical tRNA synthetases (as described above), each of which is operably linked to a promoter (constitutive or inducible, as described herein). For example, in some embodiments, the activity control component comprises nucleic acid sequences encoding for at least 2, at least 3, at least 4, at least 5, at least 6, at least 7, at least 8, at least 9, or at least 10 tRNA synthetases (as described above). In some embodiments, the activity control component comprises nucleic acid sequences encoding for 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 tRNA synthetases (as described above). In some embodiments, the activity control component comprises nucleic acid sequences encoding for 2, 3, 4, 5, 6, 7, 8, 9, or 10 tRNA synthetases (as described above).

The activity control component as described herein may comprise nucleic acid sequences encoding for at least 2, at least 3, at least 4, at least 5, at least 6, at least 7, at least 8, at least 9, or at least 10 distinct tRNA synthetases (as described above). In some embodiments, the activity control component comprises nucleic acid sequences encoding for 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 distinct tRNA synthetases (as described above). In some embodiments, the activity control component comprises nucleic acid sequences encoding for 2, 3, 4, 5, 6, 7, 8, 9, or 10 distinct tRNA synthetases (as described above).

B. Exemplary Noncanonical tRNAs

In some embodiments, the activity control component comprises a heterologous polynucleotide comprising a nucleic acid sequence encoding for a noncanonical tRNA operably linked to a promoter (constitutive or inducible, as described herein). Exemplary noncanonical tRNAs are known in the art and include, but are not limited to E. coli tRNAGln, E. coli tRNATyr, B. subtilis tRNATrp, E. coli tRNATrp, E. coli tRNALeu, M. bareri tRNAPyl, D. hafniense tRNAPyl, G. stearothermophilus tRNATyr, and M. mazei tRNAPyl. In some embodiments, the activity control component comprises a heterologous polynucleic acid comprising a nucleic acid sequence encoding a tRNA selected from the group consisting of E. coli tRNAGln, E. coli tRNATyr, B. subtilis tRNATrp, E. coli tRNATrp, E. coli tRNALeu, M. bareri tRNAPyl, D. hafniense tRNAPyl, G. stearothermophilus tRNATyr, and M. mazei tRNAPyl.

In some embodiments, a noncanonical tRNA of the activity control component described herein comprises a nucleic acid sequence having at least 80% identity (e.g., at least 80%, at least 85%, at least 90%, at least 95%, or at least 99% identity) with SEQ ID NO: 22 (“PylT (U25C)”). In some embodiments, a non-canonical tRNA comprises the nucleic acid sequence of SEQ ID NO: 22. In some embodiments, a noncanonical tRNA consists of the nucleic acid sequence of SEQ ID NO: 22.

In some embodiments, a system for introducing a noncanonical amino acid in place of a premature stop codon during mRNA translation comprises one or more heterologous polynucleotides that collectively comprise nucleic acid sequences encoding for at least two noncanonical tRNAs (as described above), each of which is operably linked to a promoter (constitutive or inducible, as described herein). For example, in some embodiments, the activity control component comprises nucleic acid sequences encoding for at least 2, at least 3, at least 4, at least 5, at least 6, at least 7, at least 8, at least 9, or at least 10 tRNAs (as described above). In some embodiments, the activity control component comprises nucleic acid sequences encoding for 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 tRNAs (as described above). In some embodiments, the activity control component comprises nucleic acid sequences encoding for 2, 3, 4, 5, 6, 7, 8, 9, or 10 tRNAs (as described above).

An activity control component described herein may comprise nucleic acid sequences encoding for at least 2, at least 3, at least 4, at least 5, at least 6, at least 7, at least 8, at least 9, or at least 10 distinct tRNA synthetases (as described above). In some embodiments, the activity control component comprises nucleic acid sequences encoding for 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 distinct tRNA synthetases (as described above). In some embodiments, the activity control component comprises nucleic acid sequences encoding for 2, 3, 4, 5, 6, 7, 8, 9, or 10 distinct tRNA synthetases (as described above).

In some embodiments, the activity control component comprises a noncanonical tRNA expression cassette comprising, from 5′ to 3′: (i) a nucleic acid sequence of a promoter (constitutive or inducible, as described herein); (ii) a nucleic acid sequence encoding for a noncanonical tRNAs (as described above); and (iii) a terminator sequence. In some embodiments, the noncanonical tRNA expression cassette comprises the nucleic acid sequence of SEQ ID NO: 23 or a nucleic acid sequence having at least at least 80% (e.g., at least 80%, at least 85%, at least 90%, at least 95%, or at least 99%) identity to the nucleic acid sequence of SEQ ID NO: 23. In some embodiments, the activity control component comprises multiple noncanonical tRNA expression cassettes. For example, in some embodiments, the activity control component comprises at least 2, at least 3, at least 4, at least 5, at least 6, at least 7, at least 8, at least 9, or at least 10 noncanonical tRNA expression cassettes. In some embodiments, the activity control component comprises 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 noncanonical tRNA expression cassettes. In some embodiments, the activity control component comprises 2, 3, 4, 5, 6, 7, 8, 9, or 10 noncanonical tRNA expression cassettes.

### C. AAV Gene Products Having Premature Stop Codons

In some embodiments, the AAV production component comprises a heterologous polynucleic acid comprising a nucleic acid sequence encoding for a gene product(s) required for AAV production, wherein the gene product(s) is modified to comprise a codon(s) that is both a premature stop codon and an amino acid codon corresponding to a noncanonical tRNA (i.e., as described in Part IA). In some embodiments, a codon for an amino acid tolerant of replacement within the nucleic acid sequence encoding for the gene product(s) is modified to comprise a codon(s) that is both a premature stop codon and an amino acid codon corresponding to a noncanonical tRNA. In some embodiments, a lysine codon within the nucleic acid sequence encoding for the gene product(s) is modified to comprise a codon(s) that is both a premature stop codon and an amino acid codon corresponding to a noncanonical tRNA. In some embodiments, the polynucleic acid encoding for the gene product(s) may comprise one or more premature stop codon(s) (e.g. 1, 2, 3, 4, 5, 6, 7, 8, 9, or 10) at position(s) corresponding to a codon for an amino acid tolerant of replacement. In some embodiments, the polynucleic acid encoding for the gene product(s) may comprise one or more premature stop codon(s) (e.g. 1, 2, 3, 4, 5, 6, 7, 8, 9, or 10) at position(s) corresponding to a lysine codon(s). In some embodiments, the polynucleic acid encoding for the gene product(s) may comprise 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 premature stop codon(s) at a position(s) corresponding to a codon for an amino acid tolerant of replacement. In some embodiments, the polynucleic acid encoding for the gene product(s) may comprise 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 premature stop codon(s) at a position(s) corresponding to lysine codon(s).

The modifier “NC,” as used herein, refers to a gene comprising a codon(s) that is both premature stop codon and codon corresponding to a noncanonical tRNA. In some embodiments, the AAV production component comprises: a nucleic acid sequence encoding for NC-Rep52 operably linked to a promoter (constitutive or inducible, as described herein); a nucleic acid sequence encoding for NC-Rep40 operably linked to a nucleic acid sequence of a promoter (constitutive or inducible, as described herein); a nucleic acid sequence encoding for NC-Rep78 operably linked to a nucleic acid sequence of a promoter (constitutive or inducible, as described herein); a nucleic acid sequence encoding for NC-Rep68 operably linked to a nucleic acid sequence of a promoter (constitutive or inducible); a nucleic acid sequence encoding for NC-Rep78+52 operably linked to a nucleic acid sequence of a promoter (constitutive or inducible, as described herein); a nucleic acid sequence encoding for NC-Rep operably linked to a nucleic acid sequence of a promoter (constitutive or inducible, as described herein); a nucleic acid sequence encoding for NC-E2A operably linked to a nucleic acid sequence of a promoter (constitutive or inducible, as described herein); a nucleic acid sequence encoding for NC-E4 ORF6 operably linked to a nucleic acid sequence of a promoter (constitutive or inducible, as described herein); a nucleic acid sequence encoding for NC-VP1 operably linked to a nucleic acid sequence of a promoter (constitutive or inducible, as described herein); a nucleic acid sequence encoding for NC-VP2 operably linked to a nucleic acid sequence of a promoter (constitutive or inducible, as described herein); a nucleic acid sequence encoding for NC-VP3 operably linked to a nucleic acid sequence of a promoter (constitutive or inducible, as described herein); a nucleic acid sequence encoding for NC-VP operably linked to a nucleic acid sequence of a promoter (constitutive or inducible, as described herein); or any combination thereof.

In some embodiments, the AAV production component comprises a nucleic acid sequence encoding for NC-Rep40 operably linked to a nucleic acid sequence of a promoter (constitutive or inducible, as described herein). The term “NC-Rep40” refers to a nucleic acid sequence encoding a polypeptide comprising at least 80% (e.g., at least 80%, at least 85%, at least 90%, at least 95%, or at least 99%) identity to the amino acid sequence of SEQ ID NO: 7, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional Rep40 polypeptide. In some embodiments, NC-Rep40 comprises one or more TAG premature stop codon mutations. In some embodiments, NC-Rep40 comprises one or more TAG premature stop codon mutations at sites identified as tolerant of amino acid substitutions (e.g. positions corresponding to E226 and D233 of SEQ ID NO: 97 as described in Urabe M et al. J Virol. 1999 April; 73 (4): 2682-93, which is incorporated by reference in its entirety). In some embodiments, the AAV production component comprises NC-Rep40 as described above.

In some embodiments, the AAV production component comprises a nucleic acid sequence encoding for NC-Rep68 operably linked to a nucleic acid sequence of a promoter (constitutive or inducible, as described herein). The term “NC-Rep68” refers to a nucleic acid sequence encoding a polypeptide comprising at least 80% (e.g., at least 80%, at least 85%, at least 90%, at least 95%, or at least 99%) identity to the amino acid sequence of SEQ ID NO: 9, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional Rep68 polypeptide. In some embodiments, NC-Rep68 comprises one or more TAG premature stop codon mutations. In some embodiments, NC-Rep68 comprises one or more TAG premature stop codon mutations at sites identified as tolerant of amino acid substitutions (e.g. positions corresponding to E17, D24, E32, K33, E34, D40, D44, E49, E57, K58, R68, E75, E86, E96, E114, R119, R122, E125, D149, E173, E184, K186, R187, H192, H295, E201, K204, E205, D212, E226, and D233 of SEQ ID NO: 97 as described in Urabe M et al. J Virol. 1999 April; 73 (4): 2682-93, which is incorporated by reference in its entirety). In some embodiments, the AAV production component comprises NC-Rep68 as described above.

In some embodiments, the AAV production component comprises a nucleic acid sequence encoding for NC-Rep78 operably linked to a nucleic acid sequence of a promoter (constitutive or inducible, as described herein). The term “NC-Rep78” refers to a nucleic acid sequence encoding a polypeptide comprising at least 80% (e.g., at least 80%, at least 85%, at least 90%, at least 95%, or at least 99%) identity to the amino acid sequence of SEQ ID NO: 8, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional Rep78 polypeptide. In some embodiments, NC-Rep78 comprises one or more TAG premature stop codon mutations. In some embodiments, NC-Rep78 comprises one or more TAG premature stop codon mutations at sites identified as tolerant of amino acid substitutions (e.g. positions corresponding to E17, D24, E32, K33, E34, D40, D44, E49, E57, K58, R68, E75, E86, E96, E114, R119, R122, E125, D149, E173, E184, K186, R187, H192, H295, E201, K204, E205, D212, E226, and D233 of SEQ ID NO: 97 as described in Urabe M et al. J Virol. 1999 April; 73 (4):2682-93, which is incorporated by reference in its entirety). In some embodiments, the NC-Rep78 nucleic acid sequence is modified to comprise TAG premature stop codons at positions corresponding to D233 and/or E17 of SEQ ID NO: 97. In some embodiments, NC-Rep78 comprises a nucleic acid sequence that is at least 80% (e.g., at least 80%, at least 85%, at least 90%, at least 95%, or at least 99%) identical to any one of SEQ ID NO: 33, 35, 37 and 113-115. In some embodiments, NC-Rep78 comprises a nucleic acid sequence comprising any one of SEQ ID NO: 33, 35, 37, and 113-115. In some embodiments, NC-Rep78 comprises of a nucleic acid sequence consisting of any one of SEQ ID NO: 33, 35, 37, and 113-115. In some embodiments, the NC-Rep78 nucleic acid sequence further comprises an internal ribosomal entry site (IRES).

In some embodiments, the AAV production component comprises a nucleic acid sequence encoding for NC-Rep68 and NC-Rep78 (NC-Rep78/68) operably linked to a nucleic acid sequence of a promoter (constitutive or inducible, as described herein). In some embodiments, the NC-Rep78/68 nucleic acid sequence is modified to comprise TAG premature stop codons at positions corresponding to D233 and/or E17 of SEQ ID NO: 97. In some embodiments, NC-Rep78/68 comprises a nucleic acid sequence that is at least 80% (e.g., at least 80%, at least 85%, at least 90%, at least 95%, or at least 99%) identical to any one of SEQ ID NO: 113-115. In some embodiments, NC-Rep78/68 comprises a nucleic acid sequence comprising any one of SEQ ID NO: 113-115. In some embodiments, NC-Rep78/68 comprises of a nucleic acid sequence consisting of any one of SEQ ID NO: 113-115. In some embodiments, the NC-Rep78/68 nucleic acid sequence further comprises an internal ribosomal entry site (IRES).

As used herein, the term “internal ribosomal entry site (IRES)” refers to a nucleic acid sequence encoding a ribosome binding site that allows for protein translation in a cap-independent manner. Exemplary IRES's include IRES (SEQ ID NO:4) and attenuated IRES (SEQ ID NO: 5). Additional IRES's will be readily known to those of skill in the art.

In some embodiments, NC-Rep78 comprises an amino acid sequence that is at least 80% (e.g., at least 80%, at least 85%, at least 90%, at least 95%, or at least 99%) identical any one of SEQ ID NO: 34, 36, and 38. In some embodiments, NC-Rep78 comprises an amino acid sequence comprising any one of SEQ ID NO: 34, 36, and 38. In some embodiments, NC-Rep78 comprises an amino acid sequence consisting of any one of SEQ ID NO: 34, 36, and 38. In some embodiments, the AAV production component comprises NC-Rep78 as described above.

In some embodiments, the AAV production component comprises a nucleic acid sequence encoding for NC-Rep52 operably linked to a nucleic acid sequence of a promoter (constitutive or inducible, as described herein). The term “NC-Rep52” refers to a nucleic acid sequence encoding a polypeptide comprising at least 80% (e.g., at least 80%, at least 85%, at least 90%, at least 95%, or at least 99%) identity to the amino acid sequence of SEQ ID NO: 6, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional Rep52 polypeptide. In some embodiments, the NC-Rep52 nucleic acid sequence comprises one or more TAG premature stop codon mutations. In some embodiments, the NC-Rep52 nucleic acid sequence comprises one or more TAG premature stop codon mutations at sites identified as tolerant of amino acid substitutions (e.g. positions corresponding to E226 and D233 of SEQ ID NO: 97 as described in Urabe M et al. J Virol. 1999 April; 73 (4): 2682-93, which is incorporated by reference in its entirety). In some embodiments, the NC-Rep52 nucleic acid sequence is modified to comprise TAG premature stop codons at positions corresponding to D233 and/or E17 of SEQ ID NO: 97. In some embodiments, the NC-Rep52 nucleic acid sequence further comprises an internal ribosomal entry site (IRES). In some embodiments, the AAV production component comprises NC-Rep52 as described above.

In some embodiments, the AAV production component comprises a nucleic acid sequence encoding for NC-Rep78 and NC-Rep52 operably linked to a nucleic acid sequence of a promoter (constitutive or inducible, as described herein). The term “NC-Rep78+52” refers to a nucleic acid sequence comprising at least 80% (e.g., at least 80%, at least 85%, at least 90%, at least 95%, or at least 99%) identity to SEQ ID NO: 25, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional Rep78 and Rep 52 polypeptide. In some embodiments, the NC-Rep78+52 nucleic acid sequence comprises one or more TAG premature stop codon mutations. In some embodiments, the NC-Rep78+52 nucleic acid sequence comprises one or more TAG premature stop codon mutations at sites identified as tolerant of amino acid substitutions. In some embodiments, the NC-Rep78+52 nucleic acid sequence comprises TAG premature stop codons at positions corresponding to D233 and/or E17 of SEQ ID NO: 97. In some embodiments, the NC-Rep78+52 nucleic acid sequence comprises point mutations that ablate the Rep68/40 splice site in addition to TAG premature stop codons at positions corresponding to D233 and/or E17 of SEQ ID NO: 97. In some embodiments, the NC-Rep78+52 nucleic acid sequence comprises at least 80% (e.g., at least 80%, at least 85%, at least 90%, at least 95%, or at least 99%) identity to any one of SEQ ID NO: 26-27. In some embodiments, the NC-Rep78+52 nucleic acid sequence comprises any one of SEQ ID NO: 26-27. In some embodiments, the NC-Rep78+52 nucleic acid sequence consists of any one of SEQ ID NO: 26-27. In some embodiments, the NC-Rep78+52 nucleic acid sequence further comprises an IRES. In some embodiments, the IRES in the NC-Rep78+52 nucleic acid sequence initiates translation of NC-Rep78 or NC-Rep52. In some embodiments, the NC-Rep78+52 nucleic acid sequence that further comprises an IRES is at least 80% (e.g., at least 80%, at least 85%, at least 90%, at least 95%, or at least 99%) identical to any one of SEQ ID NO: 31-32. In some embodiments, NC-Rep78+52 comprises a nucleic acid sequence of any one of SEQ ID NO: 31-32. In some embodiments, NC-Rep78+52 consists of a nucleic acid sequence of any one of SEQ ID NO: 31-32. In some embodiments, the AAV production component comprises NC-Rep78+52 as described above.

In some embodiments, the AAV production component comprises a nucleic acid sequence encoding for NC-Rep operably linked to a nucleic acid sequence of a promoter (constitutive or inducible, as described herein). The Rep gene comprises Rep52, Rep40, Rep78, and Rep68. The term “NC-Rep” refers to a nucleic acid sequence comprising at least 80% identity (e.g., at least 80%, at least 85%, at least 90%, at least 95%, or at least 99%) identity to SEQ ID NO: 24, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional NC-Rep polypeptide. In some embodiments, the NC-Rep nucleic acid sequence comprises one or more TAG premature stop codon mutations. In some embodiments, the NC-Rep nucleic acid sequence comprises one or more TAG premature stop codon mutations at sites identified as tolerant of amino acid substitutions. In some embodiments, the NC-Rep nucleic acid sequence is modified to comprise TAG premature stop codons at positions corresponding to D233 and/or E17 of SEQ ID NO: 97. In some embodiments, NC-Rep comprises a nucleic acid sequence comprising at least 80% (e.g., at least 80%, at least 85%, at least 90%, at least 95%, or at least 99%) identity to any one of SEQ ID NOs: 28-29 and 113. In some embodiments, NC-Rep comprises a nucleic acid sequence comprising any one of SEQ ID NOs: 28-29 and 113. In some embodiments, NC-Rep comprises a nucleic acid sequence consisting of any one of SEQ ID NOs: 28-29 and 113. In some embodiments, the AAV production component comprises NC-Rep as described above.

In some embodiments, the AAV production component comprises a nucleic acid sequence encoding for NC-E2A operably linked to a nucleic acid sequence of a promoter (constitutive or inducible, as described herein). The term “NC-E2A” refers to a nucleic acid sequence encoding a polypeptide comprising at least 80% (e.g., at least 80%, at least 85%, at least 90%, at least 95%, or at least 99%) identity to the amino acid sequence of SEQ ID NO: 10, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional E2A polypeptide. In some embodiments, the NC-E2A nucleic acid sequence comprises one or more TAG premature stop codon mutations. In some embodiments, the NC-E2A nucleic acid sequence comprises one or more TAG premature stop codon mutations at sites identified as tolerant of amino acid substitutions. In some embodiments, the AAV production component comprises NC-E2A as described above.

In some embodiments, the AAV production component comprises a nucleic acid sequence encoding for NC-E4ORF6 operably linked to a nucleic acid sequence of a promoter (constitutive or inducible, as described herein). The term “NC-E4ORF6” refers to a nucleic acid sequence encoding a polypeptide comprising at least 80% (e.g., at least 80%, at least 85%, at least 90%, at least 95%, or at least 99%) identity to the amino acid sequence of SEQ ID NO: 11, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional NC-E4ORF6 polypeptide. In some embodiments, NC-E4ORF6 has the splice site removed. In some embodiments, the NC-E4 ORF6 nucleic acid sequence comprises one or more TAG premature stop codon mutations. In some embodiments, NC-E4 ORF6 nucleic acid sequence comprises one or more TAG premature stop codon mutations at sites identified as tolerant of amino acid substitutions. In some embodiments, the AAV production component comprises NC-E4ORF6 as described above.

In some embodiments, the AAV production component comprises a nucleic acid sequence encoding for NC-VP1 operably linked to a nucleic acid sequence of a promoter (constitutive or inducible, as described herein). The term “NC-VP1” refers to a nucleic acid sequence encoding a polypeptide comprising at least 80% (e.g., at least 80%, at least 85%, at least 90%, at least 95%, or at least 99%) identity to the amino acid sequence of SEQ ID NO: 14, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional NC-VP1 polypeptide. In some embodiments, the NC-VP1 nucleic acid sequence comprises one or more TAG premature stop codon mutations. In some embodiments, NC-VP1 nucleic acid sequence comprises one or more TAG premature stop codon mutations at sites identified as tolerant of amino acid substitutions. In some embodiments, the AAV production component comprises NC-VP1 as described above.

In some embodiments, the AAV production component comprises a nucleic acid sequence encoding for NC-VP2 operably linked to a nucleic acid sequence of a promoter (constitutive or inducible, as described herein). The term “NC-VP2” refers to a nucleic acid sequence encoding a polypeptide comprising at least 80% (e.g., at least 80%, at least 85%, at least 90%, at least 95%, or at least 99%) identity to the amino acid sequence of SEQ ID NO: 15, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional NC-VP2 polypeptide. In some embodiments, the NC-VP2 nucleic acid sequence comprises one or more TAG premature stop codon mutations. In some embodiments, NC-VP2 nucleic acid sequence comprises one or more TAG premature stop codon mutations at sites identified as tolerant of amino acid substitutions. In some embodiments, the AAV production component comprises NC-VP2 as described above.

In some embodiments, the AAV production component comprises a nucleic acid sequence encoding for NC-VP3 operably linked to a nucleic acid sequence of a promoter (constitutive or inducible, as described herein). The term “NC-VP3” refers to a nucleic acid sequence encoding a polypeptide comprising at least 80% (e.g., at least 80%, at least 85%, at least 90%, at least 95%, or at least 99%) identity to the amino acid sequence of SEQ ID NO: 16, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional NC-VP3 polypeptide. In some embodiments, the NC-VP3 nucleic acid sequence comprises one or more TAG premature stop codon mutations. In some embodiments, NC-VP3 nucleic acid sequence comprises one or more TAG premature stop codon mutations at sites identified as tolerant of amino acid substitutions. In some embodiments, the AAV production component comprises NC-VP3 as described above.

In some embodiments, the AAV production component comprises a nucleic acid sequence encoding for NC-VP operably linked to a nucleic acid sequence of a promoter (constitutive or inducible, as described herein). The term “NC-VP” refers to a nucleic acid sequence encoding a polypeptide comprising at least 80% (e.g., at least 80%, at least 85%, at least 90%, at least 95%, or at least 99%) identity to the amino acid sequence of SEQ ID NO: 14, wherein at least one codon of the nucleic acid sequence is both a premature stop codon (e.g., TAG, TAA, and TGA) and a codon corresponding to a noncanonical tRNA (as described herein), and wherein incorporation of a noncanonical amino acid(s) during translation of the mRNA corresponding to the polypeptide results in the production of a functional NC-VP polypeptide. In some embodiments, the NC-VP nucleic acid sequence comprises one or more TAG premature stop codon mutations. In some embodiments, NC-VP nucleic acid sequence comprises one or more TAG premature stop codon mutations at sites identified as tolerant of amino acid substitutions. In some embodiments, the AAV production component comprises NC-VP as described above.

### III. Exemplary Embodiments of Engineered Cells Comprising an Amino Acid Incorporation System at Premature Stop Codons

In some aspects, the AAV production system further comprises an engineered cell for AAV production. In some embodiments, the engineered cell comprises the one or more polynucleic acids collectively comprising: (a) an AAV production component as described above and (b) an activity control component comprising a noncanonical tRNA synthase and conjugate noncanonical tRNA as described above. In some embodiments, the AAV production component and the activity control component are stably integrated into the genome of the engineered cell.

As used herein, the term “stably integrated” refers a heterologous nucleic acid sequence, nucleic acid molecule, construct, gene, or polynucleotide that has been inserted into the genome of an organism (e.g., an engineered cell as described herein) and is passed on to future generations after cell division. It is to be understood that any nucleic acid sequence, nucleic acid molecule, construct, gene or polynucleotide described herein may be stably integrated. A nucleic acid sequence, nucleic acid molecule, construct gene or polynucleotide may be integrated into the genome using random integration, targeted integration, or transposon-mediated integration.

In some embodiments, each of the polynucleic acids of the AAV production system comprises a selection marker. In some embodiments, each polynucleic acid of the AAV production system comprises a nucleic acid sequence of a distinct selection marker.

As used herein, the term “selection marker” refers to a protein that-when introduced into or expressed in a cell-confers a trait that is suitable for selection. As used herein, the term “selection cassette” refers to a nucleic acid sequence encoding a selection marker operably linked to a promoter (as described herein) and a terminator.

A selection marker may be a fluorescent protein. Examples of fluorescent proteins are known in the art (e.g., TagBFP, EBFP2, EGFP, EYFP, mKO2, or Sirius). See e.g., U.S. Pat. No. 5,874,304; Patent No.: EP 0969284 A1; Pub. No.: US 2010/167394 A—the entireties of which are incorporated here by reference.

Alternatively, or in addition, a selection marker may be an antibiotic resistance protein. Examples of antibiotic resistance proteins are known in the art (e.g., facilitating puromycin, hygromycin, neomycin, zeocin, blasticidin, or phleomycin selection). See e.g., Pub. No.: WO 1997/15668 A2; Pub. No.: WO 1997/43900 A1—the entireties of which are incorporated here by reference.

### A. The First Stably Integrated Nucleic Acid Molecule

In some embodiments, the engineered cell comprises one or more stably integrated nucleic acid molecules. In some embodiments, the engineered cell comprises a first stably integrated nucleic acid molecule. In some embodiments, the first stably integrated nucleic acid molecule comprises the nucleic acid sequence encoding for a noncanonical tRNA synthetase as described above. In some embodiments, the tRNA synthetase is operably linked to a promoter. In some embodiments, the first stably integrated nucleic acid molecule further comprises a nucleic acid sequence encoding a selection marker. In some embodiments, the selection marker is operably linked to a promoter.

In some embodiments, the engineered cell for AAV production comprises a MmPyrLS WT/Y384F tRNA synthase of SEQ ID NO: 21. In some embodiments, the nucleic acid sequence encoding MmPyrLS WT/Y384F is operably linked to a promoter. In some embodiments, MmPyrLS WT/Y384F is operably linked to a hEF1 promoter.

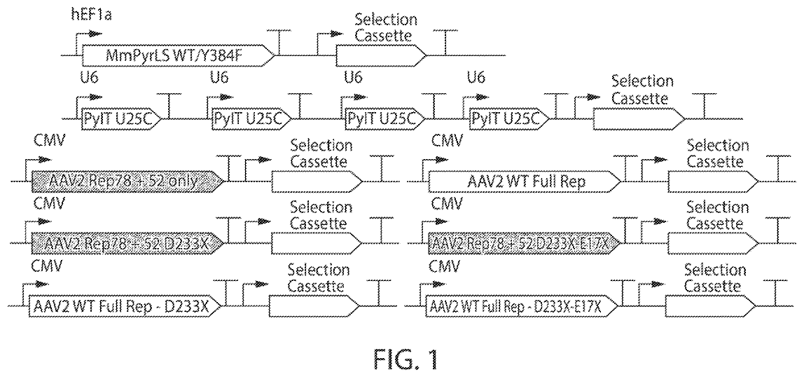

In some embodiments, the first stably integrated nucleic acid molecule as described above has the same structure as is depicted in FIG. 1.

### B. The Second Stably Integrated Nucleic Acid Molecule

In some embodiments, the engineered cell comprising one or more nucleic acid molecules comprises the first stably integrated molecule as described herein and a second stably integrated nucleic acid molecule. In some embodiments, the second stably integrated nucleic acid molecule comprises one or more nucleic acid sequences encoding one or more of any one of the tRNAs described in the application (e.g. 1, 2, 3, 4, 5, 6, 7, 8, 9, or 10 nucleic acid sequences any one of the tRNAs described in the application). In some embodiments, the nucleic acid sequence encoding each of the one or more of any one of the tRNAs described in the application is operably linked to a promoter, as described above.

In some embodiments, the second stably integrated nucleic acid molecule comprises one or more nucleic acid sequences each encoding a PylT (U25C) tRNA operably linked to a promoter. In some embodiments, the second stably integrated nucleic acid molecule comprises four nucleic acid sequences encoding the PylT (U25C) tRNAs are each operably linked to a U6 promoter. In some embodiments, the four nucleic acid sequences encoding the PylT (U25C) tRNAs are each operably linked to a U6 promoter.

In some the second stably integrated nucleic acid molecule further comprises a nucleic acid sequence encoding a selection marker. In some embodiments, the selection marker is operably linked to a promoter.

In some embodiments, the second stably integrated nucleic acid molecule as described above has the same structure as is depicted in FIG. 1.

### C. The Third Stably Integrated Nucleic Acid Molecule

In some embodiments, the engineered cell comprising one or more nucleic acid molecules comprises the first stably integrated molecule as described herein, the second stably integrated nucleic acid molecule as described herein, and a third stably integrated nucleic acid molecule.

In some embodiments, the third stably integrated nucleic acid molecule comprises a nucleic acid sequence encoding NC-Rep78+52 as described above. In some embodiments, the nucleic acid molecule encoding NC-Rep78+52 comprises a premature stop codon that also encodes a PylT (U25C) tRNA codon at position D233. In some embodiments, the nucleic acid molecule encoding NC-Rep78+52 comprises a premature stop codon that also encodes a PylT (U25C) tRNA codon at position E17. In some embodiments, the nucleic acid molecule encoding NC-Rep78+52 comprises a premature stop codon that also encodes a PylT (U25C) tRNA codon at positions D233 and E17.

In some embodiments, the third stably integrated nucleic acid molecule comprises a nucleic acid sequence encoding NC-Rep as described above. In some embodiments, the nucleic acid molecule encoding NC-Rep comprises a premature stop codon that also encodes a PylT (U25C) tRNA codon at position D233. In some embodiments, the nucleic acid molecule encoding NC-Rep comprises a premature stop codon that also encodes a PylT (U25C) tRNA codon at position E17. In some embodiments, the nucleic acid molecule encoding Rep comprises a premature stop codon that also encodes a PylT (U25C) tRNA codon at positions D233 and E17.

In some the third stably integrated nucleic acid molecule further comprises a nucleic acid sequence encoding a selection marker as described herein. In some embodiments, the selection marker is operably linked to a promoter.

In some embodiments, the third stably integrated nucleic acid molecule as described above has the same structure as any one of the diagrams in FIG. 1 depicting a mutated Rep78+52 or a mutated Full-Rep.

### D. The Fourth Stably Integrated Nucleic Acid Molecule

In some embodiments, the engineered cell comprising one or more nucleic acid molecules comprises the first stably integrated molecule as described herein, the second stably integrated nucleic acid molecule as described herein, the third stably integrated nucleic acid molecule as described herein and a fourth stably integrated nucleic acid molecule. In some embodiments, the fourth stably integrated nucleic acid molecule comprises a nucleic acid sequence encoding a transcriptional activator operably linked to a promoter as described above.

### E. The Fifth Stably Integrated Nucleic Acid Molecule

In some embodiments, the engineered cell comprising one or more nucleic acid molecules comprises the first stably integrated molecule as described herein, the second stably integrated nucleic acid molecule as described herein, the third stably integrated nucleic acid molecule as described herein, the fourth stably integrated nucleic acid molecule as described herein and a fifth stably integrated nucleic acid molecule. In some embodiments, the fifth stably integrated nucleic acid molecule comprises a nucleic acid sequence encoding E2A or NC-E2A and E4ORF6 or NC-E4ORF6 operably linked to a promoter as described above.

### F. The Sixth Stably Integrated Nucleic Acid Molecule

In some embodiments, the engineered cell comprising one or more nucleic acid molecules comprises the first stably integrated molecule as described herein, the second stably integrated nucleic acid molecule as described herein, the third stably integrated nucleic acid molecule as described herein, the fourth stably integrated nucleic acid molecule as described herein, the fifth stably integrated nucleic acid molecule as described herein and a sixth stably integrated nucleic acid molecule. In some embodiments, the sixth stably integrated nucleic acid molecule comprises a nucleic acid sequence encodi VP (CAP) gene operably linked to a promoter as described above. In some embodiments, the sixth stably integrated nucleic acid molecule comprises an AAV payload.

### IV. Methods of Using Engineered Cells for AAV Production Comprising a Non-Canonical tRNA

In some aspects, the present disclosure provides methods for producing AAV using an AAV production system comprising one or more polynucleic acids collectively comprising: (a) an AAV production component and (b) an activity control component comprising a noncanonical tRNA synthetase/tRNA as described herein. In some embodiments, the method of AAV production comprises transfecting or stably integrating into an engineered cell any combination of the one or more polynucleic acids collectively comprising an AAV production component and an activity control component as described herein. In some embodiments, the method of AAV production further comprises transfecting a nucleic acid molecule comprising a payload for AAV delivery (e.g. a therapeutic DNA sequence) as described above. In some embodiments, the engineered cell used in the method of AAV production is selected from any one of the engineered cells for AAV production comprising a noncanonical tRNA synthetase/tRNA as described herein. In some embodiments, the method comprises growing the engineered cell to a confluency that is optimal for AAV production. An optimal confluency may be dependent, for example, on the type of cell the engineered cell is derived from. The skilled person will know or be able to determine the optimal confluency for AAV production. In some embodiments, the method comprises contacting the engineered cell with an amino acid that can be charged onto the noncanonical tRNA. In some embodiments, the amino acid is H-Lys(Boc)-OH. In some embodiments, the method comprises inducing expression of the tRNA synthase and/or the conjugate tRNA using a small molecule inducer as described herein. In some embodiments, the method comprises harvesting the AAV produced from the culture of engineered cells using methods that are well known to those of skill in the art.

### V. An AAV Production System Comprising a Base Editor

In some aspects, the AAV production system comprises one or more polynucleic acids collectively comprising: (a) an AAV production component and (b) an activity control component comprising a Base Editor capable of correcting a mutation(s) in nucleic acid sequences. In some embodiments, the Base Editor replaces a premature stop codon with a canonical codon.

### A. Base Editor

As described herein, the term “Base Editor” refers to a protein or fusion protein capable of introducing single-nucleotide variants (SNVs) into DNA or RNA. Exemplary Base Editors include but are not limited to Cytosine Base Editors (CBE): BE1, BE2, HF2-BE2, BE3, HF-BE3, YE1-BE3, EE-BE3, YEE-BE3, VQR-BE3, EQR-BE3, VRER-BE3, SaKKHBE3, FNLS-BE3, RA-BE3, eA3A-HF1-BE3-2×UGI, eA3A-Hypa-BE3-2×UGI, hA3A-BE3, hA3B-BE3, hA3G-BE3, hAID-BE3, SaCas9-BE3, xCas9-BE3, ScCas9-BE3, SniperCas9-BE3, iSpyMac-BE3, Target-AID, Target-AID-NG, BE-PLUS, BE4, BE4-Gam, BE4-Max, AncBE4-Max, SaCas9BE4-Gam, evoBe4max, evoFERNY-BE4max, and Cas12a-BE; and Adenine Base Editors (ABE): ABE7.8, ABE9, ABE10, ABE.8.17, xCas9-ABE7.10, VQR-ABE, Sa (KKH)-ABE, ABEmax, ABE7.10max, ABE8e, PE1, PE2, PE3, ABE REPAIRv1, and ABE Repairv2, which are described in more detail in Porto, Elizabeth M., et al. Nature Reviews Drug Discovery 19.12 (2020): 839-859; Cox, David B T, et al. Science 358.6366 (2017): 1019-1027; Komor, Alexis C., et al. Science advances 3.8 (2017): eaao4774; and Gaudelli, Nicole M., et al. Nature biotechnology 38.7 (2020): 892-900; and Kantor A. et al. International Journal of Molecular Sciences 21.17 (2020): 6240 each of which is incorporated by reference in its entirety. In a non-limiting overview, a Base Editor is a fusion protein comprising a CRISPR Cas protein domain with a catalytically inactive exonuclease domain (e.g. dCas9 or dCas13) or a CRISPR Cas nickase protein domain (e.g. Cas9n) and one or more domains capable of modifying DNA (e.g. adenosine deaminase). The Base Editor binds to a single guide RNA (sgRNA) that comprises a nucleic acid sequence that is complementary to a target DNA or RNA sequence. The targeting of the Base Editor to DNA or RNA is determined by the type of Cas protein used (Cas9 for DNA and Cas13 for RNA). In a non-limiting example of a Base Editor, an Adenine Base Editor (ABE) comprises a Cas9n protein, an adenosine deaminase, and a single guide RNA comprising a sequence that is complementary to a target gene (e.g. a rep52 gene comprising a premature stop codon). The sgRNA directs the ABE to the target DNA sequence, the target DNA sequence is bound by Cas9n, the Cas9n nicks the target strand and the adenosine deaminase deaminates the target adenosine nucleotide converting it to an inosine, which during DNA replication is read as guanine resulting in an A-T to G-C DNA modification. Examples of codon altering mutations that can be made using Base Editors are exemplified in Table 1 and Table 2. In some embodiments, zinc-finger nucleases, transcriptional activator-like effector nucleases (TALENs), or Prime Editors may be used in the place of a Base Editor.

TABLE 1
Possible codon mutations that can be made with an
ABE.

| ABE sense strand (DNA & | | | ABE antisense strand | | |
|---|---|---|---|---|---|
| RNA editors) | | | (DNA editors) | | |
| Original | → | Mutant | Mutant | → | Mutant |
| TAA (*) | → | TGA (*) | TAA (*) | → | CAA (Q) |
| TAA (*) | → | TAG (*) | TAG (*) | → | CAG (Q) |
| TAG (*) | → | TGG (W) | TGA (*) | → | CGA (R) |
| TGA (*) | → | TGG (W) | GCT (A) | → | GCC (A) |
| GCA (A) | → | GCG (A) | TGT (C) | → | CGT (R) |
| GAT (D) | → | GGT (G) | TGT (C) | → | TGC (C) |
| GAC (D) | → | GGC (G) | TGC (C) | → | CGC (R) |
| GAA (E) | → | GGA (G) | GAT (D) | → | GAC (D) |
| GAA (E) | → | GAG (E) | TTT (F) | → | CTT (L) |
| GAG (E) | → | GGG (G) | TTT (F) | → | TCT (S) |
| GGA (G) | → | GGG (G) | TTT (F) | → | TTC (F) |
| CAT (H) | → | CGT (R) | TTC (F) | → | CTC (L) |
| CAC (H) | → | CGC (R) | TTC (F) | → | TCC (S) |
| ATT (I) | → | GTT (V) | GGT (G) | → | GGC (G) |
| ATC (I) | → | GTC (V) | CAT (H) | → | CAC (H) |
| ATA (I) | → | GTA (V) | ATT (I) | → | ACT (T) |
| ATA (I) | → | ATG (M) | ATT (I) | → | ATC (I) |
| AAA (K) | → | GAA (E) | ATC (I) | → | ACC (T) |
| AAA (K) | → | AGA (R) | ATA (I) | → | ACA (T) |
| AAA (K) | → | AAG (K) | TTA (L) | → | CTA (L) |
| AAG (K) | → | GAG (E) | TTA (L) | → | TCA (S) |
| AAG (K) | → | AGG (R) | TTG (L) | → | CTG (L) |
| TTA (L) | → | TTG (L) | TTG (L) | → | TCG (S) |
| CTA (L) | → | CTG (L) | CTT (L) | → | CCT (P) |
| ATG (M) | → | GTG (V) | CTT (L) | → | CTC (L) |
| AAT (N) | → | GAT (D) | CTC (L) | → | CCC (P) |
| AAT (N) | → | AGT (S) | CTA (L) | → | CCA (P) |
| AAC (N) | → | GAC (D) | CTG (L) | → | CCG (P) |
| AAC (N) | → | AGC (S) | ATG (M) | → | ACG (T) |
| CCA (P) | → | CCG (P) | AAT (N) | → | AAC (N) |
| CAA (Q) | → | CGA (R) | CCT (P) | → | CCC (P) |
| CAA (Q) | → | CAG (Q) | CGT (R) | → | CGC (R) |
| CAG (Q) | → | CGG (R) | TCT (S) | → | CCT (P) |
| CGA (R) | → | CGG (R) | TCT (S) | → | TCC (S) |
| AGA (R) | → | GGA (G) | TCC (S) | → | CCC (P) |
| AGA (R) | → | AGG (R) | TCA (S) | → | CCA (P) |
| AGG (R) | → | GGG (G) | TCG (S) | → | CCG (P) |
| TCA (S) | → | TCG (S) | AGT (S) | → | AGC (S) |
| AGT (S) | → | GGT (G) | ACT (T) | → | ACC (T) |
| AGC (S) | → | GGC (G) | GTT (V) | → | GCT (A) |
| ACT (T) | → | GCT (A) | GTT (V) | → | GTC (V) |
| ACC (T) | → | GCC (A) | GTC (V) | → | GCC (A) |
| ACA (T) | → | GCA (A) | GTA (V) | → | GCA (A) |
| ACA (T) | → | ACG (T) | GTG (V) | → | GCG (A) |
| ACG (T) | → | GCG (A) | TGG (W) | → | CGG (R) |
| GTA (V) | → | GTG (V) | TAT (Y) | → | CAT (H) |
| TAT (Y) | → | TGT (C) | TAT (Y) | → | TAC (Y) |
| TAC (Y) | → | TGC (C) | TAC (Y) | → | CAC (H) |

TABLE 2
Possible codon mutations that can be made with a
CBE.

| CBE Sense (DNA & | | | CBE antisense (DNA | | |
|---|---|---|---|---|---|
| RNA editors) | | | editors) | | |
| Orignal | → | Mutant | Orignal | → | Mutant |
| GCT (A) | → | GTT (V) | TAG (*) | → | TAA (*) |
| GCC (A) | → | GTC (V) | TGA (*) | → | TAA (*) |
| GCC (A) | → | GCT (A) | GCT (A) | → | ACT (T) |
| GCA (A) | → | GTA (V) | GCC (A) | → | ACC (T) |
| GCG (A) | → | GTG (V) | GCA (A) | → | ACA (T) |
| TGC (C) | → | TGT (C) | GCG (A) | → | ACG (T) |
| GAC (D) | → | GAT (D) | GCG (A) | → | GCA (A) |
| TTC (F) | → | TTT (F) | TGT (C) | → | TAT (Y) |
| GGC (G) | → | GGT (G) | TGC (C) | → | TAC (Y) |
| CAT (H) | → | TAT (Y) | GAT (D) | → | AAT (N) |
| CAC (H) | → | TAC (Y) | GAC (D) | → | AAC (N) |
| CAC (H) | → | CAT (H) | GAA (E) | → | AAA (K) |
| ATC (I) | → | ATT (I) | GAG (E) | → | AAG (K) |
| CTT (L) | → | TTT (F) | GAG (E) | → | GAA (E) |
| CTC (L) | → | TTC (F) | GGT (G) | → | AGT (S) |
| CTC (L) | → | CTT (L) | GGT (G) | → | GAT (D) |
| CTA (L) | → | TTA (L) | GGC (G) | → | AGC (S) |
| CTG (L) | → | TTG (L) | GGC (G) | → | GAC (D) |
| AAC (N) | → | AAT (N) | GGA (G) | → | AGA (R) |
| CCT (P) | → | TCT (S) | GGA (G) | → | GAA (E) |
| CCT (P) | → | CTT (L) | GGG (G) | → | AGG (R) |
| CCC (P) | → | TCC (S) | GGG (G) | → | GAG (E) |
| CCC (P) | → | CTC (L) | GGG (G) | → | GGA (G) |
| CCC (P) | → | CCT (P) | AAG (K) | → | AAA (K) |
| CCA (P) | → | TCA (S) | TTG (L) | → | TTA (L) |
| CCA (P) | → | CTA (L) | CTG (L) | → | CTA (L) |
| CCG (P) | → | TCG (S) | ATG (M) | → | ATA (I) |
| CCG (P) | → | CTG (L) | CCG (P) | → | CCA (P) |
| CAA (Q) | → | TAA (*) | CAG (Q) | → | CAA (Q) |
| CAG (Q) | → | TAG (*) | CGT (R) | → | CAT (H) |
| CGT (R) | → | TGT (C) | CGC (R) | → | CAC (H) |
| CGC (R) | → | TGC (C) | CGA (R) | → | CAA (Q) |
| CGC (R) | → | CGT (R) | CGG (R) | → | CAG (Q) |
| CGA (R) | → | TGA (*) | CGG (R) | → | CGA (R) |
| CGG (R) | → | TGG (W) | AGA (R) | → | AAA (K) |
| TCT (S) | → | TTT (F) | AGG (R) | → | AAG (K) |
| TCC (S) | → | TTC (F) | AGG (R) | → | AGA (R) |
| TCC (S) | → | TCT (S) | TCG (S) | → | TCA (S) |
| TCA (S) | → | TTA (L) | AGT (S) | → | AAT (N) |
| TCG (S) | → | TTG (L) | AGC (S) | → | AAC (N) |
| AGC (S) | → | AGT (S) | ACG (T) | → | ACA (T) |
| ACT (T) | → | ATT (I) | GTT (V) | → | ATT (I) |
| ACC (T) | → | ATC (I) | GTC (V) | → | ATC (I) |
| ACC (T) | → | ACT (T) | GTA (V) | → | ATA (I) |
| ACA (T) | → | ATA (I) | GTG (V) | → | ATG (M) |
| ACG (T) | → | ATG (M) | GTG (V) | → | GTA (V) |
| GTC (V) | → | GTT (V) | TGG (W) | → | TAG (*) |
| TAC (Y) | → | TAT (Y) | TGG (W) | → | TGA (*) |

In some embodiments, the activity control component comprises a nucleic acid sequence encoding the amino acid sequence of a Base Editor selected from the group consisting of Cas9 ABE7.10 (SEQ ID NO: 82), Cas9 ABE8.17m (SEQ ID NO: 83), Cas13 ABE REPAIRv1 (SEQ ID NO: 84), and Cas13 ABE REPAIRv2 (SEQ ID NO: 85). In some embodiments, the Base Editor is encoded by a polypeptide comprising at least 80% (e.g., at least 80%, at least 85%, at least 90%, at least 95%, or at least 99%) identity any one of SEQ ID NO: 82-85, wherein the Base Editor is still capable of editing RNA or DNA. In some embodiments, the Base Editor is encoded by a polypeptide comprising the amino acid sequence of any one of SEQ ID NO: 82-85. In some embodiments, the Base Editor is encoded by a polypeptide consisting of the amino acid sequence of any one of SEQ ID NO: 82-85.

In some embodiments, the activity control component comprises a nucleic acid sequence encoding a Base Editor (e.g. Cas9 ABE7.10, Cas9 ABE8.17m, Cas13 ABE REPAIRv1 or Cas13 ABE REPAIRv2) that is operably linked to a promoter (as described herein). In some embodiments, the promoter is a constitutively active promoter. In some embodiments, the promoter is a chemically inducible promoter. In some embodiments, the Base Editor is operably linked to a chemically inducible promoter selected from the group consisting of pTRE3G (SEQ ID NO: 1) or pTREtight (SEQ ID NO: 2). In some embodiments, the Base Editor is operably linked to a chemically inducible promoter containing at least one of VanR (SEQ ID NO: 86), TtgR (SEQ ID NO: 86), PhlF (SEQ ID NO: 86), or CymR (SEQ ID NO: 86), or the Gal4 UAS (SEQ ID NO: 86) operator sequences.

B. AAV Production Component: Genes Required for AAV Production Comprising Mutations that Decrease AAV Gene Product Activity

In some embodiments, the AAV production component comprises a heterologous polynucleic acid comprising a nucleic acid sequence encoding for a gene product(s) required for AAV production, wherein the gene product(s) is modified to comprise one or more mutations that decrease the function of the gene product as described above (e.g. 1, 2, 3, 4, 5, 6, 7, 8, 9, or 10). In some embodiments, the polynucleic acid encoding for the gene product(s) required for AAV production may comprise 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 mutations that decrease the function of the gene product.

In some embodiments, the one or more mutations are selected from the codon mutations in Table 1 and Table 2. In some embodiments, the one or more mutations comprise codon mutations that result in an amino acids of different classification being encoded compared to the wildtype encoded amino acid. In some embodiments, the different classifications of amino acids are Positively Charged: arginine, histidine, and lysine; Negatively Charged: aspartic acid and glutamic acid; Polar: Serine, Threonine, Cysteine, Tyrosine, Asparagine, and Glutamine; Nonpolar: glycine, alanine, valine, leucine, isoleucine, methionine, tryptophan, phenylalanine or proline. In some embodiments, one or more amino acid codons for a positively charged amino acid(s) is replaced with a codon for a negatively charged, nonpolar, or polar amino acid. In some embodiments, one or more amino acid codon(s) for a negatively charged amino acid is replaced with a codon for a positively charged, nonpolar, or polar amino acid. In some embodiments, one or more amino acid codon(s) for a polar amino acid is replaced with a codon for a negatively charged, positively charged, or polar amino acid. In some embodiments, one or more amino acid codon(s) for a nonpolar amino acid is replaced with a codon for a negatively charged, nonpolar, or positively charged amino acid.

In some embodiments, the AAV production component comprises a heterologous polynucleic acid comprising a nucleic acid sequence encoding for a gene product(s) required for AAV production, wherein the gene product(s) is modified to comprise a premature stop codon(s). In some embodiments, the polynucleic acid encoding for the gene product(s) may comprise a premature stop codon at a position corresponding to a tryptophan codon, a glutamine codon or an arginine codon. In some embodiments, the polynucleic acid encoding for the gene product(s) may comprise one or more premature stop codon(s) (e.g. 1, 2, 3, 4, 5, 6, 7, 8, 9, or 10) at a position corresponding to a tryptophan codon, a glutamine codon or an arginine codon. In some embodiments, the polynucleic acid encoding for the gene product(s) may comprise 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, or 6-10 premature stop codon(s) at a position(s) corresponding to tryptophan codon a glutamine codon or an arginine codon.

The modifier “DA” as used herein, refers to a gene comprising one or more mutations that decrease the activity of the product of the gene (e.g. a premature stop codon(s)). In some embodiments, one or more stop codon mutations are inserted by mutating one or more tryptophan and/or arginine codon(s) on the sense DNA strand, or one or more glutamine, arginine, and/or proline codon(s) on the antisense DNA strand to premature stop codons. In some embodiments, the AAV production component comprises: a nucleic acid sequence encoding for DA-Rep52 operably linked to a promoter (constitutive or inducible, as described herein); a nucleic acid sequence encoding for DA-Rep40 operably linked to a nucleic acid sequence of a promoter (constitutive or inducible, as described herein); a nucleic acid sequence encoding for DA-Rep78 operably linked to a nucleic acid sequence of a promoter (constitutive or inducible, as described herein); a nucleic acid sequence encoding for DA-Rep68 operably linked to a nucleic acid sequence of a promoter (constitutive or inducible); a nucleic acid sequence encoding for DA-Rep78+52 operably linked to a nucleic acid sequence of a promoter (constitutive or inducible, as described herein); a nucleic acid sequence encoding for DA-Rep operably linked to a nucleic acid sequence of a promoter (constitutive or inducible, as described herein); a nucleic acid sequence encoding for DA-E2A operably linked to a nucleic acid sequence of a promoter (constitutive or inducible, as described herein); a nucleic acid sequence encoding for DA-E4ORF6 operably linked to a nucleic acid sequence of a promoter (constitutive or inducible, as described herein); a nucleic acid sequence encoding for DA-VP1 operably linked to a promoter (constitutive or inducible, as described herein); a nucleic acid sequence encoding for DA-VP2 operably linked to a promoter (constitutive or inducible, as described herein); a nucleic acid sequence encoding for DA-VP3 operably linked to a promoter (constitutive or inducible, as described herein); a nucleic acid sequence encoding for DA-VP operably linked to a promoter (constitutive or inducible, as described herein); a nucleic acid sequence encoding for DA-L4 100K operably linked to a promoter (constitutive or inducible, as described herein); or any combination thereof.

In some embodiments, the nucleic acid sequences encoding DA-E2A, DA-E4ORF6, DA-Rep52, DA-Rep40, DA-Rep78, DA-Rep68, DA-Rep, DA-VP1, DA-VP2, DA-VP3, DA-VP, and DA-L4 100K further comprise one or more mutations to introduce a PAM sequence. In some embodiments, the nucleic acid sequences encoding DA-E2A, DA-E4ORF6, DA-Rep52, DA-Rep40, DA-Rep78, DA-Rep68, DA-Rep, DA-VP1, DA-VP2, DA-VP3, DA-VP, and DA-L4 100K further comprise one or more silent mutations to introduce a PAM sequence. In some embodiments, the PAM sequence is introduced near the mutation(s) to introduce a PAM sequence for a DNA Base editor (e.g. Cas9 containing ABEs or CBEs). In some embodiments, the PAM sequence is introduced 1, 2, 3, 4, 5, 6, 7, 8, 9, 10, 11, 12, 13, 14, 15, 16, 17, 18, 19 or 20 nucleotides upstream of the target editing site. In some embodiments, the PAM sequence is introduced within 1, 2, 3, 4, 5, 6, 7, 8, 9, 10, 11, 12, 13, 14, 15, 16, 17, 18, 19, and 20 of the targeting editing site. In some embodiments, the PAM sequence is introduced within 10-17 or 13-16 nucleotide of the target editing site. In some embodiments, one or more silent mutations are made to reduce off-target base editing within the Base Editor window.

In some embodiments, the AAV production component comprises a nucleic acid sequence encoding for DA-Rep52 operably linked to a nucleic acid sequence of a promoter (constitutive or inducible, as described herein). In some embodiments, the DA-Rep52 nucleic acid sequence encoding an amino acid sequence is operably linked to a p19 promoter. The term “DA-Rep52” refers to a nucleic acid sequence encoding a polypeptide comprising at least 80% (e.g., at least 80%, at least 85%, at least 90%, at least 95%, or at least 99%) identity to the amino acid sequence of SEQ ID NO: 6 comprising at least one mutation that decreases the activity of Rep52 (as described above). In some embodiments, DA-Rep52 comprises a nucleic acid sequence encoding a polypeptide comprising SEQ ID NO: 6 that is modified to comprise a mutation an amino acid position corresponding to M225 of SEQ ID NO: 97. In some embodiments, DA-Rep52 comprises a nucleic acid sequence encoding a polypeptide comprising SEQ ID NO: 6 that is modified to comprise a methionine to glycine mutation at amino acid position corresponding to M225 of SEQ ID NO: 97. In some embodiments, the nucleic acid sequence encoding DA-Rep52 comprises a mutation at a position corresponding to amino acid position R529 of SEQ ID NO: 97. In some embodiments, the nucleic acid sequence encoding DA-Rep52 comprises an AgG→CgC mutation at a position corresponding to amino acid position R529 of SEQ ID NO: 97. In some embodiments, DA-Rep52 comprises one or more (e.g. 1, 2, 3, 4, 5, 6, 7 or more) missense or premature stop codon mutations at or between positions corresponding to one or more of 226-227, 256-257, 259-260, 346-347, 400-401, 409-410, and 455-456 that decrease Rep52 activity. In some embodiments, at least one codon of the nucleic acid sequence is a premature stop codon (e.g., TAG, TAA, and TGA). In some embodiments, DA-Rep52 comprises a nucleic acid sequence encoding a polypeptide comprising SEQ ID NO: 6 that is modified to comprise a premature stop codon at amino acid position corresponding to Q262 or W319 of SEQ ID NO: 97. In some embodiments, DA-Rep52 comprises a nucleic acid sequence encoding a polypeptide comprising SEQ ID NO: 6 that is modified to comprise a premature stop codon at amino acid position corresponding to Q262 and W319 of SEQ ID NO: 97. In some embodiments, DA-Rep52 comprises a nucleic acid sequence encoding a polypeptide comprising at least 80% (e.g., at least 80%, at least 85%, at least 90%, at least 95%, or at least 99%) identity to the amino acid sequence of any one of SEQ ID NO: 43 or 47. In some embodiments, DA-Rep52 comprises a nucleic acid sequence encoding a polypeptide comprising the amino acid sequence of any one of SEQ ID NO: 43 or 47. In some embodiments, DA-Rep52 comprises a nucleic acid sequence encoding a polypeptide consisting of the amino acid sequence any one of SEQ ID NO: 43 or 47.

In some embodiments, the AAV production component comprises a nucleic acid sequence encoding for DA-Rep40 operably linked to a nucleic acid sequence of a promoter (constitutive or inducible, as described herein). In some embodiments, the DA-Rep40 nucleic acid sequence is operably linked to a p19 promoter. The term “DA-Rep40” refers to a nucleic acid sequence encoding a polypeptide comprising at least 80% (e.g., at least 80%, at least 85%, at least 90%, at least 95%, or at least 99%) identity to the amino acid sequence of SEQ ID NO: 7 comprising at least one mutation that decreases the activity of Rep40 (as described above). In some embodiments, DA-Rep40 comprises a nucleic acid sequence encoding a polypeptide comprising SEQ ID NO: 7 that is modified to comprise a mutation at amino acid position corresponding to M225 of SEQ ID NO: 97. In some embodiments, DA-Rep40 comprises a nucleic acid sequence encoding a polypeptide comprising SEQ ID NO: 7 that is modified to comprise a methionine to glycine mutation at amino acid position corresponding to M225 of SEQ ID NO: 97. In some embodiments, the nucleic acid sequence encoding DA-Rep40 comprises a mutation at a position corresponding to amino acid position R529 of SEQ ID NO: 97. In some embodiments, the nucleic acid sequence encoding DA-Rep40 comprises an AgG→CgC mutation at a position corresponding to amino acid position R529 of SEQ ID NO: 97. In some embodiments, DA-Rep40 comprises one or more (e.g. 1, 2, 3, 4, 5, 6, 7 or more) missense or premature stop codon mutations at or between positions corresponding to one or more of 226-227, 256-257, 259-260, 346-347, 400-401, 409-410, and 455-456 that decrease Rep40 activity. In some embodiments, at least one codon of the nucleic acid sequence is a premature stop codon (e.g., TAG, TAA, and TGA). In some embodiments, DA-Rep40 comprises a nucleic acid sequence encoding a polypeptide comprising SEQ ID NO: 7 that is modified to comprise a premature stop codon at amino acid position corresponding to Q262 or W319 of SEQ ID NO: 97. In some embodiments, DA-Rep40 comprises a nucleic acid sequence encoding a polypeptide comprising SEQ ID NO: 7 that is modified to comprise a premature stop codon at amino acid position corresponding to Q262 and W319 of SEQ ID NO: 97. In some embodiments, DA-Rep40 comprises a nucleic acid sequence encoding a polypeptide comprising at least 80% (e.g., at least 80%, at least 85%, at least 90%, at least 95%, or at least 99%) identity to any one of SEQ ID NO: 44 or 48. In some embodiments, DA-Rep40 comprises a nucleic acid sequence encoding a polypeptide comprising an amino acid sequence of any one of SEQ ID NO: 44 or 48. In some embodiments, DA-Rep40 comprises a nucleic acid sequence encoding a polypeptide comprising an amino acid sequence consisting of any one of SEQ ID NO: 44 or 48.

In some embodiments, the AAV production component comprises a nucleic acid sequence encoding for DA-Rep78 operably linked to a nucleic acid sequence of a promoter (constitutive or inducible, as described herein). In some embodiments, the DA-Rep78 nucleic acid sequence encoding is operably linked to a p19 promoter. The term “DA-Rep78” refers to a nucleic acid sequence encoding a polypeptide comprising at least 80% (e.g., at least 80%, at least 85%, at least 90%, at least 95%, or at least 99%) identity to the amino acid sequence of SEQ ID NO: 8 comprising at least one mutation that decreases the activity of Rep78 (as described above). In some embodiments, the nucleic acid sequence encoding DA-Rep78 comprises a mutation at a position corresponding to amino acid position R529 of SEQ ID NO: 97. In some embodiments, the nucleic acid sequence encoding DA-Rep78 comprises an AgG→CgC mutation at a position corresponding to amino acid position R529 of SEQ ID NO: 97. In some embodiments, DA-Rep78 comprises one or more (e.g. 1, 2, 3, 4, 5, 6, 7, 8, 9, 10, 11, 12, 13, 14, 15, 16, 17 or more) missense or premature stop codon mutations at or between positions corresponding to one or more of 56-57, 58-59, 61-62, 73-74, 76-77, 87-88, 113-114, 133-134, 164-165, 217-218, 226-227, 256-257, 259-260, 346-347, 400-401, 409-410, and 455-456 that decrease Rep78 activity. In some embodiments, at least one codon of the nucleic acid sequence is a premature stop codon (e.g., TAG, TAA, and TGA). In some embodiments, DA-Rep78 comprises a nucleic acid sequence encoding a polypeptide comprising an amino acid sequence of SEQ ID NO: 8 that is modified to comprise a premature stop codon at amino acid position corresponding to Q262 or W319 of SEQ ID NO: 97. In some embodiments, DA-Rep78 comprises a nucleic acid sequence encoding a polypeptide comprising an amino acid sequence of SEQ ID NO: 8 that is modified to comprise a premature stop codon at amino acid position corresponding to Q262 and W319 of SEQ ID NO: 97. In some embodiments, DA-Rep78 comprises a nucleic acid sequence encoding a polypeptide comprising at least 80% (e.g., at least 80%, at least 85%, at least 90%, at least 95%, or at least 99%) identity to any one of SEQ ID NO: 45 or 49. In some embodiments, DA-Rep78 comprises a nucleic acid sequence encoding a polypeptide comprising an amino acid sequence of SEQ ID NO: 45 or 49. In some embodiments, DA-Rep78 comprises a nucleic acid sequence encoding a polypeptide comprising an amino acid sequence of SEQ ID NO: 45 or 49.

In some embodiments, the AAV production component comprises a nucleic acid sequence encoding for DA-Rep68 operably linked to a nucleic acid sequence of a promoter (constitutive or inducible, as described herein). In some embodiments, the DA-Rep68 nucleic acid sequence is operably linked to a p19 promoter. The term “DA-Rep68” refers to a nucleic acid sequence encoding a polypeptide comprising at least 80% (e.g., at least 80%, at least 85%, at least 90%, at least 95%, or at least 99%) identity to the amino acid sequence of SEQ ID NO: 9 comprising at least one mutation that decreases the activity of Rep68 (as described above). In some embodiments, the nucleic acid sequence encoding DA-Rep68 comprises a mutation at a position corresponding to amino acid position R529 of SEQ ID NO: 97. In some embodiments, the nucleic acid sequence encoding DA-Rep68 comprises an AgG→CgC mutation at a position corresponding to amino acid position R529 of SEQ ID NO: 97. In some embodiments, DA-Rep68 comprises one or more (e.g. 1, 2, 3, 4, 5, 6, 7, 8, 9, 10, 11, 12, 13, 14, 15, 16, 17 or more) missense or premature stop codon mutations at or between positions corresponding to one or more of 56-57, 58-59, 61-62, 73-74, 76-77, 87-88, 113-114, 33-134, 164-165, 217-218, 226-227, 256-257, 259-260, 346-347, 400-401, 409-410, and 455-456 that decrease Rep68 activity. In some embodiments, at least one codon of the nucleic acid sequence is a premature stop codon (e.g., TAG, TAA, and TGA). In some embodiments, DA-Rep68 comprises a nucleic acid sequence encoding a polypeptide comprising an amino acid sequence of SEQ ID NO: 9 that is modified to comprise a premature stop codon at amino acid position corresponding to Q262 or W319 of SEQ ID NO: 97. In some embodiments, DA-Rep68 comprises a nucleic acid sequence encoding a polypeptide comprising an amino acid sequence of SEQ ID NO: 9 that is modified to comprise a premature stop codon at amino acid position corresponding to Q262 and W319 of SEQ ID NO: 97. In some embodiments, DA-Rep68 comprises a nucleic acid sequence encoding a polypeptide comprising least 80% (e.g., at least 80%, at least 85%, at least 90%, at least 95%, or at least 99%) identical the amino acid sequence of any one of SEQ ID NO: 46 or 50. In some embodiments, DA-Rep68 comprises a nucleic acid sequence encoding a polypeptide comprising an amino acid sequence of any one of SEQ ID NO: 46 or 50. In some embodiments, DA-Rep68 comprises a nucleic acid sequence encoding a polypeptide consisting of an amino acid sequence of SEQ ID NO: 46 or 50

In some embodiments, the AAV production component comprises a nucleic acid sequence encoding for DA-Rep operably linked to a nucleic acid sequence of a promoter (constitutive or inducible, as described herein). In some embodiments, the DA-Rep nucleic acid sequence is operably linked to a p19 promoter. The term “DA-Rep” refers to a nucleic acid sequence comprising at least 80% (e.g., at least 80%, at least 85%, at least 90%, at least 95%, or at least 99%) identity to sequence of SEQ ID NO: 24 comprising at least one mutation that decreases the activity of Rep (as described above). In some embodiments, the nucleic acid sequence encoding DA-Rep comprises a mutation at a position corresponding to amino acid position R529 of Rep (SEQ ID NO: 97). In some embodiments, the nucleic acid sequence encoding DA-Rep comprises an AgG→CgC mutation at a position corresponding to amino acid position R529 of Rep (SEQ ID NO: 97). In some embodiments, DA-Rep is modified to comprise a mutation at amino acid position corresponding to M225 of SEQ ID NO: 97. In some embodiments, DA-Rep is modified to comprise a methionine to glycine mutation at amino acid position corresponding to M225 of SEQ ID NO: 97 as described in Kyostio S R et al. J Virol. 1994 May; 68 (5): 2947-2957, which is incorporate by reference in its entirety. In some embodiments, DA-Rep is modified to comprise a mutation at amino acid position corresponding to K340 of SEQ ID NO: 97. In some embodiments, DA-Rep is modified to comprise a lysine to histidine mutation at amino acid position corresponding to K340 of SEQ ID NO: 97 as described in Smith R H et al. J Virol. 1997 June; 71 (6): 4461-4471, which is incorporated by reference in its entirety. In some embodiments, at least one codon of the nucleic acid sequence is a premature stop codon (e.g., TAG, TAA, and TGA). In some embodiments, DA-Rep comprises one or more (e.g. 1, 2, 3, 4, 5, 6, 7, 8, 9, 10, 11, 12, 13, 14, 15, 16, 17 or more) missense or premature stop codon mutations at or between positions corresponding to one or more of 56-57, 58-59, 61-62, 73-74, 76-77, 87-88, 113-114, 133-134, 164-165, 217-218, 226-227, 256-257, 259-260, 346-347, 400-401, 409-410, and 455-456 that decrease Rep activity. Yang Q et al. J Virol. 1992 October; 66 (10): 6058-6069, which is incorporated by reference in its entirety, indicates that these positions are sensitive to insertion mutations.

In some embodiments, DA-Rep comprises a nucleic acid sequence that is modified to comprise a premature stop codon at amino acid position corresponding to Q67, Q262 or W319 of SEQ ID NO: 97. In some embodiments, DA-Rep comprises a nucleic acid sequence that is modified to comprise a premature stop codons at amino acid positions corresponding to Q67, Q262 and W319 of SEQ ID NO: 97. In some embodiments, DA-Rep comprises a nucleic acid sequence comprising least 80% (e.g., at least 80%, at least 85%, at least 90%, at least 95%, or at least 99%) identity to any one of SEQ ID NO: 53-55. In some embodiments, DA-Rep comprises a nucleic acid sequence comprising any one of SEQ ID NO: 53-55. In some embodiments, DA-Rep comprises a nucleic acid sequence consisting of any one of SEQ ID NO: 53-55. In some embodiments, the AAV production component comprises a nucleic acid sequence encoding for DA-E2A operably linked to a nucleic acid sequence of a promoter (constitutive or inducible, as described herein). In some embodiments, the DA-E2A nucleic acid sequence is operably linked to a E2A promoter. The term “DA-E2A” refers to a nucleic acid sequence encoding a polypeptide comprising at least 80% (e.g., at least 80%, at least 85%, at least 90%, at least 95%, or at least 99%) identity to the amino acid sequence of SEQ ID NO: 10 comprising at least one mutation that decreases the activity of E2A (as described above). In some embodiments, at least one codon of the nucleic acid sequence is a premature stop codon (e.g., TAG, TAA, and TGA). In some embodiments, DA-E2A comprises a nucleic acid sequence encoding a polypeptide comprising an amino acid sequence of SEQ ID NO: 10 that is modified to comprise a premature stop codon at amino acid position W181 or W324. In some embodiments, DA-E2A comprises a nucleic acid sequence encoding a polypeptide comprising an amino acid sequence of SEQ ID NO: 10 that is modified to comprise premature stop codons at amino acid positions W181 and W324. In some embodiments, DA-E2A comprises a nucleic acid sequence encoding a polypeptide comprising at least 80% (e.g., at least 80%, at least 85%, at least 90%, at least 95%, or at least 99%) identity to any one of SEQ ID NO: 39-40. In some embodiments, DA-E2A comprises a nucleic acid sequence encoding a polypeptide comprising an amino acid sequence of any one of SEQ ID NO: 39-40. In some embodiments, DA-E2A comprises a nucleic acid sequence encoding a polypeptide consisting of an amino acid sequence of any one of SEQ ID NO: 39-40. In some embodiments, DA-E2A comprises a nucleic acid sequence comprising at least 80% (e.g., at least 80%, at least 85%, at least 90%, at least 95%, or at least 99%) identity to any one of SEQ ID NO: 105-106. In some embodiments, DA-E2A comprises a nucleic acid sequence of any one of SEQ ID NO: 105-106. In some embodiments, DA-E2A comprises a nucleic acid sequence consisting of any one of SEQ ID NO: 105-106.

In some embodiments, the AAV production component comprises a nucleic acid sequence encoding for DA-E4ORF6 operably linked to a nucleic acid sequence of a promoter (constitutive or inducible, as described herein). In some embodiments, the DA-E4ORF6 nucleic acid sequence is operably linked to an E4 promoter. The term “DA-E4ORF6” refers to a nucleic acid sequence encoding a polypeptide comprising at least 80% (e.g., at least 80%, at least 85%, at least 90%, at least 95%, or at least 99%) identity to the amino acid sequence of SEQ ID NO: 11 comprising at least one mutation that decreases the activity of E4ORF6 (as described above). In some embodiments, at least one codon of the nucleic acid sequence is a premature stop codon (e.g., TAG, TAA, and TGA). In some embodiments, DA-E4ORF6 comprises a nucleic acid sequence encoding a polypeptide comprising an amino acid sequence of SEQ ID NO: 11 that is modified to comprise a premature stop codon at amino acid position W77 or W192. In some embodiments, DA-E4ORF6 comprises a nucleic acid sequence encoding a polypeptide comprising an amino acid sequence of SEQ ID NO: 11 that is modified to comprise premature stop codons at amino acid positions W77 and W192. In some embodiments, DA-E4ORF6 comprises a nucleic acid sequence of SEQ ID NO: 12 that is modified to comprise a premature stop codon at amino acid positions corresponding to W77 or W192 of SEQ ID NO: 11. In some embodiments, DA-E4ORF6 comprises a nucleic acid sequence of SEQ ID NO: 12 that is modified to comprise premature stop codons at amino acid positions corresponding to W77 and W192 of SEQ ID NO: 11. In some embodiments, DA-E4ORF6 comprises a nucleic acid sequence encoding a polypeptide comprising at least 80% (e.g., at least 80%, at least 85%, at least 90%, at least 95%, or at least 99%) identity to the amino acid sequence of any one of SEQ ID NO: 41-42. In some embodiments, DA-E4ORF6 comprises a nucleic acid sequence encoding a polypeptide comprising an amino acid sequence of SEQ ID NO: 41-42. In some embodiments, DA-E4ORF6 comprises a nucleic acid sequence encoding a polypeptide consisting of an amino acid sequence of SEQ ID NO: 41-42. In some embodiments, DA-E4ORF6 comprises a nucleic acid sequence comprising at least 80% (e.g., at least 80%, at least 85%, at least 90%, at least 95%, or at least 99%) identity to any one of SEQ ID NO: 107-108. In some embodiments, DA-E4ORF6 comprises a nucleic acid sequence of any one of SEQ ID NO: 107-108. In some embodiments, DA-E4ORF6 comprises a nucleic acid sequence consisting of any one of SEQ ID NO: 107-108.

In some embodiments, the AAV production component comprises a nucleic acid sequence encoding for DA-L4 100K operably linked to a nucleic acid sequence of a promoter (constitutive or inducible, as described herein). In some embodiments, the DA-L4 100K nucleic acid sequence encoding an amino acid sequence is operably linked to a p19 promoter. The term “DA-L4 100K” refers to a nucleic acid sequence encoding a polypeptide comprising at least 80% (e.g., at least 80%, at least 85%, at least 90%, at least 95%, or at least 99%) identity to the amino acid sequence of SEQ ID NO: 112 comprising at least one mutation that decreases the activity of L4 100K (as described above). In some embodiments, at least one codon of the nucleic acid sequence is a premature stop codon (e.g., TAG, TAA, and TGA). In some embodiments, DA-L4 100K comprises a nucleic acid sequence encoding a polypeptide comprising SEQ ID NO: 112 that is modified to comprise a premature stop codon at amino acid position corresponding to W435 of SEQ ID NO: 97. In some embodiments, DA-L4 100K comprises a nucleic acid sequence encoding a polypeptide comprising at least 80% (e.g., at least 80%, at least 85%, at least 90%, at least 95%, or at least 99%) identity to the amino acid sequence of any one of SEQ ID NO: 98. In some embodiments, DA-L4 100K comprises a nucleic acid sequence encoding a polypeptide comprising the amino acid sequence of any one of SEQ ID NO: 98. In some embodiments, DA-L4 100K comprises a nucleic acid sequence encoding a polypeptide consisting of the amino acid sequence any one of SEQ ID NO: 98.

In some embodiments, DA-VARNA comprises a nucleic acid sequence comprising at least 80% (e.g., at least 80%, at least 85%, at least 90%, at least 95%, or at least 99%) identity to the amino acid sequence of SEQ ID NO: 13 further comprising a mutation that renders VARNA inactive. In some embodiments, DA-VARNA comprises a nucleic acid sequence encoding a polypeptide comprising an amino acid sequence of SEQ ID NO: 13 further comprising a mutation that renders VARNA inactive. In some embodiments, DA-VARNA consists of comprises a nucleic acid sequence encoding a polypeptide consisting of an amino acid sequence of SEQ ID NO: 13 further comprising a mutation that renders VARNA inactive.

In some embodiments, the AAV production component comprises a nucleic acid sequence encoding for DA-VP1 operably linked to a nucleic acid sequence of a promoter (constitutive or inducible, as described herein). In some embodiments, the DA-VP1 nucleic acid sequence is operably linked to a p40 promoter. The term “DA-VP1” refers to a nucleic acid sequence encoding a polypeptide comprising at least 80% (e.g., at least 80%, at least 85%, at least 90%, at least 95%, or at least 99%) identity to the amino acid sequence of SEQ ID NO: 14 comprising at least one mutation that decreases the activity of VP1 (as described above). In some embodiments, the nucleic acid sequence encoding DA-VP1 comprises a mutation at a position corresponding to amino acid position P8 of VP1 (SEQ ID NO: 14). In some embodiments, the nucleic acid sequence encoding DA-VP1 comprises a ccA (P)→ccG (P) mutation at a position corresponding to amino acid position P8 of VP1 (SEQ ID NO: 14). In some embodiments, at least one codon of the nucleic acid sequence is a premature stop codon (e.g., TAG, TAA, and TGA). In some embodiments, DA-VP1 comprises a nucleic acid sequence encoding a polypeptide comprising an amino acid sequence of SEQ ID NO: 14 that is modified to comprise a premature stop codon at amino acid position corresponding to W304 or Q598 of SEQ ID NO: 14. In some embodiments, DA-VP1 comprises a nucleic acid sequence encoding a polypeptide comprising an amino acid sequence of SEQ ID NO: 14 that is modified to comprise premature stop codons at amino acid positions W304 or Q598. In some embodiments, DA-VP1 comprises a nucleic acid sequence encoding a polypeptide comprising at least 80% (e.g., at least 80%, at least 85%, at least 90%, at least 95%, or at least 99%) identity to SEQ ID NO: 99 or 102. In some embodiments, DA-VP1 comprises a nucleic acid sequence encoding a polypeptide comprising an amino acid sequence of SEQ ID NO: 99 or 102. In some embodiments, DA-VP1 comprises a nucleic acid sequence encoding a polypeptide consisting of an amino acid sequence of SEQ ID NO: 99 or 102.

In some embodiments, the AAV production component comprises a nucleic acid sequence encoding for DA-VP2 operably linked to a nucleic acid sequence of a promoter (constitutive or inducible, as described herein). In some embodiments, the DA-VP2 nucleic acid sequence is operably linked to a p40 promoter. The term “DA-VP2” refers to a nucleic acid sequence encoding a polypeptide comprising at least 80% (e.g., at least 80%, at least 85%, at least 90%, at least 95%, or at least 99%) identity to the amino acid sequence of SEQ ID NO: 15 comprising at least one mutation that decreases the activity of VP2 (as described above). In some embodiments, at least one codon of the nucleic acid sequence is a premature stop codon (e.g., TAG, TAA, and TGA). In some embodiments, DA-VP2 comprises a nucleic acid sequence encoding a polypeptide comprising an amino acid sequence of SEQ ID NO: 15 that is modified to comprise a premature stop codon at amino acid position corresponding to W304 or Q598 of SEQ ID NO: 14. In some embodiments, DA-VP2 comprises a nucleic acid sequence encoding a polypeptide comprising an amino acid sequence of SEQ ID NO: 15 that is modified to comprise premature stop codons at amino acid positions W304 or Q598. In some embodiments, DA-VP2 comprises a nucleic acid sequence encoding a polypeptide comprising at least 80% (e.g., at least 80%, at least 85%, at least 90%, at least 95%, or at least 99%) identity to SEQ ID NO: 100 or 103. In some embodiments, DA-VP2 comprises a nucleic acid sequence encoding a polypeptide comprising an amino acid sequence of SEQ ID NO: 100 or 103. In some embodiments, DA-VP2 comprises a nucleic acid sequence encoding a polypeptide consisting of an amino acid sequence of SEQ ID NO: 100 or 103.

In some embodiments, the AAV production component comprises a nucleic acid sequence encoding for DA-VP3 operably linked to a nucleic acid sequence of a promoter (constitutive or inducible, as described herein). In some embodiments, the DA-VP3 nucleic acid sequence is operably linked to a p40 promoter. The term “DA-VP3” refers to a nucleic acid sequence encoding a polypeptide comprising at least 80% (e.g., at least 80%, at least 85%, at least 90%, at least 95%, or at least 99%) identity to the amino acid sequence of SEQ ID NO: 16 comprising at least one mutation that decreases the activity of VP3 (as described above). In some embodiments, the nucleic acid sequence encoding DA-VP3 comprises one or more mutations (e.g. 1, 2, 3, 4, 5, 6, 7, 8, 9, 10 or more) to arginine or lysine. In some embodiments, the nucleic acid sequence encoding DA-VP3 comprises one or more mutations (e.g. 1, 2, 3, 4, 5, 6, 7, 8, 9, 10 or more) from aspartic acid, glutamic acid or glycine to arginine or lysine as described in Ogden et al. Science. 2019 Nov. 29; 366 (6469): 1139-1143, which is incorporated by reference in its entirety. In some embodiments, at least one codon of the nucleic acid sequence is a premature stop codon (e.g., TAG, TAA, and TGA). In some embodiments, DA-VP3 comprises a nucleic acid sequence encoding a polypeptide comprising an amino acid sequence of SEQ ID NO: 16 that is modified to comprise a premature stop codon at amino acid positions corresponding to W304 or Q598 of SEQ ID NO: 14. In some embodiments, DA-VP3 comprises a nucleic acid sequence encoding a polypeptide comprising an amino acid sequence of SEQ ID NO: 16 that is modified to comprise a premature stop codons at amino acid position corresponding to W304 and Q598 of SEQ ID NO: 14. In some embodiments, DA-VP3 comprises a nucleic acid sequence encoding a polypeptide comprising at least 80% (e.g., at least 80%, at least 85%, at least 90%, at least 95%, or at least 99%) identity to SEQ ID NO: 101 or 104. In some embodiments, DA-VP3 comprises a nucleic acid sequence encoding a polypeptide comprising an amino acid sequence of SEQ ID NO: 101 or 104. In some embodiments, DA-VP3 comprises a nucleic acid sequence encoding a polypeptide consisting of an amino acid sequence of SEQ ID NO: 101 or 104.

In some embodiments, the AAV production component comprises a nucleic acid sequence encoding for DA-VP operably linked to a nucleic acid sequence of a promoter (constitutive or inducible, as described herein). In some embodiments, the DA-VP nucleic acid sequence is operably linked to a p40 promoter. The term “DA-VP” refers to a nucleic acid sequence comprising at least 80% (e.g., at least 80%, at least 85%, at least 90%, at least 95%, or at least 99%) identity SEQ ID NO: 116 comprising at least one mutation that decreases the activity of VP (as described above). In some embodiments, DA-VP comprises one or more non-silent mutations that are detrimental to the activity of VP as described in Ogden et al. Science. 2019 Nov. 29; 366 (6469): 1139-1143, which is incorporated by reference herein in its entirety. In some embodiments, DA-VP comprises one or more (e.g. 1, 2, 3, 4, 5, 6, 7, 8, 9, 10 or more) amino acid mutations to methionine within residues 1-200 of VP (SEQ ID NO: 14). In some embodiments, DA-VP comprises one or more (e.g. 1, 2, 3, 4, 5, 6, 7, 8, 9, 10 or more) isoleucine to methionine mutations within residues 1-200 of VP (SEQ ID NO: 14). In some embodiments, at least one codon of the nucleic acid sequence is a premature stop codon (e.g., TAG, TAA, and TGA). In some embodiments, DA-VP comprises a nucleic acid sequence of SEQ ID NO: 116 that is modified to comprise a premature stop codon at amino acid position corresponding to W304 or Q598 of SEQ ID NO: 14. In some embodiments, DA-VP comprises a nucleic acid sequence of SEQ ID NO: 116 that is modified to comprise premature stop codons at amino acid positions W304 or Q598 of SEQ ID NO: 14. In some embodiments, DA-VP comprises a nucleic acid sequence encoding a polypeptide comprising at least 80% (e.g., at least 80%, at least 85%, at least 90%, at least 95%, or at least 99%) identity to SEQ ID NO: 110 or 111. In some embodiments, DA-VP comprises a nucleic acid sequence encoding a polypeptide comprising an amino acid sequence of SEQ ID NO: 110 or 111. In some embodiments, DA-VP comprises a nucleic acid sequence encoding a polypeptide consisting of an amino acid sequence of SEQ ID NO: 110 or 111.

### C. Base Editor Single Guide RNAs

In some embodiments, the activity control component comprises one or more single guide RNAs. As described herein, the term “single guide RNA(s) or sgRNA” refer to RNA sequences capable of binding to and directing a Base Editor to a target DNA or RNA sequence (e.g. DNA or RNA encoding DA-Rep52). Single guide RNAs comprise a nucleic acid sequence referred to as a spacer or protospacer. In some embodiments, the spacer or protospacer is about 15 to 50 base pairs in length and is sufficiently complementary to the target sequence (e.g. DNA or RNA of DA-Rep52) to direct the Base Editor to the target sequence. In some embodiments, the spacer or protospacer is complementary to a target sequence that is adjacent to a protospacer adjacent motif (PAM). In some embodiments, one or more sgRNAs are sufficiently complementary to the mutation(s) within the nucleic acid sequence encoding a gene required for AAV production to direct a Base Editor to the mutation(s) for base editing of the mutation(s) to revert the mutated sequence to the wildtype sequence. In some embodiments, one or more sgRNAs are sufficiently complementary to the premature stop codon(s) within the nucleic acid sequence encoding a gene required for AAV production to direct a Base Editor to the premature stop codon(s) for base editing of the premature stop codon(s). In some embodiments, the DNA nucleic acid sequence encoding any sgRNA described herein is operably linked to a promoter (constitutive or inducible, as described herein). In some embodiments, the DNA nucleic acid sequence encoding any sgRNA described herein is operably linked to a U6 promoter.

In some embodiments, the AAV production component comprises a nucleic acid sequence encoding DA-E2A and the activity control component comprises a nucleic acid sequence encoding each of one or more sgRNAs that are sufficiently complementary to the mutation(s) within the nucleic acid sequence encoding DA-E2A to direct a Base Editor to the mutation(s) for base editing of the mutation(s) to revert the mutated sequence to the wildtype sequence as described above. In some embodiments, the AAV production component comprises a nucleic acid sequence encoding DA-E2A and the activity control component comprises a nucleic acid sequence encoding each of one or more sgRNAs that are sufficiently complementary to the premature stop codon(s) within the nucleic acid sequence encoding DA-E2A to direct a Base Editor to the premature stop codon(s) for base editing of the premature stop codon(s) to canonical codon(s) as described above. In some embodiments, the nucleic acid sequence encoding DA-E2A comprises a premature stop codon at a position corresponding to amino acid residue W181 in SEQ ID NO: 10 and the one single guide RNA comprises a spacer sufficiently complementary to a premature stop codon at a position corresponding to W181 in SEQ ID NO: 10 to direct a Base Editor to edit the premature stop codon to a tryptophan codon. In some embodiments, the nucleic acid sequence encoding DA-E2A comprises a premature stop codon at a position corresponding to amino acid W324 in SEQ ID NO: 10 and the one single guide RNA comprises a spacer sufficiently complementary to a premature stop codon at a position corresponding to W324 in SEQ ID NO: 10 to direct a Base Editor to edit the premature stop codon to a tryptophan codon. In some embodiments, the nucleic acid sequence encoding DA-E2A comprises premature stop codons at positions corresponding to amino acid residues W181 and W324 in SEQ ID NO: 10, and the activity control component comprises a first single guide RNA comprising a spacer that is sufficiently complementary to the premature stop codon at a position corresponding to W181 in SEQ ID NO: 10 to direct a Base Editor to edit the premature stop codon to a tryptophan codon, and a second single guide RNA comprising a spacer that is sufficiently complementary to the premature stop codon at a position corresponding to W324 in SEQ ID NO: 10 to direct the Base Editor to edit the premature stop codon to a tryptophan codon. In some embodiments, the one or more single guide RNAs each comprise a nucleic acid sequence comprising at least 80% (e.g., at least 80%, at least 85%, at least 90%, at least 95%, or at least 99%) identity to any one of SEQ ID NO: 56-57, 66-67, and 74-75. In some embodiments, the one or more single guide RNAs each comprise a nucleic acid sequence comprising any one of SEQ ID NO: 56-57, 66-67, and 74-75. In some embodiments, the one or more single guide RNAs each comprise a spacer that consists of any one of SEQ ID NO: 56-57, 66-67, and 74-75.

In some embodiments, the AAV production component comprises a nucleic acid sequence encoding DA-E4ORF6 and the activity control component comprises a nucleic acid sequence encoding each of one or more sgRNAs that are sufficiently complementary to the mutation(s) within the nucleic acid sequence encoding DA-E4ORF6 to direct a Base Editor to the mutation(s) for base editing of the mutation(s) to revert the mutated sequence to the wildtype sequence as described above. In some embodiments, the AAV production component comprises a nucleic acid sequence encoding DA-E4ORF6 and the activity control component comprises a nucleic acid sequence encoding each of one or more single guide RNAs that are sufficiently complementary to the premature stop codon(s) within the nucleic acid sequence encoding DA-E4ORF6 to direct a Base Editor to the premature stop codon(s) for base editing of the premature stop codon(s) to canonical codon(s) as described above. In some embodiments, the nucleic acid sequence encoding DA-E4ORF6 comprises a premature stop codon at a position corresponding to amino acid residue W77 in SEQ ID NO: 11 and the one sgRNA comprises a spacer sufficiently complementary to a premature stop codon at a position corresponding to amino acid residue W77 in SEQ ID NO: 11 to direct the Base Editor to edit the premature stop codon to a tryptophan codon. In some embodiments, the nucleic acid sequence encoding DA-E4ORF6 comprises a premature stop codon at a position corresponding to amino acid residue W192 in SEQ ID NO: 11 and the one single guide RNA comprises a spacer sufficiently complementary to a premature stop codon at a position corresponding to amino acid residue W192 in SEQ ID NO: 11 to direct a Base Editor to edit the premature stop codon tryptophan codon. In some embodiments, the nucleic acid sequence encoding DA-E4ORF6 comprises premature stop codons at positions corresponding to amino acid residues W77 and W192 in SEQ ID NO: 11, and the activity control component comprises a first single guide RNA comprising a spacer that is sufficiently complementary to the premature stop codon at a position corresponding to W77 in SEQ ID NO: 11 to direct a Base Editor to edit the premature stop codon to a tryptophan codon and a second single guide RNA comprising a spacer that is sufficiently complementary to the premature stop codon at a position corresponding to W192 in SEQ ID NO: 11 to direct a Base Editor to edit the stop codon to a tryptophan codon. In some embodiments, the one or more single guide RNAs each comprise a nucleic acid sequence comprising at least 80% (e.g., at least 80%, at least 85%, at least 90%, at least 95%, or at least 99%) identity to any one of SEQ ID NO: 58-59, 68-69, and 76-77. In some embodiments, the one or more single guide RNAs each comprise a nucleic acid sequence comprising any one of SEQ ID NO: 58-59, 68-69, and 76-77. In some embodiments, the one or more single guide RNAs each comprise a spacer that consists of any one of SEQ ID NO: 58-59, 68-69, and 76-77.

In some embodiments, the AAV production component comprises a nucleic acid sequence encoding DA-Rep52 or DA-Rep40 and the activity control component comprises a nucleic acid sequence encoding each of one or more sgRNAs that are sufficiently complementary to the mutation(s) within the nucleic acid sequence encoding DA-Rep52 or DA-Rep40 to direct a Base Editor to the mutation(s) for base editing of the mutation(s) to revert the mutated sequence to the wildtype sequence as described above. In some embodiments, the AAV production component comprises a nucleic acid sequence encoding for DA-Rep52 or DA-Rep40 and the activity control component comprises a nucleic acid sequence encoding each of one or more single guide RNAs that are sufficiently complementary to the premature stop codon(s) within the nucleic acid sequence encoding DA-Rep52 or DA-Rep40 to direct the Base Editor to the premature stop codon(s) for base editing of the premature stop codon(s) to canonical codon(s) as described above. In some embodiments, the nucleic acid sequence encoding DA-Rep52 or DA-Rep40 comprises a premature stop codon at a position corresponding to amino acid residue Q262 in SEQ ID NO: 97 and the one single guide RNA comprises a spacer sufficiently complementary to the premature stop codon at a position corresponding to Q262 in SEQ ID NO: 97 to direct the Base Editor to edit the premature stop codon. In some embodiments, the nucleic acid sequence encoding DA-Rep52 or DA-Rep40 comprises a premature stop codon at a position corresponding to amino acid residue W319 in SEQ ID NO: 97 and the one single guide RNA comprises a spacer sufficiently complementary to a premature stop codon at a position corresponding to W319 in SEQ ID NO: 97 to direct the Base Editor to edit the premature stop codon. In some embodiments, the nucleic acid sequence encoding DA-Rep52 or DA-Rep40 comprises premature stop codons at positions corresponding to amino acid residues Q262 and W319 in SEQ ID NO: 97, and the activity control component comprises a first single guide RNA comprising a spacer that is sufficiently complementary to the premature stop codon at a position corresponding to Q262 in SEQ ID NO: 97 to direct the Base Editor to edit the premature stop codon to a glutamine codon and a second single guide RNA comprising a spacer that is sufficiently complementary to the premature stop codon at a position corresponding to W319 in SEQ ID NO: 97 to direct the Base Editor to edit the premature stop codon to a tryptophan codon. In some embodiments, the one or more single guide RNAs each comprise a nucleic acid sequence that is at least 80% (e.g., at least 80%, at least 85%, at least 90%, at least 95%, or at least 99%) identical to any one of SEQ ID NO: 64-65, 73, and 81. In some embodiments, the one or more single guide RNAs each comprise a nucleic acid sequence of any one of SEQ ID NO: 64-65, 73, and 81. In some embodiments, the one or more single guide RNAs each comprise a spacer that consists of any one of SEQ ID NO: 64-65, 73, and 81.

In some embodiments, the AAV production component comprises nucleic acid sequence encoding DA-Rep52, DA-Rep40 and/or DA-Rep that is modified to comprise a mutation at amino acid position corresponding to M225 (e.g. M225G) of SEQ ID NO: 97, and the activity control component comprises a nucleic acid sequence encoding a sgRNAs that is sufficiently complementary to the mutation within the nucleic acid sequence encoding DA-Rep52, DA-Rep40 and/or DA-Rep to direct a Base Editor to the mutation(s) for base editing of the mutation(s) to revert the mutated sequence to the wildtype sequence as described above.

In some embodiments, the AAV production component comprises nucleic acid sequence encoding DA-Rep52, DA-Rep40, DA-Rep78, DA-Rep68 and/or DA-Rep that is modified to comprise a mutation at amino acid position corresponding to R529 (e.g. AgG→CgC) of SEQ ID NO: 97, and the activity control component comprises a nucleic acid sequence encoding a sgRNAs that is sufficiently complementary to the mutation within the nucleic acid sequence encoding DA-Rep52, DA-Rep40, DA-Rep78, DA-Rep68 and/or DA-Rep to direct a Base Editor to the mutation(s) for base editing of the mutation(s) to revert the mutated sequence to the wildtype sequence as described above.

In some embodiments, the AAV production component comprises a nucleic acid sequence encoding DA-Rep78, DA-Rep68 and/or DA-Rep comprising one or more (e.g. 1, 2, 3, 4, 5, 6, 7, 8, 9, 10, 11, 12, 13, 14, 15, 16, 17 or more) missense or premature stop codon mutations at or between positions corresponding to one or more of amino acid positions 56-57, 58-59, 61-62, 73-74, 76-77, 87-88, 113-114, 33-134, 164-165, 217-218, 226-227, 256-257, 259-260, 346-347, 400-401, 409-410, and 455-456 that decrease DA-Rep78, DA-Rep68 and/or DA-Rep activity, and the activity control component comprises a nucleic acid sequence encoding a sgRNAs that is sufficiently complementary to the one or more mutations within the nucleic acid sequence encoding DA-Rep78, DA-Rep68 and/or DA-Rep to direct a Base Editor to the mutation(s) for base editing of the mutation(s) to revert the mutated sequence to the wildtype sequence as described above.

In some embodiments, the AAV production component comprises a nucleic acid sequence encoding DA-Rep52 and/or DA-Rep40 comprising one or more (e.g. 1, 2, 3, 4, 5, 6, 7 or more) missense or premature stop codon mutations at or between positions corresponding to one or more of amino acid positions 226-227, 256-257 259-260, 346-347, 400-401, 409-410, and 455-456 that decrease DA-Rep78, DA-Rep68 and/or DA-Rep activity, and the activity control component comprises a nucleic acid sequence encoding a sgRNAs that is sufficiently complementary to the one or more mutation within the nucleic acid sequence encoding DA-Rep52 and/or DA-Rep40 to direct a Base Editor to the mutation(s) for base editing of the mutation(s) to revert the mutated sequence to the wildtype sequence as described above.

In some embodiments, the AAV production component comprises a nucleic acid sequence encoding DA-Rep78, DA-Rep68 or DA-Rep and the activity control component comprises a nucleic acid sequence encoding each of one or more sgRNAs that are sufficiently complementary to the mutation(s) within the nucleic acid sequence encoding DA-Rep78, DA-Rep68 or DA-Rep to direct a Base Editor to the mutation(s) for base editing of the mutation(s) to revert the mutated sequence to the wildtype sequence as described above. In some embodiments, the AAV production component comprises a nucleic acid sequence encoding DA-Rep78, DA-Rep68 or DA-Rep and the activity control component comprises a nucleic acid sequence encoding for each of one or more single guide RNAs that are sufficiently complementary to the premature stop codon(s) within the DA-Rep78, DA-Rep68 or DA-Rep to direct a Base Editor to the premature stop codon(s) for base editing of the premature stop codon(s) to canonical codon(s) as described above. In some embodiments, the nucleic acid sequence encoding DA-Rep78, DA-Rep68 or DA-Rep comprises a premature stop codon at a position corresponding to amino acid residue W67 in SEQ ID NO: 97 and the one single guide RNAs comprises a spacer sufficiently complementary to a premature stop codon at a position corresponding to W67 in SEQ ID NO: 97 to direct a Base Editor to edit the premature stop codon to a tryptophan codon. In some embodiments, the nucleic acid sequence encoding DA-Rep78, DA-Rep68 or DA-Rep comprise a premature stop codon at a position corresponding to amino acid residue Q262 in SEQ ID NO: 97 and the one single guide RNA comprises a spacer sufficiently complementary to a premature stop codon at a position corresponding to Q262 in SEQ ID NO: 97 to direct the Base Editor to edit the premature stop codon to a glutamine codon. In some embodiments, the nucleic acid sequence encoding DA-Rep78, DA-Rep68 or DA-Rep comprises a premature stop codon at a position corresponding to amino acid W319 in SEQ ID NO: 97 and the one single guide RNA comprises a spacer sufficiently complementary to a premature stop codon at a position corresponding to W319 in SEQ ID NO: 97 to direct a Base Editor to edit the premature stop codon to a tryptophan codon. In some embodiments, the nucleic acid sequence encoding DA-Rep78, DA-Rep68 or DA-Rep comprises a premature stop codons at a two or more positions corresponding to amino acid residues W67, Q262 and W319 in SEQ ID NO: 97, and the activity control component comprises two or more single guide RNAs with spacer regions sufficiently complementary to the two or more premature stop codons corresponding to amino acid residues W67, Q262 and W319 in SEQ ID NO: 97 to direct a Base Editor the edit the premature stop codons back to the original wildtype codon. In some embodiments, the one or more single guide RNAs each comprise a nucleic acid sequence that is at least 80% (e.g., at least 80%, at least 85%, at least 90%, at least 95%, or at least 99%) identical to any one of SEQ ID NO: 63-65, 72-73, and 80-81. In some embodiments, the one or more single guide RNAs each comprise a nucleic acid sequence of any one of SEQ ID NO: 63-65, 72-73, and 80-81. In some embodiments, the one or more single guide RNAs each comprise a spacer that consists of any one of SEQ ID NO: 63-65, 72-73, and 80-81.

In some embodiments, the AAV production component comprises a nucleic acid sequence encoding DA-VP1, DA-VP2, DA-VP3, and/or DA-VP and the activity control component comprises a nucleic acid sequence encoding each of one or more sgRNAs that are sufficiently complementary to the mutation(s) within the nucleic acid sequence encoding DA-VP1, DA-VP2, DA-VP3, and/or DA-VP to direct a Base Editor to the mutation(s) for base editing of the mutation(s) to revert the mutated sequence to the wildtype sequence as described above. In some embodiments, the AAV production component comprises a nucleic acid sequence encoding DA-VP1, DA-VP2, DA-VP3, and/or DA-VP and the activity control component comprises a nucleic acid sequence encoding each of one or more single guide RNAs that are sufficiently complementary to the premature stop codon(s) within the nucleic acid sequence encoding DA-VP1, DA-VP2, DA-VP3, and/or DA-VP to direct a Base Editor to the premature stop codon(s) for base editing of the premature stop codon(s) to canonical codon(s) as described above. In some embodiments, the AAV production component comprises a nucleic acid sequence encoding DA-VP1 comprising a mutation at a position corresponding to amino acid position P8 of VP1 (SEQ ID NO: 14) (e.g. ccA (P)→ccG (P)) and the activity control component comprises a nucleic acid sequence encoding a single guide RNA that is sufficiently complementary to mutation at position P8 of DA-VP1 to direct a Base Editor to the mutation for base editing. In some embodiments, the AAV production component comprises a nucleic acid sequence encoding DA-VP comprising one or more (e.g. 1, 2, 3, 4, 5, 6, 7, 8, 9, 10 or more) amino acid mutation to methionine (e.g. isoleucine to methionine mutations) within residues 1-200 of VP (SEQ ID NO: 14) and the activity control component comprises a nucleic acid sequence encoding one or more single guide RNAs that are sufficiently complementary to the one or more mutations to methionine to direct a Base Editor to the mutation(s) for base editing. In some embodiments, DA-VP comprises one or more (e.g. 1, 2, 3, 4, 5, 6, 7, 8, 9, 10 or more) isoleucine to methionine mutations within residues 1-200 of VP (SEQ ID NO: 14). In some embodiments, the AAV production component comprises a nucleic acid sequence encoding DA-VP3 comprising one or more mutations to arginine or lysine (e.g. 1, 2, 3, 4, 5, 6, 7, 8, 9, 10 or more mutations to arginine or lysine) (e.g. from aspartic acid, glutamic acid or glycine to arginine or lysine) and the activity control component comprises a nucleic acid sequence encoding one or more single guide RNAs that are sufficiently complementary to the one or more mutations to arginine or lysine to direct a Base Editor to the one or more mutations for base editing. In some embodiments, the nucleic acid sequence encoding DA-VP1, DA-VP2, DA-VP3, and/or DA-VP comprises a premature stop codon at a position corresponding to amino acid residue W304 in SEQ ID NO: 14 and the one single guide RNA comprises a spacer sufficiently complementary to a premature stop codon at a position corresponding to W304 in SEQ ID NO: 14 to direct the Base Editor to edit the premature stop codon to a tryptophan codon. In some embodiments, the nucleic acid sequence encoding DA-VP1 comprises a premature stop codon at a position corresponding to amino acid Q598 in SEQ ID NO: 14 and the one single guide RNA comprises a spacer sufficiently complementary to a premature stop codon at a position corresponding to Q598 in SEQ ID NO: 14 to direct a Base Editor to edit the premature stop codon to a glutamine stop codon. In some embodiments, the nucleic acid sequence encoding DA-VP1 comprises a premature stop codon at a positions corresponding to amino acid W304 and Q598 in SEQ ID NO: 14, and the activity control component comprises a first single guide RNA comprising a spacer that is sufficiently complementary to the premature stop codon at a position corresponding to W304 in SEQ ID NO: 14 to direct a Base Editor to edit the premature stop codon to a tryptophan codon, and a second single guide RNA comprising a spacer that is sufficiently complementary to the premature stop codon at a position corresponding to Q598 in SEQ ID NO: 14 to direct a Base Editor to edit the premature stop codon to a glutamine codon. In some embodiments, the one or more single guide RNAs each comprise a nucleic acid sequence that is at least 80% (e.g., at least 80%, at least 85%, at least 90%, at least 95%, or at least 99%) identical to any one of SEQ ID NO: 61-62, 71, and 79. In some embodiments, the one or more single guide RNAs each comprise a nucleic acid sequence of any one of SEQ ID NO: 61-62, 71, and 79. In some embodiments, the one or more single guide RNAs each comprise a spacer that consists of any one of SEQ ID NO: 61-62, 71, and 79.

In some embodiments, the AAV production component comprises a nucleic acid sequence encoding DA-L4 100K and the activity control component comprises a nucleic acid sequence encoding each of one or more sgRNAs that are sufficiently complementary to the mutation(s) within the nucleic acid sequence encoding DA-L4 100K to direct a Base Editor to the mutation(s) for base editing of the mutation(s) to revert the mutated sequence to the wildtype sequence as described above. In some embodiments, the AAV production component comprises a nucleic acid sequence encoding DA-L4 100K and the stop codon component comprises a nucleic acid sequence encoding each of one or more single guide RNAs that are sufficiently complementary to the premature stop codon(s) within the nucleic acid sequence encoding DA-L4 100K to direct a Base Editor to the premature stop codon(s) for base editing of the premature stop codon(s) to canonical codon(s) as described above. In some embodiments, the nucleic acid sequence encoding DA-L4 100K comprises a premature stop codon at a position corresponding to amino acid residue 435 in SEQ ID NO: 112 and the one single guide RNA comprises a spacer sufficiently complementary to a premature stop codon at a position corresponding to W435 in SEQ ID NO: 112 to direct the Base Editor to edit the premature stop codon to a tryptophan codon. In some embodiments, the one or more single guide RNAs each comprise a nucleic acid sequence that is at least 80% (e.g., at least 80%, at least 85%, at least 90%, at least 95%, or at least 99%) identical to any one of SEQ ID NO: 60, 70, and 78. In some embodiments, the one or more single guide RNAs each comprise a nucleic acid sequence of any one of SEQ ID NO: 60, 70, and 78. In some embodiments, the one or more single guide RNAs each comprise a spacer that consists of any one of SEQ ID NO: 60, 70, and 78.

### VI. Exemplary Embodiments of Engineered Cells for AAV Production Comprising a Base Editor

In some aspects, the AAV production system further comprises an engineered cell for AAV production. In some embodiments, the engineered cell comprises the one or more polynucleic acids collectively comprising: (a) the AAV production component and (b) an activity control component comprising a Base Editor capable of replacing earlier stop codon mutations with canonical codons.

In some embodiments, an engineered cell comprises a nucleic acid sequence encoding for a Base Editor as described herein (e.g. an ABE or a CBE), a nucleic acid sequence encoding for any one of Rep52, DA-Rep52, Rep40, or DA-Rep40 as described herein, a nucleic acid sequence encoding for any one of Rep78, DA-Rep78, Rep68, or DA-Rep68 as described herein, a nucleic acid sequence encoding for any one of E2A or DA-E2A as described herein and a nucleic acid sequence encoding for any one of E4Orf6 or DA-E4Orf6 as described herein, further comprises nucleic acid sequences encoding for each of L4 100K or DA-L4 100K; VARNA; VP1 or DA-VP1; VP2 or DA-VP2; VP3 or DA-VP3; and AAP as described herein, wherein the engineered cell comprises at least one of DA-Rep52, DA-Rep40, DA-Rep78, DA-Rep68, DA-E4Orf6, DA-L4 100K DA-VP1, DA-VP2, and DA-VP3, and wherein the cell comprises one or more single guide RNAs as described herein each comprise spacer that is sufficiently complementary to the at least one of DA-Rep52, DA-Rep40, DA-Rep78, DA-Rep68, DA-E4Orf6 DA-L4 100K DA-VP1, DA-VP2, and DA-VP3 to direct the Base Editor to edit the premature stop codon to a canonical codon (e.g. the original wildtype codon).

### A. The First Stably Integrated Nucleic Acid Molecule

In some embodiments, the engineered cell for AAV production comprises one or more stably integrated nucleic acid molecules. In some embodiments, the engineered cell for AAV production comprising one or more stably integrated nucleic acid molecules comprises a first stably integrated nucleic acid molecule. In some embodiments, the first stably integrated nucleic acid molecule comprises a nucleic acid sequence encoding for each of E2A or DA-E2A; E4Orf6 or DA-E4Orf6; L4 100K or DA-L4 100K; and VARNA or DA-VARNA as described above. In some embodiments, the nucleic acid sequences encoding for E2A or DA-E2A; E4Orf6 or DA-E4Orf6; L4 100K or DA-L4 100K; and VARNA or DA-VARNA are each operably linked to a promoter as described herein.

In some embodiments, the first stably integrated nucleic acid molecule comprises a selection marker operably linked to a promoter as described herein. In some embodiments, the first stably integrated nucleic acid molecule further comprises two CTCF insulator sequences as described herein. As used herein, the term “CTCF insulator” refers to the CCCTC-binding factor that can prevent unwanted crosstalk between genomic regions. In some embodiments, the first stably integrated nucleic acid molecule further comprises two IR/DR sequences that are capable of binding the Sleeping Beauty transposase.

In some embodiments, the first stably integrated nucleic acid molecule comprises a nucleic acid sequence encoding DA-E2A. In some embodiments, the first stably integrated nucleic acid molecule comprises a nucleic acid sequence encoding DA-E4ORF6. In some embodiments, the first stably integrated nucleic acid molecule comprises a nucleic acid sequence encoding DA-E2A and DA-E4 ORF6. In some embodiments, the first stably integrated nucleic acid molecule comprises a nucleic acid sequence encoding DA-L4 100K.

In some embodiments, the first stably integrated nucleic acid molecule comprises SEQ ID NO: 39 or SEQ ID NO: 40, SEQ ID NO: 98 or 112, SEQ ID NO: 41 or SEQ ID NO: 42, and SEQ ID NO: 13. In some embodiments, the first stably integrated nucleic acid molecule comprising SEQ ID NO: 39 or SEQ ID NO: 40, SEQ ID NO: 98 or 112, SEQ ID NO: 41 or SEQ ID NO: 42, and SEQ ID NO: 13 further comprises a selection cassette. In some embodiments, the first stably integrated nucleic acid molecule comprising SEQ ID NO: 39 or SEQ ID NO: 40, SEQ ID NO: 98 or 112 SEQ ID NO: 41 or SEQ ID NO: 42, SEQ ID NO: 13 and a selection cassette further comprises two CTCF insulators, wherein the CTCF insulators are located on the 5′ and 3′ ends of the first stably integrated nucleic acid molecule and SEQ ID NO: 39 or SEQ ID NO: 40, SEQ ID NO: 98 or 112, SEQ ID NO: 41 or SEQ ID NO: 42, SEQ ID NO: 13 and a selection cassette are located between the two CTCF insulators.

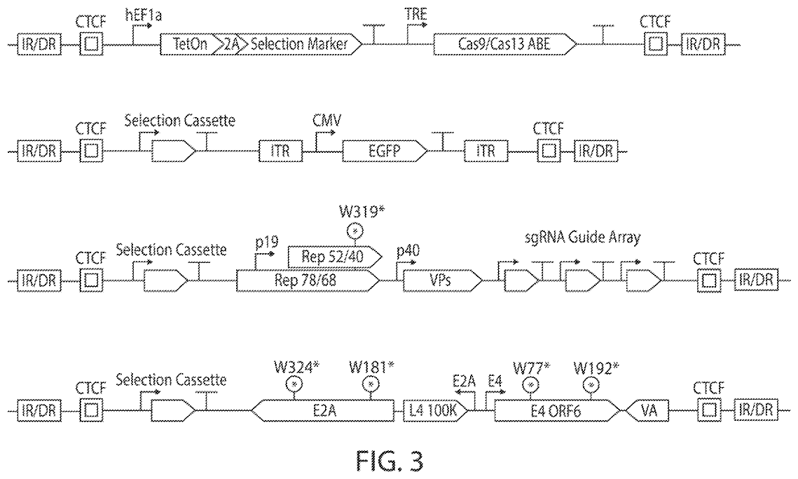

In some embodiments, the first stably integrated nucleic acid molecule as described above has the same structure as is depicted in FIG. 3.

### B. The Second Integrated Nucleic Acid Molecule

In some embodiments, the engineered cell for AAV production comprising one or more stably integrated nucleic acid molecules comprises the first stably integrated nucleic acid molecule and a second stably integrated nucleic acid molecule. In some embodiments, the second stably integrated nucleic acid molecule comprises a nucleic acid sequence encoding for each of Rep52, DA-Rep52, Rep40, or DA-Rep40; Rep78, DA-Rep78, Rep68, or DA-Rep68; VP1 or DA-VP1; VP2 or DA-VP2; and VP3 or DA-VP3 as described herein. In some embodiments, the nucleic acid sequences encoding for Rep52, DA-Rep52, Rep40, or DA-Rep40; Rep78, DA-Rep78, Rep68, or DA-Rep68; VP1 or DA-VP1; VP2 or DA-VP2; and VP3 or DA-VP3 are each operably linked to a promoter as described herein.

In some embodiments, the second stably integrated nucleic acid molecule further comprises one or more single guide RNAs (e.g. a single guide RNA array), wherein the one or more single guide RNAs each comprise a spacer region as described herein. In some embodiments, the nucleic acid sequences encoding for the one or more single guide RNAs are each operably linked to a promoter as described herein. In some embodiments, the nucleic acid sequences encoding for the one or more single guide RNAs are each operably linked to a chemically inducible promoter as described herein.

In some embodiments, the second stably integrated nucleic acid molecule further comprises a selection marker operably linked to a promoter as described herein. In some embodiments, the second stably integrated nucleic acid molecule further comprises two CTCF insulator sequences as described above. In some embodiments, the second stably integrated nucleic acid molecule further comprises two IR/DR sequences as described above.

In some embodiments, the second stably integrated nucleic acid molecule comprises DA-Rep comprising premature stop codon at a position corresponding to W319 in SEQ ID NO: 97. In some embodiments, DA-Rep is operably linked to a promoter. In some embodiments, DA-Rep is operably linked to a p19 promoter.

In some embodiments, the one or more sgRNAs each comprise a spacer region sufficiently complementary to the DA-Rep W319 a premature stop codon to direct a Base Editor to edit the premature stop codon, as described above. In some embodiments, the one or more sgRNAs additionally each comprise a spacer region sufficiently complementary to DA-E4 ORF6 premature stop codons at positions W77 and W192 to direct a Base Editor to edit the premature stop codons to Tryptophan (W) stop codons, as described above.

In some embodiments, the second stably integrated nucleic acid molecule comprises SEQ ID NOs: 14-16, 54, and 65 or 81. In some embodiments, the second stably integrated nucleic acid molecule comprising SEQ ID NOs: 14-16, 54, and 65 or 81 further comprises SEQ ID NOs: 56-59. In some embodiments, the second stably integrated nucleic acid molecule comprising SEQ ID NOs: 14-16, 54, and 65 or 81 further comprises SEQ ID NOs: 66-69. In some embodiments, the second stably integrated nucleic acid molecule comprising SEQ ID NOs: 14-16, 54, and 65 or 81 further comprises SEQ ID NOs: 74-77. In some embodiments, the second stably integrated nucleic acid molecule comprising SEQ ID NOs: 14-16, 54, and 65 or 81, SEQ ID NOs: 56-59 or SEQ ID NOs: 66-69 or SEQ ID NOs: 74-77 further comprises a selection cassette. In some embodiments, the second stably integrated nucleic acid molecule comprising SEQ ID NOs: 14-16, 54, and 65 or 81, SEQ ID NOs: 56-59 or SEQ ID NOs: 66-69 or SEQ ID NOs: 74-77 and a selection cassette further comprises two CTCF insulators, wherein the CTCF insulators are located on the 5′ and 3′ ends of the first stably integrated nucleic acid molecule and SEQ ID NOs: 14-16, 54, and 65 or 81, SEQ ID NOs: 56-59 or SEQ ID NOs: 66-69 or SEQ ID NOs: 74-77 and a selection cassette are located between the two CTCF insulators.

In some embodiments, the second stably integrated nucleic acid molecule as described above has the same structure as is depicted in FIG. 3.

### C. The Third Stably Integrated Nucleic Acid Molecule

In some embodiments, the engineered cell for AAV production comprising one or more stably integrated nucleic acid molecules comprises a first stably integrated nucleic acid molecule as described above, a second stably integrated nucleic acid molecule as described above and comprises third stably integrated nucleic acid molecule. In some embodiments, the third stably integrated nucleic acid molecule comprises a nucleic acid sequence encoding a Base Editor as described above. In some embodiments, the third stably integrated nucleic acid molecule further comprises a nucleic acid sequence encoding a transcriptional activator as described above. In some embodiments, the third stably integrated nucleic acid molecule further comprises a selection marker operably linked to a promoter as described herein. In some embodiments, the third stably integrated nucleic acid molecule further comprises two CTCF insulator sequences as described above. In some embodiments, the third stably integrated nucleic acid molecule further comprises two IR/DR sequences as described above. In some embodiments, the third stably integrated nucleic acid molecule further comprises a transcriptional activator operably linked to a promoter as described above.

In some embodiments, the third stably integrated nucleic acid molecule comprises a nucleic acid molecule encoding a Base Editor (e.g. a Cas9 ABE, a Cas9 CBE, or nucleic acid molecule encoding a Cas13 ABE) operably linked to a promoter. In some embodiments, the promoter is chemically inducible. In some embodiments the chemically inducible promoter is TRE. In some embodiments, the third stably integrated nucleic acid molecule comprises a nucleic acid sequence encoding a TetOn transcriptional activator. In some embodiments, a 2A sequence is encoded between the TetOn nucleic acid sequence and the selection marker nucleic acid sequence.

In some embodiments, the first stably integrated nucleic acid molecule comprises Cas9 ABE7.10 (SEQ ID NO: 82), Cas9 ABE8.17m (SEQ ID NO: 83), Cas13 REPAIRv1 (SEQ ID NO: 84), or Cas13 REPAIRv2 (SEQ ID NO: 85). In some embodiments, the first stably integrated nucleic acid molecule comprising Cas9 ABE7.10 (SEQ ID NO: 82), Cas9 ABE8.17m (SEQ ID NO: 83), Cas13 REPAIRv1 (SEQ ID NO: 84), or Cas13 REPAIRv2 (SEQ ID NO: 85) further comprises a TetOn promoter, a 2A peptide, and a selection marker. In some embodiments, the first stably integrated nucleic acid molecule comprising Cas9 ABE7.10 (SEQ ID NO: 82), Cas9 ABE8.17m (SEQ ID NO: 83), Cas13 REPAIRv1 (SEQ ID NO: 84), or Cas13 REPAIRv2 (SEQ ID NO: 85), a TetOn promoter, a 2A peptide, and a selection marker further comprises two CTCF insulators, wherein the CTCF insulators are located on the 5′ and 3′ ends of the first stably integrated nucleic acid molecule and Cas9 ABE7.10 (SEQ ID NO: 82), Cas9 ABE8.17m (SEQ ID NO: 83), Cas13 REPAIRv1 (SEQ ID NO: 84), or Cas13 REPAIRv2 (SEQ ID NO: 85), a TetOn promoter, a 2A peptide, and a selection marker are located between the two CTCF insulators.

In some embodiments, the third stably integrated nucleic acid molecule comprises a Base Editor comprising an Cytosine Base Editor (CBE). In some embodiments, the CBE is a Cas9 CBE or a Cas13 CBE. In some embodiments, the nucleic acid sequences encoding for the CBE is operably linked to a third chemically inducible promoter. In some embodiments, CBE is operably linked to the third chemically inducible promoter selected from the group consisting of pTRE3G, pTREtight, or a promoter containing at least one of VanR, TtgR, PhlF, or CymR, or the Gal4 UAS operator sequences.

In some embodiments, the third stably integrated nucleic acid molecule as described above has the same structure as is depicted in FIG. 3.

### D. The Fourth Stably Integrated Nucleic Acid Molecule

In some embodiments, the engineered cell for AAV production comprising one or more stably integrated nucleic acid molecules comprises a first stably integrated nucleic acid molecule as described above, a second stably integrated nucleic acid molecule as described above, a third stably integrated nucleic acid molecule and comprises a fourth stably integrated nucleic acid molecule. In some embodiments, the fourth stably integrated nucleic acid molecule comprises a nucleic acid sequence encoding each of a selection cassette, and a fluorescent protein marker (as described herein), such as EGFP, each nucleic acid sequence being operably linked to a promoter. In some embodiments, the fourth stably integrated nucleic acid molecule further comprises two inverted terminal repeat (ITR) sequences. In some embodiments, the fourth stably integrated nucleic acid molecule further comprises a payload comprising two inverted terminal repeat (ITR) sequences flanking and a gene as described above. In some embodiments, the fourth stably integrated nucleic acid molecule further comprises two CTCF insulator sequences as described above. In some embodiments, the third stably integrated nucleic acid molecule further comprises two IR/DR sequences as described above.

In some embodiments, the fourth stably integrated nucleic acid molecule as described above has the same structure as is depicted in FIG. 3.

### VII. Methods of Using Engineered Cells for AAV Production Comprising a Base Editor

In some aspects, the present disclosure provides methods for producing AAVs using an engineered cells comprising a Base Editor and/or the sgRNA(s) are operably linked to a chemically inducible promoter as described herein. In some embodiments, the method further comprises using an engineered cell comprises a nucleic acid sequence molecule encoding a nucleic acid sequence for AAV delivery. In some embodiments, the method comprises growing the engineered cell to a confluency that is optimal for AAV production. An optimal confluency will be dependent on the type of cell the engineered cell is derived from. The skilled person will know or be able to determine the optimal confluency for AAV production. In some embodiments, the method comprises contacting the engineered cell with a small molecule inducer capable of inducing expression of the Base Editor or the sgRNA(s). In some embodiments, the small molecule inducer is doxycycline, vanillate, phloretin, rapamycin, abscisic acid, gibberellic acid acetoxymethyl ester, or cumate. In some embodiments, the method comprises harvesting the AAV produced from the culture of engineered cells using methods that are well known to those of skill in the art.

### VIII. Engineered Cells

In some aspects, this disclosure is related to engineered cells (e.g. the cells engineered for AAV production). In some embodiments, the engineered cells are derived from known or existing cell lines. In some embodiments, the engineered cells are derived from the group consisting of HEK293 cells, HeLa cells, BHK cells, and Sf9 cells. In some embodiments, the engineered cells comprise nucleic acid sequences encoding genes required for AAV production and systems for regulating expression of said genes, as described herein. In some embodiments, the engineered cell comprises genomic sites for stable integration of one or more nucleic acid molecules (e.g. 1, 2, 3, 4, 5, or 6 nucleic acid molecules). These genomics sites for stable integration of nucleic acid molecules are well known to those of ordinary skill in the art. Exemplary sites for stable integration include but are not limited to AAVS1, ROSA26, CCR5, H11, and LiPS-A3S. In some embodiments, the stably integrated nucleic acid molecule is randomly integrated into the Engineered cell genome.

### IX. Kits

In some aspects, the disclosure relates to kits comprising a AAV production systems described herein in Parts I-II and V.

In some embodiments, a kit comprises one or more polynucleic acids collectively comprising an AAV production system.

In some embodiments, a kit comprises an engineered cell described in Parts III, VI and VIII.

In some embodiments, a kit comprises a polynucleotide comprising, from 5′ to 3′: (i) a nucleic acid sequence of a 5′ inverted terminal repeat; (ii) a multiple cloning site; and (iii) a nucleic acid sequence of a 3′ inverted terminal repeat. In some embodiments, the polynucleotide is a plasmid or a vector.

The central nucleic acid of a transfer polynucleic acid may comprise a nucleic acid sequence of a multiple cloning site. Exemplary multiple cloning sites are known to those having ordinary skill in the art. A multiple cloning site can be used for cloning a payload molecule (or gene of interest)—or an expression cassette encoding a payload molecule-into the transfer polynucleic acid prior to the generation of viral vectors in a host cell.

In some embodiments, a kit further comprises a small molecule inducer corresponding to a chemically inducible promoter of the AAV production system. In some embodiments, a small molecule inducer is doxycycline, vanillate, phloretin, rapamycin, abscisic acid, gibberellic acid acetoxymethyl ester, and cumate. In some embodiments, the kits may further comprise instructions for use of the cells.

In some embodiments, a kit comprises an engineered cell, wherein the engineered cell comprises the stably integrated nucleic acid molecules of section III or section VI.

In some embodiments, a kit comprises a polynucleic acid comprising a nucleic acid sequence of a transcriptional activator operably linked to a nucleic acid sequence of a promoter, wherein the transcriptional activator, when expressed in the presence of the small molecule inducer, binds to a chemically inducible promoter of the AAV production system, optionally wherein an engineered cell comprises the polynucleic acid comprising the nucleic acid sequence of the transcriptional activator. In some embodiments, the transcriptional activator is selected from the group consisting of TetOn-3G, TetOn-V16, TetOff-Advanced, VanR-VP16, TtgR-VP16, PhlF-VP16, and the cumate cTA and rcTA.

### EXAMPLES

### Example 1

### Non-Canonical Amino Acid AAV

### Description of Approach and Genetic Schematic:

Use of non-canonical amino acid (ncAA) incorporation at premature stop codons provides a translational level of control over toxic proteins. Tying protein expression to the presence of non-canonical amino acids provides inducible control of protein expression after transcription, which means that even in the presence of transcript there should be very low/no expression of target proteins. Rep78 and Rep52 ncAA stop codon mutants were generated by introducing TAG stop codons at sites previously identified as tolerant of amino acid changes. In this system the orthogonal transfer RNA (tRNA) synthetase (pylRS) and its cognate tRNA (tRNApyl), derived from the archaebacteria Methanosarcina mazei were used to incorporate H-Lys(Boc)-OH, an l-lysine derivative, into Rep proteins to induce AAV production.

### Example 2

### Base Editor AAV

### Description of Approach and Genetic Schematic:

E1 activation of cytotoxic genes in HEK293T producer lines can be avoided by reversibly disabling those genes with a premature stop codon. When protein expression is desired, an Adenine Base Editor (ABE) can perform a targeted A-to-G point mutation to revert the premature stop codon to a coding amino acid. Premature stop mutations made to tryptophan (W) codons on the sense strand can be reverted by both DNA-based Cas9 ABEs and RNA-based Cas13 ABEs. On the anti-sense strand, DNA-based Cas9 ABEs can revert premature stop codons made to glutamine (Q) and arginine residues. On the anti-sense strand, DNA-based Cas9 CBEs can revert premature stop codons made to prolin (P) residues.

It was hypothesized that premature stop codons introduced to Rep, E2A, and E4 would prevent expression of these proteins, resulting in reduced AAV titers, improved cell health, and therefore improved ability to make stable AAV producer cells. When production of AAV is desired, the ABE can be expressed by an inducible promoter upon treatment with a small molecule. For example, the tetracycline responsive elements (TRE) could induce expression of an ABE in the presence of doxycycline and a reverse tetracycline transactivator (rtTA). Single guide RNAs for the ABE are constitutively expressed by an RNA PolIII promoter, such as U6.

Table 3 indicates the specific mutations made to Rep, Cap, E2A, E4, and L4 100K coding sequences, with guide sequences for the repair of those mutations. Amino acid position numbering corresponds to the CDS indicated. Nucleotide position numbering corresponds to the complete genomes of Adenovirus type 2 (GenBank: J01917.1, NCBI: NC_001405.1) and Adeno-associated virus type 2 (GenBank: AF043303.1, NCBI: NC_001401.2), with flanking bases given as context. Additional silent mutations may have been made near the premature stop codon to introduce a PAM sequence for Cas9 ABEs, or to reduce off-target base editing within the ABE edit window.

TABLE 3
sgRNA sequences

| | | | | | Additional | Cas9 DNA |
|---|---|---|---|---|---|---|
| AA | | Additional | Reference | Flanking | mutation | Guide |
| Mutant | Nucleotide | Nuc. | Genome | Bases | reason | Sequence |
| E2A DBP | 23538C > T | | J01917.1 | ctccatgccctt | | tgcgtAggagaag |
| W181* | | | | ctccTacgca | | ggcatgg (SEQ |
| | | | | gacacgat | | ID NO: 56) |
| | | | | (SEQ ID | | |
| | | | | NO: 119) | | |
| E2A DBP | 23109C > T | 23091TG > AC | J01917.1 | gagatcACca | PAM | ccggtAgggccg |
| W324*, | | | | ccacatttcgg | | aaatgtgg (SEQ |
| Q330V | | | | cccTaccgg | | ID NO: 57) |
| | | | | (SEQ ID | | |
| | | | | NO: 120) | | |
| E4 ORF6 | 33847C > T | | J01917.1 | tcccagggaac | | atgAgttgttccct |
| W77* | | | | aacTcattect | | gggata (SEQ |
| | | | | gaatcagc | | ID NO: 58) |
| | | | | (SEQ ID | | |
| | | | | NO: 121) | | |
| E4 ORF6 | 33503C > T | | J01917.1 | cgtggccatca | | cttgtAgtatgatg |
| W192* | | | | tacTacaagc | | gccacg (SEQ |
| | | | | gcaggtaga | | ID NO: 59) |
| | | | | (SEQ ID | | |
| | | | | NO: 122) | | |
| L4 100K | 25411G > A | | J01917.1 | ggccatgggc | | cgtgtAgcagcaa |
| W435* | | | | gtgtAgcagc | | tgcctgg (SEQ |
| | | | | aatgcctgga | | ID NO: 60) |
| | | | | (SEQ ID | | |
| | | | | NO: 123) | | |
| VP1 | 3114G > A | 3132A > G | AF043303. | aactgAggatt | PAM | actgAggattccg |
| W304* | | | 1 | ccgacccaag | | acccaag (SEQ |
| R310R | | | | agGctcaac | | ID NO: 61) |
| | | | | (SEQ ID | | |
| | | | | NO: 124) | | |
| VP1 | 3994C > T | | AF043303. | gcagatgtcaa | | gccttAtgtgttga |
| Q598* | | | 1 | cacaTaaggc | | catctg (SEQ |
| | | | | gttcttcca | | ID NO: 62) |
| | | | | (SEQ ID | | |
| | | | | NO: 125) | | |
| Rep78 | 520G > A | 518A > G | AF043303. | gactttctgacg | Reduce off- | ggaGtAgcgccg |
| W67*, | | | 1 | gaGtAgcgc | target | tgtgagta (SEQ |
| E66E | | | | cgtgtgagt | | ID NO: 63) |
| | | | | (SEQ ID | | |
| | | | | NO: 126) | | |
| Rep78 | 1104C > T | 1101TCC > | AF043303. | ctccaactcgc | Reduce off- | gatttAGCTccg |
| Q262*, | | AGC | 1 | ggAGCTaa | target | cgagttgg (SEQ |
| S261S | | | | atcaaggctgc | | ID NO: 64) |
| | | | | (SEQ ID | | |
| | | | | NO: 127) | | |
| Rep78 | 1276G > A | | AF043303. | ccgtctttctgg | | ggatAggccacg |
| W319* | | | 1 | gatAggccac | | aaaaagtt (SEQ |
| | | | | gaaaaagt | | ID NO: 65) |
| | | | | (SEQ ID | | |
| | | | | NO: 128) | | |
| | | | | | Cas13 RNA | |
| | | | | AA | Guide Sequence | Cas13 RNA Guide |
| | | | | Mutant | 30 nt | Sequence 50 nt |
| | | | | E2A DBP | cttctccCacgcagaca | ctccatgcccttctccCacgca |
| | | | | W181* | cgatcggcaggct | gacacgatcggcaggctcagc |
| | | | | | (SEQ ID NO: 66) | gggttta (SEQ ID NO: |
| | | | | | | 74) |
| | | | | E2A DBP | tcggcccCaccggttct | caccacatttcggcccCaccg |
| | | | | W324*, | tcacgatcttggc | gttcttcacgatcttggccttgct |
| | | | | Q330V | (SEQ ID NO: 67) | agact (SEQ ID NO: 75) |
| | | | | E4 ORF6 | gaacaacCcattcctga | tatcccagggaacaacCcattc |
| | | | | W77* | atcagcgtaaatc | ctgaatcagcgtaaatcccaca |
| | | | | | (SEQ ID NO: 68) | ctgcag (SEQ ID NO: |
| | | | | | | 76) |
| | | | | E4 ORF6 | atcatacCacaagcgca | cacgtggccatcatacCacaa |
| | | | | W192* | ggtagattaagtg | gcgcaggtagattaagtggcga |
| | | | | | (SEQ ID NO: 69) | cccctca (SEQ ID NO: |
| | | | | | | 77) |
| | | | | L4 100K | ttgctgcCacacgccca | ctccaggcattgctgcCacacg |
| | | | | W435* | tggccgtttgcca | cccatggccgtttgccaggtgta |
| | | | | | (SEQ ID NO: 70) | gcaca (SEQ ID NO: 78) |
| | | | | VP1 | ggaatccCcagttgttg | tcttgggtcggaatccCcagtt |
| | | | | W304* | ttgatgagtcttt | gttgttgatgagtctttgccagtc |
| | | | | R310R | (SEQ ID NO: 71) | acgt (SEQ ID NO: 79) |
| | | | | VP1 | NA | NA |
| | | | | Q598* | | |
| | | | | Rep78 | acggcgcCactccgtc | cttactcacacggcgcCactcc |
| | | | | W67*, | agaaagtcgcgctg | gtcagaaagtcgcgctgcagct |
| | | | | E66E | (SEQ ID NO: 72) | tctcgg (SEQ ID NO: |
| | | | | | | 80) |
| | | | | Rep78 | NA | NA |
| | | | | Q262*, | | |
| | | | | S261S | | |
| | | | | Rep78 | cgtggccCatcccaga | gaactttttcgtggccCatccca |
| | | | | W319* | aagacggaagccgc | gaaagacggaagccgcatatt |
| | | | | | (SEQ ID NO: 73) | ggggat (SEQ ID NO: |
| | | | | | | 81) |

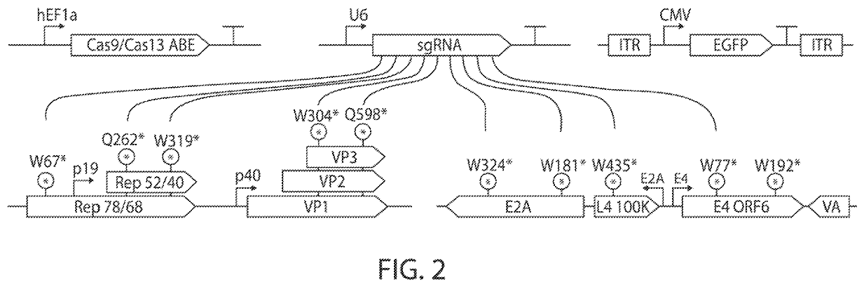

In the experiments, premature stop mutations were made to the pRepCap and pHelper standard plasmids (FIG. 2). Production of AAV with transient transfection was performed with these modified plasmids to determine the impact of the premature stop codons on AAV titer. Single mutations made to Rep or Cap were enough to diminish AAV titers. Mutations made to Rep could be recovered to ‘wild-type’ levels of AAV with co-transfection of an ABE and single guide RNA plasmid. Single mutations introduced to E2A, E4ORF6, or L4 100K individually were not enough to diminish AAV titers alone, but combinations of mutants made a larger impact. Those combinations were able to be recovered to ‘wild-type’ levels of AAV as well, when co-transfected with an ABE and guide pool. This result held when assaying the stable plasmid system (FIG. 3) in transient, displaying inducibility of AAV titers in the presence of doxycycline.

### Preliminary Data and Experiment Description:

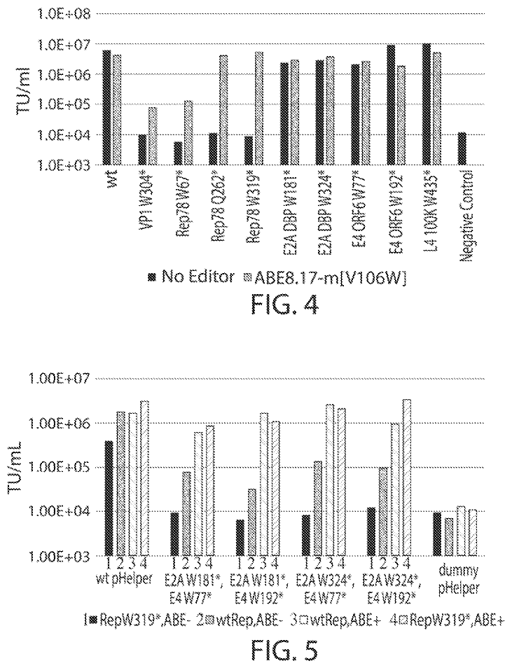

Adherent HEK293FT cells were co-transfected with EGFP-expressing transfer plasmid, pRepCap, pHelper, ABE plasmid, and single guide RNA plasmid (FIG. 2). Mutant variants of pRepCap or pHelper replaced the ‘wild type’ plasmids to test their impact on AAV titer (FIG. 4). An ABE and corresponding guide were co-transfected to determine if the ABE could restore viral titer. In samples where the ABE and guide were not tested, an inert plasmid was co-transfected to keep the amount of transfected DNA the same. Control samples containing only ‘wild type’ AAV2 pRepCap and pHelper plasmids or a negative control transfection mix without DNA were also prepared. 48 hours after transfection, AAV was harvested by four freeze thaw cycles in a dry ice isopropanol bath. Virus stock was transduced by addition of 10, 1, and 0.5 uL to 5e4 HEK293FT cells plated in a 96-well plate. 48 hours after transduction, transduced cells were harvested and percentage of EGFP positive cells was determined by flow cytometry and used to calculate transducing units per mL (TU/mL).

Next, adherent HEK293FT cells were co-transfected with combinations of premature stop mutants in pRepCap or pHelper to test their combined impact on AAV titer, and their ability to be recovered with an ABE and pool of single guide RNA plasmids (FIG. 5). 48 hours after transfection, AAV was harvested by four freeze thaw cycles in a dry ice isopropanol bath. Virus stock was serially diluted 1-, 10- and 100-fold and 10 uL of resulting viral stock was transduced by addition to 5e4 HEK293FT cells plated in a 96-well plate. 48 hours after transduction, transduced cells were harvested and percentage of EGFP positive cells was determined by flow cytometry and used to calculate transducing units per mL (TU/mL).

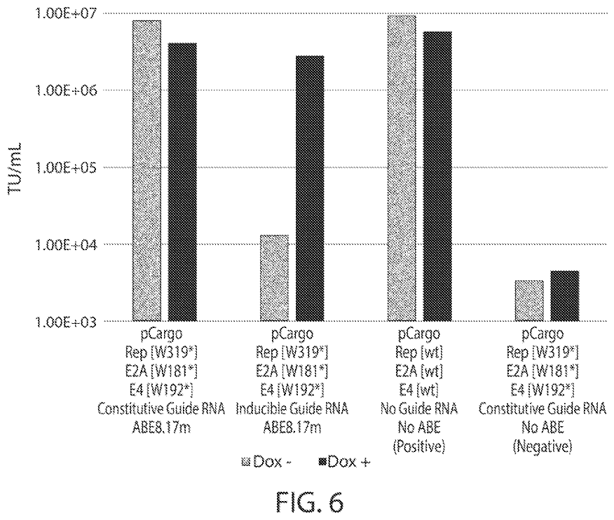

To assay the feasibility of the full stable system, adherent HEK293FT cells were co-transfected with combinations of the full stable system (FIG. 3), with or without 500 nM doxycycline, to test their combined ability to induce AAV in the presence of doxycycline (FIG. 6). 48 hours after transfection, AAV was harvested by four freeze thaw cycles in a dry ice isopropanol bath. 10 uL and 1 uL of the resulting viral stock was transduced by addition to 5e4 HEK293FT cells plated in a 96-well plate. 48 hours after transduction, transduced cells were harvested and the percentage of EGFP positive cells was determined by flow cytometry and used to calculate transducing units per mL (TU/mL).

A stable cell line containing an inducible ABE, a constitutive pool of guides, and combinations of mutant Rep, Cap, E2A, or E4 ORF6 in suspension cells will be generated for inducible AAV production (FIG. 3).

TABLE 4
Nucleic Acid and Polypeptide Sequences

| SEQ | | |
|---|---|---|
| ID | | |
| NO: | Descrip. | Sequence |
| 1 | pTREtight | ctcgagtttactccctatcagtgatagagaacgtatgtcgagtttactccctatcagtgatagagaacgatgtcgagtttact |
| | | ccctatcagtgatagagaacgtatgtcgagtttactccctatcagtgatagagaacgtatgtcgagtttactccctatcagt |
| | | gatagagaacgtatgtcgagtttatccctatcagtgatagagaacgtatgtcgagtttactccctatcagtgatagagaac |
| | | gtatgtcgaggtaggcgtgtacggtgggaggcctatataagcagagctcgtttagtgaaccgtcagatcgcctggagaa |
| | | ttcgagctcggtacccgggga |
| 2 | pTRE3G | gtttactccctatcagtgatagagaacgtatgaagagtttactccctatcagtgatagagaacgtatgcagactttactccct |
| | | atcagtgatagagaacgtataaggagtttactccctatcagtgatagagaacgtatgaccagtttactccctatcagtgata |
| | | gagaacgtatctacagtttactccctatcagtgatagagaacgtatatccagtttactccctatcagtgatagagaacgtat |
| | | aagctttaggcgtgtacggtgggcgcctataaaagcagagctcgtttagtgaaccgtcagatcgcctggagcaattcca |
| | | caacacttttgtcttataccaactttccgtaccacttcctaccctcgtaaagtcgacaccggggcccagatctatcgatcgg |
| | | ccggataacgccacc |
| 3 | bi-TRE3G | gaattctccaggcgatctgacggttcactaaacgagctctgcttatataggcctcccaccgtacacgccacctcgacata |
| | | ctcgagtttactccctatcagtgatagagaacgtatgaagagtttactccctatcagtgatagagaacgtatgcagacttta |
| | | ctccctatcagtgatagagaacgtataaggagtttactccctatcagtgatagagaacgtatgaccagtttactccctatca |
| | | gtgatagagaacgtatctacagtttactccctatcagtgatagagaacgtatatccagtttactccctatcagtgatagaga |
| | | acgtataagctttaggcgtgtacggtgggcgcctataaaagcagagctcgtttagtgaaccgtcagatcgcctggagca |
| | | attccacaacacttttgtcttataccaactttccgtaccacttcctaccctcgtaaagtcgacaccggggcccagatctccg |
| | | cggggatcc |
| 4 | IRES | cccctctccctcccccccccctaacgttactggccgaagccgcttggaataaggccggtgtgcgtttgtctatatgttatttt |
| | | ccaccatattgccgtcttttggcaatgtgagggcccggaaacctggccctgtcttcttgacgagcattcctaggggtctttc |
| | | ccctctcgccaaaggaatgcaaggtctgttgaatgtcgtgaaggaagcagttcctctggaagcttcttgaagacaaacaa |
| | | cgtctgtagcgaccctttgcaggcagcggaaccccccacctggcgacaggtgcctctgcggccaaaagccacgtgta |
| | | taagatacacctgcaaaggcggcacaaccccagtgccacgttgtgagttggatagttgtggaaagagtcaaatggctct |
| | | cctcaagcgtattcaacaaggggctgaaggatgcccagaaggtaccccattgtatgggatctgatctggggcctcggtg |
| | | cacatgctttacatgtgtttagtcgaggttaaaaaaacgtctaggccccccgaaccacggggacgtggttttcctttgaaa |
| | | aacacgatgataatatg |
| 5 | attenuated | cccccccccctaacgttactggccgaagccgcttggaataaggccggtgtgcgtttgtctatatgttattttccaccatatt |
| | IRES | gccgtcttttggcaatgtgagggcccggaaacctggccctgtcttcttgacgagcattcctaggggtctttcccctctcgc |
| | | caaaggaatgcaaggtctgttgaatgtcgtgaaggaagcagttcctctggaagcttcttgaagacaaacaacgtctgtag |
| | | cgaccctttgcaggcagcggaaccccccacctggcgacaggtgcctctgcggccaaaagccacgtgtataagataca |
| | | cctgcaaaggcggcacaaccccagtgccacgttgtgagttggatagttgtggaaagagtcaaatggctctcctcaagc |
| | | gtattcaacaaggggctgaaggatgcccagaaggtaccccattgtatgggatctgatctggggcctcggtgcacatgct |
| | | ttacatgtgtttagtcgaggttaaaaaaacgtctaggccccccgaaccacggggacgtggttttcctttgaaaaacacgat |
| | | gataatagttatc |
| 6 | Rep52 (wt) | MELVGWLVDKGITSEKQWIQEDQASYISFNAASNSRSQIKAALDNAGKIMS |
| | | LTKTAPDYLVGQQPVEDISSNRIYKILELNGYDPQYAASVFLGWATKKFGK |
| | | RNTIWLFGPATTGKTNIAEAIAHTVPFYGCVNWTNENFPFNDCVDKMVIW |
| | | WEEGKMTAKVVESAKAILGGSKVRVDQKCKSSAQIDPTPVIVTSNTNMCA |
| | | VIDGNSTTFEHQQPLQDRMFKFELTRRLDHDFGKVTKQEVKDFFRWAKDH |
| | | VVEVEHEFYVKKGGAKKRPAPSDADISEPKRVRESVAQPSTSDAEASINYA |
| | | DRYQNKCSRHVGMNLMLFPCRQCERMNQNSNICFTHGQKDCLECFPVSES |
| | | QPVSVVKKAYQKLCYIHHIMGKVPDACTACDLVNVDLDDCIFEQ |
| 7 | Rep40 (wt) | MELVGWLVDKGITSEKQWIQEDQASYISFNAASNSRSQIKAALDNAGKIMS |
| | | LTKTAPDYLVGQQPVEDISSNRIYKILELNGYDPQYAASVFLGWATKKFGK |
| | | RNTIWLFGPATTGKTNIAEAIAHTVPFYGCVNWTNENFPFNDCVDKMVIW |
| | | WEEGKMTAKVVESAKAILGGSKVRVDQKCKSSAQIDPTPVIVTSNTNMCA |
| | | VIDGNSTTFEHQQPLQDRMFKFELTRRLDHDFGKVTKQEVKDFFRWAKDH |
| | | VVEVEHEFYVKKGGAKKRPAPSDADISEPKRVRESVAQPSTSDAEASINYA |
| | | DRLARGHSL |
| 8 | Rep78 (wt) | MPGFYEIVIKVPSDLDEHLPGISDSFVNWVAEKEWELPPDSDMDLNLIEQAP |
| | | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYMELVGWLVDKGITSEKQWIQEDQASYISFN |
| | | AASNSRSQIKAALDNAGKIMSLTKTAPDYLVGQQPVEDISSNRIYKILELNG |
| | | YDPQYAASVFLGWATKKFGKRNTIWLFGPATTGKTNIAEAIAHTVPFYGCV |
| | | NWTNENFPFNDCVDKMVIWWEEGKMTAKVVESAKAILGGSKVRVDQKC |
| | | KSSAQIDPTPVIVTSNTNMCAVIDGNSTTFEHQQPLQDRMFKFELTRRLDHD |
| | | FGKVTKQEVKDFFRWAKDHVVEVEHEFYVKKGGAKKRPAPSDADISEPKR |
| | | VRESVAQPSTSDAEASINYADRYQNKCSRHVGMNLMLFPCRQCERMNQNS |
| | | NICFTHGQKDCLECFPVSESQPVSVVKKAYQKLCYIHHIMGKVPDACTACD |
| | | LVNVDLDDCIFEQ |
| 9 | Rep68 (wt) | MPGFYEIVIKVPSDLDEHLPGISDSFVNWVAEKEWELPPDSDMDLNLIEQAP |
| | | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYMELVGWLVDKGITSEKQWIQEDQASYISFN |
| | | AASNSRSQIKAALDNAGKIMSLTKTAPDYLVGQQPVEDISSNRIYKILELNG |
| | | YDPQYAASVFLGWATKKFGKRNTIWLFGPATTGKTNIAEAIAHTVPFYGCV |
| | | NWTNENFPFNDCVDKMVIWWEEGKMTAKVVESAKAILGGSKVRVDQKC |
| | | KSSAQIDPTPVIVTSNTNMCAVIDGNSTTFEHQQPLQDRMFKFELTRRLDHD |
| | | FGKVTKQEVKDFFRWAKDHVVEVEHEFYVKKGGAKKRPAPSDADISEPKR |
| | | VRESVAQPSTSDAEASINYADRLARGHSL |
| 10 | E2A (wt) | MASREEEQRETTPERGRGAARRPPTMEDVSSPSPSPPPPRAPPKKRLRRRLE |
| | | SEDEEDSSQDALVPRTPSPRPSTSTADLAIASKKKKKRPSPKPERPPSPEVIV |
| | | DSEEEREDVALQMVGFSNPPVLIKHGKGGKRTVRRLNEDDPVARGMRTQE |
| | | EKEESSEAESESTVINPLSLPIVSAWEKGMEAARALMDKYHVDNDLKANFK |
| | | LLPDQVEALAAVCKTWLNEEHRGLQLTFTSNKTFVTMMGRFLQAYLQSFA |
| | | EVTYKHHEPTGCALWLHRCAEIEGELKCLHGSIMINKEHVIEMDVTSENGQ |
| | | RALKEQSSKAKIVKNRWGRNVVQISNTDARCCVHDAACPANQFSGKSCG |
| | | MFFSEGAKAQVAFKQIKAFMQALYPNAQTGHGHLLMPLRCECNSKPGHAP |
| | | FLGRQLPKLTPFALSNAEDLDADLISDKSVLASVHHPALIVFQCCNPVYRNS |
| | | RAQGGGPNCDFKISAPDLLNALVMVRSLWSENFTELPRMVVPEFKWSTKH |
| | | QYRNVSLPVAHSDARQNPFDF |
| 11 | E4 ORF6 | MTTSGVPFGMTLRPTRSRLSRRTPYSRDRLPPFETETRATILEDHPLLPECNT |
| | (wt) | LTMHNVSYVRGLPCSVGFTLIQEWVVPWDMVLTREELVILRKCMHVCLCC |
| | | ANIDIMTSMMIHGYESWALHCHCSSPGSLQCIAGGQVLASWFRMVVDGA |
| | | MFNQRFIWYREVVNYNMPKEVMFMSSVFMRGRHLIYLRLWYDGHVGSV |
| | | VPAMSFGYSALHCGILNNIVVLCCSYCADLSEIRVRCCARRTRRLMLRAVRI |
| | | IAEETTAMLYSCRTERRRQQFIRALLQHHRPILMHDYDSTPM |
| 12 | E4 ORF6 | atgactacgtccggcgttccatttggcatgacactacgaccaacacgatctcggttgtctcggcgcactccgtacagtag |
| | (splice | ggatcgcctacctccttttgagacagagacccgcgctaccatactggaggatcatccgctgctgcccgaatgtaacactt |
| | site | tgacaatgcacaaTgtTTCCtacgtgcgaggtcttccctgcagtgtgggatttacgctgattcaggaatgggttgttcc |
| | removed) | ctgggatatggttctgacgcgggaggagcttgtaatcctgaggaagtgtatgcacgtgtgcctgtgttgtgccaacattg |
| | | atatcatgacgagcatgatgatccatggttacgagtcctgggctctccactgtcattgttccagtcccggttccctgcagtg |
| | | catagccggcgggcaggttttggccagctggtttaggatggtggtggatggcgccatgtttaatcagaggtttatatggta |
| | | ccgggaggtggtgaattacaacatgccaaaagaggtaatgtttatgtccagcgtgtttatgaggggtcgccacttaatcta |
| | | cctgcgcttgtggtatgatggccacgtgggttctgtggtccccgccatgagctttggatacagcgccttgcactgtgggat |
| | | tttgaacaatattgtggtgctgtgctgcagttactgtgctgatttaagtgagatcagggtgcgctgctgtgcccggaggac |
| | | aaggcgtctcatgctgcgggcggtgcgaatcatcgctgaggagaccactgccatgttgtattcctgcaggacggagcg |
| | | gcggcggcagcagtttattcgcgcgctgctgcagcaccaccgccctatcctgatgcacgattatgactctacccccatg |
| | | TAGtaa |
| 13 | VARNA | CGACGTAATCCGTAGATGTACCTGGACATCCAGGTGATGCCGGCGGCGG |
| | | TGGTGGAGGCGCGCGGAAAGTCGCGGACGCGGTTCCAGATGTTGCGCA |
| | | GCGGCAAAAAGTGCTCCATGGTCGGGACGCTCTGGCCGGTGAGGCGTG |
| | | CGCAGTCGTTGACGCTCTAGACCGTGCAAAAGGAGAGCCTGTAAGCGG |
| | | GCACTCTTCCGTGGTCTGGTGGATAAATTCGCAAGGGTATCATGGCGGA |
| | | CGACCGGGGTTCGAACCCCGGATCCGGCCGTCCGCCGTGATCCATGCGG |
| | | TTACCGCCCGCGTGTCGAACCCAGGTGTGCGACGTCAGACAACGGGGG |
| | | AGCGCTCCTTTTGGCTTCCTTCCAGGCGCGGCGGCTGCTGCGCTAGCTTT |
| | | TTTGGCCACTGGCCGCGCGCGGCGTAAGCGGTTAGGCTGGAAAGCGAA |
| | | AGCATTAAGTGGCTCGCTCCCTGTAGCCGGAGGGTTATTTTCCAAGGGT |
| | | TGAGTCGCAGGACCCCCGGTTCGAGTCTCGGGCCGGCCGGACTGCGGCG |
| | | AACGGGGGTTTGCCTCCCCGTCATGCAAGACCCCGCTTGCAAATTCCTC |
| | | CGGAAACAGGGACGAGCCCCTTTTTTGCTTTTCCCAGATGCATCCGGTG |
| | | CTGCGGCAGATGCGCCCCCCTCCTCAGCAGCGGCAAGAGCAAGAGCAG |
| | | CGGCAGACATGCAGGGCACCCTCCCCTTCTCCTACCGCGTCAGGAGGGG |
| | | CAACATCC |
| 14 | VP1 (wt) | MAADGYLPDWLEDTLSEGIRQWWKLKPGPPPPKPAERHKDDSRGLVLPGY |
| | | KYLGPFNGLDKGEPVNEADAAALEHDKAYDRQLDSGDNPYLKYNHADAE |
| | | FQERLKEDTSFGGNLGRAVFQAKKRVLEPLGLVEEPVKTAPGKKRPVEHSP |
| | | VEPDSSSGTGKAGQQPARKRLNFGQTGDADSVPDPQPLGQPPAAPSGLGTN |
| | | TMATGSGAPMADNNEGADGVGNSSGNWHCDSTWMGDRVITTSTRTWAL |
| | | PTYNNHLYKQISSQSGASNDNHYFGYSTPWGYFDFNRFHCHFSPRDWQRLI |
| | | NNNWGFRPKRLNFKLFNIQVKEVTQNDGTTTIANNLTSTVQVFTDSEYQLP |
| | | YVLGSAHQGCLPPFPADVFMVPQYGYLTLNNGSQAVGRSSFYCLEYFPSQ |
| | | MLRTGNNFTFSYTFEDVPFHSSYAHSQSLDRLMNPLIDQYLYYLSRTNTPSG |
| | | TTTQSRLQFSQAGASDIRDQSRNWLPGPCYRQQRVSKTSADNNNSEYSWT |
| | | GATKYHLNGRDSLVNPGPAMASHKDDEEKFFPQSGVLIFGKQGSEKTNVDI |
| | | EKVMITDEEEIRTTNPVATEQYGSVSTNLQRGNRQAATADVNTQGVLPGM |
| | | VWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKS |
| | | VNVDFTVDTNGVYSEPRPIGTRYLTRNL |
| 15 | VP2 (wt) | TAPGKKRPVEHSPVEPDSSSGTGKAGQQPARKRLNFGQTGDADSVPDPQPL |
| | | GQPPAAPSGLGTNTMATGSGAPMADNNEGADGVGNSSGNWHCDSTWMG |
| | | DRVITTSTRTWALPTYNNHLYKQISSQSGASNDNHYFGYSTPWGYFDFNRF |
| | | HCHFSPRDWQRLINNNWGFRPKRLNFKLFNIQVKEVTQNDGTTTIANNLTS |
| | | TVQVFTDSEYQLPYVLGSAHQGCLPPFPADVFMVPQYGYLTLNNGSQAVG |
| | | RSSFYCLEYFPSQMLRTGNNFTFSYTFEDVPFHSSYAHSQSLDRLMNPLIDQ |
| | | YLYYLSRTNTPSGTTTQSRLQFSQAGASDIRDQSRNWLPGPCYRQQRVSKT |
| | | SADNNNSEYSWTGATKYHLNGRDSLVNPGPAMASHKDDEEKFFPQSGVLI |
| | | FGKQGSEKTNVDIEKVMITDEEEIRTTNPVATEQYGSVSTNLQRGNRQAAT |
| | | ADVNTQGVLPGMVWQDRDVYLQGPIWAKIPHTDGHFHPSPLMGGFGLKH |
| | | PPPQILIKNTPVPANPSTTFSAAKFASFITQYSTGQVSVEIEWELQKENSKRW |
| | | NPEIQYTSNYNKSVNVDFTVDTNGVYSEPRPIGTRYLTRNL |
| 16 | VP3 (wt) | MATGSGAPMADNNEGADGVGNSSGNWHCDSTWMGDRVITTSTRTWALP |
| | | TYNNHLYKQISSQSGASNDNHYFGYSTPWGYFDFNRFHCHFSPRDWQRLIN |
| | | NNWGFRPKRLNFKLFNIQVKEVTQNDGTTTIANNLTSTVQVFTDSEYQLPY |
| | | VLGSAHQGCLPPFPADVFMVPQYGYLTLNNGSQAVGRSSFYCLEYFPSQM |
| | | LRTGNNFTFSYTFEDVPFHSSYAHSQSLDRLMNPLIDQYLYYLSRTNTPSGT |
| | | TTQSRLQFSQAGASDIRDQSRNWLPGPCYRQQRVSKTSADNNNSEYSWTG |
| | | ATKYHLNGRDSLVNPGPAMASHKDDEEKFFPQSGVLIFGKQGSEKTNVDIE |
| | | KVMITDEEEIRTTNPVATEQYGSVSTNLQRGNRQAATADVNTQGVLPGMV |
| | | WQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSV |
| | | NVDFTVDTNGVYSEPRPIGTRYLTRNL |
| 17 | AAP (wt) | LETQTQYLTPSLSDSHQQPPLVWELIRWLQAVAHQWQTITRAPTEWVIPREI |
| | | GIAIPHGWATESSPPAPEPGPCPPTTTTSTNKFPANQEPRTTITTLATAPLGGI |
| | | LTSTDSTATFHHVTGKDSSTTTGDSDPRDSTSSSLTFKSKRSRRMTVRRRLPI |
| | | TLPARFRCLLTRSTSSRTSSARRIKDASRRSQQTSSWCHSMDTSP |
| 18 | P2A | ATNFSLLKQAGDVEENPGP |
| | (without | |
| | GSG) | |
| 19 | T2A | EGRGSLLTCGDVEENPGP |
| | (without | |
| | GSG) | |
| 20 | Pyrrolysyl- | MDKKPLNTLISATGLWMSRTGTIHKIKHHEVSRSKIYIEMACGDHLVVNNS |
| | tRNA | RSSRTARALRHHKYRKTCKRCRVSDEDLNKFLTKANEDQTSVKVKVVSAP |
| | synthetase | TRTKKAMPKSVARAPKPLENTEAAQAQPSGSKFSPAIPVSTQESVSVPASVS |
| | (py1RS) | TSISSISTGATASALVKGNTNPITSMSAPVQASAPALTKSQTDRLEVLLNPKD |
| | | EISLNSGKPFRELESELLSRRKKDLQQIYAEERENYLGKLEREITRFFVDRGF |
| | | LEIKSPILIPLEYIERMGIDNDTELSKQIFRVDKNFCLRPMLAPNLYNYLRKL |
| | | DRALPDPIKIFEIGPCYRKESDGKEHLEEFTMLNFCQMGSGCTRENLESIITD |
| | | FLNHLGIDFKIVGDSCMVYGDTLDVMHGDLELSSAVVGPIPLDREWGIDKP |
| | | WIGAGFGLERLLKVKHDFKNIKRAARSESYYNGISTNL*** |
| 21 | Pyrrolysyl- | MDKKPLNTLISATGLWMSRTGTIHKIKHHEVSRSKIYIEMACGDHLVVNNS |
| | tRNA | RSSRTARALRHHKYRKTCKRCRVSDEDLNKFLTKANEDQTSVKVKVVSAP |
| | synthetase | TRTKKAMPKSVARAPKPLENTEAAQAQPSGSKFSPAIPVSTQESVSVPASVS |
| | MmPyrLS | TSISSISTGATASALVKGNTNPITSMSAPVQASAPALTKSQTDRLEVLLNPKD |
| | (Y384F) | EISLNSGKPFRELESELLSRRKKDLQQIYAEERENYLGKLEREITRFFVDRGF |
| | | LEIKSPILIPLEYIERMGIDNDTELSKQIFRVDKNFCLRPMLAPNLYNYLRKL |
| | | DRALPDPIKIFEIGPCYRKESDGKEHLEEFTMLNFCQMGSGCTRENLESIITD |
| | | FLNHLGIDFKIVGDSCMVFGDTLDVMHGDLELSSAVVGPIPLDREWGIDKP |
| | | WIGAGFGLERLLKVKHDFKNIKRAARSESYYNGISTNL*** |
| 22 | Py1T | ggaaacctgatcatgtagatcgaaCggactctaaatccgttcagccgggttagattcccggggtttccg |
| | (U25C) | |
| | tRNA | |
| | (tRNA only) | |
| 23 | Py1T | agtcagtcactagtTGGGCAGGAAGAGGGCCTATTTCCCATGATTCCTTCATATT |
| | (U25C) | TGCATATACGATACAAGGCTGTTAGAGAGATAATTAGAATTAATTTGAC |
| | RNA (U6 | TGTAAACACAAAGATATTAGTACAAAATACGTGACGTAGAAAGTAATA |
| | promoter | ATTTCTTGGGTAGTTTGCAGTTTTAAAATTATGTTTTAAAATGGACTATC |
| | and | ATATGCTTACCGTAACTTGAAAGTATTTCGATTTCTTGGCTTTATATATC |
| | terminator | TTGTGGAAAGGACGAAACACCggaaacctgatcatgtagatcgaaCggactctaaatccgttcag |
| | included | ccgggttagattcccggggtttccgGACAAGTGCGGTTTTTcctaggagtcagtc |
| | with full | |
| | tRNA) | |
| 24 | WT Rep | atgccggggttttacgagattgtgattaaggtccccagcgaccttgacgagcatctgcccggcatttctgacagctttgtg |
| | | aactgggtggccgagaaggaatgggagttgccgccagattctgacatggatctgaatctgattgagcaggcacccctg |
| | | accgtggccgagaagctgcagcgcgactttctgacggaatggcgccgtgtgagtaaggccccggaggcccttttcttt |
| | | gtgcaatttgagaagggagagagctacttccacatgcacgtgctcgtggaaaccaccggggtgaaatccatggttttgg |
| | | gacgtttcctgagtcagattcgcgaaaaactgattcagagaatttaccgcgggatcgagccgactttgccaaactggttc |
| | | gcggtcacaaagaccagaaatggcgccggaggcgggaacaaggtggtggatgagtgctacatccccaattacttgct |
| | | ccccaaaacccagcctgagctccagtgggcgtggactaatatggaacagtatttaagcgcctgtttgaatctcacggag |
| | | cgtaaacggttggtggcgcagcatctgacgcacgtgtcgcagacgcaggagcagaacaaagagaatcagaatccca |
| | | attctgatgcgccggtgatcagatcaaaaacttcagccaggtacatggagctggtcgggtggctcgtggacaagggga |
| | | ttacctcggagaagcagtggatccaggaggaccaggcctcatacatctccttcaatgcggcctccaactcgcggtccca |
| | | aatcaaggctgccttggacaatgcgggaaagattatgagcctgactaaaaccgcccccgactacctggtgggccagca |
| | | gcccgtggaggacatttccagcaatcggatttataaaattttggaactaaacgggtacgatccccaatatgcggcttccgt |
| | | ctttctgggatgggccacgaaaaagttcggcaagaggaacaccatctggctgtttgggcctgcaactaccgggaagac |
| | | caacatcgcggaggccatagcccacactgtgcccttctacgggtgcgtaaactggaccaatgagaactttcccttcaac |
| | | gactgtgtcgacaagatggtgatctggtgggaggaggggaagatgaccgccaaggtcgtggagtcggccaaagcca |
| | | ttctcggaggaagcaaggtgcgcgtggaccagaaatgcaagtcctcggcccagatagacccgactcccgtgatcgtc |
| | | acctccaacaccaacatgtgcgccgtgattgacgggaactcaacgaccttcgaacaccagcagccgttgcaagaccg |
| | | gatgttcaaatttgaactcacccgccgtctggatcatgactttgggaaggtcaccaagcaggaagtcaaagactttttccg |
| | | gtgggcaaaggatcacgtggttgaggtggagcatgaattctacgtcaaaaagggtggagccaagaaaagacccgccc |
| | | ccagtgacgcagatataagtgagcccaaacgggtgcgcgagtcagttgcgcagccatcgacgtcagacgcggaagc |
| | | ttcgatcaactacgcagacaggtaccaaaacaaatgttctcgtcacgtgggcatgaatctgatgctgtttccctgcagaca |
| | | atgcgagagaatgaatcagaattcaaatatctgcttcactcacggacagaaagactgtttagagtgctttcccgtgtcaga |
| | | atctcaacccgtttctgtcgtcaaaaaggcgtatcagaaactgtgctacattcatcatatcatgggaaaggtgccagacgc |
| | | ttgcactgcctgcgatctggtcaatgtggatttggatgactgcatctttgaacaataaatgatttaaatcaggtatggctgcc |
| | | gatggttatcttccagattggctcgaggacactctctctga |
| 25 | Rep78 + 52 | cTGGCGGGCTTCTACGAGATCGTGATCAAGGTGCCCAGCGACCTGGACG |
| | Only | AGCATCTGCCTGGCATCAGCGACAGCTTCGTGAATTGGGTCGCCGAGAA |
| | | AGAGTGGGAGCTGCCTCCTGACAGCGACITGGACCTGAACCTGATTGAG |
| | | CAGGCCCCTCTGACAGTGGCCGAGAAGCTGCAGAGGGATTTCCTGACA |
| | | GAGTGGCGGAGAGTGTCTAAGGCCCCTGAGGCTCTGTTCTTCGTGCAGT |
| | | TCGAGAAGGGCGAGAGCTACTTCCACTTACACGTGCTGGTCGAGACAAC |
| | | CGGCGTGAAGTCTTTAGTGCTGGGCAGATTCCTGAGCCAGATCAGAGAG |
| | | AAGCTGATCCAGCGGATCTACCGGGGCATCGAGCCCACACTGCCTAATT |
| | | GGTTCGCCGTGACCAAGACCAGAAACGGcGCTGGCGGCGGAAACAAGG |
| | | TGGTGGACGAGTGCTACATCCCCAACTACCTGCTGCCTAAGACACAGCC |
| | | CGAACTGCAGTGGGCCTGGACCAACTTAGAACAGTACCTGAGCGCCTGC |
| | | CTGAATCTGACCGAGCGGAAAAGACTGGTGGCCCAGCATCTGACCCAC |
| | | GTGTCCCAGACACAAGAGCAGAACAAAGAGAATCAGAACCCCAACAGC |
| | | GACGCCCCTGTGATCAGAAGCAAGACCAGCGCCAGATACATGGAACTC |
| | | GTTGGCTGGCTGGTGGACAAGGGCATCACAAGCGAGAAGCAGTGGATC |
| | | CAAGAGGACCAGGCCAGCTACATCAGCTTCAACGCCGCCTCCAACAGC |
| | | AGATCCCAGATCAAGGCCGCTCTGGACAACGCCGGCAAGATCATGAGC |
| | | CTGACAAAGACAGCCCCTGACTACCTCGTGGGCCAGCAGCCTGTGGAA |
| | | GATATCAGCAGCAACCGGATCTACAAGATCCTGGAACTGAACGGCTAC |
| | | GACCCTCAGTATGCCGCCTCTGTGTTTCTCGGCTGGGCTACCAAGAAGT |
| | | TCGGCAAGCGGAACACCATCTGGCTGTTTGGCCCTGCCACAACCGGCAA |
| | | GACCAATATCGCCGAGGCTATCGCCCACACCGTGCCTTTTTACGGCTGC |
| | | GTGAACTGGACCAATGAGAACTTCCCCTTCAACGACTGCGTGGACAAGA |
| | | TGGTCATTTGGTGGGAAGAGGGCAAGATGACCGCCAAAGTGGTGGAAA |
| | | GCGCCAAGGCCATCCTCGGCGGATCTAAAGTTCGCGTGGACCAGAAGT |
| | | GCAAGTCTAGCGCCCAGATCGACCCCACACCTGTGATCGTGACCAGCAA |
| | | CACCAACATGTGCGCCGTGATCGACGGCAACAGCACCACCTTTGAACAC |
| | | CAGCAGCCACTGCAGGACCGGATGTTCAAGTTCGAGCTGACCAGACGG |
| | | CTGGACCACGACTTCGGCAAAGTGACCAAGCAAGAAGTGAAGGACTTC |
| | | TTCCGCTGGGCCAAAGATCACGTGGTGGAAGTGGAACACGAGTTCTACG |
| | | TGAAGAAAGGCGGAGCCAAGAAGAGGCCCGCTCCTTCCGATGCCGATA |
| | | TCAGCGAGCCTAAGCGCGTGCGGGAATCTGTGGCTCAGCCTAGCACATC |
| | | TGATGCCGAGGCCAGCATCAACTACGCCGACAGATACCAGAACAAGTG |
| | | CAGCCGGCACGTGGGAATGAATCTGATGCTGTTCCCCTGTCGGCAGTGC |
| | | GAGCGGATGAACCAGAACAGCAACATCTGCTTCACCCACGGCCAGAAA |
| | | GACTGCCTGGAATGCTTCCCCGTGTCCGAGTCTCAGCCTGTGTCCGTGGT |
| | | CAAGAAGGCCTACCAGAAGCTGTGTTACATCCACCACATCATGGGCAA |
| | | AGTGCCCGATGCCTGCACCGCCTGCGATCTGGTTAATGTGGACCTGGAT |
| | | GACTGCATCTTCGAGCAGTGA |
| 26 | NC- | cTGGCGGGCTTCTACGAGATCGTGATCAAGGTGCCCAGCGACCTGGACG |
| | Rep78 + 52 | AGCATCTGCCTGGCATCAGCGACAGCTTCGTGAATTGGGTCGCCGAGAA |
| | D233X | AGAGTGGGAGCTGCCTCCTGACAGCGACTGGACCTGAACCTGATTGAG |
| | | CAGGCCCCTCTGACAGTGGCCGAGAAGCTGCAGAGGGATTTCCTGACA |
| | | GAGTGGCGGAGAGTGTCTAAGGCCCCTGAGGCTCTGTTCTTCGTGCAGT |
| | | TCGAGAAGGGCGAGAGCTACTTCCACTTACACGTGCTGGTCGAGACAAC |
| | | CGGCGTGAAGTCTTTAGTGCTGGGCAGATTCCTGAGCCAGATCAGAGAG |
| | | AAGCTGATCCAGCGGATCTACCGGGGCATCGAGCCCACACTGCCTAATT |
| | | GGTTCGCCGTGACCAAGACCAGAAACGGcGCTGGCGGCGGAAACAAGG |
| | | TGGTGGACGAGTGCTACATCCCCAACTACCTGCTGCCTAAGACACAGCC |
| | | CGAACTGCAGTGGGCCTGGACCAACTTAGAACAGTACCTGAGCGCCTGC |
| | | CTGAATCTGACCGAGCGGAAAAGACTGGTGGCCCAGCATCTGACCCAC |
| | | GTGTCCCAGACACAAGAGCAGAACAAAGAGAATCAGAACCCCAACAGC |
| | | GACGCCCCTGTGATCAGAAGCAAGACCAGCGCCAGATACATGGAACTC |
| | | GTTGGCTGGCTGGTGtagAAGGGCATCACAAGCGAGAAGCAGTGGATCC |
| | | AAGAGGACCAGGCCAGCTACATCAGCTTCAACGCCGCCTCCAACAGCA |
| | | GATCCCAGATCAAGGCCGCTCTGGACAACGCCGGCAAGATCATGAGCC |
| | | TGACAAAGACAGCCCCTGACTACCTCGTGGGCCAGCAGCCTGTGGAAG |
| | | ATATCAGCAGCAACCGGATCTACAAGATCCTGGAACTGAACGGCTACG |
| | | ACCCTCAGTATGCCGCCTCTGTGTTTCTCGGCTGGGCTACCAAGAAGTTC |
| | | GGCAAGCGGAACACCATCTGGCTGTTTGGCCCTGCCACAACCGGCAAG |
| | | ACCAATATCGCCGAGGCTATCGCCCACACCGTGCCTTTTTACGGCTGCG |
| | | TGAACTGGACCAATGAGAACTTCCCCTTCAACGACTGCGTGGACAAGAT |
| | | GGTCATTTGGTGGGAAGAGGGCAAGATGACCGCCAAAGTGGTGGAAAG |
| | | CGCCAAGGCCATCCTCGGCGGATCTAAAGTTCGCGTGGACCAGAAGTGC |
| | | AAGTCTAGCGCCCAGATCGACCCCACACCTGTGATCGTGACCAGCAACA |
| | | CCAACATGTGCGCCGTGATCGACGGCAACAGCACCACCTTTGAACACCA |
| | | GCAGCCACTGCAGGACCGGATGTTCAAGTTCGAGCTGACCAGACGGCT |
| | | GGACCACGACTTCGGCAAAGTGACCAAGCAAGAAGTGAAGGACTTCTT |
| | | CCGCTGGGCCAAAGATCACGTGGTGGAAGTGGAACACGAGTTCTACGT |
| | | GAAGAAAGGCGGAGCCAAGAAGAGGCCCGCTCCTTCCGATGCCGATAT |
| | | CAGCGAGCCTAAGCGCGTGCGGGAATCTGTGGCTCAGCCTAGCACATCT |
| | | GATGCCGAGGCCAGCATCAACTACGCCGACAGATACCAGAACAAGTGC |
| | | AGCCGGCACGTGGGAATGAATCTGATGCTGTTCCCCTGTCGGCAGTGCG |
| | | AGCGGATGAACCAGAACAGCAACATCTGCTTCACCCACGGCCAGAAAG |
| | | ACTGCCTGGAATGCTTCCCCGTGTCCGAGTCTCAGCCTGTGTCCGTGGTC |
| | | AAGAAGGCCTACCAGAAGCTGTGTTACATCCACCACATCATGGGCAAA |
| | | GTGCCCGATGCCTGCACCGCCTGCGATCTGGTTAATGTGGACCTGGATG |
| | | ACTGCATCTTCGAGCAGTGA |
| 27 | NC- | cTGGCGGGCTTCTACGAGATCGTGATCAAGGTGCCCAGCGACCTGGACta |
| | Rep78 + 52 | gCATCTGCCTGGCATCAGCGACAGCTTCGTGAATTGGGTCGCCGAGAAA |
| | E233X, E17X | GAGTGGGAGCTGCCTCCTGACAGCGACITGGACCTGAACCTGATTGAGC |
| | | AGGCCCCTCTGACAGTGGCCGAGAAGCTGCAGAGGGATTTCCTGACAG |
| | | AGTGGCGGAGAGTGTCTAAGGCCCCTGAGGCTCTGTTCTTCGTGCAGTT |
| | | CGAGAAGGGCGAGAGCTACTTCCACTTACACGTGCTGGTCGAGACAAC |
| | | CGGCGTGAAGTCTTTAGTGCTGGGCAGATTCCTGAGCCAGATCAGAGAG |
| | | AAGCTGATCCAGCGGATCTACCGGGGCATCGAGCCCACACTGCCTAATT |
| | | GGTTCGCCGTGACCAAGACCAGAAACGGcGCTGGCGGCGGAAACAAGG |
| | | TGGTGGACGAGTGCTACATCCCCAACTACCTGCTGCCTAAGACACAGCC |
| | | CGAACTGCAGTGGGCCTGGACCAACTTAGAACAGTACCTGAGCGCCTGC |
| | | CTGAATCTGACCGAGCGGAAAAGACTGGTGGCCCAGCATCTGACCCAC |
| | | GTGTCCCAGACACAAGAGCAGAACAAAGAGAATCAGAACCCCAACAGC |
| | | GACGCCCCTGTGATCAGAAGCAAGACCAGCGCCAGATACATGGAACTC |
| | | GTTGGCTGGCTGGTGtagAAGGGCATCACAAGCGAGAAGCAGTGGATCC |
| | | AAGAGGACCAGGCCAGCTACATCAGCTTCAACGCCGCCTCCAACAGCA |
| | | GATCCCAGATCAAGGCCGCTCTGGACAACGCCGGCAAGATCATGAGCC |
| | | TGACAAAGACAGCCCCTGACTACCTCGTGGGCCAGCAGCCTGTGGAAG |
| | | ATATCAGCAGCAACCGGATCTACAAGATCCTGGAACTGAACGGCTACG |
| | | ACCCTCAGTATGCCGCCTCTGTGTTTCTCGGCTGGGCTACCAAGAAGTTC |
| | | GGCAAGCGGAACACCATCTGGCTGTTTGGCCCTGCCACAACCGGCAAG |
| | | ACCAATATCGCCGAGGCTATCGCCCACACCGTGCCTTTTTACGGCTGCG |
| | | TGAACTGGACCAATGAGAACTTCCCCTTCAACGACTGCGTGGACAAGAT |
| | | GGTCATTTGGTGGGAAGAGGGCAAGATGACCGCCAAAGTGGTGGAAAG |
| | | CGCCAAGGCCATCCTCGGCGGATCTAAAGTTCGCGTGGACCAGAAGTGC |
| | | AAGTCTAGCGCCCAGATCGACCCCACACCTGTGATCGTGACCAGCAACA |
| | | CCAACATGTGCGCCGTGATCGACGGCAACAGCACCACCTTTGAACACCA |
| | | GCAGCCACTGCAGGACCGGATGTTCAAGTTCGAGCTGACCAGACGGCT |
| | | GGACCACGACTTCGGCAAAGTGACCAAGCAAGAAGTGAAGGACTTCTT |
| | | CCGCTGGGCCAAAGATCACGTGGTGGAAGTGGAACACGAGTTCTACGT |
| | | GAAGAAAGGCGGAGCCAAGAAGAGGCCCGCTCCTTCCGATGCCGATAT |
| | | CAGCGAGCCTAAGCGCGTGCGGGAATCTGTGGCTCAGCCTAGCACATCT |
| | | GATGCCGAGGCCAGCATCAACTACGCCGACAGATACCAGAACAAGTGC |
| | | AGCCGGCACGTGGGAATGAATCTGATGCTGTTCCCCTGTCGGCAGTGCG |
| | | AGCGGATGAACCAGAACAGCAACATCTGCTTCACCCACGGCCAGAAAG |
| | | ACTGCCTGGAATGCTTCCCCGTGTCCGAGTCTCAGCCTGTGTCCGTGGTC |
| | | AAGAAGGCCTACCAGAAGCTGTGTTACATCCACCACATCATGGGCAAA |
| | | GTGCCCGATGCCTGCACCGCCTGCGATCTGGTTAATGTGGACCTGGATG |
| | | ACTGCATCTTCGAGCAGTGA |
| 28 | NC-Rep | tgatctgcgcagccgccatgccggggttttacgagattgtgattaaggtccccagcgaccttgacgagcatctgcccgg |
| | D233X | catttctgacagctttgtgaactgggtggccgagaaggaatgggagttgccgccagattctgacatggatctgaatctga |
| | | ttgagcaggcacccctgaccgtggccgagaagctgcagcgcgactttctgacggaatggcgccgtgtgagtaaggcc |
| | | ccggaggcccttttctttgtgcaatttgagaagggagagagctacttccacatgcacgtgctcgtggaaaccaccgggg |
| | | tgaaatccatggttttgggacgtttcctgagtcagattcgcgaaaaactgattcagagaatttaccgcgggatcgagccg |
| | | actttgccaaactggttcgcggtcacaaagaccagaaatggcgccggagggggaacaaggtggtggatgagtgcta |
| | | catccccaattacttgctccccaaaacccagcctgagctccagtgggcgtggactaatatggaacagtatttaagcgcct |
| | | gtttgaatctcacggagcgtaaacggttggtggcgcagcatctgacgcacgtgtcgcagacgcaggagcagaacaaa |
| | | gagaatcagaatcccaattctgatgcgccggtgatcagatcaaaaacttcagccaggtacatggagctggtcgggtgg |
| | | ctcgtgTAGaaggggattacctcggagaagcagtggatccaggaggaccaggcctcatacatctccttcaatgcgg |
| | | cctccaactcgcggtcccaaatcaaggctgccttggacaatgcgggaaagattatgagcctgactaaaaccgcccccg |
| | | actacctggtgggccagcagcccgtggaggacatttccagcaatcggatttataaaattttggaactaaacgggtacgat |
| | | ccccaatatgcggcttccgtctttctgggatgggccacgaaaaagttcggcaagaggaacaccatctggctgtttgggc |
| | | ctgcaactaccgggaagaccaacatcgcggaggccatagcccacactgtgcccttctacgggtgcgtaaactggacc |
| | | aatgagaactttcccttcaacgactgtgtcgacaagatggtgatctggtgggaggaggggaagatgaccgccaaggtc |
| | | gtggagtcggccaaagccattctcggaggaagcaaggtgcgcgtggaccagaaatgcaagtcctcggcccagatag |
| | | acccgactcccgtgatcgtcacctccaacaccaacatgtgcgccgtgattgacgggaactcaacgaccttcgaacacc |
| | | agcagccgttgcaagaccggatgttcaaatttgaactcacccgccgtctggatcatgactttgggaaggtcaccaagca |
| | | ggaagtcaaagactttttccggtgggcaaaggatcacgtggttgaggtggagcatgaattctacgtcaaaaagggtgga |
| | | gccaagaaaagacccgcccccagtgacgcagatataagtgagcccaaacgggtgcgcgagtcagttgcgcagccat |
| | | cgacgtcagacgcggaagcttcgatcaactacgcagacaggtaccaaaacaaatgttctcgtcacgtgggcatgaatct |
| | | gatgctgtttccctgcagacaatgcgagagaatgaatcagaattcaaatatctgcttcactcacggacagaaagactgttt |
| | | agagtgctttcccgtgtcagaatctcaacccgtttctgtcgtcaaaaaggcgtatcagaaactgtgctacattcatcatatc |
| | | atgggaaaggtgccagacgcttgcactgcctgcgatctggtcaatgtggatttggatgactgcatctttgaacaataaatg |
| | | atttaaatcaggtatggctgccgatggttatcttccagattggctcgaggacactctctctga |
| 29 | NC-Rep | tgatctgcgcagccgccatgccggggttttacgagattgtgattaaggtccccagcgaccttgacTAGcatctgcccg |
| | D233X, E17X | gcatttctgacagctttgtgaactgggtggccgagaaggaatgggagttgccgccagattctgacatggatctgaatctg |
| | | attgagcaggcacccctgaccgtggccgagaagctgcagcgcgactttctgacggaatggcgccgtgtgagtaaggc |
| | | cccggaggcccttttctttgtgcaatttgagaagggagagagctacttccacatgcacgtgctcgtggaaaccaccggg |
| | | gtgaaatccatggttttgggacgtttcctgagtcagattcgcgaaaaactgattcagagaatttaccgcgggatcgagcc |
| | | gactttgccaaactggttcgcggtcacaaagaccagaaatggcgccggaggcgggaacaaggtggtggatgagtgct |
| | | acatccccaattacttgctccccaaaacccagcctgagctccagtgggcgtggactaatatggaacagtatttaagcgcc |
| | | tgtttgaatctcacggagcgtaaacggttggtggcgcagcatctgacgcacgtgtcgcagacgcaggagcagaacaa |
| | | agagaatcagaatcccaattctgatgcgccggtgatcagatcaaaaacttcagccaggtacatggagctggtcgggtg |
| | | gctcgtgTAGaaggggattacctcggagaagcagtggatccaggaggaccaggcctcatacatctccttcaatgcg |
| | | gcctccaactcgcggtcccaaatcaaggctgccttggacaatgcgggaaagattatgagcctgactaaaaccgccccc |
| | | gactacctggtgggccagcagcccgtggaggacatttccagcaatcggatttataaaattttggaactaaacgggtacg |
| | | atccccaatatgcggcttccgtctttctgggatgggccacgaaaaagttcggcaagaggaacaccatctggctgtttggg |
| | | cctgcaactaccgggaagaccaacatcgcggaggccatagcccacactgtgcccttctacgggtgcgtaaactggac |
| | | caatgagaactttcccttcaacgactgtgtcgacaagatggtgatctggtgggaggaggggaagatgaccgccaaggt |
| | | cgtggagtcggccaaagccattctcggaggaagcaaggtgcgcgtggaccagaaatgcaagtcctcggcccagata |
| | | gacccgactcccgtgatcgtcacctccaacaccaacatgtgcgccgtgattgacgggaactcaacgaccttcgaacac |
| | | cagcagccgttgcaagaccggatgttcaaatttgaactcacccgccgtctggatcatgactttgggaaggtcaccaagc |
| | | aggaagtcaaagactttttccggtgggcaaaggatcacgtggttgaggtggagcatgaattctacgtcaaaaagggtgg |
| | | agccaagaaaagacccgcccccagtgacgcagatataagtgagcccaaacgggtgcgcgagtcagttgcgcagcca |
| | | tcgacgtcagacgcggaagcttcgatcaactacgcagacaggtaccaaaacaaatgttctcgtcacgtgggcatgaatc |
| | | tgatgctgtttccctgcagacaatgcgagagaatgaatcagaattcaaatatctgcttcactcacggacagaaagactgtt |
| | | tagagtgctttcccgtgtcagaatctcaacccgtttctgtcgtcaaaaaggcgtatcagaaactgtgctacattcatcatatc |
| | | atgggaaaggtgccagacgcttgcactgcctgcgatctggtcaatgtggatttggatgactgcatctttgaacaataaatg |
| | | atttaaatcaggtatggctgccgatggttatcttccagattggctcgaggacactctctctga |
| 30 | Rep52 IRES | GCCGCCACCATGGAATTAGTGGGCTGGTTGGTCGATAAAGGCATCACAA |
| | Rep78 Only | GCGAAAAACAATGGATTCAAGAAGATCAAGCGAGCTATATTAGTTTTA |
| | | ACGCCGCTAGTAATAGCAGAAGTCAGATTAAAGCCGCTCTCGATAACGC |
| | | CGGCAAAATCATGTCTTTAACCAAGACAGCTCCTGATTATTTAGTCGGG |
| | | CAACAACCTGTCGAGGACATCAGTTCTAACAGAATCTACAAGATCCTCG |
| | | AATTGAATGGCTATGACCCTCAGTACGCCGCCAGTGTGTTCTTAGGCTG |
| | | GGCTACCAAGAAATTTGGGAAACGCAATACAATTTGGTTATTCGGCCCC |
| | | GCCACCACAGGCAAAACAAATATTGCCGAAGCTATCGCTCATACCGTCC |
| | | CTTTCTATGGCTGTGTGAATTGGACAAACGAAAATTTCCCTTTTAATGAT |
| | | TGCGTGGATAAAATGGTCATTTGGTGGGAAGAAGGCAAAATGACAGCT |
| | | AAAGTGGTCGAAAGCGCTAAGGCTATCTTGGGCGGCTCTAAAGTCAGA |
| | | GTCGATCAAAAGTGTAAAAGTAGCGCTCAAATCGATCCCACCCCTGTCA |
| | | TTGTGACAAGTAATACAAATATGTGTGCTGTCATCGATGGCAATAGCAC |
| | | CACATTTGAGCATCAACAACCCCTCCAGGATAGAATGTTTAAGTTCGAG |
| | | TTGACAAGAAGATTAGACCACGATTTCGGCAAAGTGACAAAACAAGAG |
| | | GTGAAGGATTTCTTTAGATGGGCCAAAGACCATGTCGTGGAAGTCGAAC |
| | | ACGAGTTTTATGTGAAGAAAGGCGGCGCTAAAAAGCGGCCTGCTCCTTC |
| | | CGATGCCGACATCTCCGAACCTAAGAGAGTCAGAGAAAGCGTGGCCCA |
| | | ACCCAGCACCAGCGATGCCGAGGCCAGCATTAATTATGCCGATCGCTAT |
| | | CAGAATAAGTGCAGCAGACATGTCGGGATGAACTTAATGTTATTCCCTT |
| | | GTCGGCAGTGTGAACGGATGAACCAAAACAGCAACATTTGTTTTACCCA |
| | | CGGACAAAAGGATTGCCTGGAATGTTTCCCTGTCAGCGAGAGCCAGCCT |
| | | GTGAGCGTGGTGAAGAAAGCCTACCAAAAGTTATGTTATATCCACCACA |
| | | TTATGGGCAAAGTCCCCGATGCCTGTACCGCTTGTGACTTAGTGAACGT |
| | | AGACCTCGACGATTGTATTTTCGAGCAGTGAtaaGcccctctccctcccccccccctaac |
| | | gttactggccgaagccgcttggaataaggccggtgtgcgtttgtctatatgttattttccaccatattgccgtcttttggcaat |
| | | gtgagggcccggaaacctggccctgtcttcttgacgagcattcctaggggtctttcccctctcgccaaaggaatgcaag |
| | | gtctgttgaatgtcgtgaaggaagcagttcctctggaagcttcttgaagacaaacaacgtctgtagcgaccctttgcagg |
| | | cagcggaaccccccacctggcgacaggtgcctctgcggccaaaagccacgtgtataagatacacctgcaaaggcgg |
| | | cacaaccccagtgccacgttgtgagttggatagttgtggaaagagtcaaatggctctcctcaagcgtattcaacaaggg |
| | | gctgaaggatgcccagaaggtaccccattgtatgggatctgatctggggcctcggtgcacatgctttacatgtgtttagtc |
| | | gaggttaaaaaaacgtctaggccccccgaaccacggggacgtggttttcctttgaaaaacacgatgataatatgCCT |
| | | GGCTTCTACGAGATCGTGATCAAGGTGCCCAGCGACCTGGACGAGCATC |
| | | TGCCTGGCATCAGCGACAGCTTCGTGAATTGGGTCGCCGAGAAAGAGTG |
| | | GGAGCTGCCTCCTGACAGCGACATGGACCTGAACCTGATTGAGCAGGCC |
| | | CCTCTGACAGTGGCCGAGAAGCTGCAGAGGGATTTCCTGACAGAGTGG |
| | | CGGAGAGTGTCTAAGGCCCCTGAGGCTCTGTTCTTCGTGCAGTTCGAGA |
| | | AGGGCGAGAGCTACTTCCACATGCACGTGCTGGTCGAGACAACCGGCG |
| | | TGAAGTCTATGGTGCTGGGCAGATTCCTGAGCCAGATCAGAGAGAAGCT |
| | | GATCCAGCGGATCTACCGGGGCATCGAGCCCACACTGCCTAATTGGTTC |
| | | GCCGTGACCAAGACCAGAAACGGTGCTGGCGGCGGAAACAAGGTGGTG |
| | | GACGAGTGCTACATCCCCAACTACCTGCTGCCTAAGACACAGCCCGAAC |
| | | TGCAGTGGGCCTGGACCAACATGGAACAGTACCTGAGCGCCTGCCTGA |
| | | ATCTGACCGAGCGGAAAAGACTGGTGGCCCAGCATCTGACCCACGTGTC |
| | | CCAGACACAAGAGCAGAACAAAGAGAATCAGAACCCCAACAGCGACG |
| | | CCCCTGTGATCAGAAGCAAGACCAGCGCCAGATACGGaGAACTCGTTGG |
| | | CTGGCTGGTGGACAAGGGCATCACAAGCGAGAAGCAGTGGATCCAAGA |
| | | GGACCAGGCCAGCTACATCAGCTTCAACGCCGCCTCCAACAGCAGATCC |
| | | CAGATCAAGGCCGCTCTGGACAACGCCGGCAAGATCATGAGCCTGACA |
| | | AAGACAGCCCCTGACTACCTCGTGGGCCAGCAGCCTGTGGAAGATATCA |
| | | GCAGCAACCGGATCTACAAGATCCTGGAACTGAACGGCTACGACCCTC |
| | | AGTATGCCGCCTCTGTGTTTCTCGGCTGGGCTACCAAGAAGTTCGGCAA |
| | | GCGGAACACCATCTGGCTGTTTGGCCCTGCCACAACCGGCAAGACCAAT |
| | | ATCGCCGAGGCTATCGCCCACACCGTGCCTTTTTACGGCTGCGTGAACT |
| | | GGACCAATGAGAACTTCCCCTTCAACGACTGCGTGGACAAGATGGTCAT |
| | | TTGGTGGGAAGAGGGCAAGATGACCGCCAAAGTGGTGGAAAGCGCCAA |
| | | GGCCATCCTCGGCGGATCTAAAGTTCGCGTGGACCAGAAGTGCAAGTCT |
| | | AGCGCCCAGATCGACCCCACACCTGTGATCGTGACCAGCAACACCAAC |
| | | ATGTGCGCCGTGATCGACGGCAACAGCACCACCTTTGAACACCAGCAGC |
| | | CACTGCAGGACCGGATGTTCAAGTTCGAGCTGACCAGACGGCTGGACC |
| | | ACGACTTCGGCAAAGTGACCAAGCAAGAAGTGAAGGACTTCTTCCGCT |
| | | GGGCCAAAGATCACGTGGTGGAAGTGGAACACGAGTTCTACGTGAAGA |
| | | AAGGCGGAGCCAAGAAGAGGCCCGCTCCTTCCGATGCCGATATCAGCG |
| | | AGCCTAAGCGCGTGCGGGAATCTGTGGCTCAGCCTAGCACATCTGATGC |
| | | CGAGGCCAGCATCAACTACGCCGACAGATACCAGAACAAGTGCAGCCG |
| | | GCACGTGGGAATGAATCTGATGCTGTTCCCCTGTCGGCAGTGCGAGCGG |
| | | ATGAACCAGAACAGCAACATCTGCTTCACCCACGGCCAGAAAGACTGC |
| | | CTGGAATGCTTCCCCGTGTCCGAGTCTCAGCCTGTGTCCGTGGTCAAGA |
| | | AGGCCTACCAGAAGCTGTGTTACATCCACCACATCATGGGCAAAGTGCC |
| | | CGATGCCTGCACCGCCTGCGATCTGGTTAATGTGGACCTGGATGACTGC |
| | | ATCTTCGAGCAGTGA |
| 31 | NC-Rep52 | GCCGCCACCATGGAATTAGTGGGCTGGTTGGTCtagAAAGGCATCACAAG |
| | IRES NC- | CGAAAAACAATGGATTCAAGAAGATCAAGCGAGCTATATTAGTTTTAAC |
| | NC-Rep78 | GCCGCTAGTAATAGCAGAAGTCAGATTAAAGCCGCTCTCGATAACGCCG |
| | D233X Only | GCAAAATCATGTCTTTAACCAAGACAGCTCCTGATTATTTAGTCGGGCA |
| | | ACAACCTGTCGAGGACATCAGTTCTAACAGAATCTACAAGATCCTCGAA |
| | | TTGAATGGCTATGACCCTCAGTACGCCGCCAGTGTGTTCTTAGGCTGGG |
| | | CTACCAAGAAATTTGGGAAACGCAATACAATTTGGTTATTCGGCCCCGC |
| | | CACCACAGGCAAAACAAATATTGCCGAAGCTATCGCTCATACCGTCCCT |
| | | TTCTATGGCTGTGTGAATTGGACAAACGAAAATTTCCCTTTTAATGATTG |
| | | CGTGGATAAAATGGTCATTTGGTGGGAAGAAGGCAAAATGACAGCTAA |
| | | AGTGGTCGAAAGCGCTAAGGCTATCTTGGGCGGCTCTAAAGTCAGAGTC |
| | | GATCAAAAGTGTAAAAGTAGCGCTCAAATCGATCCCACCCCTGTCATTG |
| | | TGACAAGTAATACAAATATGTGTGCTGTCATCGATGGCAATAGCACCAC |
| | | ATTTGAGCATCAACAACCCCTCCAGGATAGAATGTTTAAGTTCGAGTTG |
| | | ACAAGAAGATTAGACCACGATTTCGGCAAAGTGACAAAACAAGAGGTG |
| | | AAGGATTTCTTTAGATGGGCCAAAGACCATGTCGTGGAAGTCGAACACG |
| | | AGTTTTATGTGAAGAAAGGCGGCGCTAAAAAGCGGCCTGCTCCTTCCGA |
| | | TGCCGACATCTCCGAACCTAAGAGAGTCAGAGAAAGCGTGGCCCAACC |
| | | CAGCACCAGCGATGCCGAGGCCAGCATTAATTATGCCGATCGCTATCAG |
| | | AATAAGTGCAGCAGACATGTCGGGATGAACTTAATGTTATTCCCTTGTC |
| | | GGCAGTGTGAACGGATGAACCAAAACAGCAACATTTGTTTTACCCACGG |
| | | ACAAAAGGATTGCCTGGAATGTTTCCCTGTCAGCGAGAGCCAGCCTGTG |
| | | AGCGTGGTGAAGAAAGCCTACCAAAAGTTATGTTATATCCACCACATTA |
| | | TGGGCAAAGTCCCCGATGCCTGTACCGCTTGTGACTTAGTGAACGTAGA |
| | | CCTCGACGATTGTATTTTCGAGCAGTGAtaaGcccctctccctcccccccccctaacgttact |
| | | ggccgaagccgcttggaataaggccggtgtgcgtttgtctatatgttattttccaccatattgccgtcttttggcaatgtgag |
| | | ggcccggaaacctggccctgtcttcttgacgagcattcctaggggtctttcccctctcgccaaaggaatgcaaggtctgtt |
| | | gaatgtcgtgaaggaagcagttcctctggaagcttcttgaagacaaacaacgtctgtagcgaccctttgcaggcagcgg |
| | | aaccccccacctggcgacaggtgcctctgcggccaaaagccacgtgtataagatacacctgcaaaggcggcacaac |
| | | cccagtgccacgttgtgagttggatagttgtggaaagagtcaaatggctctcctcaagcgtattcaacaaggggctgaa |
| | | ggatgcccagaaggtaccccattgtatgggatctgatctggggcctcggtgcacatgctttacatgtgtttagtcgaggtt |
| | | aaaaaaacgtctaggccccccgaaccacggggacgtggttttcctttgaaaaacacgatgataatatgCCTGGCT |
| | | TCTACGAGATCGTGATCAAGGTGCCCAGCGACCTGGACGAGCATCTGCC |
| | | TGGCATCAGCGACAGCTTCGTGAATTGGGTCGCCGAGAAAGAGTGGGA |
| | | GCTGCCTCCTGACAGCGACATGGACCTGAACCTGATTGAGCAGGCCCCT |
| | | CTGACAGTGGCCGAGAAGCTGCAGAGGGATTTCCTGACAGAGTGGCGG |
| | | AGAGTGTCTAAGGCCCCTGAGGCTCTGTTCTTCGTGCAGTTCGAGAAGG |
| | | GCGAGAGCTACTTCCACATGCACGTGCTGGTCGAGACAACCGGCGTGA |
| | | AGTCTATGGTGCTGGGCAGATTCCTGAGCCAGATCAGAGAGAAGCTGAT |
| | | CCAGCGGATCTACCGGGGCATCGAGCCCACACTGCCTAATTGGTTCGCC |
| | | GTGACCAAGACCAGAAACGGTGCTGGCGGCGGAAACAAGGTGGTGGAC |
| | | GAGTGCTACATCCCCAACTACCTGCTGCCTAAGACACAGCCCGAACTGC |
| | | AGTGGGCCTGGACCAACATGGAACAGTACCTGAGCGCCTGCCTGAATCT |
| | | GACCGAGCGGAAAAGACTGGTGGCCCAGCATCTGACCCACGTGTCCCA |
| | | GACACAAGAGCAGAACAAAGAGAATCAGAACCCCAACAGCGACGCCCC |
| | | TGTGATCAGAAGCAAGACCAGCGCCAGATACGGaGAACTCGTTGGCTGG |
| | | CTGGTGtagAAGGGCATCACAAGCGAGAAGCAGTGGATCCAAGAGGACC |
| | | AGGCCAGCTACATCAGCTTCAACGCCGCCTCCAACAGCAGATCCCAGAT |
| | | CAAGGCCGCTCTGGACAACGCCGGCAAGATCATGAGCCTGACAAAGAC |
| | | AGCCCCTGACTACCTCGTGGGCCAGCAGCCTGTGGAAGATATCAGCAGC |
| | | AACCGGATCTACAAGATCCTGGAACTGAACGGCTACGACCCTCAGTATG |
| | | CCGCCTCTGTGTTTCTCGGCTGGGCTACCAAGAAGTTCGGCAAGCGGAA |
| | | CACCATCTGGCTGTTTGGCCCTGCCACAACCGGCAAGACCAATATCGCC |
| | | GAGGCTATCGCCCACACCGTGCCTTTTTACGGCTGCGTGAACTGGACCA |
| | | ATGAGAACTTCCCCTTCAACGACTGCGTGGACAAGATGGTCATTTGGTG |
| | | GGAAGAGGGCAAGATGACCGCCAAAGTGGTGGAAAGCGCCAAGGCCAT |
| | | CCTCGGCGGATCTAAAGTTCGCGTGGACCAGAAGTGCAAGTCTAGCGCC |
| | | CAGATCGACCCCACACCTGTGATCGTGACCAGCAACACCAACATGTGCG |
| | | CCGTGATCGACGGCAACAGCACCACCTTTGAACACCAGCAGCCACTGCA |
| | | GGACCGGATGTTCAAGTTCGAGCTGACCAGACGGCTGGACCACGACTTC |
| | | GGCAAAGTGACCAAGCAAGAAGTGAAGGACTTCTTCCGCTGGGCCAAA |
| | | GATCACGTGGTGGAAGTGGAACACGAGTTCTACGTGAAGAAAGGCGGA |
| | | GCCAAGAAGAGGCCCGCTCCTTCCGATGCCGATATCAGCGAGCCTAAGC |
| | | GCGTGCGGGAATCTGTGGCTCAGCCTAGCACATCTGATGCCGAGGCCAG |
| | | CATCAACTACGCCGACAGATACCAGAACAAGTGCAGCCGGCACGTGGG |
| | | AATGAATCTGATGCTGTTCCCCTGTCGGCAGTGCGAGCGGATGAACCAG |
| | | AACAGCAACATCTGCTTCACCCACGGCCAGAAAGACTGCCTGGAATGCT |
| | | TCCCCGTGTCCGAGTCTCAGCCTGTGTCCGTGGTCAAGAAGGCCTACCA |
| | | GAAGCTGTGTTACATCCACCACATCATGGGCAAAGTGCCCGATGCCTGC |
| | | ACCGCCTGCGATCTGGTTAATGTGGACCTGGATGACTGCATCTTCGAGC |
| | | AGTGA |
| 32 | NC-Rep52 | GCCGCCACCATGGAATTAGTGGGCTGGTTGGTCtagAAAGGCATCACAAG |
| | IRES NC- | CGAAAAACAATGGATTCAAGAAGATCAAGCGAGCTATATTAGTTTTAAC |
| | Rep78 | GCCGCTAGTAATAGCAGAAGTCAGATTAAAGCCGCTCTCGATAACGCCG |
| | D233X, E17X | GCAAAATCATGTCTTTAACCAAGACAGCTCCTGATTATTTAGTCGGGCA |
| | Only | ACAACCTGTCGAGGACATCAGTTCTAACAGAATCTACAAGATCCTCGAA |
| | | TTGAATGGCTATGACCCTCAGTACGCCGCCAGTGTGTTCTTAGGCTGGG |
| | | CTACCAAGAAATTTGGGAAACGCAATACAATTTGGTTATTCGGCCCCGC |
| | | CACCACAGGCAAAACAAATATTGCCGAAGCTATCGCTCATACCGTCCCT |
| | | TTCTATGGCTGTGTGAATTGGACAAACGAAAATTTCCCTTTTAATGATTG |
| | | CGTGGATAAAATGGTCATTTGGTGGGAAGAAGGCAAAATGACAGCTAA |
| | | AGTGGTCGAAAGCGCTAAGGCTATCTTGGGCGGCTCTAAAGTCAGAGTC |
| | | GATCAAAAGTGTAAAAGTAGCGCTCAAATCGATCCCACCCCTGTCATTG |
| | | TGACAAGTAATACAAATATGTGTGCTGTCATCGATGGCAATAGCACCAC |
| | | ATTTGAGCATCAACAACCCCTCCAGGATAGAATGTTTAAGTTCGAGTTG |
| | | ACAAGAAGATTAGACCACGATTTCGGCAAAGTGACAAAACAAGAGGTG |
| | | AAGGATTTCTTTAGATGGGCCAAAGACCATGTCGTGGAAGTCGAACACG |
| | | AGTTTTATGTGAAGAAAGGCGGCGCTAAAAAGCGGCCTGCTCCTTCCGA |
| | | TGCCGACATCTCCGAACCTAAGAGAGTCAGAGAAAGCGTGGCCCAACC |
| | | CAGCACCAGCGATGCCGAGGCCAGCATTAATTATGCCGATCGCTATCAG |
| | | AATAAGTGCAGCAGACATGTCGGGATGAACTTAATGTTATTCCCTTGTC |
| | | GGCAGTGTGAACGGATGAACCAAAACAGCAACATTTGTTTTACCCACGG |
| | | ACAAAAGGATTGCCTGGAATGTTTCCCTGTCAGCGAGAGCCAGCCTGTG |
| | | AGCGTGGTGAAGAAAGCCTACCAAAAGTTATGTTATATCCACCACATTA |
| | | TGGGCAAAGTCCCCGATGCCTGTACCGCTTGTGACTTAGTGAACGTAGA |
| | | CCTCGACGATTGTATTTTCGAGCAGTGAtaaGcccctctccctcccccccccctaacgttact |
| | | ggccgaagccgcttggaataaggccggtgtgcgtttgtctatatgttattttccaccatattgccgtcttttggcaatgtgag |
| | | ggcccggaaacctggccctgtcttcttgacgagcattcctaggggtctttcccctctcgccaaaggaatgcaaggtctgtt |
| | | gaatgtcgtgaaggaagcagttcctctggaagcttcttgaagacaaacaacgtctgtagcgaccctttgcaggcagcgg |
| | | aaccccccacctggcgacaggtgcctctgcggccaaaagccacgtgtataagatacacctgcaaaggcggcacaac |
| | | cccagtgccacgttgtgagttggatagttgtggaaagagtcaaatggctctcctcaagcgtattcaacaaggggctgaa |
| | | ggatgcccagaaggtaccccattgtatgggatctgatctggggcctcggtgcacatgctttacatgtgtttagtcgaggtt |
| | | aaaaaaacgtctaggccccccgaaccacggggacgtggttttcctttgaaaaacacgatgataatatgCCTGGCT |
| | | TCTACGAGATCGTGATCAAGGTGCCCAGCGACCTGGACtagCATCTGCCT |
| | | GGCATCAGCGACAGCTTCGTGAATTGGGTCGCCGAGAAAGAGTGGGAG |
| | | CTGCCTCCTGACAGCGACATGGACCTGAACCTGATTGAGCAGGCCCCTC |
| | | TGACAGTGGCCGAGAAGCTGCAGAGGGATTTCCTGACAGAGTGGCGGA |
| | | GAGTGTCTAAGGCCCCTGAGGCTCTGTTCTTCGTGCAGTTCGAGAAGGG |
| | | CGAGAGCTACTTCCACATGCACGTGCTGGTCGAGACAACCGGCGTGAA |
| | | GTCTATGGTGCTGGGCAGATTCCTGAGCCAGATCAGAGAGAAGCTGATC |
| | | CAGCGGATCTACCGGGGCATCGAGCCCACACTGCCTAATTGGTTCGCCG |
| | | TGACCAAGACCAGAAACGGTGCTGGCGGCGGAAACAAGGTGGTGGACG |
| | | AGTGCTACATCCCCAACTACCTGCTGCCTAAGACACAGCCCGAACTGCA |
| | | GTGGGCCTGGACCAACATGGAACAGTACCTGAGCGCCTGCCTGAATCTG |
| | | ACCGAGCGGAAAAGACTGGTGGCCCAGCATCTGACCCACGTGTCCCAG |
| | | ACACAAGAGCAGAACAAAGAGAATCAGAACCCCAACAGCGACGCCCCT |
| | | GTGATCAGAAGCAAGACCAGCGCCAGATACGGaGAACTCGTTGGCTGGC |
| | | TGGTGtagAAGGGCATCACAAGCGAGAAGCAGTGGATCCAAGAGGACCA |
| | | GGCCAGCTACATCAGCTTCAACGCCGCCTCCAACAGCAGATCCCAGATC |
| | | AAGGCCGCTCTGGACAACGCCGGCAAGATCATGAGCCTGACAAAGACA |
| | | GCCCCTGACTACCTCGTGGGCCAGCAGCCTGTGGAAGATATCAGCAGCA |
| | | ACCGGATCTACAAGATCCTGGAACTGAACGGCTACGACCCTCAGTATGC |
| | | CGCCTCTGTGTTTCTCGGCTGGGCTACCAAGAAGTTCGGCAAGCGGAAC |
| | | ACCATCTGGCTGTTTGGCCCTGCCACAACCGGCAAGACCAATATCGCCG |
| | | AGGCTATCGCCCACACCGTGCCTTTTTACGGCTGCGTGAACTGGACCAA |
| | | TGAGAACTTCCCCTTCAACGACTGCGTGGACAAGATGGTCATTTGGTGG |
| | | GAAGAGGGCAAGATGACCGCCAAAGTGGTGGAAAGCGCCAAGGCCATC |
| | | CTCGGCGGATCTAAAGTTCGCGTGGACCAGAAGTGCAAGTCTAGCGCCC |
| | | AGATCGACCCCACACCTGTGATCGTGACCAGCAACACCAACATGTGCGC |
| | | CGTGATCGACGGCAACAGCACCACCTTTGAACACCAGCAGCCACTGCA |
| | | GGACCGGATGTTCAAGTTCGAGCTGACCAGACGGCTGGACCACGACTTC |
| | | GGCAAAGTGACCAAGCAAGAAGTGAAGGACTTCTTCCGCTGGGCCAAA |
| | | GATCACGTGGTGGAAGTGGAACACGAGTTCTACGTGAAGAAAGGCGGA |
| | | GCCAAGAAGAGGCCCGCTCCTTCCGATGCCGATATCAGCGAGCCTAAGC |
| | | GCGTGCGGGAATCTGTGGCTCAGCCTAGCACATCTGATGCCGAGGCCAG |
| | | CATCAACTACGCCGACAGATACCAGAACAAGTGCAGCCGGCACGTGGG |
| | | AATGAATCTGATGCTGTTCCCCTGTCGGCAGTGCGAGCGGATGAACCAG |
| | | AACAGCAACATCTGCTTCACCCACGGCCAGAAAGACTGCCTGGAATGCT |
| | | TCCCCGTGTCCGAGTCTCAGCCTGTGTCCGTGGTCAAGAAGGCCTACCA |
| | | GAAGCTGTGTTACATCCACCACATCATGGGCAAAGTGCCCGATGCCTGC |
| | | ACCGCCTGCGATCTGGTTAATGTGGACCTGGATGACTGCATCTTCGAGC |
| | | AGTGA |
| 33 | NC-Rep78 | atgCCTGGCTTCTACGAGATCGTGATCAAGGTGCCCAGCGACCTGGACGA |
| | D233X | GCATCTGCCTGGCATCAGCGACAGCTTCGTGAATTGGGTCGCCGAGAAA |
| | | GAGTGGGAGCTGCCTCCTGACAGCGACATGGACCTGAACCTGATTGAGC |
| | | AGGCCCCTCTGACAGTGGCCGAGAAGCTGCAGAGGGATTTCCTGACAG |
| | | AGTGGCGGAGAGTGTCTAAGGCCCCTGAGGCTCTGTTCTTCGTGCAGTT |
| | | CGAGAAGGGCGAGAGCTACTTCCACATGCACGTGCTGGTCGAGACAAC |
| | | CGGCGTGAAGTCTATGGTGCTGGGCAGATTCCTGAGCCAGATCAGAGA |
| | | GAAGCTGATCCAGCGGATCTACCGGGGCATCGAGCCCACACTGCCTAAT |
| | | TGGTTCGCCGTGACCAAGACCAGAAACGGTGCTGGCGGCGGAAACAAG |
| | | GTGGTGGACGAGTGCTACATCCCCAACTACCTGCTGCCTAAGACACAGC |
| | | CCGAACTGCAGTGGGCCTGGACCAACATGGAACAGTACCTGAGCGCCT |
| | | GCCTGAATCTGACCGAGCGGAAAAGACTGGTGGCCCAGCATCTGACCC |
| | | ACGTGTCCCAGACACAAGAGCAGAACAAAGAGAATCAGAACCCCAACA |
| | | GCGACGCCCCTGTGATCAGAAGCAAGACCAGCGCCAGATACGGaGAAC |
| | | TCGTTGGCTGGCTGGTGtagAAGGGCATCACAAGCGAGAAGCAGTGGATC |
| | | CAAGAGGACCAGGCCAGCTACATCAGCTTCAACGCCGCCTCCAACAGC |
| | | AGATCCCAGATCAAGGCCGCTCTGGACAACGCCGGCAAGATCATGAGC |
| | | CTGACAAAGACAGCCCCTGACTACCTCGTGGGCCAGCAGCCTGTGGAA |
| | | GATATCAGCAGCAACCGGATCTACAAGATCCTGGAACTGAACGGCTAC |
| | | GACCCTCAGTATGCCGCCTCTGTGTTTCTCGGCTGGGCTACCAAGAAGT |
| | | TCGGCAAGCGGAACACCATCTGGCTGTTTGGCCCTGCCACAACCGGCAA |
| | | GACCAATATCGCCGAGGCTATCGCCCACACCGTGCCTTTTTACGGCTGC |
| | | GTGAACTGGACCAATGAGAACTTCCCCTTCAACGACTGCGTGGACAAGA |
| | | TGGTCATTTGGTGGGAAGAGGGCAAGATGACCGCCAAAGTGGTGGAAA |
| | | GCGCCAAGGCCATCCTCGGCGGATCTAAAGTTCGCGTGGACCAGAAGT |
| | | GCAAGTCTAGCGCCCAGATCGACCCCACACCTGTGATCGTGACCAGCAA |
| | | CACCAACATGTGCGCCGTGATCGACGGCAACAGCACCACCTTTGAACAC |
| | | CAGCAGCCACTGCAGGACCGGATGTTCAAGTTCGAGCTGACCAGACGG |
| | | CTGGACCACGACTTCGGCAAAGTGACCAAGCAAGAAGTGAAGGACTTC |
| | | TTCCGCTGGGCCAAAGATCACGTGGTGGAAGTGGAACACGAGTTCTACG |
| | | TGAAGAAAGGCGGAGCCAAGAAGAGGCCCGCTCCTTCCGATGCCGATA |
| | | TCAGCGAGCCTAAGCGCGTGCGGGAATCTGTGGCTCAGCCTAGCACATC |
| | | TGATGCCGAGGCCAGCATCAACTACGCCGACAGATACCAGAACAAGTG |
| | | CAGCCGGCACGTGGGAATGAATCTGATGCTGTTCCCCTGTCGGCAGTGC |
| | | GAGCGGATGAACCAGAACAGCAACATCTGCTTCACCCACGGCCAGAAA |
| | | GACTGCCTGGAATGCTTCCCCGTGTCCGAGTCTCAGCCTGTGTCCGTGGT |
| | | CAAGAAGGCCTACCAGAAGCTGTGTTACATCCACCACATCATGGGCAA |
| | | AGTGCCCGATGCCTGCACCGCCTGCGATCTGGTTAATGTGGACCTGGAT |
| | | GACTGCATCTTCGAGCAGTGA |
| 34 | NC-Rep78 | MPGFYEIVIKVPSDLDEHLPGISDSFVNWVAEKEWELPPDSDMDLNLIEQAP |
| | D233X | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYGELVGWLV*KGITSEKQWIQEDQASYISFN |
| | | AASNSRSQIKAALDNAGKIMSLTKTAPDYLVGQQPVEDISSNRIYKILELNG |
| | | YDPQYAASVFLGWATKKFGKRNTIWLFGPATTGKTNIAEAIAHTVPFYGCV |
| | | NWTNENFPFNDCVDKMVIWWEEGKMTAKVVESAKAILGGSKVRVDQKC |
| | | KSSAQIDPTPVIVTSNTNMCAVIDGNSTTFEHQQPLQDRMFKFELTRRLDHD |
| | | FGKVTKQEVKDFFRWAKDHVVEVEHEFYVKKGGAKKRPAPSDADISEPKR |
| | | VRESVAQPSTSDAEASINYADRYQNKCSRHVGMNLMLFPCRQCERMNQNS |
| | | NICFTHGQKDCLECFPVSESQPVSVVKKAYQKLCYIHHIMGKVPDACTACD |
| | | LVNVDLDDCIFEQ* |
| 35 | NC-Rep78 | atgCCTGGCTTCTACGAGATCGTGATCAAGGTGCCCAGCGACCTGGACtag |
| | E17X | CATCTGCCTGGCATCAGCGACAGCTTCGTGAATTGGGTCGCCGAGAAAG |
| | | AGTGGGAGCTGCCTCCTGACAGCGACATGGACCTGAACCTGATTGAGCA |
| | | GGCCCCTCTGACAGTGGCCGAGAAGCTGCAGAGGGATTTCCTGACAGA |
| | | GTGGCGGAGAGTGTCTAAGGCCCCTGAGGCTCTGTTCTTCGTGCAGTTC |
| | | GAGAAGGGCGAGAGCTACTTCCACATGCACGTGCTGGTCGAGACAACC |
| | | GGCGTGAAGTCTATGGTGCTGGGCAGATTCCTGAGCCAGATCAGAGAG |
| | | AAGCTGATCCAGCGGATCTACCGGGGCATCGAGCCCACACTGCCTAATT |
| | | GGTTCGCCGTGACCAAGACCAGAAACGGTGCTGGCGGCGGAAACAAGG |
| | | TGGTGGACGAGTGCTACATCCCCAACTACCTGCTGCCTAAGACACAGCC |
| | | CGAACTGCAGTGGGCCTGGACCAACATGGAACAGTACCTGAGCGCCTG |
| | | CCTGAATCTGACCGAGCGGAAAAGACTGGTGGCCCAGCATCTGACCCA |
| | | CGTGTCCCAGACACAAGAGCAGAACAAAGAGAATCAGAACCCCAACAG |
| | | CGACGCCCCTGTGATCAGAAGCAAGACCAGCGCCAGATACGGaGAACTC |
| | | GTTGGCTGGCTGGTGGACAAGGGCATCACAAGCGAGAAGCAGTGGATC |
| | | CAAGAGGACCAGGCCAGCTACATCAGCTTCAACGCCGCCTCCAACAGC |
| | | AGATCCCAGATCAAGGCCGCTCTGGACAACGCCGGCAAGATCATGAGC |
| | | CTGACAAAGACAGCCCCTGACTACCTCGTGGGCCAGCAGCCTGTGGAA |
| | | GATATCAGCAGCAACCGGATCTACAAGATCCTGGAACTGAACGGCTAC |
| | | GACCCTCAGTATGCCGCCTCTGTGTTTCTCGGCTGGGCTACCAAGAAGT |
| | | TCGGCAAGCGGAACACCATCTGGCTGTTTGGCCCTGCCACAACCGGCAA |
| | | GACCAATATCGCCGAGGCTATCGCCCACACCGTGCCTTTTTACGGCTGC |
| | | GTGAACTGGACCAATGAGAACTTCCCCTTCAACGACTGCGTGGACAAGA |
| | | TGGTCATTTGGTGGGAAGAGGGCAAGATGACCGCCAAAGTGGTGGAAA |
| | | GCGCCAAGGCCATCCTCGGCGGATCTAAAGTTCGCGTGGACCAGAAGT |
| | | GCAAGTCTAGCGCCCAGATCGACCCCACACCTGTGATCGTGACCAGCAA |
| | | CACCAACATGTGCGCCGTGATCGACGGCAACAGCACCACCTTTGAACAC |
| | | CAGCAGCCACTGCAGGACCGGATGTTCAAGTTCGAGCTGACCAGACGG |
| | | CTGGACCACGACTTCGGCAAAGTGACCAAGCAAGAAGTGAAGGACTTC |
| | | TTCCGCTGGGCCAAAGATCACGTGGTGGAAGTGGAACACGAGTTCTACG |
| | | TGAAGAAAGGCGGAGCCAAGAAGAGGCCCGCTCCTTCCGATGCCGATA |
| | | TCAGCGAGCCTAAGCGCGTGCGGGAATCTGTGGCTCAGCCTAGCACATC |
| | | TGATGCCGAGGCCAGCATCAACTACGCCGACAGATACCAGAACAAGTG |
| | | CAGCCGGCACGTGGGAATGAATCTGATGCTGTTCCCCTGTCGGCAGTGC |
| | | GAGCGGATGAACCAGAACAGCAACATCTGCTTCACCCACGGCCAGAAA |
| | | GACTGCCTGGAATGCTTCCCCGTGTCCGAGTCTCAGCCTGTGTCCGTGGT |
| | | CAAGAAGGCCTACCAGAAGCTGTGTTACATCCACCACATCATGGGCAA |
| | | AGTGCCCGATGCCTGCACCGCCTGCGATCTGGTTAATGTGGACCTGGAT |
| | | GACTGCATCTTCGAGCAGTGA |
| 36 | NC-Rep78 | MPGFYEIVIKVPSDLD*HLPGISDSFVNWVAEKEWELPPDSDMDLNLIEQAP |
| | E17X | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYGELVGWLVDKGITSEKQWIQEDQASYISFN |
| | | AASNSRSQIKAALDNAGKIMSLTKTAPDYLVGQQPVEDISSNRIYKILELNG |
| | | YDPQYAASVFLGWATKKFGKRNTIWLFGPATTGKTNIAEAIAHTVPFYGCV |
| | | NWTNENFPFNDCVDKMVIWWEEGKMTAKVVESAKAILGGSKVRVDQKC |
| | | KSSAQIDPTPVIVTSNTNMCAVIDGNSTTFEHQQPLQDRMFKFELTRRLDHD |
| | | FGKVTKQEVKDFFRWAKDHVVEVEHEFYVKKGGAKKRPAPSDADISEPKR |
| | | VRESVAQPSTSDAEASINYADRYQNKCSRHVGMNLMLFPCRQCERMNQNS |
| | | NICFTHGQKDCLECFPVSESQPVSVVKKAYQKLCYIHHIMGKVPDACTACD |
| | | LVNVDLDDCIFEQ* |
| 37 | NC-Rep78 | atgCCTGGCTTCTACGAGATCGTGATCAAGGTGCCCAGCGACCTGGACtag |
| | D233X; | CATCTGCCTGGCATCAGCGACAGCTTCGTGAATTGGGTCGCCGAGAAAG |
| | E17X | AGTGGGAGCTGCCTCCTGACAGCGACATGGACCTGAACCTGATTGAGCA |
| | | GGCCCCTCTGACAGTGGCCGAGAAGCTGCAGAGGGATTTCCTGACAGA |
| | | GTGGCGGAGAGTGTCTAAGGCCCCTGAGGCTCTGTTCTTCGTGCAGTTC |
| | | GAGAAGGGCGAGAGCTACTTCCACATGCACGTGCTGGTCGAGACAACC |
| | | GGCGTGAAGTCTATGGTGCTGGGCAGATTCCTGAGCCAGATCAGAGAG |
| | | AAGCTGATCCAGCGGATCTACCGGGGCATCGAGCCCACACTGCCTAATT |
| | | GGTTCGCCGTGACCAAGACCAGAAACGGTGCTGGCGGCGGAAACAAGG |
| | | TGGTGGACGAGTGCTACATCCCCAACTACCTGCTGCCTAAGACACAGCC |
| | | CGAACTGCAGTGGGCCTGGACCAACATGGAACAGTACCTGAGCGCCTG |
| | | CCTGAATCTGACCGAGCGGAAAAGACTGGTGGCCCAGCATCTGACCCA |
| | | CGTGTCCCAGACACAAGAGCAGAACAAAGAGAATCAGAACCCCAACAG |
| | | CGACGCCCCTGTGATCAGAAGCAAGACCAGCGCCAGATACGGaGAACTC |
| | | GTTGGCTGGCTGGTGtagAAGGGCATCACAAGCGAGAAGCAGTGGATCC |
| | | AAGAGGACCAGGCCAGCTACATCAGCTTCAACGCCGCCTCCAACAGCA |
| | | GATCCCAGATCAAGGCCGCTCTGGACAACGCCGGCAAGATCATGAGCC |
| | | TGACAAAGACAGCCCCTGACTACCTCGTGGGCCAGCAGCCTGTGGAAG |
| | | ATATCAGCAGCAACCGGATCTACAAGATCCTGGAACTGAACGGCTACG |
| | | ACCCTCAGTATGCCGCCTCTGTGTTTCTCGGCTGGGCTACCAAGAAGTTC |
| | | GGCAAGCGGAACACCATCTGGCTGTTTGGCCCTGCCACAACCGGCAAG |
| | | ACCAATATCGCCGAGGCTATCGCCCACACCGTGCCTTTTTACGGCTGCG |
| | | TGAACTGGACCAATGAGAACTTCCCCTTCAACGACTGCGTGGACAAGAT |
| | | GGTCATTTGGTGGGAAGAGGGCAAGATGACCGCCAAAGTGGTGGAAAG |
| | | CGCCAAGGCCATCCTCGGCGGATCTAAAGTTCGCGTGGACCAGAAGTGC |
| | | AAGTCTAGCGCCCAGATCGACCCCACACCTGTGATCGTGACCAGCAACA |
| | | CCAACATGTGCGCCGTGATCGACGGCAACAGCACCACCTTTGAACACCA |
| | | GCAGCCACTGCAGGACCGGATGTTCAAGTTCGAGCTGACCAGACGGCT |
| | | GGACCACGACTTCGGCAAAGTGACCAAGCAAGAAGTGAAGGACTTCTT |
| | | CCGCTGGGCCAAAGATCACGTGGTGGAAGTGGAACACGAGTTCTACGT |
| | | GAAGAAAGGCGGAGCCAAGAAGAGGCCCGCTCCTTCCGATGCCGATAT |
| | | CAGCGAGCCTAAGCGCGTGCGGGAATCTGTGGCTCAGCCTAGCACATCT |
| | | GATGCCGAGGCCAGCATCAACTACGCCGACAGATACCAGAACAAGTGC |
| | | AGCCGGCACGTGGGAATGAATCTGATGCTGTTCCCCTGTCGGCAGTGCG |
| | | AGCGGATGAACCAGAACAGCAACATCTGCTTCACCCACGGCCAGAAAG |
| | | ACTGCCTGGAATGCTTCCCCGTGTCCGAGTCTCAGCCTGTGTCCGTGGTC |
| | | AAGAAGGCCTACCAGAAGCTGTGTTACATCCACCACATCATGGGCAAA |
| | | GTGCCCGATGCCTGCACCGCCTGCGATCTGGTTAATGTGGACCTGGATG |
| | | ACTGCATCTTCGAGCAGTGA |
| 38 | NC-Rep78 | MPGFYEIVIKVPSDLD*HLPGISDSFVNWVAEKEWELPPDSDMDLNLIEQAP |
| | D233X; | LTVAEKLQRDFLTEWRRVSKAPEALFFVQFEKGESYFHMHVLVETTGVKS |
| | E17X | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYGELVGWLV*KGITSEKQWIQEDQASYISFN |
| | | AASNSRSQIKAALDNAGKIMSLTKTAPDYLVGQQPVEDISSNRIYKILELNG |
| | | YDPQYAASVFLGWATKKFGKRNTIWLFGPATTGKTNIAEAIAHTVPFYGCV |
| | | NWTNENFPFNDCVDKMVIWWEEGKMTAKVVESAKAILGGSKVRVDQKC |
| | | KSSAQIDPTPVIVTSNTNMCAVIDGNSTTFEHQQPLQDRMFKFELTRRLDHD |
| | | FGKVTKQEVKDFFRWAKDHVVEVEHEFYVKKGGAKKRPAPSDADISEPKR |
| | | VRESVAQPSTSDAEASINYADRYQNKCSRHVGMNLMLFPCRQCERMNQNS |
| | | NICFTHGQKDCLECFPVSESQPVSVVKKAYQKLCYIHHIMGKVPDACTACD |
| | | LVNVDLDDCIFEQ* |
| 39 | DA-E2A | MASREEEQRETTPERGRGAARRPPTMEDVSSPSPSPPPPRAPPKKRLRRRLE |
| | (W181*) | SEDEEDSSQDALVPRTPSPRPSTSTADLAIASKKKKKRPSPKPERPPSPEVIV |
| | | DSEEEREDVALQMVGFSNPPVLIKHGKGGKRTVRRLNEDDPVARGMRTQE |
| | | EKEESSEAESESTVINPLSLPIVSA*EKGMEAARALMDKYHVDNDLKANFK |
| | | LLPDQVEALAAVCKTWLNEEHRGLQLTFTSNKTFVTMMGRFLQAYLQSFA |
| | | EVTYKHHEPTGCALWLHRCAEIEGELKCLHGSIMINKEHVIEMDVTSENGQ |
| | | RALKEQSSKAKIVKNRWGRNVVQISNTDARCCVHDAACPANQFSGKSCG |
| | | MFFSEGAKAQVAFKQIKAFMQALYPNAQTGHGHLLMPLRCECNSKPGHAP |
| | | FLGRQLPKLTPFALSNAEDLDADLISDKSVLASVHHPALIVFQCCNPVYRNS |
| | | RAQGGGPNCDFKISAPDLLNALVMVRSLWSENFTELPRMVVPEFKWSTKH |
| | | QYRNVSLPVAHSDARQNPFDF |
| 40 | DA-E2A | MASREEEQRETTPERGRGAARRPPTMEDVSSPSPSPPPPRAPPKKRLRRRLE |
| | (W324*) | SEDEEDSSQDALVPRTPSPRPSTSTADLAIASKKKKKRPSPKPERPPSPEVIV |
| | | DSEEEREDVALQMVGFSNPPVLIKHGKGGKRTVRRLNEDDPVARGMRTQE |
| | | EKEESSEAESESTVINPLSLPIVSAWEKGMEAARALMDKYHVDNDLKANFK |
| | | LLPDQVEALAAVCKTWLNEEHRGLQLTFTSNKTFVTMMGRFLQAYLQSFA |
| | | EVTYKHHEPTGCALWLHRCAEIEGELKCLHGSIMINKEHVIEMDVTSENGQ |
| | | RALKEQSSKAKIVKNR*GRNVVVISNTDARCCVHDAACPANQFSGKSCGM |
| | | FFSEGAKAQVAFKQIKAFMQALYPNAQTGHGHLLMPLRCECNSKPGHAPF |
| | | LGRQLPKLTPFALSNAEDLDADLISDKSVLASVHHPALIVFQCCNPVYRNSR |
| | | AQGGGPNCDFKISAPDLLNALVMVRSLWSENFTELPRMVVPEFKWSTKHQ |
| | | YRNVSLPVAHSDARQNPFDF |
| 41 | DA- | MTTSGVPFGMTLRPTRSRLSRRTPYSRDRLPPFETETRATILEDHPLLPECNT |
| | E4ORF6 | LTMHNVSYVRGLPCSVGFTLIQE*VVPWDMVLTREELVILRKCMHVCLCC |
| | (W77*) | ANIDIMTSMMIHGYESWALHCHCSSPGSLQCIAGGQVLASWFRMVVDGA |
| | | MFNQRFIWYREVVNYNMPKEVMFMSSVFMRGRHLIYLRLWYDGHVGSV |
| | | VPAMSFGYSALHCGILNNIVVLCCSYCADLSEIRVRCCARRTRRLMLRAVRI |
| | | IAEETTAMLYSCRTERRRQQFIRALLQHHRPILMHDYDSTPM |
| 42 | DA- | MTTSGVPFGMTLRPTRSRLSRRTPYSRDRLPPFETETRATILEDHPLLPECNT |
| | E4ORF6 | LTMHNVSYVRGLPCSVGFTLIQEWVVPWDMVLTREELVILRKCMHVCLCC |
| | (W192*) | ANIDIMTSMMIHGYESWALHCHCSSPGSLQCIAGGQVLASWFRMVVDGA |
| | | MFNQRFIWYREVVNYNMPKEVMFMSSVFMRGRHLIYLRL*YDGHVGSVV |
| | | PAMSFGYSALHCGILNNIVVLCCSYCADLSEIRVRCCARRTRRLMLRAVRII |
| | | AEETTAMLYSCRTERRRQQFIRALLQHHRPILMHDYDSTPM |
| 43 | DA-Rep52 | MELVGWLVDKGITSEKQWIQEDQASYISFNAASNSRS*IKAALDNAGKIMS |
| | (Q262*) | LTKTAPDYLVGQQPVEDISSNRIYKILELNGYDPQYAASVFLGWATKKFGK |
| | | RNTIWLFGPATTGKTNIAEAIAHTVPFYGCVNWTNENFPFNDCVDKMVIW |
| | | WEEGKMTAKVVESAKAILGGSKVRVDQKCKSSAQIDPTPVIVTSNTNMCA |
| | | VIDGNSTTFEHQQPLQDRMFKFELTRRLDHDFGKVTKQEVKDFFRWAKDH |
| | | VVEVEHEFYVKKGGAKKRPAPSDADISEPKRVRESVAQPSTSDAEASINYA |
| | | DRYQNKCSRHVGMNLMLFPCRQCERMNQNSNICFTHGQKDCLECFPVSES |
| | | QPVSVVKKAYQKLCYIHHIMGKVPDACTACDLVNVDLDDCIFEQ |
| 44 | DA-Rep40 | MELVGWLVDKGITSEKQWIQEDQASYISFNAASNSRS*IKAALDNAGKIMS |
| | (Q262*) | LTKTAPDYLVGQQPVEDISSNRIYKILELNGYDPQYAASVFLGWATKKFGK |
| | | RNTIWLFGPATTGKTNIAEAIAHTVPFYGCVNWTNENFPFNDCVDKMVIW |
| | | WEEGKMTAKVVESAKAILGGSKVRVDQKCKSSAQIDPTPVIVTSNTNMCA |
| | | VIDGNSTTFEHQQPLQDRMFKFELTRRLDHDFGKVTKQEVKDFFRWAKDH |
| | | VVEVEHEFYVKKGGAKKRPAPSDADISEPKRVRESVAQPSTSDAEASINYA |
| | | DRLARGHSL |
| 45 | DA-Rep78 | MPGFYEIVIKVPSDLDEHLPGISDSFVNWVAEKEWELPPDSDMDLNLIEQAP |
| | (Q262*) | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYMELVGWLVDKGITSEKQWIQEDQASYISFN |
| | | AASNSRS*IKAALDNAGKIMSLTKTAPDYLVGQQPVEDISSNRIYKILELNG |
| | | YDPQYAASVFLGWATKKFGKRNTIWLFGPATTGKTNIAEAIAHTVPFYGCV |
| | | NWTNENFPFNDCVDKMVIWWEEGKMTAKVVESAKAILGGSKVRVDQKC |
| | | KSSAQIDPTPVIVTSNTNMCAVIDGNSTTFEHQQPLQDRMFKFELTRRLDHD |
| | | FGKVTKQEVKDFFRWAKDHVVEVEHEFYVKKGGAKKRPAPSDADISEPKR |
| | | VRESVAQPSTSDAEASINYADRYQNKCSRHVGMNLMLFPCRQCERMNQNS |
| | | NICFTHGQKDCLECFPVSESQPVSVVKKAYQKLCYIHHIMGKVPDACTACD |
| | | LVNVDLDDCIFEQ |
| 46 | DA-Rep68 | MPGFYEIVIKVPSDLDEHLPGISDSFVNWVAEKEWELPPDSDMDLNLIEQAP |
| | (Q262*) | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYMELVGWLVDKGITSEKQWIQEDQASYISFN |
| | | AASNSRS*IKAALDNAGKIMSLTKTAPDYLVGQQPVEDISSNRIYKILELNG |
| | | YDPQYAASVFLGWATKKFGKRNTIWLFGPATTGKTNIAEAIAHTVPFYGCV |
| | | NWTNENFPFNDCVDKMVIWWEEGKMTAKVVESAKAILGGSKVRVDQKC |
| | | KSSAQIDPTPVIVTSNTNMCAVIDGNSTTFEHQQPLQDRMFKFELTRRLDHD |
| | | FGKVTKQEVKDFFRWAKDHVVEVEHEFYVKKGGAKKRPAPSDADISEPKR |
| | | VRESVAQPSTSDAEASINYADRLARGHSL |
| 47 | DA-Rep52 | MELVGWLVDKGITSEKQWIQEDQASYISFNAASNSRSQIKAALDNAGKIMS |
| | (W319*) | LTKTAPDYLVGQQPVEDISSNRIYKILELNGYDPQYAASVFLG*ATKKFGKR |
| | | NTIWLFGPATTGKTNIAEAIAHTVPFYGCVNWTNENFPFNDCVDKMVIWW |
| | | EEGKMTAKVVESAKAILGGSKVRVDQKCKSSAQIDPTPVIVTSNTNMCAVI |
| | | DGNSTTFEHQQPLQDRMFKFELTRRLDHDFGKVTKQEVKDFFRWAKDHV |
| | | VEVEHEFYVKKGGAKKRPAPSDADISEPKRVRESVAQPSTSDAEASINYAD |
| | | RYQNKCSRHVGMNLMLFPCRQCERMNQNSNICFTHGQKDCLECFPVSESQ |
| | | PVSVVKKAYQKLCYIHHIMGKVPDACTACDLVNVDLDDCIFEQ |
| 48 | DA-Rep40 | MELVGWLVDKGITSEKQWIQEDQASYISFNAASNSRSQIKAALDNAGKIMS |
| | (W319*) | LTKTAPDYLVGQQPVEDISSNRIYKILELNGYDPQYAASVFLG*ATKKFGKR |
| | | NTIWLFGPATTGKTNIAEAIAHTVPFYGCVNWTNENFPFNDCVDKMVIWW |
| | | EEGKMTAKVVESAKAILGGSKVRVDQKCKSSAQIDPTPVIVTSNTNMCAVI |
| | | DGNSTTFEHQQPLQDRMFKFELTRRLDHDFGKVTKQEVKDFFRWAKDHV |
| | | VEVEHEFYVKKGGAKKRPAPSDADISEPKRVRESVAQPSTSDAEASINYAD |
| | | RLARGHSL |
| 49 | DA-Rep78 | MPGFYEIVIKVPSDLDEHLPGISDSFVNWVAEKEWELPPDSDMDLNLIEQAP |
| | (W319*) | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYMELVGWLVDKGITSEKQWIQEDQASYISFN |
| | | AASNSRSQIKAALDNAGKIMSLTKTAPDYLVGQQPVEDISSNRIYKILELNG |
| | | YDPQYAASVFLG*ATKKFGKRNTIWLFGPATTGKTNIAEAIAHTVPFYGCV |
| | | NWTNENFPFNDCVDKMVIWWEEGKMTAKVVESAKAILGGSKVRVDQKC |
| | | KSSAQIDPTPVIVTSNTNMCAVIDGNSTTFEHQQPLQDRMFKFELTRRLDHD |
| | | FGKVTKQEVKDFFRWAKDHVVEVEHEFYVKKGGAKKRPAPSDADISEPKR |
| | | VRESVAQPSTSDAEASINYADRYQNKCSRHVGMNLMLFPCRQCERMNQNS |
| | | NICFTHGQKDCLECFPVSESQPVSVVKKAYQKLCYIHHIMGKVPDACTACD |
| | | LVNVDLDDCIFEQ |
| 50 | DA-Rep68 | MPGFYEIVIKVPSDLDEHLPGISDSFVNWVAEKEWELPPDSDMDLNLIEQAP |
| | (W319*) | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYMELVGWLVDKGITSEKQWIQEDQASYISFN |
| | | AASNSRSQIKAALDNAGKIMSLTKTAPDYLVGQQPVEDISSNRIYKILELNG |
| | | YDPQYAASVFLG*ATKKFGKRNTIWLFGPATTGKTNIAEAIAHTVPFYGCV |
| | | NWTNENFPFNDCVDKMVIWWEEGKMTAKVVESAKAILGGSKVRVDQKC |
| | | KSSAQIDPTPVIVTSNTNMCAVIDGNSTTFEHQQPLQDRMFKFELTRRLDHD |
| | | FGKVTKQEVKDFFRWAKDHVVEVEHEFYVKKGGAKKRPAPSDADISEPKR |
| | | VRESVAQPSTSDAEASINYADRLARGHSL |
| 51 | DA-Rep78 | MPGFYEIVIKVPSDLDEHLPGISDSFVNWVAEKEWELPPDSDMDLNLIEQAP |
| | (W67*) | LTVAEKLQRDFLTE*RRVSKAPEALFFVQFEKGESYFHMHVLVETTGVKSM |
| | | VLGRFLSQIREKLIQRIYRGIEPTLPNWFAVTKTRNGAGGGNKVVDECYIPN |
| | | YLLPKTQPELQWAWTNMEQYLSACLNLTERKRLVAQHLTHVSQTQEQNK |
| | | ENQNPNSDAPVIRSKTSARYMELVGWLVDKGITSEKQWIQEDQASYISFNA |
| | | ASNSRSQIKAALDNAGKIMSLTKTAPDYLVGQQPVEDISSNRIYKILELNGY |
| | | DPQYAASVFLGWATKKFGKRNTIWLFGPATTGKTNIAEAIAHTVPFYGCVN |
| | | WTNENFPFNDCVDKMVIWWEEGKMTAKVVESAKAILGGSKVRVDQKCKS |
| | | SAQIDPTPVIVTSNTNMCAVIDGNSTTFEHQQPLQDRMFKFELTRRLDHDFG |
| | | KVTKQEVKDFFRWAKDHVVEVEHEFYVKKGGAKKRPAPSDADISEPKRV |
| | | RESVAQPSTSDAEASINYADRYQNKCSRHVGMNLMLFPCRQCERMNQNSN |
| | | ICFTHGQKDCLECFPVSESQPVSVVKKAYQKLCYIHHIMGKVPDACTACDL |
| | | VNVDLDDCIFEQ |
| 52 | DA-Rep68 | MPGFYEIVIKVPSDLDEHLPGISDSFVNWVAEKEWELPPDSDMDLNLIEQAP |
| | (W67*) | LTVAEKLQRDFLTE*RRVSKAPEALFFVQFEKGESYFHMHVLVETTGVKSM |
| | | VLGRFLSQIREKLIQRIYRGIEPTLPNWFAVTKTRNGAGGGNKVVDECYIPN |
| | | YLLPKTQPELQWAWTNMEQYLSACLNLTERKRLVAQHLTHVSQTQEQNK |
| | | ENQNPNSDAPVIRSKTSARYMELVGWLVDKGITSEKQWIQEDQASYISFNA |
| | | ASNSRSQIKAALDNAGKIMSLTKTAPDYLVGQQPVEDISSNRIYKILELNGY |
| | | DPQYAASVFLGWATKKFGKRNTIWLFGPATTGKTNIAEAIAHTVPFYGCVN |
| | | WTNENFPFNDCVDKMVIWWEEGKMTAKVVESAKAILGGSKVRVDQKCKS |
| | | SAQIDPTPVIVTSNTNMCAVIDGNSTTFEHQQPLQDRMFKFELTRRLDHDFG |
| | | KVTKQEVKDFFRWAKDHVVEVEHEFYVKKGGAKKRPAPSDADISEPKRV |
| | | RESVAQPSTSDAEASINYADRLARGHSL |
| 53 | DA-Rep | ATGCCGGGGTTTTACGAGATTGTGATTAAGGTCCCCAGCGACCTTGACG |
| | (Q262*) | AGCATCTGCCCGGCATTTCTGACAGCTTTGTGAACTGGGTGGCCGAGAA |
| | | GGAATGGGAGTTGCCGCCAGATTCTGACATGGATCTGAATCTGATTGAG |
| | | CAGGCACCCCTGACCGTGGCCGAGAAGCTGCAGCGCGACTTTCTGACGG |
| | | AATGGCGCCGTGTGAGTAAGGCCCCGGAGGCCCTTTTCTTTGTGCAATT |
| | | TGAGAAGGGAGAGAGCTACTTCCACATGCACGTGCTCGTGGAAACCAC |
| | | CGGGGTGAAATCCATGGTTTTGGGACGTTTCCTGAGTCAGATTCGCGAA |
| | | AAACTGATTCAGAGAATTTACCGCGGGATCGAGCCGACTTTGCCAAACT |
| | | GGTTCGCGGTCACAAAGACCAGAAATGGCGCCGGAGGCGGGAACAAGG |
| | | TGGTGGATGAGTGCTACATCCCCAATTACTTGCTCCCCAAAACCCAGCC |
| | | TGAGCTCCAGTGGGCGTGGACTAATATGGAACAGTATTTAAGCGCCTGT |
| | | TTGAATCTCACGGAGCGTAAACGGTTGGTGGCGCAGCATCTGACGCACG |
| | | TGTCGCAGACGCAGGAGCAGAACAAAGAGAATCAGAATCCCAATTCTG |
| | | ATGCGCCGGTGATCAGATCAAAAACTTCAGCCAGGTACATGGAGCTGGT |
| | | CGGGTGGCTCGTGGACAAGGGGATTACCTCGGAGAAGCAGTGGATCCA |
| | | GGAGGACCAGGCCTCATACATCTCCTTCAATGCGGCCTCCAACTCGCGG |
| | | AGCTAAATCAAGGCTGCCTTGGACAATGCGGGAAAGATTATGAGCCTG |
| | | ACTAAAACCGCCCCCGACTACCTGGTGGGCCAGCAGCCCGTGGAGGAC |
| | | ATTTCCAGCAATCGGATTTATAAAATTTTGGAACTAAACGGGTACGATC |
| | | CCCAATATGCGGCTTCCGTCTTTCTGGGATGGGCCACGAAAAAGTTCGG |
| | | CAAGAGGAACACCATCTGGCTGTTTGGGCCTGCAACTACCGGGAAGAC |
| | | CAACATCGCGGAGGCCATAGCCCACACTGTGCCCTTCTACGGGTGCGTA |
| | | AACTGGACCAATGAGAACTTTCCCTTCAACGACTGTGTCGACAAGATGG |
| | | TGATCTGGTGGGAGGAGGGGAAGATGACCGCCAAGGTCGTGGAGTCGG |
| | | CCAAAGCCATTCTCGGAGGAAGCAAGGTGCGCGTGGACCAGAAATGCA |
| | | AGTCCTCGGCCCAGATAGACCCGACTCCCGTGATCGTCACCTCCAACAC |
| | | CAACATGTGCGCCGTGATTGACGGGAACTCAACGACCTTCGAACACCAG |
| | | CAGCCGTTGCAAGACCGGATGTTCAAATTTGAACTCACCCGCCGTCTGG |
| | | ATCATGACTTTGGGAAGGTCACCAAGCAGGAAGTCAAAGACTTTTTCCG |
| | | GTGGGCAAAGGATCACGTGGTTGAGGTGGAGCATGAATTCTACGTCAA |
| | | AAAGGGTGGAGCCAAGAAAAGACCCGCCCCCAGTGACGCAGATATAAG |
| | | TGAGCCCAAACGGGTGCGCGAGTCAGTTGCGCAGCCATCGACGTCAGA |
| | | CGCGGAAGCTTCGATCAACTACGCAGACAGGTACCAAAACAAATGTTCT |
| | | CGTCACGTGGGCATGAATCTGATGCTGTTTCCCTGCAGACAATGCGAGA |
| | | GAATGAATCAGAATTCAAATATCTGCTTCACTCACGGACAGAAAGACTG |
| | | TTTAGAGTGCTTTCCCGTGTCAGAATCTCAACCCGTTTCTGTCGTCAAAA |
| | | AGGCGTATCAGAAACTGTGCTACATTCATCATATCATGGGAAAGGTGCC |
| | | AGACGCTTGCACTGCCTGCGATCTGGTCAATGTGGATTTGGATGACTGC |
| | | ATCTTTGAACAATAAATGATTTAAATCAGGTATGGCTGCCGATGGTTAT |
| | | CTTCCAGATTGGCTCGAGGACACTCTCTCTGA |
| 54 | DA-Rep | ATGCCGGGGTTTTACGAGATTGTGATTAAGGTCCCCAGCGACCTTGACG |
| | (W319*) | AGCATCTGCCCGGCATTTCTGACAGCTTTGTGAACTGGGTGGCCGAGAA |
| | | GGAATGGGAGTTGCCGCCAGATTCTGACATGGATCTGAATCTGATTGAG |
| | | CAGGCACCCCTGACCGTGGCCGAGAAGCTGCAGCGCGACTTTCTGACGG |
| | | AATGGCGCCGTGTGAGTAAGGCCCCGGAGGCCCTTTTCTTTGTGCAATT |
| | | TGAGAAGGGAGAGAGCTACTTCCACATGCACGTGCTCGTGGAAACCAC |
| | | CGGGGTGAAATCCATGGTTTTGGGACGTTTCCTGAGTCAGATTCGCGAA |
| | | AAACTGATTCAGAGAATTTACCGCGGGATCGAGCCGACTTTGCCAAACT |
| | | GGTTCGCGGTCACAAAGACCAGAAATGGCGCCGGAGGCGGGAACAAGG |
| | | TGGTGGATGAGTGCTACATCCCCAATTACTTGCTCCCCAAAACCCAGCC |
| | | TGAGCTCCAGTGGGCGTGGACTAATATGGAACAGTATTTAAGCGCCTGT |
| | | TTGAATCTCACGGAGCGTAAACGGTTGGTGGCGCAGCATCTGACGCACG |
| | | TGTCGCAGACGCAGGAGCAGAACAAAGAGAATCAGAATCCCAATTCTG |
| | | ATGCGCCGGTGATCAGATCAAAAACTTCAGCCAGGTACATGGAGCTGGT |
| | | CGGGTGGCTCGTGGACAAGGGGATTACCTCGGAGAAGCAGTGGATCCA |
| | | GGAGGACCAGGCCTCATACATCTCCTTCAATGCGGCCTCCAACTCGCGG |
| | | TCCCAAATCAAGGCTGCCTTGGACAATGCGGGAAAGATTATGAGCCTGA |
| | | CTAAAACCGCCCCCGACTACCTGGTGGGCCAGCAGCCCGTGGAGGACA |
| | | TTTCCAGCAATCGGATTTATAAAATTTTGGAACTAAACGGGTACGATCC |
| | | CCAATATGCGGCTTCCGTCTTTCTGGGATAGGCCACGAAAAAGTTCGGC |
| | | AAGAGGAACACCATCTGGCTGTTTGGGCCTGCAACTACCGGGAAGACC |
| | | AACATCGCGGAGGCCATAGCCCACACTGTGCCCTTCTACGGGTGCGTAA |
| | | ACTGGACCAATGAGAACTTTCCCTTCAACGACTGTGTCGACAAGATGGT |
| | | GATCTGGTGGGAGGAGGGGAAGATGACCGCCAAGGTCGTGGAGTCGGC |
| | | CAAAGCCATTCTCGGAGGAAGCAAGGTGCGCGTGGACCAGAAATGCAA |
| | | GTCCTCGGCCCAGATAGACCCGACTCCCGTGATCGTCACCTCCAACACC |
| | | AACATGTGCGCCGTGATTGACGGGAACTCAACGACCTTCGAACACCAGC |
| | | AGCCGTTGCAAGACCGGATGTTCAAATTTGAACTCACCCGCCGTCTGGA |
| | | TCATGACTTTGGGAAGGTCACCAAGCAGGAAGTCAAAGACTTTTTCCGG |
| | | TGGGCAAAGGATCACGTGGTTGAGGTGGAGCATGAATTCTACGTCAAA |
| | | AAGGGTGGAGCCAAGAAAAGACCCGCCCCCAGTGACGCAGATATAAGT |
| | | GAGCCCAAACGGGTGCGCGAGTCAGTTGCGCAGCCATCGACGTCAGAC |
| | | GCGGAAGCTTCGATCAACTACGCAGACAGGTACCAAAACAAATGTTCTC |
| | | GTCACGTGGGCATGAATCTGATGCTGTTTCCCTGCAGACAATGCGAGAG |
| | | AATGAATCAGAATTCAAATATCTGCTTCACTCACGGACAGAAAGACTGT |
| | | TTAGAGTGCTTTCCCGTGTCAGAATCTCAACCCGTTTCTGTCGTCAAAAA |
| | | GGCGTATCAGAAACTGTGCTACATTCATCATATCATGGGAAAGGTGCCA |
| | | GACGCTTGCACTGCCTGCGATCTGGTCAATGTGGATTTGGATGACTGCA |
| | | TCTTTGAACAATAAATGATTTAAATCAGGTATGGCTGCCGATGGTTATC |
| | | TTCCAGATTGGCTCGAGGACACTCTCTCTGA |
| 55 | DA-Rep | ATGCCGGGGTTTTACGAGATTGTGATTAAGGTCCCCAGCGACCTTGACG |
| | (W67*) | AGCATCTGCCCGGCATTTCTGACAGCTTTGTGAACTGGGTGGCCGAGAA |
| | | GGAATGGGAGTTGCCGCCAGATTCTGACATGGATCTGAATCTGATTGAG |
| | | CAGGCACCCCTGACCGTGGCCGAGAAGCTGCAGCGCGACTTTCTGACGG |
| | | AGTAGCGCCGTGTGAGTAAGGCCCCGGAGGCCCTTTTCTTTGTGCAATT |
| | | TGAGAAGGGAGAGAGCTACTTCCACATGCACGTGCTCGTGGAAACCAC |
| | | CGGGGTGAAATCCATGGTTTTGGGACGTTTCCTGAGTCAGATTCGCGAA |
| | | AAACTGATTCAGAGAATTTACCGCGGGATCGAGCCGACTTTGCCAAACT |
| | | GGTTCGCGGTCACAAAGACCAGAAATGGCGCCGGAGGCGGGAACAAGG |
| | | TGGTGGATGAGTGCTACATCCCCAATTACTTGCTCCCCAAAACCCAGCC |
| | | TGAGCTCCAGTGGGCGTGGACTAATATGGAACAGTATTTAAGCGCCTGT |
| | | TTGAATCTCACGGAGCGTAAACGGTTGGTGGCGCAGCATCTGACGCACG |
| | | TGTCGCAGACGCAGGAGCAGAACAAAGAGAATCAGAATCCCAATTCTG |
| | | ATGCGCCGGTGATCAGATCAAAAACTTCAGCCAGGTACATGGAGCTGGT |
| | | CGGGTGGCTCGTGGACAAGGGGATTACCTCGGAGAAGCAGTGGATCCA |
| | | GGAGGACCAGGCCTCATACATCTCCTTCAATGCGGCCTCCAACTCGCGG |
| | | TCCCAAATCAAGGCTGCCTTGGACAATGCGGGAAAGATTATGAGCCTGA |
| | | CTAAAACCGCCCCCGACTACCTGGTGGGCCAGCAGCCCGTGGAGGACA |
| | | TTTCCAGCAATCGGATTTATAAAATTTTGGAACTAAACGGGTACGATCC |
| | | CCAATATGCGGCTTCCGTCTTTCTGGGATGGGCCACGAAAAAGTTCGGC |
| | | AAGAGGAACACCATCTGGCTGTTTGGGCCTGCAACTACCGGGAAGACC |
| | | AACATCGCGGAGGCCATAGCCCACACTGTGCCCTTCTACGGGTGCGTAA |
| | | ACTGGACCAATGAGAACTTTCCCTTCAACGACTGTGTCGACAAGATGGT |
| | | GATCTGGTGGGAGGAGGGGAAGATGACCGCCAAGGTCGTGGAGTCGGC |
| | | CAAAGCCATTCTCGGAGGAAGCAAGGTGCGCGTGGACCAGAAATGCAA |
| | | GTCCTCGGCCCAGATAGACCCGACTCCCGTGATCGTCACCTCCAACACC |
| | | AACATGTGCGCCGTGATTGACGGGAACTCAACGACCTTCGAACACCAGC |
| | | AGCCGTTGCAAGACCGGATGTTCAAATTTGAACTCACCCGCCGTCTGGA |
| | | TCATGACTTTGGGAAGGTCACCAAGCAGGAAGTCAAAGACTTTTTCCGG |
| | | TGGGCAAAGGATCACGTGGTTGAGGTGGAGCATGAATTCTACGTCAAA |
| | | AAGGGTGGAGCCAAGAAAAGACCCGCCCCCAGTGACGCAGATATAAGT |
| | | GAGCCCAAACGGGTGCGCGAGTCAGTTGCGCAGCCATCGACGTCAGAC |
| | | GCGGAAGCTTCGATCAACTACGCAGACAGGTACCAAAACAAATGTTCTC |
| | | GTCACGTGGGCATGAATCTGATGCTGTTTCCCTGCAGACAATGCGAGAG |
| | | AATGAATCAGAATTCAAATATCTGCTTCACTCACGGACAGAAAGACTGT |
| | | TTAGAGTGCTTTCCCGTGTCAGAATCTCAACCCGTTTCTGTCGTCAAAAA |
| | | GGCGTATCAGAAACTGTGCTACATTCATCATATCATGGGAAAGGTGCCA |
| | | GACGCTTGCACTGCCTGCGATCTGGTCAATGTGGATTTGGATGACTGCA |
| | | TCTTTGAACAATAAATGATTTAAATCAGGTATGGCTGCCGATGGTTATC |
| | | TTCCAGATTGGCTCGAGGACACTCTCTCTGA |
| 56 | DNA Guide | tgcgtAggagaagggcatgg |
| | E2A DBP | |
| | W181* | |
| 57 | DNA Guide | ccggtAgggccgaaatgtgg |
| | E2A DBP | |
| | W324*, | |
| | Q330V | |
| 58 | DNA Guide | atgAgttgttccctgggata |
| | E4 ORF6 | |
| | W77* | |
| 59 | DNA Guide | cttgtAgtatgatggccacg |
| | E4 ORF6 | |
| | W192* | |
| 60 | DNA Guide | cgtgtAgcagcaatgcctgg |
| | L4 100K | |
| | W435* | |
| 61 | DNA Guide | actgAggattccgacccaag |
| | VP1 | |
| | W304*, | |
| | R310R | |
| 62 | DNA Guide | gccttAtgtgttgacatctg |
| | VP1 Q598* | |
| 63 | DNA Guide | ggaGtAgcgccgtgtgagta |
| | Rep78 | |
| | W67*, E66E | |
| 64 | DNA Guide | gatttAGCTccgcgagttgg |
| | Rep78 | |
| | Q262*, | |
| | S261S | |
| 65 | DNA Guide | ggatAggccacgaaaaagtt |
| | Rep78 | |
| | W319* | |
| 66 | RNA Guide | cttctccCacgcagacacgatcggcaggct |
| | 30 nt E2A | |
| | DBP W181* | |
| 67 | RNA Guide | tcggcccCaccggttcttcacgatcttggc |
| | 30 nt E2A | |
| | DBP | |
| | W324*, | |
| | Q330V | |
| 68 | RNA Guide | gaacaacCcattcctgaatcagcgtaaatc |
| | 30 nt E4 | |
| | ORF6 W77* | |
| 69 | RNA Guide | atcatacCacaagcgcaggtagattaagtg |
| | 30 nt E4 | |
| | ORF6 | |
| | W192* | |
| 70 | RNA Guide | ttgctgcCacacgcccatggccgtttgcca |
| | 30 nt L4 | |
| | 100K | |
| | W435* | |
| 71 | RNA Guide | ggaatccCcagttgttgttgatgagtcttt |
| | 30 nt VP1 | |
| | W304*, | |
| | R310R | |
| 72 | RNA Guide | acggcgcCactccgtcagaaagtcgcgctg |
| | 30 nt Rep78 | |
| | W67*, E66E | |
| 73 | RNA Guide | cgtggccCatcccagaaagacggaagccgc |
| | 30 nt Rep78 | |
| | W319* | |
| 74 | RNA Guide | ctccatgcccttctccCacgcagacacgatcggcaggctcagcgggttta |
| | 50 nt E2A | |
| | DBP W181* | |
| 75 | RNA Guide | caccacatttcggcccCaccggttcttcacgatcttggccttgctagact |
| | 50 nt E2A | |
| | DBP | |
| | W324*, | |
| | Q330V | |
| 76 | RNA Guide | tatcccagggaacaacCcattcctgaatcagegtaaatcccacactgcag |
| | 50 nt E4 | |
| | ORF6 W77* | |
| 77 | RNA Guide | cacgtggccatcatacCacaagcgcaggtagattaagtggcgacccctca |
| | 50 nt E4 | |
| | ORF6 | |
| | W192* | |
| 78 | RNA Guide | ctccaggcattgctgcCacacgcccatggccgtttgccaggtgtagcaca |
| | 50 nt L4 | |
| | 100K | |
| | W435* | |
| 79 | RNA Guide | tcttgggtcggaatccCcagttgttgttgatgagtctttgccagtcacgt |
| | 50 nt VP1 | |
| | W304*, | |
| | R310R | |
| 80 | RNA Guide | cttactcacacggcgcCactccgtcagaaagtcgcgctgcagcttctcgg |
| | 50 nt Rep78 | |
| | W67*, E66E | |
| 81 | RNA Guide | gaactttttcgtggccCatcccagaaagacggaagccgcatattggggat |
| | 50 nt Rep78 | |
| | W319* | |
| 82 | Cas9 ABE | MSEVEFSHEYWMRHALTLAKRAWDEREVPVGAVLVHNNRVIGEGWNRPI |
| | ABE7.10 | GRHDPTAHAEIMALRQGGLVMQNYRLIDATLYVTLEPCVMCAGAMIHSRI |
| | | GRVVFGARDAKTGAAGSLMDVLHHPGMNHRVEITEGILADECAALLSDFF |
| | | RMRRQEIKAQKKAQSSTDSGGSSGGSSGSETPGTSESATPESSGGSSGGSSE |
| | | VEFSHEYWMRHALTLAKRARDEREVPVGAVLVLNNRVIGEGWNRAIGLH |
| | | DPTAHAEIMALRQGGLVMQNYRLIDATLYVTFEPCVMCAGAMIHSRIGRV |
| | | VFGVRNAKTGAAGSLMDVLHYPGMNHRVEITEGILADECAALLCYFFRMP |
| | | RQVFNAQKKAQSSTDSGGSSGGSSGSETPGTSESATPESSGGSSGGSDKKYS |
| | | IGLAIGTNSVGWAVITDEYKVPSKKFKVLGNTDRHSIKKNLIGALLFDSGET |
| | | AEATRLKRTARRRYTRRKNRICYLQEIFSNEMAKVDDSFFHRLEESFLVEED |
| | | KKHERHPIFGNIVDEVAYHEKYPTIYHLRKKLVDSTDKADLRLIYLALAHM |
| | | IKFRGHFLIEGDLNPDNSDVDKLFIQLVQTYNQLFEENPINASGVDAKAILSA |
| | | RLSKSRRLENLIAQLPGEKKNGLFGNLIALSLGLTPNFKSNFDLAEDAKLQL |
| | | SKDTYDDDLDNLLAQIGDQYADLFLAAKNLSDAILLSDILRVNTEITKAPLS |
| | | ASMIKRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHLGELH |
| | | AILRRQEDFYPFLKDNREKIEKILTFRIPYYVGPLARGNSRFAWMTRKSEETI |
| | | TPWNFEEVVDKGASAQSFIERMTNFDKNLPNEKVLPKHSLLYEYFTVYNEL |
| | | TKVKYVTEGMRKPAFLSGEQKKAIVDLLFKTNRKVTVKQLKEDYFKKIEC |
| | | FDSVEISGVEDRFNASLGTYHDLLKIIKDKDFLDNEENEDILEDIVLTLTLFE |
| | | DREMIEERLKTYAHLFDDKVMKQLKRRRYTGWGRLSRKLINGIRDKQSGK |
| | | TILDFLKSDGFANRNFMQLIHDDSLTFKEDIQKAQVSGQGDSLHEHIANLAG |
| | | SPAIKKGILQTVKVVDELVKVMGRHKPENIVIEMARENQTTQKGQKNSRER |
| | | MKRIEEGIKELGSQILKEHPVENTQLQNEKLYLYYLQNGRDMYVDQELDIN |
| | | RLSDYDVDHIVPQSFLKDDSIDNKVLTRSDKNRGKSDNVPSEEVVKKMKN |
| | | YWRQLLNAKLITQRKFDNLTKAERGGLSELDKAGFIKRQLVETRQITKHVA |
| | | QILDSRMNTKYDENDKLIREVKVITLKSKLVSDFRKDFQFYKVREINNYHH |
| | | AHDAYLNAVVGTALIKKYPKLESEFVYGDYKVYDVRKMIAKSEQEIGKAT |
| | | AKYFFYSNIMNFFKTEITLANGEIRKRPLIETNGETGEIVWDKGRDFATVRK |
| | | VLSMPQVNIVKKTEVQTGGFSKESILPKRNSDKLIARKKDWDPKKYGGFDS |
| | | PTVAYSVLVVAKVEKGKSKKLKSVKELLGITIMERSSFEKNPIDFLEAKGYK |
| | | EVKKDLIIKLPKYSLFELENGRKRMLASAGELQKGNELALPSKYVNFLYLA |
| | | SHYEKLKGSPEDNEQKQLFVEQHKHYLDEIIEQISEFSKRVILADANLDKVL |
| | | SAYNKHRDKPIREQAENIIHLFTLTNLGAPAAFKYFDTTIDRKRYTSTKEVL |
| | | DATLIHQSITGLYETRIDLSQLGGDSGGSPKKKRKV |
| 83 | Cas9 ABE | MSEVEFSHEYWMRHALTLAKRARDEREVPVGAVLVLNNRVIGEGWNRAI |
| | ABE8.17m | GLHDPTAHAEIMALRQGGLVMQNYRLIDATLYSTFEPCVMCAGAMIHSRI |
| | [V106W] | GRVVFGWRNAKTGAAGSLMDVLHYPGMNHRVEITEGILADECAALLCYFF |
| | | RMPRRVFNAQKKAQSSTDSGGSSGGSSGSETPGTSESATPESSGGSSGGSDK |
| | | KYSIGLAIGTNSVGWAVITDEYKVPSKKFKVLGNTDRHSIKKNLIGALLFDS |
| | | GETAEATRLKRTARRRYTRRKNRICYLQEIFSNEMAKVDDSFFHRLEESFLV |
| | | EEDKKHERHPIFGNIVDEVAYHEKYPTIYHLRKKLVDSTDKADLRLIYLAL |
| | | AHMIKFRGHFLIEGDLNPDNSDVDKLFIQLVQTYNQLFEENPINASGVDAK |
| | | AILSARLSKSRRLENLIAQLPGEKKNGLFGNLIALSLGLTPNFKSNFDLAEDA |
| | | KLQLSKDTYDDDLDNLLAQIGDQYADLFLAAKNLSDAILLSDILRVNTEITK |
| | | APLSASMIKRYDEHHQDLTLLKALVRQQLPEKYKEIFFDQSKNGYAGYIDG |
| | | GASQEEFYKFIKPILEKMDGTEELLVKLNREDLLRKQRTFDNGSIPHQIHLG |
| | | ELHAILRRQEDFYPFLKDNREKIEKILTFRIPYYVGPLARGNSRFAWMTRKS |
| | | EETITPWNFEEVVDKGASAQSFIERMTNFDKNLPNEKVLPKHSLLYEYFTV |
| | | YNELTKVKYVTEGMRKPAFLSGEQKKAIVDLLFKTNRKVTVKQLKEDYFK |
| | | KIECFDSVEISGVEDRFNASLGTYHDLLKIIKDKDFLDNEENEDILEDIVLTLT |
| | | LFEDREMIEERLKTYAHLFDDKVMKQLKRRRYTGWGRLSRKLINGIRDKQ |
| | | SGKTILDFLKSDGFANRNFMQLIHDDSLTFKEDIQKAQVSGQGDSLHEHIAN |
| | | LAGSPAIKKGILQTVKVVDELVKVMGRHKPENIVIEMARENQTTQKGQKN |
| | | SRERMKRIEEGIKELGSQILKEHPVENTQLQNEKLYLYYLQNGRDMYVDQE |
| | | LDINRLSDYDVDHIVPQSFLKDDSIDNKVLTRSDKNRGKSDNVPSEEVVKK |
| | | MKNYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVREIN |
| | | NYHHAHDAYLNAVVGTALIKKYPKLESEFVYGDYKVYDVRKMIAKSEQEI |
| | | GKATAKYFFYSNIMNFFKTEITLANGEIRKRPLIETNGETGEIVWDKGRDFA |
| | | TVRKVLSMPQVNIVKKTEVQTGGFSKESILPKRNSDKLIARKKDWDPKKYG |
| | | GFDSPTVAYSVLVVAKVEKGKSKKLKSVKELLGITIMERSSFEKNPIDFLEA |
| | | KGYKEVKKDLIIKLPKYSLFELENGRKRMLASAGELQKGNELALPSKYVNF |
| | | LYLASHYEKLKGSPEDNEQKQLFVEQHKHYLDEIIEQISEFSKRVILADANL |
| | | DKVLSAYNKHRDKPIREQAENIIHLFTLTNLGAPAAFKYFDTTIDRKRYTST |
| | | KEVLDATLIHQSITGLYETRIDLSQLGGDEGADKRTADGSEFESPKKKRKV |
| 84 | Cas13 ABE | MNIPALVENQKKYFGTYSVMAMLNAQTVLDHIQKVADIEGEQNENNENL |
| | REPAIRv1 | WFHPVMSHLYNAKNGYDKQPEKTMFIIERLQSYFPFLKIMAENQREYSNG |
| | | KYKQNRVEVNSNDIFEVLKRAFGVLKMYRDLTNAYKTYEEKLNDGCEFLT |
| | | STEQPLSGMINNYYTVALRNMNERYGYKTEDLAFIQDKRFKFVKDAYGKK |
| | | KSQVNTGFFLSLQDYNGDTQKKLHLSGVGIALLICLFLDKQYINIFLSRLPIF |
| | | SSYNAQSEERRIIIRSFGINSIKLPKDRIHSEKSNKSVAMDMLNEVKRCPDEL |
| | | FTTLSAEKQSRFRIISDDHNEVLMKRSSDRFVPLLLQYIDYGKLFDHIRFHVN |
| | | MGKLRYLLKADKTCIDGQTRVRVIEQPLNGFGRLEEAETMRKQENGTFGN |
| | | SGIRIRDFENMKRDDANPANYPYIVDTYTHYILENNKVEMFINDKEDSAPLL |
| | | PVIEDDRYVVKTIPSCRMSTLEIPAMAFHMFLFGSKKTEKLIVDVHNRYKRL |
| | | FQAMQKEEVTAENIASFGIAESDLPQKILDLISGNAHGKDVDAFIRLTVDDM |
| | | LTDTERRIKRFKDDRKSIRSADNKMGKRGFKQISTGKLADFLAKDIVLFQPS |
| | | VNDGENKITGLNYRIMQSAIAVYDSGDDYEAKQQFKLMFEKARLIGKGTT |
| | | EPHPFLYKVFARSIPANAVEFYERYLIERKFYLTGLSNEIKKGNRVDVPFIRR |
| | | DQNKWKTPAMKTLGRIYSEDLPVELPRQMFDNEIKSHLKSLPQMEGIDFNN |
| | | ANVTYLIAEYMKRVLDDDFQTFYQWNRNYRYMDMLKGEYDRKGSLQHC |
| | | FTSVEEREGLWKERASRTERYRKQASNKIRSNRQMRNASSEEIETILDKRLS |
| | | NSRNEYQKSEKVIRRYRVQDALLFLLAKKTLTELADFDGERFKLKEIMPDA |
| | | EKGILSEIMPMSFTFEKGGKKYTITSEGMKLKNYGDFFVLASDKRIGNLLEL |
| | | VGSDIVSKEDIMEEFNKYDQCRPEISSIVFNLEKWAFDTYPELSARVDREEK |
| | | VDFKSILKILLNNKNINKEQSDILRKIRNAFDANNYPDKGVVEIKALPEIAMS |
| | | IKKAFGEYAIMKGSLQLPPLERLTLGSGGGGSQLHLPQVLADAVSRLVLGK |
| | | FGDLTDNFSSPHARRKVLAGVVMTTGTDVKDAKVISVSTGTKCINGEYMS |
| | | DRGLALNDCHAEIISRRSLLRFLYTQLELYLNNKDDQKRSIFQKSERGGFRL |
| | | KENVQFHLYISTSPCGDARIFSPHEPILEEPADRHPNRKARGQLRTKIESGQG |
| | | TIPVRSNASIQTWDGVLQGERLLTMSCSDKIARWNVVGIQGSLLSIFVEPIYF |
| | | SSIILGSLYHGDHLSRAMYQRISNIEDLPPLYTLNKPLLSGISNAEARQPGKA |
| | | PNFSVNWTVGDSAIEVINATTGKDELGRASRLCKHALYCRWMRVHGKVPS |
| | | HLLRSKITKPNVYHESKLAAKEYQAAKARLFTAFIKAGLGAWVEKPTEQD |
| | | QFSLT |
| 85 | Cas13 ABE | MNIPALVENQKKYFGTYSVMAMLNAQTVLDHIQKVADIEGEQNENNENL |
| | REPAIRv2 | WFHPVMSHLYNAKNGYDKQPEKTMFIIERLQSYFPFLKIMAENQREYSNG |
| | | KYKQNRVEVNSNDIFEVLKRAFGVLKMYRDLTNAYKTYEEKLNDGCEFLT |
| | | STEQPLSGMINNYYTVALRNMNERYGYKTEDLAFIQDKRFKFVKDAYGKK |
| | | KSQVNTGFFLSLQDYNGDTQKKLHLSGVGIALLICLFLDKQYINIFLSRLPIF |
| | | SSYNAQSEERRIIIRSFGINSIKLPKDRIHSEKSNKSVAMDMLNEVKRCPDEL |
| | | FTTLSAEKQSRFRIISDDHNEVLMKRSSDRFVPLLLQYIDYGKLFDHIRFHVN |
| | | MGKLRYLLKADKTCIDGQTRVRVIEQPLNGFGRLEEAETMRKQENGTFGN |
| | | SGIRIRDFENMKRDDANPANYPYIVDTYTHYILENNKVEMFINDKEDSAPLL |
| | | PVIEDDRYVVKTIPSCRMSTLEIPAMAFHMFLFGSKKTEKLIVDVHNRYKRL |
| | | FQAMQKEEVTAENIASFGIAESDLPQKILDLISGNAHGKDVDAFIRLTVDDM |
| | | LTDTERRIKRFKDDRKSIRSADNKMGKRGFKQISTGKLADFLAKDIVLFQPS |
| | | VNDGENKITGLNYRIMQSAIAVYDSGDDYEAKQQFKLMFEKARLIGKGTT |
| | | EPHPFLYKVFARSIPANAVEFYERYLIERKFYLTGLSNEIKKGNRVDVPFIRR |
| | | DQNKWKTPAMKTLGRIYSEDLPVELPRQMFDNEIKSHLKSLPQMEGIDFNN |
| | | ANVTYLIAEYMKRVLDDDFQTFYQWNRNYRYMDMLKGEYDRKGSLQHC |
| | | FTSVEEREGLWKERASRTERYRKQASNKIRSNRQMRNASSEEIETILDKRLS |
| | | NSRNEYQKSEKVIRRYRVQDALLFLLAKKTLTELADFDGERFKLKEIMPDA |
| | | EKGILSEIMPMSFTFEKGGKKYTITSEGMKLKNYGDFFVLASDKRIGNLLEL |
| | | VGSDIVSKEDIMEEFNKYDQCRPEISSIVFNLEKWAFDTYPELSARVDREEK |
| | | VDFKSILKILLNNKNINKEQSDILRKIRNAFDANNYPDKGVVEIKALPEIAMS |
| | | IKKAFGEYAIMKGSLQLPPLERLTLGSGGGGSQLHLPQVLADAVSRLVLGK |
| | | FGDLTDNFSSPHARRKVLAGVVMTTGTDVKDAKVISVSTGGKCINGEYMS |
| | | DRGLALNDCHAEIISRRSLLRFLYTQLELYLNNKDDQKRSIFQKSERGGFRL |
| | | KENVQFHLYISTSPCGDARIFSPHEPILEEPADRHPNRKARGQLRTKIESGQG |
| | | TIPVRSNASIQTWDGVLQGERLLTMSCSDKIARWNVVGIQGSLLSIFVEPIYF |
| | | SSIILGSLYHGDHLSRAMYQRISNIEDLPPLYTLNKPLLSGISNAEARQPGKA |
| | | PNFSVNWTVGDSAIEVINATTGKDELGRASRLCKHALYCRWMRVHGKVPS |
| | | HLLRSKITKPNVYHESKLAAKEYQAAKARLFTAFIKAGLGAWVEKPTEQD |
| | | QFSLT |
| 86 | VanR | ATTGGATCCAAT |
| | operator | |
| 87 | TtgR | TATTTACAAACAACCATGAATGTAAGTA |
| | operator | |
| 88 | Gal4 UAS | CGGAGTACTGTCCTCCGA |
| | (for CID | |
| | systems) | |
| 89 | PhlF | ATGATACGAAACGTACCGTATCGTTAAGGT |
| | operator | |
| 90 | CymR | agaaacaaaccaacctgtctgtatta |
| | operator v1 | |
| 91 | CymR | aacaaacagacaatctggtctgtttgta |
| | operator v2 | |
| 92 | TetOff- | MSRLDKSKVINSALELLEVGIEGLTTRKLAQKLGVEQPTLYWHVKNKRA |
| | Advanced | LLDALAIEMLDRHHTHFCPLEGESWQDFLRNNAKSFRCALLSHRDGAKVH |
| | | LGTRPTEKQYETLENQLAFLCQQGFSLENALYALSAVGHFTLGCVLEDQEH |
| | | QVAKEERETPTTDSMPPLLRQAIELFDHQGAEPAFLFGLELIICGLEKQLKCE |
| | | SGGPADALDDFDLDMLPADALDDFDLDMLPADALDDFDLDMLPG |
| 93 | VanR-VP16 | MDMPRIKPGQRVMMALRKMIASGEIKSGERIAEIPTAAALGVSRMPVRIAL |
| | | RSLEQEGLVVRLGARGYAARGVSSDQIRDAIEVRGVLEGFAARRLAERGM |
| | | TAETHARFVVLIAEGEALFAAGRLNGEDLDRYAAYNQAFHDTLVSAAGNG |
| | | AVESALARNGFEPFAAAGALALDLMDLSAEYEHLLAAHRQHQAVLDAVS |
| | | CGDAEGAERIMRDHALAAIRNAKVFEAAASAGAPLGAAWSIRADSGGGGP |
| | | TDALDDFDLDMLPADALDDFDLDMLPADALDDFDLDMLPGPPKKKRKV |
| 94 | TtgR-VP16 | MVRRTKEEAQETRAQIIEAAERAFYKRGVARTTLADIAELAGVTRGAIYWH |
| | | FNNKAELVQALLDSLHETHDHLARASESEDEVDPLGCMRKLLLQVFNELV |
| | | LDARTRRINEILHHKCEFTDDMCEIRQQHQSAVLDCHKGITLTLANVVRRG |
| | | QLPGELDAERAAVAMFAYVDGLIRRWLLLPDSVDLLGDVEKWVDTGLDM |
| | | LRLSPALRKSGGGGPTDALDDFDLDMLPADALDDFDLDMLPADALDDFDL |
| | | DMLPGPPKKKRKV |
| 95 | PhlF-VP16 | MARTPSRSSIGSLRSPHTHKAILTSTIEILKECGYSGLSIESVARRAGAGKPTI |
| | | YRWWTNKAALIAEVYENEIEQVRKFPDLGSFKADLDFLLHNLWKVWRETI |
| | | CGEAFRCVIAEAQLDPVTLTQLKDQFMERRREIPKKLVEDAISNGELPKDIN |
| | | RELLLDMIFGFCWYRLLTEQLTVEQDIEEFTFLLINGVCPGTQCSGGGGPTD |
| | | ALDDFDLDMLPADALDDFDLDMLPADALDDFDLDMLPGPPKKKRKV |
| 96 | cTA | MSPKRRTQAERAMETQGKLIAAALGVLREKGYAGFRIADVPGAAGVSRGA |
| | | QSHHFPTKLELLLATFEWLYEQITERSRARLAKLKPEDDVIQQMLDDAAEF |
| | | FLDDDFSIGLDLIVAADRDPALREGIQRTVERNRFVVEDMWLGVLVSRGLS |
| | | RDDAEDILWLIFNSVRGLVVRSLWQKDKERFERVRNSTLEIARERYAKFKR |
| | | SGGGGPTDALDDFDLDMLPADALDDFDLDMLPADALDDFDLDMLPGPPK |
| | | KKRKV |
| 97 | Rep WT | MPGFYEIVIKVPSDLDEHLPGISDSFVNWVAEKEWELPPDSDMDLNLIEQAP |
| | | LTVAEKLQRDFLTEWRRVSKAPEALFFVQFEKGESYFHMHVLVETTGVKS |
| | | MVLGRFLSQIREKLIQRIYRGIEPTLPNWFAVTKTRNGAGGGNKVVDECYIP |
| | | NYLLPKTQPELQWAWTNMEQYLSACLNLTERKRLVAQHLTHVSQTQEQN |
| | | KENQNPNSDAPVIRSKTSARYMELVGWLVDKGITSEKQWIQEDQASYISFN |
| | | AASNSRSQIKAALDNAGKIMSLTKTAPDYLVGQQPVEDISSNRIYKILELNG |
| | | YDPQYAASVFLGWATKKFGKRNTIWLFGPATTGKTNIAEAIAHTVPFYGCV |
| | | NWTNENFPFNDCVDKMVIWWEEGKMTAKVVESAKAILGGSKVRVDQKC |
| | | KSSAQIDPTPVIVTSNTNMCAVIDGNSTTFEHQQPLQDRMFKFELTRRLDHD |
| | | FGKVTKQEVKDFFRWAKDHVVEVEHEFYVKKGGAKKRPAPSDADISEPKR |
| | | VRESVAQPSTSDAEASINYADRYQNKCSRHVGMNLMLFPCRQCERMNQNS |
| | | NICFTHGQKDCLECFPVSESQPVSVVKKAYQKLCYIHHIMGKVPDACTACD |
| | | LVNVDLDDCIFEQ-MI-IRYGCRWLSSRLARGHSL- |
| 98 | DA-L4 100K | MESVEKEDSLTAPFEFATTASTDAANAPTTFPVEAPPLEEEEVIIEQDPGFVSE |
| | (W435*) | DDEDRSVPTEDKKQDQDDAEANEEQVGRGDQRHGDYLDVGDDVLLKHLQ |
| | | RQCAIICDALQERSDVPLAIADVSLAYERHLFSPRVPPKRQENGTCEPNPRLN |
| | | FYPVFAVPEVLATYHIFFQNCKIPLSCRANRSRADKQLALRQGAVIPDIASLD |
| | | EVPKIFEGLGRDEKRAANALQQENSENESHCGVLVELEGDNARLAVLKRSIE |
| | | VTHFAYPALNLPPKVMSTVMSELIVRRARPLERDANLQEQTEEGLPAVGDEQ |
| | | LARWLETREPADLEERRKLMMAAVLVTVELECMQRFFADPEMQRKLEETL |
| | | HYTFRQGYVRQACKISNVELCNLVSYLGILHENRLGQNVLHSTLKGEARRD |
| | | YVRDCVYLFLCYTWQTAMGV*QQCLEERNLKELQKLLKQNLKDLWTAFNE |
| | | RSVAAHLADIIFPERLLKTLQQGLPDFTSQSMLQNFRNFILERSGILPATCCAL |
| | | PSDFVPIKYRECPPPLWGHCYLLQLANYLAYHSDIMEDVSGDGLLECHCRCN |
| | | LCTPHRSLVCNSQLLSESQIIGTFELQGPSPDEKSAAPGLKLTPGLWTSAYLRK |
| | | FVPEDYHAHEIRFYEDQSRPPNAELTACVITQGHILGQLQAINKARQEFLLRK |
| | | GRGVYLDPQSGEELNPIPPPPQPYQQPRALASQDGTQKEAAAAAAATHGRG |
| | | GILGQSGRGGFGRGGGDDGRLGQPRRSFRGRRGVRRNTVTLGRIPLAGAPEI |
| | | GNRSQHRYNLRSSGAAGTACSPTQP |
| 99 | DA-VP1 | MAADGYLPDWLEDTLSEGIRQWWKLKPGPPPPKPAERHKDDSRGLVLPGYK |
| | (W304*) | YLGPFNGLDKGEPVNEADAAALEHDKAYDRQLDSGDNPYLKYNHADAEFQ |
| | | ERLKEDTSFGGNLGRAVFQAKKRVLEPLGLVEEPVKTAPGKKRPVEHSPVEP |
| | | DSSSGTGKAGQQPARKRLNFGQTGDADSVPDPQPLGQPPAAPSGLGTNTMA |
| | | TGSGAPMADNNEGADGVGNSSGNWHCDSTWMGDRVITTSTRTWALPTYN |
| | | NHLYKQISSQSGASNDNHYFGYSTPWGYFDFNRFHCHFSPRDWQRLINNN*G |
| | | FRPKRLNFKLFNIQVKEVTQNDGTTTIANNLTSTVQVFTDSEYQLPYVLGSAH |
| | | QGCLPPFPADVFMVPQYGYLTLNNGSQAVGRSSFYCLEYFPSQMLRTGNNFT |
| | | FSYTFEDVPFHSSYAHSQSLDRLMNPLIDQYLYYLSRTNTPSGTTTQSRLQFS |
| | | QAGASDIRDQSRNWLPGPCYRQQRVSKTSADNNNSEYSWTGATKYHLNGR |
| | | DSLVNPGPAMASHKDDEEKFFPQSGVLIFGKQGSEKTNVDIEKVMITDEEEIR |
| | | TTNPVATEQYGSVSTNLQRGNRQAATADVNTQGVLPGMVWQDRDVYLQG |
| | | PIWAKIPHTDGHFHPSPLMGGFGLKHPPPQILIKNTPVPANPSTTFSAAKFASFI |
| | | TQYSTGQVSVEIEWELQKENSKRWNPEIQYTSNYNKSVNVDFTVDTNGVYS |
| | | EPRPIGTRYLTRNL |
| 100 | DA-VP2 | TAPGKKRPVEHSPVEPDSSSGTGKAGQQPARKRLNFGQTGDADSVPDPQPLG |
| | (W304*) | QPPAAPSGLGTNTMATGSGAPMADNNEGADGVGNSSGNWHCDSTWMGDR |
| | | VITTSTRTWALPTYNNHLYKQISSQSGASNDNHYFGYSTPWGYFDFNRFHCH |
| | | FSPRDWQRLINNN*GFRPKRLNFKLFNIQVKEVTQNDGTTTIANNLTSTVQVF |
| | | TDSEYQLPYVLGSAHQGCLPPFPADVFMVPQYGYLTLNNGSQAVGRSSFYC |
| | | LEYFPSQMLRTGNNFTFSYTFEDVPFHSSYAHSQSLDRLMNPLIDQYLYYLSR |
| | | TNTPSGTTTQSRLQFSQAGASDIRDQSRNWLPGPCYRQQRVSKTSADNNNSE |
| | | YSWTGATKYHLNGRDSLVNPGPAMASHKDDEEKFFPQSGVLIFGKQGSEKT |
| | | NVDIEKVMITDEEEIRTTNPVATEQYGSVSTNLQRGNRQAATADVNTQGVLP |
| | | GMVWQDRDVYLQGPIWAKIPHTDGHFHPSPLMGGFGLKHPPPQILIKNTPVP |
| | | ANPSTTFSAAKFASFITQYSTGQVSVEIEWELQKENSKRWNPEIQYTSNYNKS |
| | | VNVDFTVDTNGVYSEPRPIGTRYLTRNL |
| 101 | \|DA-VP3 | MATGSGAPMADNNEGADGVGNSSGNWHCDSTWMGDRVITTSTRTWALPT |
| | (W304*) | YNNHLYKQISSQSGASNDNHYFGYSTPWGYFDFNRFHCHFSPRDWQRLINN |
| | | N*GFRPKRLNFKLFNIQVKEVTQNDGTTTIANNLTSTVQVFTDSEYQLPYVLG |
| | | SAHQGCLPPFPADVFMVPQYGYLTLNNGSQAVGRSSFYCLEYFPSQMLRTG |
| | | NNFTFSYTFEDVPFHSSYAHSQSLDRLMNPLIDQYLYYLSRTNTPSGTTTQSR |
| | | LQFSQAGASDIRDQSRNWLPGPCYRQQRVSKTSADNNNSEYSWTGATKYHL |
| | | NGRDSLVNPGPAMASHKDDEEKFFPQSGVLIFGKQGSEKTNVDIEKVMITDE |
| | | EEIRTTNPVATEQYGSVSTNLQRGNRQAATADVNTQGVLPGMVWQDRDVY |
| | | LQGPIWAKIPHTDGHFHPSPLMGGFGLKHPPPQILIKNTPVPANPSTTFSAAKF |
| | | ASFITQYSTGQVSVEIEWELQKENSKRWNPEIQYTSNYNKSVNVDFTVDTNG |
| | | VYSEPRPIGTRYLTRNL |
| 102 | DA-VP1 | MAADGYLPDWLEDTLSEGIRQWWKLKPGPPPPKPAERHKDDSRGLVLPGYK |
| | (Q598*) | YLGPFNGLDKGEPVNEADAAALEHDKAYDRQLDSGDNPYLKYNHADAEFQ |
| | | ERLKEDTSFGGNLGRAVFQAKKRVLEPLGLVEEPVKTAPGKKRPVEHSPVEP |
| | | DSSSGTGKAGQQPARKRLNFGQTGDADSVPDPQPLGQPPAAPSGLGTNTMA |
| | | TGSGAPMADNNEGADGVGNSSGNWHCDSTWMGDRVITTSTRTWALPTYN |
| | | NHLYKQISSQSGASNDNHYFGYSTPWGYFDFNRFHCHFSPRDWQRLINNNW |
| | | GFRPKRLNFKLFNIQVKEVTQNDGTTTIANNLTSTVQVFTDSEYQLPYVLGSA |
| | | HQGCLPPFPADVFMVPQYGYLTLNNGSQAVGRSSFYCLEYFPSQMLRTGNN |
| | | FTFSYTFEDVPFHSSYAHSQSLDRLMNPLIDQYLYYLSRTNTPSGTTTQSRLQ |
| | | FSQAGASDIRDQSRNWLPGPCYRQQRVSKTSADNNNSEYSWTGATKYHLNG |
| | | RDSLVNPGPAMASHKDDEEKFFPQSGVLIFGKQGSEKTNVDIEKVMITDEEEI |
| | | RTTNPVATEQYGSVSTNLQRGNRQAATADVNT*GVLPGMVWQDRDVYLQG |
| | | PIWAKIPHTDGHFHPSPLMGGFGLKHPPPQILIKNTPVPANPSTTFSAAKFASFI |
| | | TQYSTGQVSVEIEWELQKENSKRWNPEIQYTSNYNKSVNVDFTVDTNGVYS |
| | | EPRPIGTRYLTRNL |
| 103 | DA-VP2 | TAPGKKRPVEHSPVEPDSSSGTGKAGQQPARKRLNFGQTGDADSVPDPQPLG |
| | (Q598*) | QPPAAPSGLGTNTMATGSGAPMADNNEGADGVGNSSGNWHCDSTWMGDR |
| | | VITTSTRTWALPTYNNHLYKQISSQSGASNDNHYFGYSTPWGYFDFNRFHCH |
| | | FSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYC |
| | | LEYFPSQMLRTGNNFTFSYTFEDVPFHSSYAHSQSLDRLMNPLIDQYLYYLSR |
| | | TNTPSGTTTQSRLQFSQAGASDIRDQSRNWLPGPCYRQQRVSKTSADNNNSE |
| | | YSWTGATKYHLNGRDSLVNPGPAMASHKDDEEKFFPQSGVLIFGKQGSEKT |
| | | NVDIEKVMITDEEEIRTTNPVATEQYGSVSTNLQRGNRQAATADVNT*GVLP |
| | | GMVWQDRDVYLQGPIWAKIPHTDGHFHPSPLMGGFGLKHPPPQILIKNTPVP |
| | | ANPSTTFSAAKFASFITQYSTGQVSVEIEWELQKENSKRWNPEIQYTSNYNKS |
| | | VNVDFTVDTNGVYSEPRPIGTRYLTRNL |
| 104 | DA-VP3 | MATGSGAPMADNNEGADGVGNSSGNWHCDSTWMGDRVITTSTRTWALPT |
| | (Q598*) | YNNHLYKQISSQSGASNDNHYFGYSTPWGYFDFNRFHCHFSPRDWQRLINN |
| | | NWGFRPKRLNFKLFNIQVKEVTQNDGTTTIANNLTSTVQVFTDSEYQLPYVL |
| | | GSAHQGCLPPFPADVFMVPQYGYLTLNNGSQAVGRSSFYCLEYFPSQMLRT |
| | | GNNFTFSYTFEDVPFHSSYAHSQSLDRLMNPLIDQYLYYLSRTNTPSGTTTQS |
| | | RLQFSQAGASDIRDQSRNWLPGPCYRQQRVSKTSADNNNSEYSWTGATKYH |
| | | LNGRDSLVNPGPAMASHKDDEEKFFPQSGVLIFGKQGSEKTNVDIEKVMITD |
| | | EEEIRTTNPVATEQYGSVSTNLQRGNRQAATADVNT*GVLPGMVWQDRDV |
| | | YLQGPIWAKIPHTDGHFHPSPLMGGFGLKHPPPQILIKNTPVPANPSTTFSAAK |
| | | FASFITQYSTGQVSVEIEWELQKENSKRWNPEIQYTSNYNKSVNVDFTVDTN |
| | | GVYSEPRPIGTRYLTRNL |
| 105 | DA-E2A | ATGGCCAGTCGGGAAGAGGAGCAGCGCGAAACCACCCCCGAGCGCGGAC |
| | (W181*) | GCGGTGCGGCGCGACGTCCACCAACCATGGAGGACGTGTCGTCCCCGTCG |
| | | CCGTCGCCGCCGCCTCCCCGCGCGCCCCCAAAAAAGCGGCTGAGGCGGC |
| | | GTCTCGAGTCCGAGGACGAAGAAGACTCGTCACAAGATGCGCTGGTGCC |
| | | GCGCACACCCAGCCCGCGGCCATCGACCTCGACGGCGGATTTGGCCATTG |
| | | CGTCCAAAAAGAAAAAGAAGCGCCCCTCTCCCAAGCCCGAGCGCCCGCC |
| | | ATCCCCAGAGGTGATCGTGGACAGCGAGGAAGAAAGAGAAGATGTGGCG |
| | | CTACAAATGGTGGGTTTCAGCAACCCACCGGTGCTAATCAAGCACGGCAA |
| | | GGGAGGTAAGCGCACGGTGCGGCGGCTGAATGAAGACGACCCAGTGGCG |
| | | CGGGGTATGCGGACGCAAGAGGAAAAGGAAGAGTCCAGTGAAGCGGAA |
| | | AGTGAAAGCACGGTGATAAACCCGCTGAGCCTGCCGATCGTGTCTGCGTa |
| | | GGAGAAGGGCATGGAGGCTGCGCGCGCGTTGATGGACAAGTACCACGTG |
| | | GATAACGATCTAAAGGCAAACTTCAAGCTACTGCCTGACCAAGTGGAAG |
| | | CTCTGGCGGCCGTATGCAAGACCTGGCTAAACGAGGAGCACCGCGGGTT |
| | | GCAGCTGACCTTCACCAGCAACAAGACCTTTGTGACGATGATGGGGCGAT |
| | | TCCTGCAGGCGTACCTGCAGTCGTTTGCAGAGGTAACCTACAAGCACCAC |
| | | GAGCCCACGGGCTGCGCGTTGTGGCTGCACCGCTGCGCTGAGATCGAAG |
| | | GCGAGCTTAAGTGTCTACACGGGAGCATTATGATAAATAAGGAGCACGT |
| | | GATTGAAATGGATGTGACGAGCGAAAACGGGCAGCGCGCGCTGAAGGAG |
| | | CAGTCTAGCAAGGCCAAGATCGTGAAGAACCGGTGGGGCCGAAATGTGG |
| | | TGCAGATCTCCAACACCGACGCAAGGTGCTGCGTGCATGACGCGGCCTGT |
| | | CCGGCCAATCAGTTTTCCGGCAAGTCTTGCGGCATGTTCTTCTCTGAAGGC |
| | | GCAAAGGCTCAGGTGGCTTTTAAGCAGATCAAGGCTTTCATGCAGGCGCT |
| | | GTATCCTAACGCCCAGACCGGGCACGGTCACCTTCTGATGCCACTACGGT |
| | | GCGAGTGCAACTCAAAGCCTGGGCATGCACCCTTTTTGGGAAGGCAGCTA |
| | | CCAAAGTTGACTCCGTTCGCCCTGAGCAACGCGGAGGACCTGGACGCGG |
| | | ATCTGATCTCCGACAAGAGCGTGCTGGCCAGCGTGCACCACCCGGCGCTG |
| | | ATAGTGTTCCAGTGCTGCAACCCTGTGTATCGCAACTCGCGCGCGCAGGG |
| | | CGGAGGCCCCAACTGCGACTTCAAGATATCGGCGCCCGACCTGCTAAACG |
| | | CGTTGGTGATGGTGCGCAGCCTGTGGAGTGAAAACTTCACCGAGCTGCCG |
| | | CGGATGGTTGTGCCTGAGTTTAAGTGGAGCACTAAACACCAGTATCGCAA |
| | | CGTGTCCCTGCCAGTGGCGCATAGCGATGCGCGGCAGAACCCCTTTGATT |
| | | TTTAA |
| 106 | DA-E2A | ATGGCCAGTCGGGAAGAGGAGCAGCGCGAAACCACCCCCGAGCGCGGAC |
| | (W324*) | GCGGTGCGGCGCGACGTCCACCAACCATGGAGGACGTGTCGTCCCCGTCG |
| | | CCGTCGCCGCCGCCTCCCCGCGCGCCCCCAAAAAAGCGGCTGAGGCGGC |
| | | GTCTCGAGTCCGAGGACGAAGAAGACTCGTCACAAGATGCGCTGGTGCC |
| | | GCGCACACCCAGCCCGCGGCCATCGACCTCGACGGCGGATTTGGCCATTG |
| | | CGTCCAAAAAGAAAAAGAAGCGCCCCTCTCCCAAGCCCGAGCGCCCGCC |
| | | ATCCCCAGAGGTGATCGTGGACAGCGAGGAAGAAAGAGAAGATGTGGCG |
| | | CTACAAATGGTGGGTTTCAGCAACCCACCGGTGCTAATCAAGCACGGCAA |
| | | GGGAGGTAAGCGCACGGTGCGGCGGCTGAATGAAGACGACCCAGTGGCG |
| | | CGGGGTATGCGGACGCAAGAGGAAAAGGAAGAGTCCAGTGAAGCGGAA |
| | | AGTGAAAGCACGGTGATAAACCCGCTGAGCCTGCCGATCGTGTCTGCGTG |
| | | GGAGAAGGGCATGGAGGCTGCGCGCGCGTTGATGGACAAGTACCACGTG |
| | | GATAACGATCTAAAGGCAAACTTCAAGCTACTGCCTGACCAAGTGGAAG |
| | | CTCTGGCGGCCGTATGCAAGACCTGGCTAAACGAGGAGCACCGCGGGTT |
| | | GCAGCTGACCTTCACCAGCAACAAGACCTTTGTGACGATGATGGGGCGAT |
| | | TCCTGCAGGCGTACCTGCAGTCGTTTGCAGAGGTAACCTACAAGCACCAC |
| | | GAGCCCACGGGCTGCGCGTTGTGGCTGCACCGCTGCGCTGAGATCGAAG |
| | | GCGAGCTTAAGTGTCTACACGGGAGCATTATGATAAATAAGGAGCACGT |
| | | GATTGAAATGGATGTGACGAGCGAAAACGGGCAGCGCGCGCTGAAGGAG |
| | | CAGTCTAGCAAGGCCAAGATCGTGAAGAACCGGTaGGGCCGAAATGTGG |
| | | TGgtGATCTCCAACACCGACGCAAGGTGCTGCGTGCATGACGCGGCCTGTC |
| | | CGGCCAATCAGTTTTCCGGCAAGTCTTGCGGCATGTTCTTCTCTGAAGGC |
| | | GCAAAGGCTCAGGTGGCTTTTAAGCAGATCAAGGCTTTCATGCAGGCGCT |
| | | GTATCCTAACGCCCAGACCGGGCACGGTCACCTTCTGATGCCACTACGGT |
| | | GCGAGTGCAACTCAAAGCCTGGGCATGCACCCTTTTTGGGAAGGCAGCTA |
| | | CCAAAGTTGACTCCGTTCGCCCTGAGCAACGCGGAGGACCTGGACGCGG |
| | | ATCTGATCTCCGACAAGAGCGTGCTGGCCAGCGTGCACCACCCGGCGCTG |
| | | ATAGTGTTCCAGTGCTGCAACCCTGTGTATCGCAACTCGCGCGCGCAGGG |
| | | CGGAGGCCCCAACTGCGACTTCAAGATATCGGCGCCCGACCTGCTAAACG |
| | | CGTTGGTGATGGTGCGCAGCCTGTGGAGTGAAAACTTCACCGAGCTGCCG |
| | | CGGATGGTTGTGCCTGAGTTTAAGTGGAGCACTAAACACCAGTATCGCAA |
| | | CGTGTCCCTGCCAGTGGCGCATAGCGATGCGCGGCAGAACCCCTTTGATT |
| | | TTTAA |
| 107 | DA-E4ORF6 | ATGACTACGTCCGGCGTTCCATTTGGCATGACACTACGACCAACACGATC |
| | (W77*) | TCGGTTGTCTCGGCGCACTCCGTACAGTAGGGATCGCCTACCTCCTTTTGA |
| | | GACAGAGACCCGCGCTACCATACTGGAGGATCATCCGCTGCTGCCCGAAT |
| | | GTAACACTTTGACAATGCACAACGTGAGTTACGTGCGAGGTCTTCCCTGC |
| | | AGTGTGGGATTTACGCTGATTCAGGAATGaGTTGTTCCCTGGGATATGGTT |
| | | CTGACGCGGGAGGAGCTTGTAATCCTGAGGAAGTGTATGCACGTGTGCCT |
| | | GTGTTGTGCCAACATTGATATCATGACGAGCATGATGATCCATGGTTACG |
| | | AGTCCTGGGCTCTCCACTGTCATTGTTCCAGTCCCGGTTCCCTGCAGTGCA |
| | | TAGCCGGCGGGCAGGTTTTGGCCAGCTGGTTTAGGATGGTGGTGGATGGC |
| | | GCCATGTTTAATCAGAGGTTTATATGGTACCGGGAGGTGGTGAATTACAA |
| | | CATGCCAAAAGAGGTAATGTTTATGTCCAGCGTGTTTATGAGGGGTCGCC |
| | | ACTTAATCTACCTGCGCTTGTGGTATGATGGCCACGTGGGTTCTGTGGTCC |
| | | CCGCCATGAGCTTTGGATACAGCGCCTTGCACTGTGGGATTTTGAACAAT |
| | | ATTGTGGTGCTGTGCTGCAGTTACTGTGCTGATTTAAGTGAGATCAGGGT |
| | | GCGCTGCTGTGCCCGGAGGACAAGGCGTCTCATGCTGCGGGCGGTGCGA |
| | | ATCATCGCTGAGGAGACCACTGCCATGTTGTATTCCTGCAGGACGGAGCG |
| | | GCGGCGGCAGCAGTTTATTCGCGCGCTGCTGCAGCACCACCGCCCTATCC |
| | | TGATGCACGATTATGACTCTACCCCCATGTAG |
| 108 | DA-E4ORF6 | ATGACTACGTCCGGCGTTCCATTTGGCATGACACTACGACCAACACGATC |
| | (W192*) | TCGGTTGTCTCGGCGCACTCCGTACAGTAGGGATCGCCTACCTCCTTTTGA |
| | | GACAGAGACCCGCGCTACCATACTGGAGGATCATCCGCTGCTGCCCGAAT |
| | | GTAACACTTTGACAATGCACAACGTGAGTTACGTGCGAGGTCTTCCCTGC |
| | | AGTGTGGGATTTACGCTGATTCAGGAATGGGTTGTTCCCTGGGATATGGT |
| | | TCTGACGCGGGAGGAGCTTGTAATCCTGAGGAAGTGTATGCACGTGTGCC |
| | | TGTGTTGTGCCAACATTGATATCATGACGAGCATGATGATCCATGGTTAC |
| | | GAGTCCTGGGCTCTCCACTGTCATTGTTCCAGTCCCGGTTCCCTGCAGTGC |
| | | ATAGCCGGCGGGCAGGTTTTGGCCAGCTGGTTTAGGATGGTGGTGGATGG |
| | | CGCCATGTTTAATCAGAGGTTTATATGGTACCGGGAGGTGGTGAATTACA |
| | | ACATGCCAAAAGAGGTAATGTTTATGTCCAGCGTGTTTATGAGGGGTCGC |
| | | CACTTAATCTACCTGCGCTTGTaGTATGATGGCCACGTGGGTTCTGTGGTC |
| | | CCCGCCATGAGCTTTGGATACAGCGCCTTGCACTGTGGGATTTTGAACAA |
| | | TATTGTGGTGCTGTGCTGCAGTTACTGTGCTGATTTAAGTGAGATCAGGG |
| | | TGCGCTGCTGTGCCCGGAGGACAAGGCGTCTCATGCTGCGGGCGGTGCGA |
| | | ATCATCGCTGAGGAGACCACTGCCATGTTGTATTCCTGCAGGACGGAGCG |
| | | GCGGCGGCAGCAGTTTATTCGCGCGCTGCTGCAGCACCACCGCCCTATCC |
| | | TGATGCACGATTATGACTCTACCCCCATGTAG |
| 109 | DA-L4 100K | ATGGAGTCAGTCGAGAAGGAGGACAGCCTAACCGCCCCCTTTGAGTTCGC |
| | (W435*) | CACCACCGCCTCCACCGATGCCGCCAACGCGCCTACCACCTTCCCCGTCG |
| | | AGGCACCCCCGCTTGAGGAGGAGGAAGTGATTATCGAGCAGGACCCAGG |
| | | TTTTGTAAGCGAAGACGACGAGGATCGCTCAGTACCAACAGAGGATAAA |
| | | AAGCAAGACCAGGACGACGCAGAGGCAAACGAGGAACAAGTCGGGCGG |
| | | GGGGACCAAAGGCATGGCGACTACCTAGATGTGGGAGACGACGTGCTGT |
| | | TGAAGCATCTGCAGCGCCAGTGCGCCATTATCTGCGACGCGTTGCAAGAG |
| | | CGCAGCGATGTGCCCCTCGCCATAGCGGATGTCAGCCTTGCCTACGAACG |
| | | CCACCTGTTCTCACCGCGCGTACCCCCCAAACGCCAAGAAAACGGCACAT |
| | | GCGAGCCCAACCCGCGCCTCAACTTCTACCCCGTATTTGCCGTGCCAGAG |
| | | GTGCTTGCCACCTATCACATCTTTTTCCAAAACTGCAAGATACCCCTATCC |
| | | TGCCGTGCCAACCGCAGCCGAGCGGACAAGCAGCTGGCCTTGCGGCAGG |
| | | GCGCTGTCATACCTGATATCGCCTCGCTCGACGAAGTGCCAAAAATCTTT |
| | | GAGGGTCTTGGACGCGACGAGAAACGCGCGGCAAACGCTCTGCAACAAG |
| | | AAAACAGCGAAAATGAAAGTCACTGTGGAGTGCTGGTGGAACTTGAGGG |
| | | TGACAACGCGCGCCTAGCCGTGCTGAAACGCAGCATCGAGGTCACCCACT |
| | | TTGCCTACCCGGCACTTAACCTACCCCCCAAGGTTATGAGCACAGTCATG |
| | | AGCGAGCTGATCGTGCGCCGTGCACGACCCCTGGAGAGGGATGCAAACT |
| | | TGCAAGAACAAACCGAGGAGGGCCTACCCGCAGTTGGCGATGAGCAGCT |
| | | GGCGCGCTGGCTTGAGACGCGCGAGCCTGCCGACTTGGAGGAGCGACGC |
| | | AAGCTAATGATGGCCGCAGTGCTTGTTACCGTGGAGCTTGAGTGCATGCA |
| | | GCGGTTCTTTGCTGACCCGGAGATGCAGCGCAAGCTAGAGGAAACGTTGC |
| | | ACTACACCTTTCGCCAGGGCTACGTGCGCCAGGCCTGCAAAATTTCCAAC |
| | | GTGGAGCTCTGCAACCTGGTCTCCTACCTTGGAATTTTGCACGAAAACCG |
| | | CCTCGGGCAAAACGTGCTTCATTCCACGCTCAAGGGCGAGGCGCGCCGCG |
| | | ACTACGTCCGCGACTGCGTTTACTTATTTCTGTGCTACACCTGGCAAACGG |
| | | CCATGGGCGTGTaGCAGCAATGCCTGGAGGAGCGCAACCTAAAGGAGCT |
| | | GCAGAAGCTGCTAAAGCAAAACTTGAAGGACCTATGGACGGCCTTCAAC |
| | | GAGCGCTCCGTGGCCGCGCACCTGGCGGACATTATCTTCCCCGAACGCCT |
| | | GCTTAAAACCCTGCAACAGGGTCTGCCAGACTTCACCAGTCAAAGCATGT |
| | | TGCAAAACTTTAGGAACTTTATCCTAGAGCGTTCAGGAATTCTGCCCGCC |
| | | ACCTGCTGTGCGCTTCCTAGCGACTTTGTGCCCATTAAGTACCGTGAATGC |
| | | CCTCCGCCGCTTTGGGGTCACTGCTACCTTCTGCAGCTAGCCAACTACCTT |
| | | GCCTACCACTCCGACATCATGGAAGACGTGAGCGGTGACGGCCTACTGG |
| | | AGTGTCACTGTCGCTGCAACCTATGCACCCCGCACCGCTCCCTGGTCTGC |
| | | AATTCGCAACTGCTTAGCGAAAGTCAAATTATCGGTACCTTTGAGCTGCA |
| | | GGGTCCCTCGCCTGACGAAAAGTCCGCGGCTCCGGGGTTGAAACTCACTC |
| | | CGGGGCTGTGGACGTCGGCTTACCTTCGCAAATTTGTACCTGAGGACTAC |
| | | CACGCCCACGAGATTAGGTTCTACGAAGACCAATCCCGCCCGCCAAATGC |
| | | GGAGCTTACCGCCTGCGTCATTACCCAGGGCCACATCCTTGGCCAATTGC |
| | | AAGCCATCAACAAAGCCCGCCAAGAGTTTCTGCTACGAAAGGGACGGGG |
| | | GGTTTACCTGGACCCCCAGTCCGGCGAGGAGCTCAACCCAATCCCCCCGC |
| | | CGCCGCAGCCCTATCAGCAGCCGCGGGCCCTTGCTTCCCAGGATGGCACC |
| | | CAAAAAGAAGCTGCAGCTGCCGCCGCCGCCACCCACGGACGAGGAGGAA |
| | | TACTGGGACAGTCAGGCAGAGGAGGTTTTGGACGAGGAGGAGGAGATGA |
| | | TGGAAGACTGGGACAGCCTAGACGAAGCTTCCGAGGCCGAAGAGGTGTC |
| | | AGACGAAACACCGTCACCCTCGGTCGCATTCCCCTCGCCGGCGCCCCAGA |
| | | AATTGGCAACCGTTCCCAGCATCGCTACAACCTCCGCTCCTCAGGCGCCG |
| | | CCGGCACTGCCTGTTCGCCGACCCAACCGTAG |
| 110 | DA-VP | ATGGCTGCCGATGGTTATCTTCCAGATTGGCTCGAGGACACTCTCTCTGA |
| | (W304*) | AGGAATAAGACAGTGGTGGAAGCTCAAACCTGGCCCACCACCACCAAAG |
| | | CCCGCAGAGCGGCATAAGGACGACAGCAGGGGTCTTGTGCTTCCTGGGT |
| | | ACAAGTACCTCGGACCCTTCAACGGACTCGACAAGGGAGAGCCGGTCAA |
| | | CGAGGCAGACGCCGCGGCCCTCGAGCACGACAAAGCCTACGACCGGCAG |
| | | CTCGACAGCGGAGACAACCCGTACCTCAAGTACAACCACGCCGACGCGG |
| | | AGTTTCAGGAGCGCCTTAAAGAAGATACGTCTTTTGGGGGCAACCTCGGA |
| | | CGAGCAGTCTTCCAGGCGAAAAAGAGGGTTCTTGAACCTCTGGGCCTGGT |
| | | TGAGGAACCTGTTAAGACGGCTCCGGGAAAAAAGAGGCCGGTAGAGCAC |
| | | TCTCCTGTGGAGCCAGACTCCTCCTCGGGAACCGGAAAGGCGGGCCAGC |
| | | AGCCTGCAAGAAAAAGATTGAATTTTGGTCAGACTGGAGACGCAGACTC |
| | | AGTACCTGACCCCCAGCCTCTCGGACAGCCACCAGCAGCCCCCTCTGGTC |
| | | TGGGAACTAATACGATGGCTACAGGCAGTGGCGCACCAATGGCAGACAA |
| | | TAACGAGGGCGCCGACGGAGTGGGTAATTCCTCGGGAAATTGGCATTGC |
| | | GATTCCACATGGATGGGCGACAGAGTCATCACCACCAGCACCCGAACCT |
| | | GGGCCCTGCCCACCTACAACAACCACCTCTACAAACAAATTTCCAGCCAA |
| | | TCAGGAGCCTCGAACGACAATCACTACTTTGGCTACAGCACCCCTTGGGG |
| | | GTATTTTGACTTCAACAGATTCCACTGCCACTTTTCACCACGTGACTGGCA |
| | | AAGACTCATCAACAACAACTGaGGATTCCGACCCAAGAGgCTCAACTTCA |
| | | AGCTCTTTAACATTCAAGTCAAAGAGGTCACGCAGAATGACGGTACGAC |
| | | GACGATTGCCAATAACCTTACCAGCACGGTTCAGGTGTTTACTGACTCGG |
| | | AGTACCAGCTCCCGTACGTCCTCGGCTCGGCGCATCAAGGATGCCTCCCG |
| | | CCGTTCCCAGCAGACGTCTTCATGGTGCCACAGTATGGATACCTCACCCT |
| | | GAACAACGGGAGTCAGGCAGTAGGACGCTCTTCATTTTACTGCCTGGAGT |
| | | ACTTTCCTTCTCAGATGCTGCGTACCGGAAACAACTTTACCTTCAGCTACA |
| | | CTTTTGAGGACGTTCCTTTCCACAGCAGCTACGCTCACAGCCAGAGTCTG |
| | | GACCGTCTCATGAATCCTCTCATCGACCAGTACCTGTATTACTTGAGCAG |
| | | AACAAACACTCCAAGTGGAACCACCACGCAGTCAAGGCTTCAGTTTTCTC |
| | | AGGCCGGAGCGAGTGACATTCGGGACCAGTCTAGGAACTGGCTTCCTGG |
| | | ACCCTGTTACCGCCAGCAGCGAGTATCAAAGACATCTGCGGATAACAAC |
| | | AACAGTGAATACTCGTGGACTGGAGCTACCAAGTACCACCTCAATGGCA |
| | | GAGACTCTCTGGTGAATCCGGGCCCGGCCATGGCAAGCCACAAGGACGA |
| | | TGAAGAAAAGTTTTTTCCTCAGAGCGGGGTTCTCATCTTTGGGAAGCAAG |
| | | GCTCAGAGAAAACAAATGTGGACATTGAAAAGGTCATGATTACAGACGA |
| | | AGAGGAAATCAGGACAACCAATCCCGTGGCTACGGAGCAGTATGGTTCT |
| | | GTATCTACCAACCTCCAGAGAGGCAACAGACAAGCAGCTACCGCAGATG |
| | | TCAACACACAAGGCGTTCTTCCAGGCATGGTCTGGCAGGACAGAGATGTG |
| | | TACCTTCAGGGGCCCATCTGGGCAAAGATTCCACACACGGACGGACATTT |
| | | TCACCCCTCTCCCCTCATGGGTGGATTCGGACTTAAACACCCTCCTCCACA |
| | | GATTCTCATCAAGAACACCCCGGTACCTGCGAATCCTTCGACCACCTTCA |
| | | GTGCGGCAAAGTTTGCTTCCTTCATCACACAGTACTCCACGGGACAGGTC |
| | | AGCGTGGAGATCGAGTGGGAGCTGCAGAAGGAAAACAGCAAACGCTGG |
| | | AATCCCGAAATTCAGTACACTTCCAACTACAACAAGTCTGTTAATGTGGA |
| | | CTTTACTGTGGACACTAATGGCGTGTATTCAGAGCCTCGCCCCATTGGCA |
| | | CCAGATACCTGACTCGTAATCTGTAA |
| 111 | DA-VP | ATGGCTGCCGATGGTTATCTTCCAGATTGGCTCGAGGACACTCTCTCTGA |
| | (Q598*) | AGGAATAAGACAGTGGTGGAAGCTCAAACCTGGCCCACCACCACCAAAG |
| | | CCCGCAGAGCGGCATAAGGACGACAGCAGGGGTCTTGTGCTTCCTGGGT |
| | | ACAAGTACCTCGGACCCTTCAACGGACTCGACAAGGGAGAGCCGGTCAA |
| | | CGAGGCAGACGCCGCGGCCCTCGAGCACGACAAAGCCTACGACCGGCAG |
| | | CTCGACAGCGGAGACAACCCGTACCTCAAGTACAACCACGCCGACGCGG |
| | | AGTTTCAGGAGCGCCTTAAAGAAGATACGTCTTTTGGGGGCAACCTCGGA |
| | | CGAGCAGTCTTCCAGGCGAAAAAGAGGGTTCTTGAACCTCTGGGCCTGGT |
| | | TGAGGAACCTGTTAAGACGGCTCCGGGAAAAAAGAGGCCGGTAGAGCAC |
| | | TCTCCTGTGGAGCCAGACTCCTCCTCGGGAACCGGAAAGGCGGGCCAGC |
| | | AGCCTGCAAGAAAAAGATTGAATTTTGGTCAGACTGGAGACGCAGACTC |
| | | AGTACCTGACCCCCAGCCTCTCGGACAGCCACCAGCAGCCCCCTCTGGTC |
| | | TGGGAACTAATACGATGGCTACAGGCAGTGGCGCACCAATGGCAGACAA |
| | | TAACGAGGGCGCCGACGGAGTGGGTAATTCCTCGGGAAATTGGCATTGC |
| | | GATTCCACATGGATGGGCGACAGAGTCATCACCACCAGCACCCGAACCT |
| | | GGGCCCTGCCCACCTACAACAACCACCTCTACAAACAAATTTCCAGCCAA |
| | | TCAGGAGCCTCGAACGACAATCACTACTTTGGCTACAGCACCCCTTGGGG |
| | | GTATTTTGACTTCAACAGATTCCACTGCCACTTTTCACCACGTGACTGGCA |
| | | AAGACTCATCAACAACAACTGGGGATTCCGACCCAAGAGACTCAACTTC |
| | | AAGCTCTTTAACATTCAAGTCAAAGAGGTCACGCAGAATGACGGTACGA |
| | | CGACGATTGCCAATAACCTTACCAGCACGGTTCAGGTGTTTACTGACTCG |
| | | GAGTACCAGCTCCCGTACGTCCTCGGCTCGGCGCATCAAGGATGCCTCCC |
| | | GCCGTTCCCAGCAGACGTCTTCATGGTGCCACAGTATGGATACCTCACCC |
| | | TGAACAACGGGAGTCAGGCAGTAGGACGCTCTTCATTTTACTGCCTGGAG |
| | | TACTTTCCTTCTCAGATGCTGCGTACCGGAAACAACTTTACCTTCAGCTAC |
| | | ACTTTTGAGGACGTTCCTTTCCACAGCAGCTACGCTCACAGCCAGAGTCT |
| | | GGACCGTCTCATGAATCCTCTCATCGACCAGTACCTGTATTACTTGAGCA |
| | | GAACAAACACTCCAAGTGGAACCACCACGCAGTCAAGGCTTCAGTTTTCT |
| | | CAGGCCGGAGCGAGTGACATTCGGGACCAGTCTAGGAACTGGCTTCCTG |
| | | GACCCTGTTACCGCCAGCAGCGAGTATCAAAGACATCTGCGGATAACAA |
| | | CAACAGTGAATACTCGTGGACTGGAGCTACCAAGTACCACCTCAATGGCA |
| | | GAGACTCTCTGGTGAATCCGGGCCCGGCCATGGCAAGCCACAAGGACGA |
| | | TGAAGAAAAGTTTTTTCCTCAGAGCGGGGTTCTCATCTTTGGGAAGCAAG |
| | | GCTCAGAGAAAACAAATGTGGACATTGAAAAGGTCATGATTACAGACGA |
| | | AGAGGAAATCAGGACAACCAATCCCGTGGCTACGGAGCAGTATGGTTCT |
| | | GTATCTACCAACCTCCAGAGAGGCAACAGACAAGCAGCTACCGCAGATG |
| | | TCAACACAtAAGGCGTTCTTCCAGGCATGGTCTGGCAGGACAGAGATGTG |
| | | TACCTTCAGGGGCCCATCTGGGCAAAGATTCCACACACGGACGGACATTT |
| | | TCACCCCTCTCCCCTCATGGGTGGATTCGGACTTAAACACCCTCCTCCACA |
| | | GATTCTCATCAAGAACACCCCGGTACCTGCGAATCCTTCGACCACCTTCA |
| | | GTGCGGCAAAGTTTGCTTCCTTCATCACACAGTACTCCACGGGACAGGTC |
| | | AGCGTGGAGATCGAGTGGGAGCTGCAGAAGGAAAACAGCAAACGCTGG |
| | | AATCCCGAAATTCAGTACACTTCCAACTACAACAAGTCTGTTAATGTGGA |
| | | CTTTACTGTGGACACTAATGGCGTGTATTCAGAGCCTCGCCCCATTGGCA |
| | | CCAGATACCTGACTCGTAATCTGTAA |
| 112 | L4 100K (wt) | MESVEKEDSLTAPFEFATTASTDAANAPTTFPVEAPPLEEEEVIIEQDPGFVSE |
| | | DDEDRSVPTEDKKQDQDDAEANEEQVGRGDQRHGDYLDVGDDVLLKHLQ |
| | | RQCAIICDALQERSDVPLAIADVSLAYERHLFSPRVPPKRQENGTCEPNPRLN |
| | | FYPVFAVPEVLATYHIFFQNCKIPLSCRANRSRADKQLALRQGAVIPDIASLD |
| | | EVPKIFEGLGRDEKRAANALQQENSENESHCGVLVELEGDNARLAVLKRSIE |
| | | VTHFAYPALNLPPKVMSTVMSELIVRRARPLERDANLQEQTEEGLPAVGDEQ |
| | | LARWLETREPADLEERRKLMMAAVLVTVELECMQRFFADPEMQRKLEETL |
| | | HYTFRQGYVRQACKISNVELCNLVSYLGILHENRLGQNVLHSTLKGEARRD |
| | | YVRDCVYLFLCYTWQTAMGVWQQCLEERNLKELQKLLKQNLKDLWTAFN |
| | | ERSVAAHLADIIFPERLLKTLQQGLPDFTSQSMLQNFRNFILERSGILPATCCA |
| | | LPSDFVPIKYRECPPPLWGHCYLLQLANYLAYHSDIMEDVSGDGLLECHCRC |
| | | NLCTPHRSLVCNSQLLSESQIIGTFELQGPSPDEKSAAPGLKLTPGLWTSAYLR |
| | | KFVPEDYHAHEIRFYEDQSRPPNAELTACVITQGHILGQLQAINKARQEFLLR |
| | | KGRGVYLDPQSGEELNPIPPPPQPYQQPRALASQDGTQKEAAAAAAATHGR |
| | | GGILGQSGRGGFGRGGGDDGRLGQPRRSFRGRRGVRRNTVTLGRIPLAGAPE |
| | | IGNRSQHRYNLRSSGAAGTACSPTQP |
| 113 | DA-Rep | gccaccatggagctggtcgggggctcgtgTAGaaggggattacctcggagaagcagtggatccaggaggaccagg |
| | D233X both | cctcatacatctccttcaatgcggcctccaactcgcggtcccaaatcaaggctgccttggacaatgcgggaaagattatga |
| | Rep52/40 and | gcctgactaaaaccgcccccgactacctggtgggccagcagcccgtggaggacatttccagcaatcggatttataaaattt |
| | Rep78/68: | tggaactaaacgggtacgatccccaatatgcggcttccgtctttctgggatgggccacgaaaaagttcggcaagaggaac |
| | (Rep52/40- | accatctggctgtttgggcctgcaactaccgggaagaccaacatcgcggaggccatagcccacactgtgcccttctacgg |
| | IRES- | gtgcgtaaactggaccaatgagaactttcccttcaacgactgtgtcgacaagatggtgatctggtgggaggaggggaaga |
| | Rep78/68- | tgaccgccaaggtcgtggagtcggccaaagccattctcggaggaagcaaggtgcgcgtggaccagaaatgcaagtcct |
| | SV40 poly A) | cggcccagatagacccgactcccgtgatcgtcacctccaacaccaacatgtgcgccgtgattgacgggaactcaacgac |
| | | cttcgaacaccagcagccgttgcaagaccggatgttcaaatttgaactcacccgccgtctggatcatgactttgggaaggt |
| | | caccaagcaggaagtcaaagactttttccggtgggcaaaggatcacgtggttgaggtggagcatgaattctacgtcaaaa |
| | | agggtggagccaagaaaagacccgcccccagtgacgcagatataagtgagcccaaacgggtgcgcgagtcagttgcg |
| | | cagccatcgacgtcagacgcggaagcttcgatcaactacgcagacaggtaccaaaacaaatgttctcgtcacgtgggcat |
| | | gaatctgatgctgtttccctgcagacaatgcgagagaatgaatcagaattcaaatatctgcttcactcacggacagaaagac |
| | | tgtttagagtgctttcccgtgtcagaatctcaacccgtttctgtcgtcaaaaaggcgtatcagaaactgtgctacattcatcata |
| | | tcatgggaaaggtgccagacgcttgcactgcctgcgatctggtcaatgtggatttggatgactgcatctttgaacaataaatg |
| | | atttaaatcaggtatggctgccgatggttatcttccagattggctcgaggacactctctctgagataactgagggatagaattc |
| | | cgccccccccccctaacgttactggccgaagccgcttggaataaggccggtgtgcgtttgtctatatgttattttccaccatat |
| | | tgccgtcttttggcaatgtgagggcccggaaacctggccctgtcttcttgacgagcattcctaggggtctttcccctctcgcc |
| | | aaaggaatgcaaggtctgttgaatgtcgtgaaggaagcagttcctctggaagcttcttgaagacaaacaacgtctgtagcg |
| | | accctttgcaggcagcggaaccccccacctggcgacaggtgcctctgcggccaaaagccacgtgtataagatacacctg |
| | | caaaggcggcacaaccccagtgccacgttgtgagttggatagttgtggaaagagtcaaatggctctcctcaagcgtattca |
| | | acaaggggctgaaggatgcccagaaggtaccccattgtatgggatctgatctggggcctcggtgcacatgctttacatgtg |
| | | tttagtcgaggttaaaaaaacgtctaggccccccgaaccacggggacgtggttttcctttgaaaaacacgatgataatagtt |
| | | atcgccgccATGCCGGGGTTTTACGAGATTGTGATTAAGGTCCCCAGCGACCT |
| | | TGACGAGCATCTGCCCGGCATTTCTGACAGCTTTGTGAACTGGGTGGCCG |
| | | AGAAGGAATGGGAGTTGCCGCCAGATTCTGACATGGATCTGAATCTGATT |
| | | GAGCAGGCACCCCTGACCGTGGCCGAGAAGCTGCAGCGCGACTTTCTGA |
| | | CGGAATGGCGCCGTGTGAGTAAGGCCCCGGAGGCCCTTTTCTTTGTGCAA |
| | | TTTGAGAAGGGAGAGAGCTACTTCCACATGCACGTGCTCGTGGAAACCAC |
| | | CGGGGTGAAATCCATGGTTTTGGGACGTTTCCTGAGTCAGATTCGCGAAA |
| | | AACTGATTCAGAGAATTTACCGCGGGATCGAGCCGACTTTGCCAAACTGG |
| | | TTCGCGGTCACAAAGACACGGAACGGCGCCGGGGGAGGAAACAAAGTTG |
| | | TTGATGAGTGCTACATCCCCAATTACTTGCTCCCCAAAACCCAGCCTGAG |
| | | CTCCAATGGGCATGGACCAACATGGAACAGTACCTGTCtGCCTGTTTGAAT |
| | | CTCACGGAGCGTAAACGGTTGGTGGCGCAGCATCTGACGCACGTGTCGCA |
| | | GACGCAGGAGCAGAACAAAGAGAATCAGAATCCCAATTCTGATGCGCCG |
| | | GTGATCAGATCAAAAACTTCAGCCAGGTACATGGAGCTGGTCGGGTGGCT |
| | | CGTGTAGAAGGGGATTACCTCGGAGAAGCAGTGGATCCAGGAGGACCAG |
| | | GCCTCATACATCTCCTTCAATGCGGCCTCCAACTCGCGGTCCCAAATCAA |
| | | GGCTGCCTTGGACAATGCGGGAAAGATTATGAGCCTGACTAAAACCGCC |
| | | CCCGACTACCTGGTGGGCCAGCAGCCCGTGGAGGACATTTCCAGCAATCG |
| | | GATTTATAAAATTTTGGAACTAAACGGGTACGATCCCCAATATGCGGCTT |
| | | CCGTCTTTCTGGGATGGGCCACGAAAAAGTTCGGCAAGAGGAACACCAT |
| | | CTGGCTGTTTGGGCCTGCAACTACCGGGAAGACCAACATCGCGGAGGCC |
| | | ATAGCCCACACTGTGCCCTTCTACGGGTGCGTAAACTGGACCAATGAGAA |
| | | CTTTCCCTTCAACGACTGTGTCGACAAGATGGTGATCTGGTGGGAGGAGG |
| | | GGAAGATGACCGCCAAGGTCGTGGAGTCGGCCAAAGCCATTCTCGGAGG |
| | | AAGCAAGGTGCGCGTGGACCAGAAATGCAAGTCCTCGGCCCAGATAGAC |
| | | CCGACTCCCGTGATCGTCACCTCCAACACCAACATGTGCGCCGTGATTGA |
| | | CGGGAACTCAACGACCTTCGAACACCAGCAGCCGTTGCAAGACCGGATG |
| | | TTCAAATTTGAACTCACCCGCCGTCTGGATCATGACTTTGGGAAGGTCAC |
| | | CAAGCAGGAAGTCAAAGACTTTTTCCGGTGGGCAAAGGATCACGTGGTT |
| | | GAGGTGGAGCATGAATTCTACGTCAAAAAGGGTGGAGCCAAGAAAAGAC |
| | | CCGCCCCCAGTGACGCAGATATAAGTGAGCCCAAACGGGTGCGCGAGTC |
| | | AGTTGCGCAGCCATCGACGTCAGACGCGGAAGCTTCGATCAACTACGCA |
| | | GACAGGTACCAAAACAAATGTTCTCGTCACGTGGGCATGAATCTGATGCT |
| | | GTTTCCCTGCAGACAATGCGAGAGAATGAATCAGAATTCAAATATCTGCT |
| | | TCACTCACGGACAGAAAGACTGTTTAGAGTGCTTTCCCGTGTCAGAATCT |
| | | CAACCCGTTTCTGTCGTCAAAAAGGCGTATCAGAAACTGTGCTACATTCA |
| | | TCATATCATGGGAAAGGTGCCAGACGCTTGCACTGCCTGCGATCTGGTCA |
| | | ATGTGGATTTGGATGACTGCATCTTTGAACAATAAatgatttaaatcaggtatggctgccg |
| | | atggttatcttccagattggctcgaggacactctctctgagttatcatttaaatggcgcgcccacgtgggtaccgcggccgc |
| | | ggggatccagacatgataagatacattgatgagtttggacaaaccacaactagaatgcagtgaaaaaaatgctttatttgtg |
| | | aaatttgtgatgctattgctttatttgtaaccattataagctgcaataaacaagttaacaacaacaattgcattcattttatgtttca |
| | | ggttcagggggaggtgtgggaggttttttcggatcctctagagtcgacctgcaggca |
| 114 | DA-Rep | gccaccatggagctggtcgggtggctcgtgGACaaggggattacctcggagaagcagtggatccaggaggaccag |
| | D233X | gcctcatacatctccttcaatgcggcctccaactcgcggtcccaaatcaaggctgccttggacaatgcgggaaagattatg |
| | Rep78/68 | agcctgactaaaaccgcccccgactacctggtgggccagcagcccgtggaggacatttccagcaatcggatttataaaat |
| | only: | tttggaactaaacgggtacgatccccaatatgcggcttccgtctttctgggatgggccacgaaaaagttcggcaagaggaa |
| | (Rep52/40- | caccatctggctgtttgggcctgcaactaccgggaagaccaacatcgcggaggccatagcccacactgtgcccttctacg |
| | IRES- | ggtgcgtaaactggaccaatgagaactttcccttcaacgactgtgtcgacaagatggtgatctggtgggaggaggggaag |
| | Rep78/68- | atgaccgccaaggtcgtggagtcggccaaagccattctcggaggaagcaaggtgcgcgtggaccagaaatgcaagtcc |
| | SV40 poly A) | tcggcccagatagacccgactcccgtgatcgtcacctccaacaccaacatgtgcgccgtgattgacgggaactcaacga |
| | | ccttcgaacaccagcagccgttgcaagaccggatgttcaaatttgaactcacccgccgtctggatcatgactttgggaagg |
| | | tcaccaagcaggaagtcaaagactttttccggtgggcaaaggatcacgtggttgaggtggagcatgaattctacgtcaaaa |
| | | agggtggagccaagaaaagacccgcccccagtgacgcagatataagtgagcccaaacgggtgcgcgagtcagttgcg |
| | | cagccatcgacgtcagacgcggaagcttcgatcaactacgcagacaggtaccaaaacaaatgttctcgtcacgtgggcat |
| | | gaatctgatgctgtttccctgcagacaatgcgagagaatgaatcagaattcaaatatctgcttcactcacggacagaaagac |
| | | tgtttagagtgctttcccgtgtcagaatctcaacccgtttctgtcgtcaaaaaggcgtatcagaaactgtgctacattcatcata |
| | | tcatgggaaaggtgccagacgcttgcactgcctgcgatctggtcaatgtggatttggatgactgcatctttgaacaataaatg |
| | | atttaaatcaggtatggctgccgatggttatcttccagattggctcgaggacactctctctgagataactgagggatagaattc |
| | | cgccccccccccctaacgttactggccgaagccgcttggaataaggccggtgtgcgtttgtctatatgttattttccaccatat |
| | | tgccgtcttttggcaatgtgagggcccggaaacctggccctgtcttcttgacgagcattcctaggggtctttcccctctcgcc |
| | | aaaggaatgcaaggtctgttgaatgtcgtgaaggaagcagttcctctggaagcttcttgaagacaaacaacgtctgtagcg |
| | | accctttgcaggcagcggaaccccccacctggcgacaggtgcctctgcggccaaaagccacgtgtataagatacacctg |
| | | caaaggcggcacaaccccagtgccacgttgtgagttggatagttgtggaaagagtcaaatggctctcctcaagcgtattca |
| | | acaaggggctgaaggatgcccagaaggtaccccattgtatgggatctgatctggggcctcggtgcacatgctttacatgtg |
| | | tttagtcgaggttaaaaaaacgtctaggccccccgaaccacggggacgtggttttcctttgaaaaacacgatgataatagtt |
| | | atcgccgccATGCCGGGGTTTTACGAGATTGTGATTAAGGTCCCCAGCGACCT |
| | | TGACGAGCATCTGCCCGGCATTTCTGACAGCTTTGTGAACTGGGTGGCCG |
| | | AGAAGGAATGGGAGTTGCCGCCAGATTCTGACATGGATCTGAATCTGATT |
| | | GAGCAGGCACCCCTGACCGTGGCCGAGAAGCTGCAGCGCGACTTTCTGA |
| | | CGGAATGGCGCCGTGTGAGTAAGGCCCCGGAGGCCCTTTTCTTTGTGCAA |
| | | TTTGAGAAGGGAGAGAGCTACTTCCACATGCACGTGCTCGTGGAAACCAC |
| | | CGGGGTGAAATCCATGGTTTTGGGACGTTTCCTGAGTCAGATTCGCGAAA |
| | | AACTGATTCAGAGAATTTACCGCGGGATCGAGCCGACTTTGCCAAACTGG |
| | | TTCGCGGTCACAAAGACACGGAACGGCGCCGGGGGAGGAAACAAAGTTG |
| | | TTGATGAGTGCTACATCCCCAATTACTTGCTCCCCAAAACCCAGCCTGAG |
| | | CTCCAATGGGCATGGACCAACATGGAACAGTACCTGTCtGCCTGTTTGAAT |
| | | CTCACGGAGCGTAAACGGTTGGTGGCGCAGCATCTGACGCACGTGTCGCA |
| | | GACGCAGGAGCAGAACAAAGAGAATCAGAATCCCAATTCTGATGCGCCG |
| | | GTGATCAGATCAAAAACTTCAGCCAGGTACATGGAGCTGGTCGGGTGGCT |
| | | CGTGTAGAAGGGGATTACCTCGGAGAAGCAGTGGATCCAGGAGGACCAG |
| | | GCCTCATACATCTCCTTCAATGCGGCCTCCAACTCGCGGTCCCAAATCAA |
| | | GGCTGCCTTGGACAATGCGGGAAAGATTATGAGCCTGACTAAAACCGCC |
| | | CCCGACTACCTGGTGGGCCAGCAGCCCGTGGAGGACATTTCCAGCAATCG |
| | | GATTTATAAAATTTTGGAACTAAACGGGTACGATCCCCAATATGCGGCTT |
| | | CCGTCTTTCTGGGATGGGCCACGAAAAAGTTCGGCAAGAGGAACACCAT |
| | | CTGGCTGTTTGGGCCTGCAACTACCGGGAAGACCAACATCGCGGAGGCC |
| | | ATAGCCCACACTGTGCCCTTCTACGGGTGCGTAAACTGGACCAATGAGAA |
| | | CTTTCCCTTCAACGACTGTGTCGACAAGATGGTGATCTGGTGGGAGGAGG |
| | | GGAAGATGACCGCCAAGGTCGTGGAGTCGGCCAAAGCCATTCTCGGAGG |
| | | AAGCAAGGTGCGCGTGGACCAGAAATGCAAGTCCTCGGCCCAGATAGAC |
| | | CCGACTCCCGTGATCGTCACCTCCAACACCAACATGTGCGCCGTGATTGA |
| | | CGGGAACTCAACGACCTTCGAACACCAGCAGCCGTTGCAAGACCGGATG |
| | | TTCAAATTTGAACTCACCCGCCGTCTGGATCATGACTTTGGGAAGGTCAC |
| | | CAAGCAGGAAGTCAAAGACTTTTTCCGGTGGGCAAAGGATCACGTGGTT |
| | | GAGGTGGAGCATGAATTCTACGTCAAAAAGGGTGGAGCCAAGAAAAGAC |
| | | CCGCCCCCAGTGACGCAGATATAAGTGAGCCCAAACGGGTGCGCGAGTC |
| | | AGTTGCGCAGCCATCGACGTCAGACGCGGAAGCTTCGATCAACTACGCA |
| | | GACAGGTACCAAAACAAATGTTCTCGTCACGTGGGCATGAATCTGATGCT |
| | | GTTTCCCTGCAGACAATGCGAGAGAATGAATCAGAATTCAAATATCTGCT |
| | | TCACTCACGGACAGAAAGACTGTTTAGAGTGCTTTCCCGTGTCAGAATCT |
| | | CAACCCGTTTCTGTCGTCAAAAAGGCGTATCAGAAACTGTGCTACATTCA |
| | | TCATATCATGGGAAAGGTGCCAGACGCTTGCACTGCCTGCGATCTGGTCA |
| | | ATGTGGATTTGGATGACTGCATCTTTGAACAATAAatgatttaaatcaggtatggctgccg |
| | | atggttatcttccagattggctcgaggacactctctctgagttatcatttaaatggcgcgcccacgtgggtaccgcggccgc |
| | | ggggatccagacatgataagatacattgatgagtttggacaaaccacaactagaatgcagtgaaaaaaatgctttatttgtg |
| | | aaatttgtgatgctattgctttatttgtaaccattataagctgcaataaacaagttaacaacaacaattgcattcattttatgtttca |
| | | ggttcagggggaggtgtgggaggttttttcggatcctctagagtcgacctgcaggca |
| 115 | DA-Rep | gccaccatggagctggtcgggtggctcgtgGACaaggggattacctcggagaagcagtggatccaggaggaccag |
| | E17X | gcctcatacatctccttcaatgcggcctccaactcgcggtcccaaatcaaggctgccttggacaatgcgggaaagattatg |
| | Rep78/68 | agcctgactaaaaccgcccccgactacctggtgggccagcagcccgtggaggacatttccagcaatcggatttataaaat |
| | only: | tttggaactaaacgggtacgatccccaatatgcggcttccgtctttctgggatgggccacgaaaaagttcggcaagaggaa |
| | (Rep52/40- | caccatctggctgtttgggcctgcaactaccgggaagaccaacatcgcggaggccatagcccacactgtgcccttctacg |
| | IRES- | ggtgcgtaaactggaccaatgagaactttcccttcaacgactgtgtcgacaagatggtgatctggtgggaggaggggaag |
| | Rep78/68- | atgaccgccaaggtcgtggagtcggccaaagccattctcggaggaagcaaggtgcgcgtggaccagaaatgcaagtcc |
| | SV40 polyA) | tcggcccagatagacccgactcccgtgatcgtcacctccaacaccaacatgtgcgccgtgattgacgggaactcaacga |
| | | ccttcgaacaccagcagccgttgcaagaccggatgttcaaatttgaactcacccgccgtctggatcatgactttgggaagg |
| | | tcaccaagcaggaagtcaaagactttttccggtgggcaaaggatcacgtggttgaggtggagcatgaattctacgtcaaaa |
| | | agggtggagccaagaaaagacccgcccccagtgacgcagatataagtgagcccaaacgggtgcgcgagtcagttgcg |
| | | cagccatcgacgtcagacgcggaagcttcgatcaactacgcagacaggtaccaaaacaaatgttctcgtcacgtgggcat |
| | | gaatctgatgctgtttccctgcagacaatgcgagagaatgaatcagaattcaaatatctgcttcactcacggacagaaagac |
| | | tgtttagagtgctttcccgtgtcagaatctcaacccgtttctgtcgtcaaaaaggcgtatcagaaactgtgctacattcatcata |
| | | tcatgggaaaggtgccagacgcttgcactgcctgcgatctggtcaatgtggatttggatgactgcatctttgaacaataaatg |
| | | atttaaatcaggtatggctgccgatggttatcttccagattggctcgaggacactctctctgagataactgagggatagaattc |
| | | cgccccccccccctaacgttactggccgaagccgcttggaataaggccggtgtgcgtttgtctatatgttattttccaccatat |
| | | tgccgtcttttggcaatgtgagggcccggaaacctggccctgtcttcttgacgagcattcctaggggtctttcccctctcgcc |
| | | aaaggaatgcaaggtctgttgaatgtcgtgaaggaagcagttcctctggaagcttcttgaagacaaacaacgtctgtagcg |
| | | accctttgcaggcagcggaaccccccacctggcgacaggtgcctctgcggccaaaagccacgtgtataagatacacctg |
| | | caaaggcggcacaaccccagtgccacgttgtgagttggatagttgtggaaagagtcaaatggctctcctcaagcgtattca |
| | | acaaggggctgaaggatgcccagaaggtaccccattgtatgggatctgatctggggcctcggtgcacatgctttacatgtg |
| | | tttagtcgaggttaaaaaaacgtctaggccccccgaaccacggggacgtggttttcctttgaaaaacacgatgataatagtt |
| | | atcgccgccATGCCGGGGTTTTACGAGATTGTGATTAAGGTCCCCAGCGACCT |
| | | TGACTAGCATCTGCCCGGCATTTCTGACAGCTTTGTGAACTGGGTGGCCG |
| | | AGAAGGAATGGGAGTTGCCGCCAGATTCTGACATGGATCTGAATCTGATT |
| | | GAGCAGGCACCCCTGACCGTGGCCGAGAAGCTGCAGCGCGACTTTCTGA |
| | | CGGAATGGCGCCGTGTGAGTAAGGCCCCGGAGGCCCTTTTCTTTGTGCAA |
| | | TTTGAGAAGGGAGAGAGCTACTTCCACATGCACGTGCTCGTGGAAACCAC |
| | | CGGGGTGAAATCCATGGTTTTGGGACGTTTCCTGAGTCAGATTCGCGAAA |
| | | AACTGATTCAGAGAATTTACCGCGGGATCGAGCCGACTTTGCCAAACTGG |
| | | TTCGCGGTCACAAAGACACGGAACGGCGCCGGGGGAGGAAACAAAGTTG |
| | | TTGATGAGTGCTACATCCCCAATTACTTGCTCCCCAAAACCCAGCCTGAG |
| | | CTCCAATGGGCATGGACCAACATGGAACAGTACCTGTCtGCCTGTTTGAAT |
| | | CTCACGGAGCGTAAACGGTTGGTGGCGCAGCATCTGACGCACGTGTCGCA |
| | | GACGCAGGAGCAGAACAAAGAGAATCAGAATCCCAATTCTGATGCGCCG |
| | | GTGATCAGATCAAAAACTTCAGCCAGGTACATGGAGCTGGTCGGGTGGCT |
| | | CGTGGACAAGGGGATTACCTCGGAGAAGCAGTGGATCCAGGAGGACCAG |
| | | GCCTCATACATCTCCTTCAATGCGGCCTCCAACTCGCGGTCCCAAATCAA |
| | | GGCTGCCTTGGACAATGCGGGAAAGATTATGAGCCTGACTAAAACCGCC |
| | | CCCGACTACCTGGTGGGCCAGCAGCCCGTGGAGGACATTTCCAGCAATCG |
| | | GATTTATAAAATTTTGGAACTAAACGGGTACGATCCCCAATATGCGGCTT |
| | | CCGTCTTTCTGGGATGGGCCACGAAAAAGTTCGGCAAGAGGAACACCAT |
| | | CTGGCTGTTTGGGCCTGCAACTACCGGGAAGACCAACATCGCGGAGGCC |
| | | ATAGCCCACACTGTGCCCTTCTACGGGTGCGTAAACTGGACCAATGAGAA |
| | | CTTTCCCTTCAACGACTGTGTCGACAAGATGGTGATCTGGTGGGAGGAGG |
| | | GGAAGATGACCGCCAAGGTCGTGGAGTCGGCCAAAGCCATTCTCGGAGG |
| | | AAGCAAGGTGCGCGTGGACCAGAAATGCAAGTCCTCGGCCCAGATAGAC |
| | | CCGACTCCCGTGATCGTCACCTCCAACACCAACATGTGCGCCGTGATTGA |
| | | CGGGAACTCAACGACCTTCGAACACCAGCAGCCGTTGCAAGACCGGATG |
| | | TTCAAATTTGAACTCACCCGCCGTCTGGATCATGACTTTGGGAAGGTCAC |
| | | CAAGCAGGAAGTCAAAGACTTTTTCCGGTGGGCAAAGGATCACGTGGTT |
| | | GAGGTGGAGCATGAATTCTACGTCAAAAAGGGTGGAGCCAAGAAAAGAC |
| | | CCGCCCCCAGTGACGCAGATATAAGTGAGCCCAAACGGGTGCGCGAGTC |
| | | AGTTGCGCAGCCATCGACGTCAGACGCGGAAGCTTCGATCAACTACGCA |
| | | GACAGGTACCAAAACAAATGTTCTCGTCACGTGGGCATGAATCTGATGCT |
| | | GTTTCCCTGCAGACAATGCGAGAGAATGAATCAGAATTCAAATATCTGCT |
| | | TCACTCACGGACAGAAAGACTGTTTAGAGTGCTTTCCCGTGTCAGAATCT |
| | | CAACCCGTTTCTGTCGTCAAAAAGGCGTATCAGAAACTGTGCTACATTCA |
| | | TCATATCATGGGAAAGGTGCCAGACGCTTGCACTGCCTGCGATCTGGTCA |
| | | ATGTGGATTTGGATGACTGCATCTTTGAACAATAAatgatttaaatcaggtatggctgccg |
| | | atggttatcttccagattggctcgaggacactctctctgagttatcatttaaatggcgcgcccacgtgggtaccgcggccgc |
| | | ggggatccagacatgataagatacattgatgagtttggacaaaccacaactagaatgcagtgaaaaaaatgctttatttgtg |
| | | aaatttgtgatgctattgctttatttgtaaccattataagctgcaataaacaagttaacaacaacaattgcattcattttatgtttca |
| | | ggttcagggggaggtgtgggaggttttttcggatcctctagagtcgacctgcaggca |
| 116 | VP (wt) | ATGGCTGCCGATGGTTATCTTCCAGATTGGCTCGAGGACACTCTCTCTGA |
| | | AGGAATAAGACAGTGGTGGAAGCTCAAACCTGGCCCACCACCACCAAAG |
| | | CCCGCAGAGCGGCATAAGGACGACAGCAGGGGTCTTGTGCTTCCTGGGT |
| | | ACAAGTACCTCGGACCCTTCAACGGACTCGACAAGGGAGAGCCGGTCAA |
| | | CGAGGCAGACGCCGCGGCCCTCGAGCACGACAAAGCCTACGACCGGCAG |
| | | CTCGACAGCGGAGACAACCCGTACCTCAAGTACAACCACGCCGACGCGG |
| | | AGTTTCAGGAGCGCCTTAAAGAAGATACGTCTTTTGGGGGCAACCTCGGA |
| | | CGAGCAGTCTTCCAGGCGAAAAAGAGGGTTCTTGAACCTCTGGGCCTGGT |
| | | TGAGGAACCTGTTAAGACGGCTCCGGGAAAAAAGAGGCCGGTAGAGCAC |
| | | TCTCCTGTGGAGCCAGACTCCTCCTCGGGAACCGGAAAGGCGGGCCAGC |
| | | AGCCTGCAAGAAAAAGATTGAATTTTGGTCAGACTGGAGACGCAGACTC |
| | | AGTACCTGACCCCCAGCCTCTCGGACAGCCACCAGCAGCCCCCTCTGGTC |
| | | TGGGAACTAATACGATGGCTACAGGCAGTGGCGCACCAATGGCAGACAA |
| | | TAACGAGGGCGCCGACGGAGTGGGTAATTCCTCGGGAAATTGGCATTGC |
| | | GATTCCACATGGATGGGCGACAGAGTCATCACCACCAGCACCCGAACCT |
| | | GGGCCCTGCCCACCTACAACAACCACCTCTACAAACAAATTTCCAGCCAA |
| | | TCAGGAGCCTCGAACGACAATCACTACTTTGGCTACAGCACCCCTTGGGG |
| | | GTATTTTGACTTCAACAGATTCCACTGCCACTTTTCACCACGTGACTGGCA |
| | | AAGACTCATCAACAACAACTGGGGATTCCGACCCAAGAGACTCAACTTC |
| | | AAGCTCTTTAACATTCAAGTCAAAGAGGTCACGCAGAATGACGGTACGA |
| | | CGACGATTGCCAATAACCTTACCAGCACGGTTCAGGTGTTTACTGACTCG |
| | | GAGTACCAGCTCCCGTACGTCCTCGGCTCGGCGCATCAAGGATGCCTCCC |
| | | GCCGTTCCCAGCAGACGTCTTCATGGTGCCACAGTATGGATACCTCACCC |
| | | TGAACAACGGGAGTCAGGCAGTAGGACGCTCTTCATTTTACTGCCTGGAG |
| | | TACTTTCCTTCTCAGATGCTGCGTACCGGAAACAACTTTACCTTCAGCTAC |
| | | ACTTTTGAGGACGTTCCTTTCCACAGCAGCTACGCTCACAGCCAGAGTCT |
| | | GGACCGTCTCATGAATCCTCTCATCGACCAGTACCTGTATTACTTGAGCA |
| | | GAACAAACACTCCAAGTGGAACCACCACGCAGTCAAGGCTTCAGTTTTCT |
| | | CAGGCCGGAGCGAGTGACATTCGGGACCAGTCTAGGAACTGGCTTCCTG |
| | | GACCCTGTTACCGCCAGCAGCGAGTATCAAAGACATCTGCGGATAACAA |
| | | CAACAGTGAATACTCGTGGACTGGAGCTACCAAGTACCACCTCAATGGCA |
| | | GAGACTCTCTGGTGAATCCGGGCCCGGCCATGGCAAGCCACAAGGACGA |
| | | TGAAGAAAAGTTTTTTCCTCAGAGCGGGGTTCTCATCTTTGGGAAGCAAG |
| | | GCTCAGAGAAAACAAATGTGGACATTGAAAAGGTCATGATTACAGACGA |
| | | AGAGGAAATCAGGACAACCAATCCCGTGGCTACGGAGCAGTATGGTTCT |
| | | GTATCTACCAACCTCCAGAGAGGCAACAGACAAGCAGCTACCGCAGATG |
| | | TCAACACACAAGGCGTTCTTCCAGGCATGGTCTGGCAGGACAGAGATGTG |
| | | TACCTTCAGGGGCCCATCTGGGCAAAGATTCCACACACGGACGGACATTT |
| | | TCACCCCTCTCCCCTCATGGGTGGATTCGGACTTAAACACCCTCCTCCACA |
| | | GATTCTCATCAAGAACACCCCGGTACCTGCGAATCCTTCGACCACCTTCA |
| | | GTGCGGCAAAGTTTGCTTCCTTCATCACACAGTACTCCACGGGACAGGTC |
| | | AGCGTGGAGATCGAGTGGGAGCTGCAGAAGGAAAACAGCAAACGCTGG |
| | | AATCCCGAAATTCAGTACACTTCCAACTACAACAAGTCTGTTAATGTGGA |
| | | CTTTACTGTGGACACTAATGGCGTGTATTCAGAGCCTCGCCCCATTGGCA |
| | | CCAGATACCTGACTCGTAATCTGTAA |
| 117 | VP (wt) | MAADGYLPDWLEDTLSEGIRQWWKLKPGPPPPKPAERHKDDSRGLVLPGYK |
| | Translated | YLGPFNGLDKGEPVNEADAAALEHDKAYDRQLDSGDNPYLKYNHADAEFQ |
| | (same as SEQ | ERLKEDTSFGGNLGRAVFQAKKRVLEPLGLVEEPVKTAPGKKRPVEHSPVE |
| | ID NO: 14 | PDSSSGTGKAGQQPARKRLNFGQTGDADSVPDPQPLGQPPAAPSGLGTN |
| | with | TMATGSGAPMADNNEGADGVGNSSGNWHCDSTWMGDRVITTSTRTWALPT |
| | different | YNNHLYKQISSQSGASNDNHYFGYSTPWGYFDFNRFHCHFSPRDWQRLINN |
| | VP protein | NWGFRPKRLNFKLFNIQVKEVTQNDGTTTIANNLTSTVQVFTDSEYQLPYVL |
| | identified) | GSAHQGCLPPFPADVFMVPQYGYLTLNNGSQAVGRSSFYCLEYFPSQMLRT |
| | Underline: | GNNFTFSYTFEDVPFHSSYAHSQSLDRLMNPLIDQYLYYLSRTNTPSGTTTQS |
| | VP1 | RLQFSQAGASDIRDQSRNWLPGPCYRQQRVSKTSADNNNSEYSWTGATKYH |
| | Bold: VP2 | LNGRDSLVNPGPAMASHKDDEEKFFPQSGVLIFGKQGSEKTNVDIEKVMITD |
| | Italic: VP3 | EEEIRTTNPVATEQYGSVSTNLQRGNRQAATADVNTQGVLPGMVWQDRDVY |
| | | LQGPIWAKIPHTDGHFHPSPLMGGFGLKHPPPQILIKNTPVPANPSTTFSAAK |
| | | FASFITQYSTGQVSVEIEWELQKENSKRWNPEIQYTSNYNKSVNVDFTVDTN |
| | | GVYSEPRPIGTRYLTRNL* |
| 118 | rcTA | MVIMSPKRRTQAERAMETQGKLIAAALGVLREKGYAGFRIADVPGAAGVSR |
| | (example | GAQSHHFPTKLELLLATFEWLYEQITERSRARLAKLKPEDDVIQQMLDDAAE |
| | sequence with | FFLDDDFSIGLDLIVAADRDPVLREGIQRTVERNRFVVGDIWLGVLVSRGLSR |
| | reverse CymR | DDAEDILWLIFNSVRGLVVRSLWQKDKERFERVRNSTLEIARERYAKFKRSG |
| | fused to 3x | GGGPTDALDDFDLDMLPADALDDFDLDMLPADALDDFDLDMLPGPPKKKR |
| | VP16 and a | KV** |
| | NLS) | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. An engineered cell for AAV production, comprising one or more stably integrated nucleic acid molecules collectively comprising a nucleic acid sequence encoding for each of: a noncanonical tRNA synthetase; a noncanonical tRNA corresponding to the noncanonical tRNA synthetase; NC-Rep 78; and NC-Rep52; each of which is operably linked to a promoter; wherein the nucleic acid sequence encoding NC-Rep78 and the nucleic acid sequence encoding NC-Rep52 each comprises a codon that is both a premature stop codon and an amino acid codon corresponding to the noncanonical tRNA.

2. The engineered cell of claim 1, wherein the one or more stably integrated nucleic acid molecules comprises a first stably integrated nucleic acid molecule comprising the nucleic acid sequence encoding for the noncanonical tRNA synthetase.

3. The engineered cell of claim 2, wherein the noncanonical tRNA synthetase is Pyrrolysyl-tRNA synthetase (pylRS).

4. The engineered cell of claim 3, wherein pylRS comprises the amino acid sequence of any one of SEQ ID NOs: 20 and 21.

5. The engineered cell of claim 4, wherein PylRS comprises the amino acid sequence of SEQ ID NO: 21.

6. The engineered cell of any one of claims 2-5, wherein the first stably integrated nucleic acid molecule further comprises a selection marker that is operably linked to a promoter.

7. The engineered cell of any one of claims 1-6, wherein the one or more stably integrated nucleic acid molecules comprises a second stably integrated nucleic acid molecule comprising the nucleic acid sequence encoding for the noncanonical tRNA.

8. The engineered cell of any one of claims 1-7, wherein the noncanonical tRNA charges H-Lys(Boc)-OH.

9. The engineered cell of claim 7 or claim 8, wherein the noncanonical tRNA is PylT U25C.

10. The engineered cell of claim 9, wherein PylT U25C comprises the nucleic acid sequence of SEQ ID NO: 22.

11. The engineered cell of claim 9 or claim 10, wherein the second stably integrated nucleic acid molecule comprises four nucleic acid sequences, each comprising the nucleic acid sequences encoding for PylT U25C and each operably linked to a promoter.

12. The engineered cell of any one of claims 7-11, wherein the second stably integrated nucleic acid molecule further comprises a selection marker that is operably linked to a promoter.

13. The engineered cell of any one of claims 1-12, wherein the one or more stably integrated nucleic acid molecules comprises a third stably integrated nucleic acid molecule comprising the nucleic acid sequences encoding for NC-Rep78 and NC-Rep52.

14. The engineered cell of claim 13, wherein: NC-Rep78 comprises a premature stop codon at position 17; NC-Rep52 comprises a premature stop codon at position 233; or a combination thereof.

15. The engineered cell of claim 13 or claim 14, wherein the noncanonical tRNA synthetase is pylRS and the noncanonical tRNA is PylT U25C.

16. The engineered cell of any one of claims 13-15, wherein the nucleic acid sequence encoding NC-Rep78 and the nucleic acid sequence encoding NC-Rep52 are encoded as a single transcript.

17. The engineered cell of claim 16, wherein the single transcript comprises a nucleic acid sequence encoding for an amino acid sequence of any one of SEQ ID NOs: 26-27.

18. The engineered cell of any one of claims 13-17, wherein the third stably integrated nucleic acid molecule further comprises: a nucleic acid sequence encoding for NC-Rep40; a nucleic acid sequence encoding for NC-Rep68; or both.

19. The engineered cell of any one of claims 1-18, wherein the engineered cell is HEK293 cell, HeLa cell, BHK cell, or SB9 cell.

20. A kit comprising the engineered cell of any one of claims 1-19.

21. The kit of claim 20 further comprising a polynucleotide comprising, from 5′ to 3′: (i) a nucleic acid sequence of a 5′ inverted terminal repeat; (ii) a multiple cloning site; and (iii) a nucleic acid sequence of a 3′ inverted terminal repeat.

22. The kit of claim 22, wherein the polynucleotide is a plasmid or a vector.

23. A method for AAV production, comprising contacting the engineered cell of any of claims 1-19 with a noncanonical amino acid.

24. The method of 23, wherein the noncanonical amino acid is H-Lys(Boc)-OH.

25. An engineered cell for AAV production, comprising one or more stably integrated nucleic acid molecules collectively comprising a nucleic acid sequence encoding for each of: Rep52, DA-Rep52, Rep40, or DA-Rep40; Rep78, DA-Rep78, Rep68, or DA-Rep68; E2A or DA-E2A; E4ORF6 or DA-E4ORF6; VARNA or DA-VARNA; VP1 or DA-VP1; VP2 or DA-VP2; VP3 or DA-VP3; AAP; and L4 100K or DA-L4 100K and an Base Editor each nucleic acid molecule being operably linked to a promoter; wherein the cell comprises the nucleic acid sequence of at least one of DA-Rep52, DA-Rep40, DA-Rep78, DA-Rep68, DA-E2A, DA-E4ORF6, DA-VP1, DA-VP2, DA-V3, and DA-L4 100K; wherein the nucleic acid sequences of DA-Rep52, DA-Rep40, DA-Rep78, DA-Rep68, DA-E2A, DA-E4ORF6, DA-VP1, DA-VP2, DA-V3, and DA-L4 100K each comprises a modified codon.

26. The engineered cell of claim 25, wherein the modified codon encodes for a missense codon, and wherein deamination of a cytosine or a adenine in the modified codon converts the encoded amino acid into another amino acid.

27. The engineered cell of claim 25, wherein the modified codon encodes for a premature stop codon, and wherein deamination of a adenine in the modified codon converts the modified codon into a tryptophan codon, glutamine codon or arginine.

28. The engineered cell of claim 25, wherein the modified codon encodes for a premature stop codon, and wherein deamination of a cytosine in the modified codon converts the encoded amino acid into a proline.

29. The engineered cell of any one of claims 25-28, wherein the one or more stably integrated nucleic acid molecules comprise a nucleic acid sequence encoding one or more CTCF insulators.

30. The engineered cell of any one of claims 25-29, wherein the one or more stably integrated nucleic acid molecules comprises a first stably integrated nucleic acid molecule comprising the nucleic acid sequence encoding DA-E2A, the nucleic acid sequence encoding DA-E4ORF6, and the nucleic acid sequence encoding VARNA.

31. The engineered cell of claim 30, wherein the first stably integrated nucleic acid molecule further comprises a nucleic acid sequence encoding L4 100K or DA-L4 100K.

32. The engineered cell of claim 30 or claim 31, wherein the first stably integrated nucleic acid molecule further comprises a selection marker that is operably linked to a promoter.

33. The engineered cell of any one of claims 30-32, wherein the nucleic acid sequence of DA-E2A comprises one or more mutations to adenine or cytosine resulting in one or more premature stop codons.

34. The engineered cell of any one of claims 31-33, wherein the nucleic acid sequence encoding for DA-E2A comprises the amino acid sequence of SEQ ID NOs: 39, or 40.

35. The engineered cell of any one of claims 31-34, wherein positions 181 and/or 324 of DA-E2A (SEQ ID NOs: 39 or 40) correspond with mutations to adenine resulting in premature stop codons.

36. The engineered cell of any one of claims 31-35, wherein the nucleic acid sequence of DA-E4ORF6 comprises one or more mutations to adenine resulting in one or more premature stop codons.

37. The engineered cell of any one of claims 31-36, wherein the nucleic acid sequence encoding for DA-E4ORF6 comprises the amino acid sequence of SEQ ID NOs: 41 or 42.

38. The engineered cell of any one of claims 31-37, wherein positions 77 and/or 192 of DA-E4ORF6 (SEQ ID NOs: 41, or 42) correspond with a modified codon comprising an adenine resulting in a premature stop codon.

39. The engineered cell of any one of claims 25-38, wherein the one or more stably integrated nucleic acid molecules comprises a second stably integrated nucleic acid molecule comprising the nucleic acid sequence encoding DA-Rep52 or DA-Rep40, the nucleic acid sequence encoding DA-Rep78 or DA-Rep68, the nucleic acid sequence encoding VP1 or DA-VP1, the nucleic acid sequence encoding VP2 or DA-VP2, and the nucleic acid sequence encoding VP3 or DA-VP3.

40. The engineered cell of claim 39, wherein the second integrated nucleic acid molecule further comprises a selection marker that is operably linked to a promoter.

41. The engineered cell of any one of claims 39-40, wherein the second stably integrated nucleic acid molecule comprises the nucleic acid sequence encoding for DA-Rep52 or DA-Rep40.

42. The engineered cell of claim 41, wherein the nucleic acid sequence encoding for DA-Rep52 comprises an amino acid sequence of SEQ ID NOs: 43 or 47.

43. The engineered cell of claim 41, wherein the nucleic acid sequence encoding for DA-Rep40 comprises an amino acid sequence of SEQ ID NOs: 44 or 48.

44. The engineered cell of any one of claims 39-43, wherein the second stably integrated nucleic acid molecule comprises a nucleic acid sequence encoding for DA-Rep78 or DA-Rep68.

45. The engineered cell of claim 44, wherein the nucleic acid sequence encoding for DA-Rep78 comprises an amino acid sequence of any one of SEQ ID NOs: 45, 49 and 51.

46. The engineered cell of claim 45, wherein the nucleic acid sequence encoding for DA-Rep68 comprises an amino acid sequence of SEQ ID NOs: 46, 50 or 52.

47. The engineered cell of any one of claims 39-46, wherein the second stably integrated nucleic acid molecule comprises an amino acid sequence encoding for Rep52 or DA-Rep52; Rep40 or DA-Rep40; Rep68 or DA-Rep68; and Rep78 or DA-Rep78.

48. The engineered cell of claim 47, wherein the nucleic acid sequence encoding for Rep52 or DA-Rep52; Rep40 or DA-Rep40; Rep68 or DA-Rep68; and Rep78 or DA-Rep78 comprises a nucleic acid sequence of any one of SEQ ID NOs: 53-55, 113-115.

49. The engineered cell of claim 48, wherein the nucleic acid sequence encoding for DA-Rep52, DA-Rep40, DA-Rep68 and DA-Rep78 comprises one or more mutations to adenine or cytosine resulting in one or more premature stop codons.

50. The engineered cell of claim 49, wherein one adenine mutation in the nucleotide sequence is at a position that corresponds to amino acid positions 67, 262, and/or 319 of DA-Rep78 (SEQ ID NOs: 45, 49 and 51).

51. The engineered cell of any one of claims 39-50, wherein the second stably integrated nucleic molecule further comprises a nucleic acid sequence encoding for one or more sgRNAs.

52. The engineered cell of claim 51, wherein the one or more sgRNAs each comprise a nucleic acid sequence that is complementary to the nucleic acid sequences comprising one or more mutations to adenine or cytosine.

53. The engineered cell of claim 51 or claim 52, wherein the one or more sgRNAs each comprise a nucleic acid sequence of any one of SEQ ID NOs: 56-81.

54. The engineered cell of any one of claims 51-53, wherein the one or more sgRNAs are operably linked to a chemically inducible promoter.

55. The engineered cell of claim 54, wherein the chemically inducible promoter is selected from the group consisting of pTRE3G, pTREtight, or a promoter containing at least one of VanR, TtgR, PhlF, CymR, or the Gal4 UAS operator sequences.

56. The engineered cell of claim 55, wherein the nucleic acid sequence encoding the chemically inducible promoter is any one of SEQ ID NOs: 1 and 2 or comprises any one of SEQ ID NOs: 86-91.

57. The engineered cell of any one of claims 39-56, wherein the second stably integrated nucleic acid molecule comprises nucleic acid sequences encoding for VP1 or DA-VP1, VP2 or DA-VP2, and VP3 or DA-VP3.

58. The engineered cell of claim 57, wherein the nucleic acid sequence encoding for VP1 comprises the amino acid sequence of SEQ ID NO: 14.

59. The engineered cell of claim 58, wherein the nucleic acid sequence encoding for DA-VP1 comprises the amino acid sequence of SEQ ID NO: 99 or 102.

60. The engineered cell of claim 59 or claim 60, wherein the nucleic acid sequence encoding for VP2 comprises the amino acid sequence of SEQ ID NO: 15.

61. The engineered cell of claim 57 or claim 59, wherein the nucleic acid sequence encoding for DA-VP2 comprises the amino acid sequence of SEQ ID NO: 100 or 103.

62. The engineered cell of claim 57 or claim 61, wherein the nucleic acid sequence encoding for VP3 comprises the amino acid sequence of SEQ ID NO: 16.

63. The engineered cell of claim 57 or claim 60, wherein the nucleic acid sequence encoding for DA-VP3 comprises the amino acid sequence of SEQ ID NO: 101 or 104.

64. The engineered cell of any one of claims 57-63, wherein the second stably integrated nucleic acid molecule comprises a nucleic acid sequence encoding for AAP.

65. The engineered cell of claim 64, wherein the nucleic acid sequence encoding for AAP comprises the amino acid sequence of SEQ ID NO: 17.

66. The engineered cell of any one of claims 25-65, wherein the one or more stably integrated nucleic acid molecules comprises a third stably integrated nucleic acid molecule comprising a nucleic acid sequences encoding for a transcriptional activator that, when expressed in the presence of a small molecule inducer, binds to a chemically inducible promoter of the engineered cell, and the nucleic acid sequences encoding for a Base Editor.

67. The engineered cell of claim 66, wherein the third stably integrated nucleic acid molecule further comprises a selection marker that is operably linked to a promoter.

68. The engineered cell of claim 66 or 67, wherein the Base Editor is an Adenine Base Editor (ABE) or Cytosine Base Editor (CBE).

69. The engineered cell of claim 68, wherein the ABE is a Cas9 ABE or a Cas13 ABE, or wherein the CBE is a Cas9-CBE or a Cas13 CBE.

70. The engineered cell of claim 69, wherein the Cas9 ABE is encoded for by an amino acid sequence comprising SEQ ID NO: 82 or 83.

71. The engineered cell of claim 70, wherein the Cas13 ABE is encoded for by an amino acid sequence comprising SEQ ID NO: 84 or 85.

72. The engineered cell of any one of claims 66-71, wherein the nucleic acid sequences encoding for the ABE is operably linked to a third chemically inducible promoter.

73. The engineered cell of any one of claims 66-72, wherein the third stably integrated nucleic acid molecule further comprises a chemically inducible promoter selected from the group consisting of pTRE3G, pTREtight, or a promoter containing at least one of VanR, TtgR, PhlF, or CymR, or the Gal4 UAS operator sequences.

74. The engineered cell of claim B73, wherein the nucleic acid sequence encoding the third chemically inducible promoter is any one of SEQ ID NOs: 1 and 2 or comprises any one of SEQ ID NOs: 86-91.

75. The engineered cell of any one of claims 66-74, wherein the transcriptional activator is selected from the group consisting of TetOn-3G, TetOn-V16, TetOff-Advanced, VanR-VP16, TtgR-VP16, PhlF-VP16, and the cumate cTA and rcTA.

76. The engineered cell of any one of claims 66-75, wherein the small molecule inducer is selected from the group consisting of doxycycline, vanillate, phloretin, rapamycin, abscisic acid, gibberellic acid acetoxymethyl ester, and cumate.

77. The engineered cell of any one of claims 66-76, wherein the transcriptional activator is TetOn 3G and the small molecule inducer is doxycycline.

78. The engineered cell of any one of claims 25-77, wherein the engineered cell is HEK293 cell or HeLa cell.

79. A kit comprising the engineered cell of any one of claims 25-78.

80. The kit of claim 79 further comprising a polynucleotide comprising, from 5′ to 3′: (i) a nucleic acid sequence of a 5′ inverted terminal repeat; (ii) a multiple cloning site; and (iii) a nucleic acid sequence of a 3′ inverted terminal repeat.

81. The kit of claim 80, wherein the polynucleotide is a plasmid or a vector.

82. A method for AAV production, comprising contacting the engineered cell of any one of claims 25-78 with a small molecule inducer that binds to the chemically inducible promoter.

83. The method of claim 82, wherein the small molecule inducer is selected from the group consisting of doxycycline, vanillate, phloretin, rapamycin, abscisic acid, gibberellic acid acetoxymethyl ester, and cumate.


SEQUENCE LISTING (128 sequences)

SEQ ID NO: 1 - DNA, 346 bp, Artificial Sequence
  Synthetic
ctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattcgagctcggtacccgggga

SEQ ID NO: 2 - DNA, 423 bp, Artificial Sequence
  Synthetic
gtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctatcgatcggccggataacgccacc

SEQ ID NO: 3 - DNA, 496 bp, Artificial Sequence
  Synthetic
gaattctccaggcgatctgacggttcactaaacgagctctgcttatataggcctcccaccgtacacgccacctcgacatactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctccgcggggatcc

SEQ ID NO: 4 - DNA, 578 bp, Artificial Sequence
  Synthetic
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatg

SEQ ID NO: 5 - DNA, 571 bp, Artificial Sequence
  Synthetic
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatagttatc

SEQ ID NO: 6 - protein, 397 aa, Unknown
  Adeno-associated virus
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 7 - protein, 312 aa, Unknown
  Adeno-associated virus
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 8 - protein, 621 aa, Unknown
  Adeno-associated virus
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 9 - protein, 536 aa, Unknown
  Adeno-associated virus
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 10 - protein, 529 aa, Unknown
  Adeno-associated virus
MASREEEQRETTPERGRGAARRPPTMEDVSSPSPSPPPPRAPPKKRLRRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 11 - protein, 294 aa, Unknown
  Adeno-associated virus
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 12 - DNA, 888 bp, Unknown
  Adeno-associated virus
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaatgtttcctacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagtaa

SEQ ID NO: 13 - DNA, 738 bp, Unknown
  Adeno-associated virus
cgacgtaatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatcc

SEQ ID NO: 14 - protein, 735 aa, Unknown [sequence omitted]

SEQ ID NO: 15 - protein, 598 aa, Unknown
  Adeno-associated virus
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - protein, 533 aa, Unknown
  Adeno-associated virus
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 17 - protein, 204 aa, Unknown
  Adeno-associated virus
LETQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 18 - protein, 19 aa, Unknown
  Adeno-associated virus
ATNFSLLKQAGDVEENPGP

SEQ ID NO: 19 - protein, 18 aa, Artificial Sequence
  Synthetic
EGRGSLLTCGDVEENPGP

SEQ ID NO: 20 - protein, 454 aa, Methanosarcina mazeii
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 21 - protein, 454 aa, Artificial Sequence
  Synthetic
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVFGDTLDVMHGDLELSSAVVGPIPLDREWGIDKPWIGAGFGLERLLKVKHDFKNIKRAARSESYYNGISTNL

SEQ ID NO: 22 - DNA, 69 bp, Methanosarcina mazeii
ggaaacctgatcatgtagatcgaacggactctaaatccgttcagccgggttagattcccggggtttccg

SEQ ID NO: 23 - DNA, 372 bp, Artificial Sequence
  Synthetic
agtcagtcactagttgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaaacctgatcatgtagatcgaacggactctaaatccgttcagccgggttagattcccggggtttccggacaagtgcggtttttcctaggagtcagtc

SEQ ID NO: 24 - DNA, 1932 bp, Unknown
  Adeno-associated virus
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 25 - DNA, 1866 bp, Unknown
  Adeno-associated virus
ctggcgggcttctacgagatcgtgatcaaggtgcccagcgacctggacgagcatctgcctggcatcagcgacagcttcgtgaattgggtcgccgagaaagagtgggagctgcctcctgacagcgacttggacctgaacctgattgagcaggcccctctgacagtggccgagaagctgcagagggatttcctgacagagtggcggagagtgtctaaggcccctgaggctctgttcttcgtgcagttcgagaagggcgagagctacttccacttacacgtgctggtcgagacaaccggcgtgaagtctttagtgctgggcagattcctgagccagatcagagagaagctgatccagcggatctaccggggcatcgagcccacactgcctaattggttcgccgtgaccaagaccagaaacggcgctggcggcggaaacaaggtggtggacgagtgctacatccccaactacctgctgcctaagacacagcccgaactgcagtgggcctggaccaacttagaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacatggaactcgttggctggctggtggacaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 26 - DNA, 1866 bp, Artificial Sequence
  Synthetic
ctggcgggcttctacgagatcgtgatcaaggtgcccagcgacctggacgagcatctgcctggcatcagcgacagcttcgtgaattgggtcgccgagaaagagtgggagctgcctcctgacagcgacttggacctgaacctgattgagcaggcccctctgacagtggccgagaagctgcagagggatttcctgacagagtggcggagagtgtctaaggcccctgaggctctgttcttcgtgcagttcgagaagggcgagagctacttccacttacacgtgctggtcgagacaaccggcgtgaagtctttagtgctgggcagattcctgagccagatcagagagaagctgatccagcggatctaccggggcatcgagcccacactgcctaattggttcgccgtgaccaagaccagaaacggcgctggcggcggaaacaaggtggtggacgagtgctacatccccaactacctgctgcctaagacacagcccgaactgcagtgggcctggaccaacttagaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacatggaactcgttggctggctggtgtagaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 27 - DNA, 1866 bp, Artificial Sequence
  Synthetic
ctggcgggcttctacgagatcgtgatcaaggtgcccagcgacctggactagcatctgcctggcatcagcgacagcttcgtgaattgggtcgccgagaaagagtgggagctgcctcctgacagcgacttggacctgaacctgattgagcaggcccctctgacagtggccgagaagctgcagagggatttcctgacagagtggcggagagtgtctaaggcccctgaggctctgttcttcgtgcagttcgagaagggcgagagctacttccacttacacgtgctggtcgagacaaccggcgtgaagtctttagtgctgggcagattcctgagccagatcagagagaagctgatccagcggatctaccggggcatcgagcccacactgcctaattggttcgccgtgaccaagaccagaaacggcgctggcggcggaaacaaggtggtggacgagtgctacatccccaactacctgctgcctaagacacagcccgaactgcagtgggcctggaccaacttagaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacatggaactcgttggctggctggtgtagaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 28 - DNA, 1949 bp, Artificial Sequence
  Synthetic
tgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtgtagaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 29 - DNA, 1949 bp, Artificial Sequence
  Synthetic
tgatctgcgcagccgccatgccggggttttacgagattgtgattaaggtccccagcgaccttgactagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtgtagaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 30 - DNA, 3648 bp, Artificial Sequence
  Synthetic
gccgccaccatggaattagtgggctggttggtcgataaaggcatcacaagcgaaaaacaatggattcaagaagatcaagcgagctatattagttttaacgccgctagtaatagcagaagtcagattaaagccgctctcgataacgccggcaaaatcatgtctttaaccaagacagctcctgattatttagtcgggcaacaacctgtcgaggacatcagttctaacagaatctacaagatcctcgaattgaatggctatgaccctcagtacgccgccagtgtgttcttaggctgggctaccaagaaatttgggaaacgcaatacaatttggttattcggccccgccaccacaggcaaaacaaatattgccgaagctatcgctcataccgtccctttctatggctgtgtgaattggacaaacgaaaatttcccttttaatgattgcgtggataaaatggtcatttggtgggaagaaggcaaaatgacagctaaagtggtcgaaagcgctaaggctatcttgggcggctctaaagtcagagtcgatcaaaagtgtaaaagtagcgctcaaatcgatcccacccctgtcattgtgacaagtaatacaaatatgtgtgctgtcatcgatggcaatagcaccacatttgagcatcaacaacccctccaggatagaatgtttaagttcgagttgacaagaagattagaccacgatttcggcaaagtgacaaaacaagaggtgaaggatttctttagatgggccaaagaccatgtcgtggaagtcgaacacgagttttatgtgaagaaaggcggcgctaaaaagcggcctgctccttccgatgccgacatctccgaacctaagagagtcagagaaagcgtggcccaacccagcaccagcgatgccgaggccagcattaattatgccgatcgctatcagaataagtgcagcagacatgtcgggatgaacttaatgttattcccttgtcggcagtgtgaacggatgaaccaaaacagcaacatttgttttacccacggacaaaaggattgcctggaatgtttccctgtcagcgagagccagcctgtgagcgtggtgaagaaagcctaccaaaagttatgttatatccaccacattatgggcaaagtccccgatgcctgtaccgcttgtgacttagtgaacgtagacctcgacgattgtattttcgagcagtgataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgcctggcttctacgagatcgtgatcaaggtgcccagcgacctggacgagcatctgcctggcatcagcgacagcttcgtgaattgggtcgccgagaaagagtgggagctgcctcctgacagcgacatggacctgaacctgattgagcaggcccctctgacagtggccgagaagctgcagagggatttcctgacagagtggcggagagtgtctaaggcccctgaggctctgttcttcgtgcagttcgagaagggcgagagctacttccacatgcacgtgctggtcgagacaaccggcgtgaagtctatggtgctgggcagattcctgagccagatcagagagaagctgatccagcggatctaccggggcatcgagcccacactgcctaattggttcgccgtgaccaagaccagaaacggtgctggcggcggaaacaaggtggtggacgagtgctacatccccaactacctgctgcctaagacacagcccgaactgcagtgggcctggaccaacatggaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacggagaactcgttggctggctggtggacaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 31 - DNA, 3648 bp, Artificial Sequence
  Synthetic
gccgccaccatggaattagtgggctggttggtctagaaaggcatcacaagcgaaaaacaatggattcaagaagatcaagcgagctatattagttttaacgccgctagtaatagcagaagtcagattaaagccgctctcgataacgccggcaaaatcatgtctttaaccaagacagctcctgattatttagtcgggcaacaacctgtcgaggacatcagttctaacagaatctacaagatcctcgaattgaatggctatgaccctcagtacgccgccagtgtgttcttaggctgggctaccaagaaatttgggaaacgcaatacaatttggttattcggccccgccaccacaggcaaaacaaatattgccgaagctatcgctcataccgtccctttctatggctgtgtgaattggacaaacgaaaatttcccttttaatgattgcgtggataaaatggtcatttggtgggaagaaggcaaaatgacagctaaagtggtcgaaagcgctaaggctatcttgggcggctctaaagtcagagtcgatcaaaagtgtaaaagtagcgctcaaatcgatcccacccctgtcattgtgacaagtaatacaaatatgtgtgctgtcatcgatggcaatagcaccacatttgagcatcaacaacccctccaggatagaatgtttaagttcgagttgacaagaagattagaccacgatttcggcaaagtgacaaaacaagaggtgaaggatttctttagatgggccaaagaccatgtcgtggaagtcgaacacgagttttatgtgaagaaaggcggcgctaaaaagcggcctgctccttccgatgccgacatctccgaacctaagagagtcagagaaagcgtggcccaacccagcaccagcgatgccgaggccagcattaattatgccgatcgctatcagaataagtgcagcagacatgtcgggatgaacttaatgttattcccttgtcggcagtgtgaacggatgaaccaaaacagcaacatttgttttacccacggacaaaaggattgcctggaatgtttccctgtcagcgagagccagcctgtgagcgtggtgaagaaagcctaccaaaagttatgttatatccaccacattatgggcaaagtccccgatgcctgtaccgcttgtgacttagtgaacgtagacctcgacgattgtattttcgagcagtgataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgcctggcttctacgagatcgtgatcaaggtgcccagcgacctggacgagcatctgcctggcatcagcgacagcttcgtgaattgggtcgccgagaaagagtgggagctgcctcctgacagcgacatggacctgaacctgattgagcaggcccctctgacagtggccgagaagctgcagagggatttcctgacagagtggcggagagtgtctaaggcccctgaggctctgttcttcgtgcagttcgagaagggcgagagctacttccacatgcacgtgctggtcgagacaaccggcgtgaagtctatggtgctgggcagattcctgagccagatcagagagaagctgatccagcggatctaccggggcatcgagcccacactgcctaattggttcgccgtgaccaagaccagaaacggtgctggcggcggaaacaaggtggtggacgagtgctacatccccaactacctgctgcctaagacacagcccgaactgcagtgggcctggaccaacatggaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacggagaactcgttggctggctggtgtagaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 32 - DNA, 3648 bp, Artificial Sequence
  Synthetic
gccgccaccatggaattagtgggctggttggtctagaaaggcatcacaagcgaaaaacaatggattcaagaagatcaagcgagctatattagttttaacgccgctagtaatagcagaagtcagattaaagccgctctcgataacgccggcaaaatcatgtctttaaccaagacagctcctgattatttagtcgggcaacaacctgtcgaggacatcagttctaacagaatctacaagatcctcgaattgaatggctatgaccctcagtacgccgccagtgtgttcttaggctgggctaccaagaaatttgggaaacgcaatacaatttggttattcggccccgccaccacaggcaaaacaaatattgccgaagctatcgctcataccgtccctttctatggctgtgtgaattggacaaacgaaaatttcccttttaatgattgcgtggataaaatggtcatttggtgggaagaaggcaaaatgacagctaaagtggtcgaaagcgctaaggctatcttgggcggctctaaagtcagagtcgatcaaaagtgtaaaagtagcgctcaaatcgatcccacccctgtcattgtgacaagtaatacaaatatgtgtgctgtcatcgatggcaatagcaccacatttgagcatcaacaacccctccaggatagaatgtttaagttcgagttgacaagaagattagaccacgatttcggcaaagtgacaaaacaagaggtgaaggatttctttagatgggccaaagaccatgtcgtggaagtcgaacacgagttttatgtgaagaaaggcggcgctaaaaagcggcctgctccttccgatgccgacatctccgaacctaagagagtcagagaaagcgtggcccaacccagcaccagcgatgccgaggccagcattaattatgccgatcgctatcagaataagtgcagcagacatgtcgggatgaacttaatgttattcccttgtcggcagtgtgaacggatgaaccaaaacagcaacatttgttttacccacggacaaaaggattgcctggaatgtttccctgtcagcgagagccagcctgtgagcgtggtgaagaaagcctaccaaaagttatgttatatccaccacattatgggcaaagtccccgatgcctgtaccgcttgtgacttagtgaacgtagacctcgacgattgtattttcgagcagtgataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatgcctggcttctacgagatcgtgatcaaggtgcccagcgacctggactagcatctgcctggcatcagcgacagcttcgtgaattgggtcgccgagaaagagtgggagctgcctcctgacagcgacatggacctgaacctgattgagcaggcccctctgacagtggccgagaagctgcagagggatttcctgacagagtggcggagagtgtctaaggcccctgaggctctgttcttcgtgcagttcgagaagggcgagagctacttccacatgcacgtgctggtcgagacaaccggcgtgaagtctatggtgctgggcagattcctgagccagatcagagagaagctgatccagcggatctaccggggcatcgagcccacactgcctaattggttcgccgtgaccaagaccagaaacggtgctggcggcggaaacaaggtggtggacgagtgctacatccccaactacctgctgcctaagacacagcccgaactgcagtgggcctggaccaacatggaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacggagaactcgttggctggctggtgtagaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 33 - DNA, 1866 bp, Artificial Sequence
  Synthetic
atgcctggcttctacgagatcgtgatcaaggtgcccagcgacctggacgagcatctgcctggcatcagcgacagcttcgtgaattgggtcgccgagaaagagtgggagctgcctcctgacagcgacatggacctgaacctgattgagcaggcccctctgacagtggccgagaagctgcagagggatttcctgacagagtggcggagagtgtctaaggcccctgaggctctgttcttcgtgcagttcgagaagggcgagagctacttccacatgcacgtgctggtcgagacaaccggcgtgaagtctatggtgctgggcagattcctgagccagatcagagagaagctgatccagcggatctaccggggcatcgagcccacactgcctaattggttcgccgtgaccaagaccagaaacggtgctggcggcggaaacaaggtggtggacgagtgctacatccccaactacctgctgcctaagacacagcccgaactgcagtgggcctggaccaacatggaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacggagaactcgttggctggctggtgtagaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 34 - protein, 620 aa, Artificial Sequence
  Synthetic
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYGELVGWLVKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 35 - DNA, 1866 bp, Artificial Sequence
  Synthetic
atgcctggcttctacgagatcgtgatcaaggtgcccagcgacctggactagcatctgcctggcatcagcgacagcttcgtgaattgggtcgccgagaaagagtgggagctgcctcctgacagcgacatggacctgaacctgattgagcaggcccctctgacagtggccgagaagctgcagagggatttcctgacagagtggcggagagtgtctaaggcccctgaggctctgttcttcgtgcagttcgagaagggcgagagctacttccacatgcacgtgctggtcgagacaaccggcgtgaagtctatggtgctgggcagattcctgagccagatcagagagaagctgatccagcggatctaccggggcatcgagcccacactgcctaattggttcgccgtgaccaagaccagaaacggtgctggcggcggaaacaaggtggtggacgagtgctacatccccaactacctgctgcctaagacacagcccgaactgcagtgggcctggaccaacatggaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacggagaactcgttggctggctggtggacaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 36 - protein, 620 aa, Artificial Sequence
  Synthetic
MPGFYEIVIKVPSDLDHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYGELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 37 - DNA, 1866 bp, Artificial Sequence
  Synthetic
atgcctggcttctacgagatcgtgatcaaggtgcccagcgacctggactagcatctgcctggcatcagcgacagcttcgtgaattgggtcgccgagaaagagtgggagctgcctcctgacagcgacatggacctgaacctgattgagcaggcccctctgacagtggccgagaagctgcagagggatttcctgacagagtggcggagagtgtctaaggcccctgaggctctgttcttcgtgcagttcgagaagggcgagagctacttccacatgcacgtgctggtcgagacaaccggcgtgaagtctatggtgctgggcagattcctgagccagatcagagagaagctgatccagcggatctaccggggcatcgagcccacactgcctaattggttcgccgtgaccaagaccagaaacggtgctggcggcggaaacaaggtggtggacgagtgctacatccccaactacctgctgcctaagacacagcccgaactgcagtgggcctggaccaacatggaacagtacctgagcgcctgcctgaatctgaccgagcggaaaagactggtggcccagcatctgacccacgtgtcccagacacaagagcagaacaaagagaatcagaaccccaacagcgacgcccctgtgatcagaagcaagaccagcgccagatacggagaactcgttggctggctggtgtagaagggcatcacaagcgagaagcagtggatccaagaggaccaggccagctacatcagcttcaacgccgcctccaacagcagatcccagatcaaggccgctctggacaacgccggcaagatcatgagcctgacaaagacagcccctgactacctcgtgggccagcagcctgtggaagatatcagcagcaaccggatctacaagatcctggaactgaacggctacgaccctcagtatgccgcctctgtgtttctcggctgggctaccaagaagttcggcaagcggaacaccatctggctgtttggccctgccacaaccggcaagaccaatatcgccgaggctatcgcccacaccgtgcctttttacggctgcgtgaactggaccaatgagaacttccccttcaacgactgcgtggacaagatggtcatttggtgggaagagggcaagatgaccgccaaagtggtggaaagcgccaaggccatcctcggcggatctaaagttcgcgtggaccagaagtgcaagtctagcgcccagatcgaccccacacctgtgatcgtgaccagcaacaccaacatgtgcgccgtgatcgacggcaacagcaccacctttgaacaccagcagccactgcaggaccggatgttcaagttcgagctgaccagacggctggaccacgacttcggcaaagtgaccaagcaagaagtgaaggacttcttccgctgggccaaagatcacgtggtggaagtggaacacgagttctacgtgaagaaaggcggagccaagaagaggcccgctccttccgatgccgatatcagcgagcctaagcgcgtgcgggaatctgtggctcagcctagcacatctgatgccgaggccagcatcaactacgccgacagataccagaacaagtgcagccggcacgtgggaatgaatctgatgctgttcccctgtcggcagtgcgagcggatgaaccagaacagcaacatctgcttcacccacggccagaaagactgcctggaatgcttccccgtgtccgagtctcagcctgtgtccgtggtcaagaaggcctaccagaagctgtgttacatccaccacatcatgggcaaagtgcccgatgcctgcaccgcctgcgatctggttaatgtggacctggatgactgcatcttcgagcagtga

SEQ ID NO: 38 - protein, 619 aa, Artificial Sequence
  Synthetic
MPGFYEIVIKVPSDLDHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYGELVGWLVKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 39 - protein, 528 aa, Artificial Sequence
  Synthetic
MASREEEQRETTPERGRGAARRPPTMEDVSSPSPSPPPPRAPPKKRLRRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 40 - protein, 528 aa, Artificial Sequence
  Synthetic
MASREEEQRETTPERGRGAARRPPTMEDVSSPSPSPPPPRAPPKKRLRRRLESEDEEDSSQDALVPRTPSPRPSTSTADLAIASKKKKKRPSPKPERPPSPEVIVDSEEEREDVALQMVGFSNPPVLIKHGKGGKRTVRRLNEDDPVARGMRTQEEKEESSEAESESTVINPLSLPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRGRNVVVISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 41 - protein, 293 aa, Artificial Sequence
  Synthetic
MTTSGVPFGMTLRPTRSRLSRRTPYSRDRLPPFETETRATILEDHPLLPECNTLTMHNVSYVRGLPCSVGFTLIQEVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 42 - protein, 293 aa, Artificial Sequence
  Synthetic
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 43 - protein, 396 aa, Artificial Sequence
  Synthetic
MELVGWLVDKGITSEKQWIQEDQASYISFNAASNSRSIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 44 - protein, 311 aa, Artificial Sequence
  Synthetic
MELVGWLVDKGITSEKQWIQEDQASYISFNAASNSRSIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 45 - protein, 620 aa, Artificial Sequence
  Synthetic
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 46 - protein, 535 aa, Artificial Sequence
  Synthetic
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 47 - protein, 396 aa, Artificial Sequence
  Synthetic
MELVGWLVDKGITSEKQWIQEDQASYISFNAASNSRSQIKAALDNAGKIMSLTKTAPDYLVGQQPVEDISSNRIYKILELNGYDPQYAASVFLGATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 48 - protein, 311 aa, Artificial Sequence
  Synthetic
MELVGWLVDKGITSEKQWIQEDQASYISFNAASNSRSQIKAALDNAGKIMSLTKTAPDYLVGQQPVEDISSNRIYKILELNGYDPQYAASVFLGATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 49 - protein, 620 aa, Artificial Sequence
  Synthetic
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 50 - protein, 535 aa, Artificial Sequence
  Synthetic
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 51 - protein, 620 aa, Artificial Sequence
  Synthetic
MPGFYEIVIKVPSDLDEHLPGISDSFVNWVAEKEWELPPDSDMDLNLIEQAPLTVAEKLQRDFLTERRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 52 - protein, 535 aa, Artificial Sequence
  Synthetic
MPGFYEIVIKVPSDLDEHLPGISDSFVNWVAEKEWELPPDSDMDLNLIEQAPLTVAEKLQRDFLTERRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 53 - DNA, 1932 bp, Artificial Sequence
  Synthetic
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggagctaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 54 - DNA, 1932 bp, Artificial Sequence
  Synthetic
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggataggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 55 - DNA, 1932 bp, Artificial Sequence
  Synthetic
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggagtagcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 56 - DNA, 20 bp, Artificial Sequence
  Synthetic
tgcgtaggagaagggcatgg

SEQ ID NO: 57 - DNA, 20 bp, Artificial Sequence
  Synthetic
ccggtagggccgaaatgtgg

SEQ ID NO: 58 - DNA, 20 bp, Artificial Sequence
  Synthetic
atgagttgttccctgggata

SEQ ID NO: 59 - DNA, 20 bp, Artificial Sequence
  Synthetic
cttgtagtatgatggccacg

SEQ ID NO: 60 - DNA, 20 bp, Artificial Sequence
  Synthetic
cgtgtagcagcaatgcctgg

SEQ ID NO: 61 - DNA, 20 bp, Artificial Sequence
  Synthetic
actgaggattccgacccaag

SEQ ID NO: 62 - DNA, 20 bp, Artificial Sequence
  Synthetic
gccttatgtgttgacatctg

SEQ ID NO: 63 - DNA, 20 bp, Artificial Sequence
  Synthetic
ggagtagcgccgtgtgagta

SEQ ID NO: 64 - DNA, 20 bp, Artificial Sequence
  Synthetic
gatttagctccgcgagttgg

SEQ ID NO: 65 - DNA, 20 bp, Artificial Sequence
  Synthetic
ggataggccacgaaaaagtt

SEQ ID NO: 66 - DNA, 30 bp, Artificial Sequence
  Synthetic
cttctcccacgcagacacgatcggcaggct

SEQ ID NO: 67 - DNA, 30 bp, Artificial Sequence
  Synthetic
tcggccccaccggttcttcacgatcttggc

SEQ ID NO: 68 - DNA, 30 bp, Artificial Sequence
  Synthetic
gaacaacccattcctgaatcagcgtaaatc

SEQ ID NO: 69 - DNA, 30 bp, Artificial Sequence
  Synthetic
atcataccacaagcgcaggtagattaagtg

SEQ ID NO: 70 - DNA, 30 bp, Artificial Sequence
  Synthetic
ttgctgccacacgcccatggccgtttgcca

SEQ ID NO: 71 - DNA, 30 bp, Artificial Sequence
  Synthetic
ggaatccccagttgttgttgatgagtcttt

SEQ ID NO: 72 - DNA, 30 bp, Artificial Sequence
  Synthetic
acggcgccactccgtcagaaagtcgcgctg

SEQ ID NO: 73 - DNA, 30 bp, Artificial Sequence
  Synthetic
cgtggcccatcccagaaagacggaagccgc

SEQ ID NO: 74 - DNA, 50 bp, Artificial Sequence
  Synthetic
ctccatgcccttctcccacgcagacacgatcggcaggctcagcgggttta

SEQ ID NO: 75 - DNA, 50 bp, Artificial Sequence
  Synthetic
caccacatttcggccccaccggttcttcacgatcttggccttgctagact

SEQ ID NO: 76 - DNA, 50 bp, Artificial Sequence
  Synthetic
tatcccagggaacaacccattcctgaatcagcgtaaatcccacactgcag

SEQ ID NO: 77 - DNA, 50 bp, Artificial Sequence
  Synthetic
cacgtggccatcataccacaagcgcaggtagattaagtggcgacccctca

SEQ ID NO: 78 - DNA, 50 bp, Artificial Sequence
  Synthetic
ctccaggcattgctgccacacgcccatggccgtttgccaggtgtagcaca

SEQ ID NO: 79 - DNA, 50 bp, Artificial Sequence
  Synthetic
tcttgggtcggaatccccagttgttgttgatgagtctttgccagtcacgt

SEQ ID NO: 80 - DNA, 50 bp, Artificial Sequence
  Synthetic
cttactcacacggcgccactccgtcagaaagtcgcgctgcagcttctcgg

SEQ ID NO: 81 - DNA, 50 bp, Artificial Sequence
  Synthetic
gaactttttcgtggcccatcccagaaagacggaagccgcatattggggat

SEQ ID NO: 82 - protein, 1775 aa, Artificial Sequence
  Synthetic
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSPKKKRKV

SEQ ID NO: 83 - protein, 1588 aa, Artificial Sequence
  Synthetic
MSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGWRNAKTGAAGSLMDVLHYPGMNHRVEITEGILADECAALLCYFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 84 - protein, 1495 aa, Artificial Sequence
  Synthetic
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLPPLERLTLGSGGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 85 - protein, 1495 aa, Artificial Sequence
  Synthetic
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLPPLERLTLGSGGGGSQLHLPQVLADAVSRLVLGKFGDLTDNFSSPHARRKVLAGVVMTTGTDVKDAKVISVSTGGKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 86 - DNA, 12 bp, Artificial Sequence
  Synthetic
attggatccaat

SEQ ID NO: 87 - DNA, 28 bp, Artificial Sequence
  Synthetic
tatttacaaacaaccatgaatgtaagta

SEQ ID NO: 88 - DNA, 18 bp, Artificial Sequence
  Synthetic
cggagtactgtcctccga

SEQ ID NO: 89 - DNA, 30 bp, Artificial Sequence
  Synthetic
atgatacgaaacgtaccgtatcgttaaggt

SEQ ID NO: 90 - DNA, 26 bp, Artificial Sequence
  Synthetic
agaaacaaaccaacctgtctgtatta

SEQ ID NO: 91 - DNA, 28 bp, Artificial Sequence
  Synthetic
aacaaacagacaatctggtctgtttgta

SEQ ID NO: 92 - protein, 248 aa, Artificial Sequence
  Synthetic
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 93 - protein, 298 aa, Artificial Sequence
  Synthetic
MDMPRIKPGQRVMMALRKMIASGEIKSGERIAEIPTAAALGVSRMPVRIALRSLEQEGLVVRLGARGYAARGVSSDQIRDAIEVRGVLEGFAARRLAERGMTAETHARFVVLIAEGEALFAAGRLNGEDLDRYAAYNQAFHDTLVSAAGNGAVESALARNGFEPFAAAGALALDLMDLSAEYEHLLAAHRQHQAVLDAVSCGDAEGAERIMRDHALAAIRNAKVFEAAASAGAPLGAAWSIRADSGGGGPTDALDDFDLDMLPADALDDFDLDMLPADALDDFDLDMLPGPPKKKRKV

SEQ ID NO: 94 - protein, 264 aa, Artificial Sequence
  Synthetic
MVRRTKEEAQETRAQIIEAAERAFYKRGVARTTLADIAELAGVTRGAIYWHFNNKAELVQALLDSLHETHDHLARASESEDEVDPLGCMRKLLLQVFNELVLDARTRRINEILHHKCEFTDDMCEIRQQHQSAVLDCHKGITLTLANVVRRGQLPGELDAERAAVAMFAYVDGLIRRWLLLPDSVDLLGDVEKWVDTGLDMLRLSPALRKSGGGGPTDALDDFDLDMLPADALDDFDLDMLPADALDDFDLDMLPGPPKKKRKV

SEQ ID NO: 95 - protein, 254 aa, Artificial Sequence
  Synthetic
MARTPSRSSIGSLRSPHTHKAILTSTIEILKECGYSGLSIESVARRAGAGKPTIYRWWTNKAALIAEVYENEIEQVRKFPDLGSFKADLDFLLHNLWKVWRETICGEAFRCVIAEAQLDPVTLTQLKDQFMERRREIPKKLVEDAISNGELPKDINRELLLDMIFGFCWYRLLTEQLTVEQDIEEFTFLLINGVCPGTQCSGGGGPTDALDDFDLDMLPADALDDFDLDMLPADALDDFDLDMLPGPPKKKRKV

SEQ ID NO: 96 - protein, 257 aa, Artificial Sequence
  Synthetic
MSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPALREGIQRTVERNRFVVEDMWLGVLVSRGLSRDDAEDILWLIFNSVRGLVVRSLWQKDKERFERVRNSTLEIARERYAKFKRSGGGGPTDALDDFDLDMLPADALDDFDLDMLPADALDDFDLDMLPGPPKKKRKV

SEQ ID NO: 97 - protein, 641 aa, Unknown
  Adeno-associated virus
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQMIIRYGCRWLSSRLARGHSL

SEQ ID NO: 98 - protein, 804 aa, Artificial Sequence
  Synthetic
MESVEKEDSLTAPFEFATTASTDAANAPTTFPVEAPPLEEEEVIIEQDPGFVSEDDEDRSVPTEDKKQDQDDAEANEEQVGRGDQRHGDYLDVGDDVLLKHLQRQCAIICDALQERSDVPLAIADVSLAYERHLFSPRVPPKRQENGTCEPNPRLNFYPVFAVPEVLATYHIFFQNCKIPLSCRANRSRADKQLALRQGAVIPDIASLDEVPKIFEGLGRDEKRAANALQQENSENESHCGVLVELEGDNARLAVLKRSIEVTHFAYPALNLPPKVMSTVMSELIVRRARPLERDANLQEQTEEGLPAVGDEQLARWLETREPADLEERRKLMMAAVLVTVELECMQRFFADPEMQRKLEETLHYTFRQGYVRQACKISNVELCNLVSYLGILHENRLGQNVLHSTLKGEARRDYVRDCVYLFLCYTWQTAMGVQQCLEERNLKELQKLLKQNLKDLWTAFNERSVAAHLADIIFPERLLKTLQQGLPDFTSQSMLQNFRNFILERSGILPATCCALPSDFVPIKYRECPPPLWGHCYLLQLANYLAYHSDIMEDVSGDGLLECHCRCNLCTPHRSLVCNSQLLSESQIIGTFELQGPSPDEKSAAPGLKLTPGLWTSAYLRKFVPEDYHAHEIRFYEDQSRPPNAELTACVITQGHILGQLQAINKARQEFLLRKGRGVYLDPQSGEELNPIPPPPQPYQQPRALASQDGTQKEAAAAAAATHGRGGILGQSGRGGFGRGGGDDGRLGQPRRSFRGRRGVRRNTVTLGRIPLAGAPEIGNRSQHRYNLRSSGAAGTACSPTQP

SEQ ID NO: 99 - protein, 734 aa, Artificial Sequence
  Synthetic
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 100 - protein, 597 aa, Artificial Sequence
  Synthetic
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 101 - protein, 532 aa, Artificial Sequence
  Synthetic
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 102 - protein, 734 aa, Artificial Sequence
  Synthetic
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 103 - protein, 597 aa, Artificial Sequence
  Synthetic
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 104 - protein, 532 aa, Artificial Sequence
  Synthetic
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 105 - DNA, 1590 bp, Artificial Sequence
  Synthetic
atggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtaggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaa

SEQ ID NO: 106 - DNA, 1590 bp, Artificial Sequence
  Synthetic
atggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtagggccgaaatgtggtggtgatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaa

SEQ ID NO: 107 - DNA, 885 bp, Artificial Sequence
  Synthetic
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgagttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtag

SEQ ID NO: 108 - DNA, 885 bp, Artificial Sequence
  Synthetic
atgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtagtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtag

SEQ ID NO: 109 - DNA, 2418 bp, Artificial Sequence
  Synthetic
atggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtagcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtag

SEQ ID NO: 110 - DNA, 2208 bp, Artificial Sequence
  Synthetic
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactgaggattccgacccaagaggctcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 111 - DNA, 2208 bp, Artificial Sequence
  Synthetic
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacataaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 112 - protein, 805 aa, Unknown
  Adeno-associated virus
MESVEKEDSLTAPFEFATTASTDAANAPTTFPVEAPPLEEEEVIIEQDPGFVSEDDEDRSVPTEDKKQDQDDAEANEEQVGRGDQRHGDYLDVGDDVLLKHLQRQCAIICDALQERSDVPLAIADVSLAYERHLFSPRVPPKRQENGTCEPNPRLNFYPVFAVPEVLATYHIFFQNCKIPLSCRANRSRADKQLALRQGAVIPDIASLDEVPKIFEGLGRDEKRAANALQQENSENESHCGVLVELEGDNARLAVLKRSIEVTHFAYPALNLPPKVMSTVMSELIVRRARPLERDANLQEQTEEGLPAVGDEQLARWLETREPADLEERRKLMMAAVLVTVELECMQRFFADPEMQRKLEETLHYTFRQGYVRQACKISNVELCNLVSYLGILHENRLGQNVLHSTLKGEARRDYVRDCVYLFLCYTWQTAMGVWQQCLEERNLKELQKLLKQNLKDLWTAFNERSVAAHLADIIFPERLLKTLQQGLPDFTSQSMLQNFRNFILERSGILPATCCALPSDFVPIKYRECPPPLWGHCYLLQLANYLAYHSDIMEDVSGDGLLECHCRCNLCTPHRSLVCNSQLLSESQIIGTFELQGPSPDEKSAAPGLKLTPGLWTSAYLRKFVPEDYHAHEIRFYEDQSRPPNAELTACVITQGHILGQLQAINKARQEFLLRKGRGVYLDPQSGEELNPIPPPPQPYQQPRALASQDGTQKEAAAAAAATHGRGGILGQSGRGGFGRGGGDDGRLGQPRRSFRGRRGVRRNTVTLGRIPLAGAPEIGNRSQHRYNLRSSGAAGTACSPTQP

SEQ ID NO: 113 - DNA, 4068 bp, Artificial Sequence
  Synthetic
gccaccatggagctggtcgggtggctcgtgtagaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgagataactgagggatagaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatagttatcgccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagacacggaacggcgccgggggaggaaacaaagttgttgatgagtgctacatccccaattacttgctccccaaaacccagcctgagctccaatgggcatggaccaacatggaacagtacctgtctgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtgtagaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgagttatcatttaaatggcgcgcccacgtgggtaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggca

SEQ ID NO: 114 - DNA, 4068 bp, Artificial Sequence
  Synthetic
gccaccatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgagataactgagggatagaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatagttatcgccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagacacggaacggcgccgggggaggaaacaaagttgttgatgagtgctacatccccaattacttgctccccaaaacccagcctgagctccaatgggcatggaccaacatggaacagtacctgtctgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtgtagaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgagttatcatttaaatggcgcgcccacgtgggtaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggca

SEQ ID NO: 115 - DNA, 4068 bp, Artificial Sequence
  Synthetic
gccaccatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgagataactgagggatagaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatagttatcgccgccatgccggggttttacgagattgtgattaaggtccccagcgaccttgactagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagacacggaacggcgccgggggaggaaacaaagttgttgatgagtgctacatccccaattacttgctccccaaaacccagcctgagctccaatgggcatggaccaacatggaacagtacctgtctgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgagttatcatttaaatggcgcgcccacgtgggtaccgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggca

SEQ ID NO: 116 - DNA, 2208 bp, Unknown
  Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 117 - protein, 735 aa, Unknown [sequence omitted]

SEQ ID NO: 118 - protein, 260 aa, Artificial Sequence
  Synthetic
MVIMSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSIGLDLIVAADRDPVLREGIQRTVERNRFVVGDIWLGVLVSRGLSRDDAEDILWLIFNSVRGLVVRSLWQKDKERFERVRNSTLEIARERYAKFKRSGGGGPTDALDDFDLDMLPADALDDFDLDMLPADALDDFDLDMLPGPPKKKRKV

SEQ ID NO: 119 - DNA, 30 bp, Artificial Sequence
  Synthetic
ctccatgcccttctcctacgcagacacgat

SEQ ID NO: 120 - DNA, 30 bp, Artificial Sequence
  Synthetic
gagatcaccaccacatttcggccctaccgg

SEQ ID NO: 121 - DNA, 30 bp, Artificial Sequence
  Synthetic
tcccagggaacaactcattcctgaatcagc

SEQ ID NO: 122 - DNA, 30 bp, Artificial Sequence
  Synthetic
cgtggccatcatactacaagcgcaggtaga

SEQ ID NO: 123 - DNA, 30 bp, Artificial Sequence
  Synthetic
ggccatgggcgtgtagcagcaatgcctgga

SEQ ID NO: 124 - DNA, 30 bp, Artificial Sequence
  Synthetic
aactgaggattccgacccaagaggctcaac

SEQ ID NO: 125 - DNA, 30 bp, Artificial Sequence
  Synthetic
gcagatgtcaacacataaggcgttcttcca

SEQ ID NO: 126 - DNA, 30 bp, Artificial Sequence
  Synthetic
gactttctgacggagtagcgccgtgtgagt

SEQ ID NO: 127 - DNA, 30 bp, Artificial Sequence
  Synthetic
ctccaactcgcggagctaaatcaaggctgc

SEQ ID NO: 128 - DNA, 30 bp, Artificial Sequence
  Synthetic
ccgtctttctgggataggccacgaaaaagt